(19) United States
(12) Patent Application Publication
(10) Pub. No.: US 2024/0246963 A1
(43) Pub. Date: Jul. 25, 2024

(54) BIFUNCTIONAL COMPOUNDS AND PHARMACEUTICAL USES THEREOF

(71) Applicants: RISEN (SUZHOU) PHARMA TECH CO., LTD., Suzhou (CN); RISEN (SHANGHAI) PHARMACEUTICAL ENGINEERING CO LTD, Shanghai (CN)

(72) Inventors: Jiasheng LU, Suzhou (CN); Xiang JI, Suzhou (CN); Qiguo ZHANG, Suzhou (CN); Xiaolin HE, Suzhou (CN); Yanpeng WU, Suzhou (CN); Bin ZONG, Suzhou (CN); Yuhui CHEN, Suzhou (CN); Chuanhao HUANG, Suzhou (CN); Yongyue CHEN, Suzhou (CN); Tianlun ZHOU, Suzhou (CN); Jian GE, Suzhou (CN); Xianqi KONG, Dollard-des-Ormeaux (CA); Xiangsheng YE, Suzhou (CN); Dawei CHEN, Suzhou (CN)

(21) Appl. No.: 18/533,634

(22) Filed: Dec. 8, 2023

(30) Foreign Application Priority Data
Dec. 8, 2022 (CN) 202211574358.6

(51) Int. Cl. C07D 471/04; A61K 45/06; C07D 519/00; C07K 16/28

(52) U.S. Cl. CPC C07D 471/04; A61K 45/06; C07D 519/00; C07K 16/2818

(57) ABSTRACT

The disclosure relates to bifunctional KRAS-modulating compounds having the structure K-L-T, where K is a targeting group that binds specifically to a KRAS protein (mutant or wild-type), T is an E3-ligase binding group, and L is absent or is a bivalent linking group that connects K and T together via a covalent linkage. Compounds and pharmaceutical compositions thereof can promote degradation of KRAS protein (mutant or wild-type) in a cell and are thus useful for treating, inhibiting, and preventing KRAS-associated diseases, disorders and conditions, including cancers.

### CROSS-REFERENCE TO RELATED APPLICATIONS

This application claims the benefit of priority from Chinese Patent Application No. CN202211574358.6, filed Dec. 8, 2022, which is hereby incorporated by reference in its entirety.

### FIELD

The present disclosure relates to bifunctional KRAS-modulating compounds, pharmaceutical compositions thereof, and uses thereof for treating, inhibiting and/or preventing KRAS-associated diseases, disorders and conditions, including cancers, tumors and hyperplastic or hyperproliferative disorders.

### BACKGROUND

The Kirsten Rat Sarcoma Viral Oncogene Homolog (K-Ras) gene belongs to the Ras family of oncogenes and is one of the most common gene mutations in human cancers. Its encoded protein (KRAS) is part of the RAS/MAPK signal transduction pathway which regulates cell growth and differentiation. KRAS is a small GTPase, a class of enzymes which convert the nucleotide guanosine triphosphate (GTP) into guanosine diphosphate (GDP). It is turned on (activated) by binding to GTP and turned off (inactivated) by converting the GTP to GDP. In this way KRAS acts as a molecular on/off switch. In most cells, KRAS is inactivated. When activated, it can activate several downstream signaling pathways including the MAPK signal transduction pathway, the PI3K signal transduction pathway and the Ral-GEFs signal transduction pathway. These signal transduction pathways play an important role in promoting cell survival, proliferation, and cytokine release, thus affecting tumor occurrence and development.

Many K-Ras gene mutations are missense mutations occurring in codon 12, which results in changing the glycine at position 12 (G12) to another amino acid. Replacements with cysteine, aspartic acid, arginine, and valine (KRAS-G12C, KRAS-G12D, KRAS-G12R, and KRAS-G12V, respectively) are the most common KRAS mutations in patients. In addition to the wild-type KRAS, other KRAS gene mutations include KRAS G12A, KRAS G12S, KRAS G13D, or KRAS Q61H, among others (Liu, Pingyu et al., Acta Pharmaceutica Sinica. B (2019), 9(5), 871-879). In human cancers, KRAS gene mutations are observed in nearly 90% of pancreatic cancers, approximately 30% to 40% of colorectal cancers, about 17% of endometrial cancers, and roughly 15% to 20% of lung cancers (mostly non-small cell lung cancer, NSCLC). They are also found in cancers such as biliary tract cancer, cervical cancer, bladder cancer, liver cancer, and breast cancer, among others. K-RAS gene mutations are thus found at high rates in many different types of cancer.

Inhibitors of KRAS-G12D have been described (see, for example, International (PCT) Application Publication Nos. WO2021041671 and WO2021106231). In 2021, The U.S. Food and Drug Administration (FDA) approved sotorasib as the first KRAS-G12C blocking drug for the treatment of adult patients with NSCLC. The KRAS-G12C inhibitor adagrasib was also approved by the U.S. FDA in 2022 for treatment of NSCLC. However, existing KRAS inhibitors face significant limitations. One of the biggest obstacles to KRAS inhibitor treatment is the emergence of drug resistance. While the biological basis of acquired drug resistance is not well understood, it has been suggested that several factors may play a role, including cellular heterogeneity in tumors; the activation of wild-type RAS by multiple receptor tyrosine kinases (RTKs) rather than a single RTK; and secondary gene mutations (see, e.g., Liu et al., Cancer Gene Therapy 2022, 29:875-878).

There is a need therefore for new inhibitors that can maintain efficacy and avoid or overcome the difficulties of acquired drug resistance. One method for avoiding or overcoming drug resistance is to promote degradation of the target protein, rather than simply inhibiting its biological activity through direct binding. One such method for enhancing protein degradation is through use of Proteolysis targeting chimeras, or Protacs (see, for example, Angew. Chem. Int. Ed. 2016, 55, 807-810; J. Med. Chem. 2018, 61, 444-452). A Protac is not a traditional enzyme inhibitor but rather acts by inducing intracellular protein hydrolysis (proteolysis). Such targeted protein degradation has emerged as a new paradigm to manipulate cellular proteostasis. In general, proteolysis targeting chimeras (Protacs) are bifunctional small molecules composed of two active domains and optionally a linker. One of the two active domains binds to E3 ubiquitin ligase, and the other to a target protein of interest. A Protac can thus remove a target protein of interest by binding to the target protein and recruiting an E3 ligase thereto, which catalyzes ubiquitination and leads to subsequent degradation of the target protein. Compared to traditional inhibitors that may need to inhibit enzymatic activity of a target protein, Protacs need only to bind specifically to the target protein to be effective.

There is a need for KRAS inhibitors effective for the treatment or prevention of KRAS-related diseases or disorders, including those associated with various KRAS mutations.

### SUMMARY

The present disclosure relates to bifunctional compounds and compositions comprising the compounds that inhibit the KRAS protein. Specifically, the disclosure provides proteolysis targeting chimera (Protac) compounds that bind to both the target protein of interest (e.g., a KRAS protein) and to an E3 ligase. By binding to both molecules, these compounds can recruit the E3 ligase to the target protein of interest, promoting its ubiquitination and subsequent degradation. Target proteins of interest herein include, without limitation, KRAS G12A, G12C, G12D, G12V, G12R, G12S, GT3D, and/or Q61H mutant proteins, and wild-type KRAS protein.

The present disclosure also relates to the use of such compounds and compositions for the treatment and/or prevention of diseases, disorders and conditions mediated, in whole or in part, by KRAS, e.g., by a mutant KRAS protein or the wild-type KRAS protein. KRAS inhibitors have been linked to the treatment of many hyperplastic and hyperproliferative diseases and disorders, including cancers and tumors. In particular embodiments, the KRAS inhibitor compounds and compositions described herein can act to modulate degradation of a KRAS protein and are thus useful as therapeutic or prophylactic agents when such degradation is desirable, e.g., for tumors and cancers associated with one of the various KRas mutations and/or KRAS mutant proteins.

In a first broad aspect, there are provided compounds of Formula (A) and pharmaceutically acceptable salts, esters, hydrates, solvates, or stereoisomers thereof:

K-L-T (A)

where: K is a targeting group that binds specifically to a target protein of interest (e.g., a KRAS protein, including both mutant proteins and wild-type); T is an E3-ligase binding group; and L is absent or is a bivalent linking group that connects K and T together via a covalent linkage.

In certain embodiments of compounds of Formula (A), the target protein of interest is KRAS, e.g., KRAS-G12A, KRAS-G12C, KRAS-G12D, KRAS-G12V, KRAS-G12R, KRAS-G12S, KRAS-G13D, KRAS-Q61H, and/or wild-type KRAS. In such embodiments, K is a KRAS targeting group, i.e., a targeting group that binds specifically to the KRAS protein (mutant or wild-type).

In certain embodiments of compounds of Formula (A), the targeting group K is a KRAS targeting group having the structure of Formula (I), or a pharmaceutically acceptable salt, ester, hydrate, solvate or stereoisomer thereof:

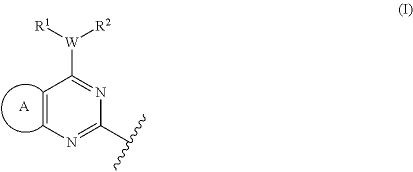

where:

A is a substituted or unsubstituted aromatic ring, heteroaromatic ring, carbocyclic ring, or carbon heterocyclic ring;

W is C, O or N, wherein, when W is O, R¹ is absent and R² is independently H or alkyl; when W is C, R¹ and R² are independently H, hydroxyl, halogen, alkyl, alkoxy, or alkanoyl; and when W is N, R¹ and R² are independently H, substituted or unsubstituted alkyl, or alkanoyl; or,

R¹, R² and the W linked to them form a substituted or unsubstituted aryl, heteroaryl, cycloalkyl, heterocycloalkyl, or one of the following groups:

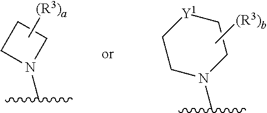

wherein Y¹ is O, N, C, —CH₂CH₂—, —CH═CH—, —CH—, —OCH₂—, or absent, and R³ can be substituted from any substitutable position on the ring and the position where H is located on Y¹; a is an integer from 0 to 6 (i.e., a is 0, 1, 2, 3, 4, 5, or 6); b is an integer from 0 to 8 (i.e., b is 0, 1, 2, 3, 4, 5, 6, 7, or 8); and

R³ is independently H, alkyl, hydroxy, halogen, amino, —CF₃, —NH(C₁-C₃ alkyl), —N(C₁-C₃ alkyl)₂, ═O, —CN, —O—(C₁-C₃ alkyl), —(C₁-C₃ alkyl)-OH, —C(═O)OH, —C(═O)(C₁-C₃ alkyl), —C(═O)O(C₁-C₃ alkyl), aryl, arylalkyl, cycloalkyl, or heterocycloalkyl; or,

any two R³s linked to the same atom and the ring to which they are linked form a spiro ring in which R¹ and R² constitute one ring, and the two R³s linked to the same atom constitute the other ring; wherein the ring formed by any two R³s linked to the same atom is an alkyl ring or a heteroalkyl ring, and the spiro ring is optionally substituted by alkyl, hydroxy, halogen, amino, ═O, or —CN; or,

any two adjacent R³s and the ring to which they are linked form a fused ring in which R¹ and R² constitute one ring, and the two adjacent R³s constitute the other ring; wherein the ring formed by any two adjacent R³s is an alkyl ring or a heteroalkyl ring, and the fused ring is optionally substituted by alkyl, hydroxy, halogen, amino, ═O, or —CN; or,

any two non-adjacent R³s and R¹ and R² constitute a bridged ring with a C₁-C₂ bridge in which R¹ and R² form the ring, and the two non-adjacent R³s are bound together to form the bridged bond.

In some embodiments of compounds of Formula (A), the targeting group K is a KRAS targeting group having the structure of Formula (I-a), or a pharmaceutically acceptable salt, ester, hydrate, solvate or stereoisomer thereof:

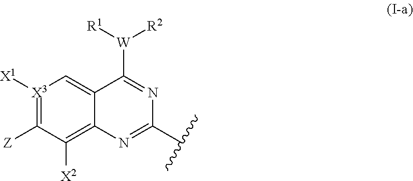

wherein:

X¹ and X² are independently H, F, Cl, CF₃, NH₂, or substituted or unsubstituted C₁-C₄ alkyl (e.g., C₁, C₂, C₃, or C₄ alkyl);

X³ is C or N;

Z is substituted or unsubstituted cycloalkyl, substituted or unsubstituted aryl, or substituted or unsubstituted polycyclic aromatic hydrocarbon;

wherein, when X³ is C, X¹ is halogen (such as F, Cl), CH₃, CF₃ or NH₂.

In some embodiments of structures of Formula (I-a), Z is

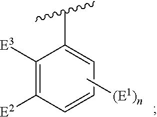

wherein, E¹ is hydrogen, hydroxy, amino, halogen atom, C₁-C₃ alkyl (e.g., methyl, ethyl, propyl, isopropyl), or absent, and E¹ can substitute any substitutable position on the ring; n is any integer from 0 to 3 (i.e., n is 0, 1, 2, or 3); and E² and E³ are independently hydroxy, amino, halogen (e.g., Cl, F), substituted or unsubstituted C₁-C₄ alkyl (e.g., substituted or unsubstituted methyl, ethyl, propyl, isopropyl, alkynyl, alkenyl, e.g., CF₃); or, E², E³ and substituted or unsubstituted phenyl ring to which they are linked form a substituted or unsubstituted bicyclic, tricyclic, fused, spirocyclic, or bridged ring.

In some embodiments of structures of Formula (I-a), Z is selected from one of the following structures:

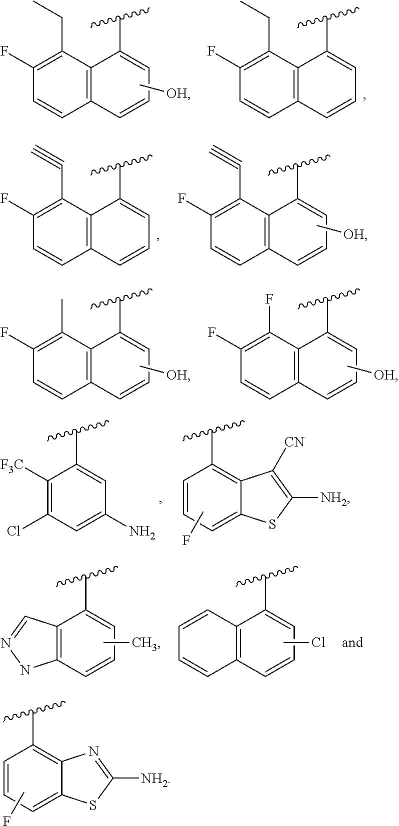

In other embodiments of structures of Formula (I-a), Z is

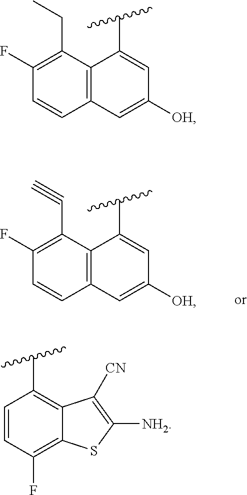 [SMILES: *c1cc(O)cc2ccc(F)c(CC)c12 ; *c1cc(O)cc2ccc(F)c(C#C)c12 ; *c1ccc(F)c2sc(N)c(C#N)c12]

In some embodiments of structures of Formula (I-a), the structure formed by R¹ and R² when bonded to W is selected from:

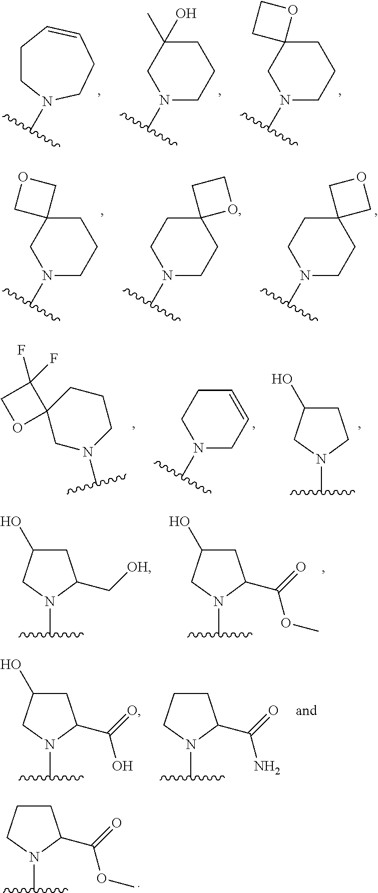

In some embodiments of compounds of Formula (A), the targeting group K is a KRAS targeting group having the structure of Formula (I-b), or a pharmaceutically acceptable salt, ester, hydrate, solvate or stereoisomer thereof:

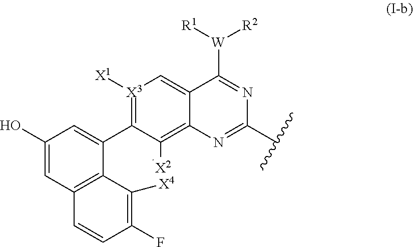

wherein:

X¹ and X² are independently H, F, Cl, CF₃, NH₃, or substituted or unsubstituted C₁-C₄ alkyl (e.g., C₁, C₂, C₃ or C₄ alkyl);

X³ is C or N;

X⁴ is substituted or unsubstituted C₁-C₅ alkyl (i.e., C₁, C₂, C₃, C₄ or C₅ alkyl) or substituted or unsubstituted vinyl or ethynyl, and

R¹ and R² are as defined above.

In some embodiments of compounds of Formula (A), the targeting group K is a KRAS targeting group having the structure of Formula (I-c), or a pharmaceutically acceptable salt, ester, hydrate, solvate or stereoisomer thereof:

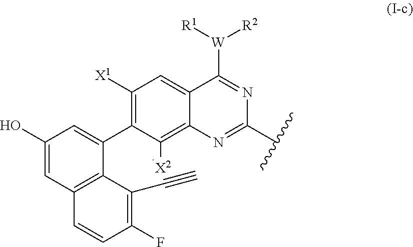

wherein:

X¹ and X² are independently H, F, Cl, CF₃, NH₃, or substituted or unsubstituted C₁-C₄ alkyl (i.e., C₁, C₂, C₃ or C₄ alkyl); and

R¹ and R² are as defined above.

In some embodiments of compounds of Formula (A), the targeting group K is a KRAS targeting group having the structure of Formula (I-d), or a pharmaceutically acceptable salt, ester, hydrate, solvate or stereoisomer thereof:

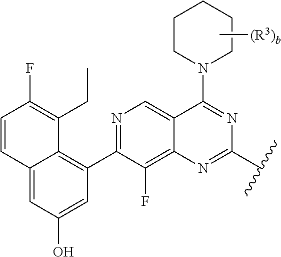

wherein:

any two R³s linked to the same atom and the ring to which they are linked form a spiro ring, where the spiro ring formed by the two R³s is an oxoalkyl ring, and the spiro ring can be substituted by alkyl, hydroxyl, halogen, amino, ═O, or —CN. In some such embodiments, R³ and the ring linked to them form

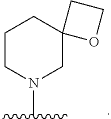 [SMILES: *N1CCCC2(CCO2)C1]

In some embodiments of compounds of Formula (A), the targeting group K is a KRAS targeting group having the structure of Formula (I-e), or a pharmaceutically acceptable salt, ester, hydrate, solvate or stereoisomer thereof:

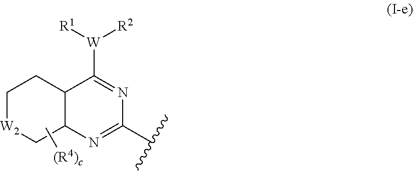

wherein:

W₂ is —CH₂— or —NH—;

R⁴ is H, F, Cl, CF₃, NH₃, or substituted or unsubstituted aryl, cycloalkyl, or heterocycloalkyl; or,

any two R⁴s linked to the same atom and the ring to which they are linked form a substituted or unsubstituted spiro ring, e.g., any two R⁴s linked to the same atom combine to form a substituted or unsubstituted cycloalkyl or heterocycloalkyl;

c is an integer from 0-4 (i.e., c is 0, 1, 2, 3, or 4); and

R¹ and R² are as defined above.

In some embodiments of compounds of Formula (A), the targeting group K is a KRAS targeting group having the structure of Formula (I-f), or a pharmaceutically acceptable salt, ester, hydrate, solvate or stereoisomer thereof:

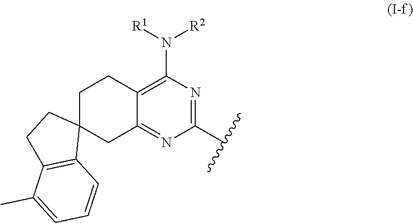

wherein:

R¹ and R² are as defined above.

In some embodiments of compounds of Formula (A), the targeting group K is:

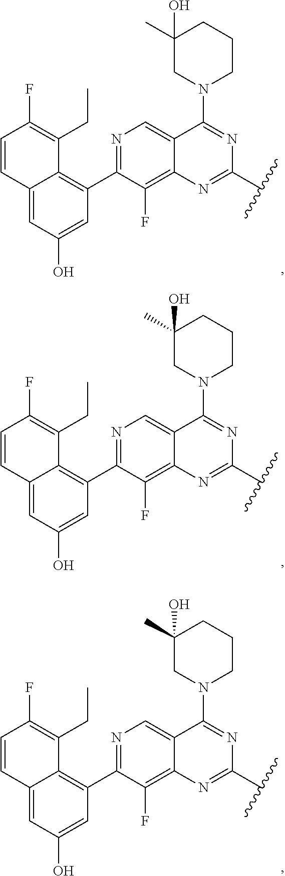 [SMILES: *c1nc(N2CCCC(C)(O)C2)c2cnc(-c3cc(O)cc4ccc(F)c(CC)c34)c(F)c2n1 ; *c1nc(N2CCC[C@](C)(O)C2)c2cnc(-c3cc(O)cc4ccc(F)c(CC)c34)c(F)c2n1 ; *c1nc(N2CCC[C@@](C)(O)C2)c2cnc(-c3cc(O)cc4ccc(F)c(CC)c34)c(F)c2n1]

or a pharmaceutically acceptable salt, ester, hydrate, solvate or stereoisomer thereof.

In some embodiments of compounds of Formula (A), the targeting group K is:

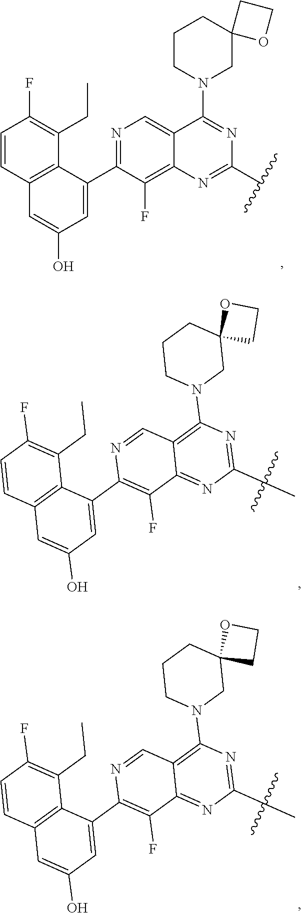 [SMILES: *c1nc(N2CCCC3(CCO3)C2)c2cnc(-c3cc(O)cc4ccc(F)c(CC)c34)c(F)c2n1 ; *c1nc(N2CCC[C@]3(CCO3)C2)c2cnc(-c3cc(O)cc4ccc(F)c(CC)c34)c(F)c2n1 ; *c1nc(N2CCC[C@@]3(CCO3)C2)c2cnc(-c3cc(O)cc4ccc(F)c(CC)c34)c(F)c2n1]

or a pharmaceutically acceptable salt, ester, hydrate, solvate or stereoisomer thereof.

In some embodiments of compounds of Formula (A), the targeting group K is a structure shown in Table 1.

TABLE 1
Exemplary targeting groups (K) in accordance with certain
embodiments of the disclosure.

| Cpd | |
|---|---|
| No | Structure |
| 1 a | 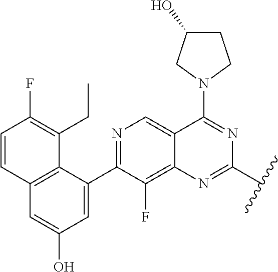 [SMILES: *c1nc(N2CC[C@@H](O)C2)c2cnc(-c3cc(O)cc4ccc(F)c(CC)c34)c(F)c2n1] |
| 2 a | 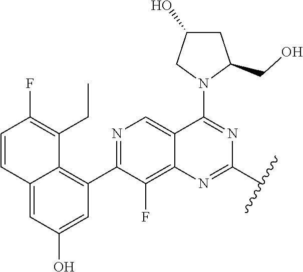 [SMILES: *c1nc(N2C[C@H](O)C[C@H]2CO)c2cnc(-c3cc(O)cc4ccc(F)c(CC)c34)c(F)c2n1] |
| 3 a | 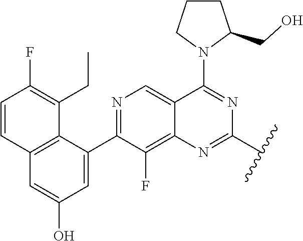 [SMILES: *c1nc(N2CCC[C@H]2CO)c2cnc(-c3cc(O)cc4ccc(F)c(CC)c34)c(F)c2n1] |
| 4 a | 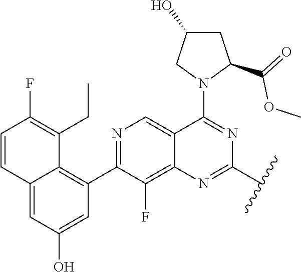 [SMILES: *c1nc(N2C[C@H](O)C[C@H]2C(=O)OC)c2cnc(-c3cc(O)cc4ccc(F)c(CC)c34)c(F)c2n1] |
| 5 a | 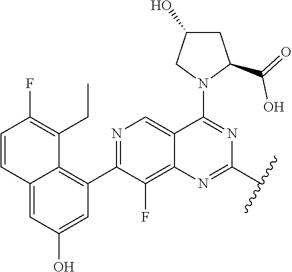 [SMILES: *c1nc(N2C[C@H](O)C[C@H]2C(=O)O)c2cnc(-c3cc(O)cc4ccc(F)c(CC)c34)c(F)c2n1] |
| 6 a | 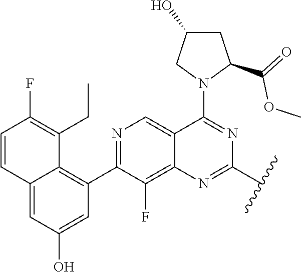 [SMILES: *c1nc(N2C[C@H](O)C[C@H]2C(=O)OC)c2cnc(-c3cc(O)cc4ccc(F)c(CC)c34)c(F)c2n1] |
| 7 a | 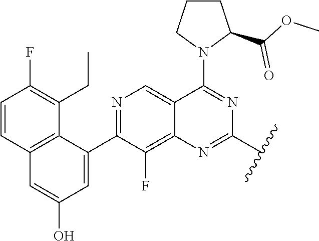 [SMILES: *c1nc(N2CCC[C@H]2C(=O)OC)c2cnc(-c3cc(O)cc4ccc(F)c(CC)c34)c(F)c2n1] |
| 8 a | 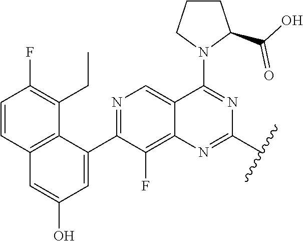 [SMILES: *c1nc(N2CCC[C@H]2C(=O)O)c2cnc(-c3cc(O)cc4ccc(F)c(CC)c34)c(F)c2n1] |
| 9 a | 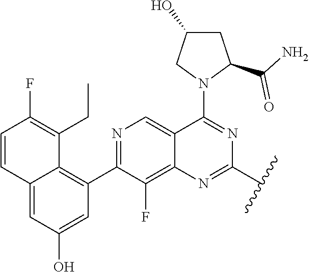 [SMILES: *c1nc(N2C[C@H](O)C[C@H]2C(N)=O)c2cnc(-c3cc(O)cc4ccc(F)c(CC)c34)c(F)c2n1] |
| 10 a | 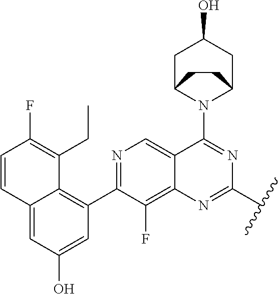 [SMILES: *c1nc(N2[C@@H]3CC[C@H]2C[C@@H](O)C3)c2cnc(-c3cc(O)cc4ccc(F)c(CC)c34)c(F)c2n1] |
| 11 a | 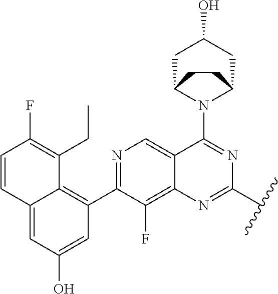 [SMILES: *c1nc(N2[C@@H]3CC[C@H]2C[C@H](O)C3)c2cnc(-c3cc(O)cc4ccc(F)c(CC)c34)c(F)c2n1] |
| 12 a | 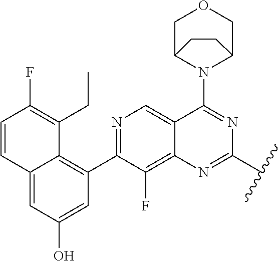 [SMILES: *c1nc(N2C3CCC2COC3)c2cnc(-c3cc(O)cc4ccc(F)c(CC)c34)c(F)c2n1] |
| 13 a | 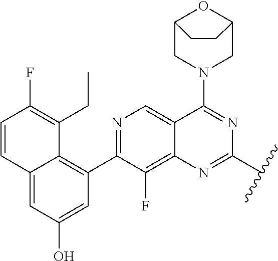 [SMILES: *c1nc(N2CC3CCC(C2)O3)c2cnc(-c3cc(O)cc4ccc(F)c(CC)c34)c(F)c2n1] |
| 14 a | 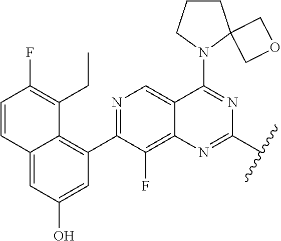 [SMILES: *c1nc(N2CCCC23COC3)c2cnc(-c3cc(O)cc4ccc(F)c(CC)c34)c(F)c2n1] |
| 15 a | 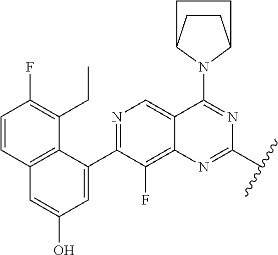 [SMILES: *c1nc(N2C3CCC2CC3)c2cnc(-c3cc(O)cc4ccc(F)c(CC)c34)c(F)c2n1] |
| 16 a | 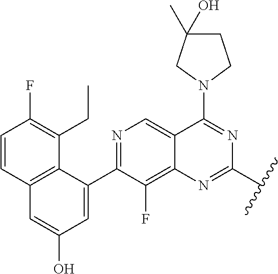 [SMILES: *c1nc(N2CCC(C)(O)C2)c2cnc(-c3cc(O)cc4ccc(F)c(CC)c34)c(F)c2n1] |
| 17 a | 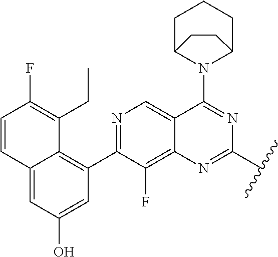 [SMILES: *c1nc(N2C3CCCC2CC3)c2cnc(-c3cc(O)cc4ccc(F)c(CC)c34)c(F)c2n1] |
| 18 a | 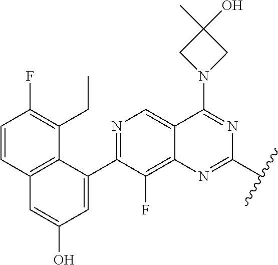 [SMILES: *c1nc(N2CC(C)(O)C2)c2cnc(-c3cc(O)cc4ccc(F)c(CC)c34)c(F)c2n1] |
| 19 a | 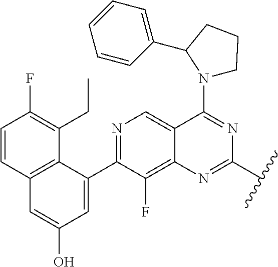 [SMILES: *c1nc(N2CCCC2c2ccccc2)c2cnc(-c3cc(O)cc4ccc(F)c(CC)c34)c(F)c2n1] |
| 20 a | 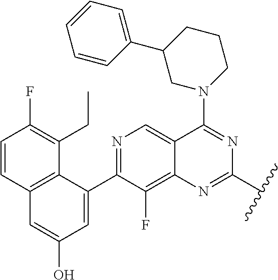 [SMILES: *c1nc(N2CCCC(c3ccccc3)C2)c2cnc(-c3cc(O)cc4ccc(F)c(CC)c34)c(F)c2n1] |
| 21 a | 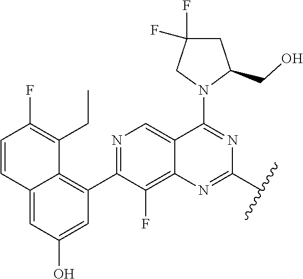 [SMILES: *c1nc(N2CC(F)(F)C[C@H]2CO)c2cnc(-c3cc(O)cc4ccc(F)c(CC)c34)c(F)c2n1] |
| 22 a | 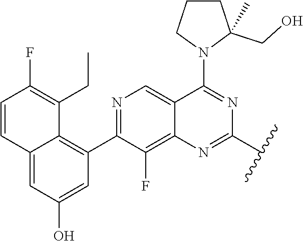 [SMILES: *c1nc(N2CCC[C@@]2(C)CO)c2cnc(-c3cc(O)cc4ccc(F)c(CC)c34)c(F)c2n1] |
| 23 a | 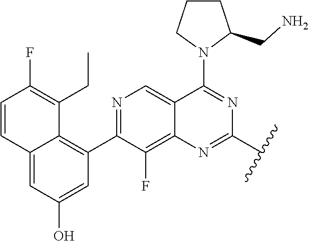 [SMILES: *c1nc(N2CCC[C@H]2CN)c2cnc(-c3cc(O)cc4ccc(F)c(CC)c34)c(F)c2n1] |
| 24 a | 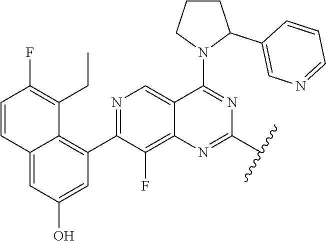 [SMILES: *c1nc(N2CCCC2c2cccnc2)c2cnc(-c3cc(O)cc4ccc(F)c(CC)c34)c(F)c2n1] |
| 25 a | 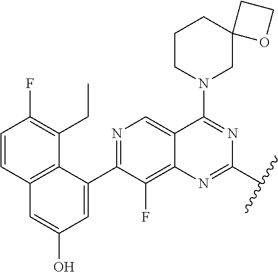 [SMILES: *c1nc(N2CCCC3(CCO3)C2)c2cnc(-c3cc(O)cc4ccc(F)c(CC)c34)c(F)c2n1] |
| 26 a | 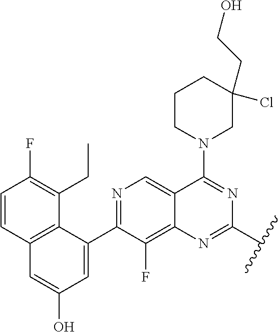 [SMILES: *c1nc(N2CCCC(Cl)(CCO)C2)c2cnc(-c3cc(O)cc4ccc(F)c(CC)c34)c(F)c2n1] |
| 27 a | 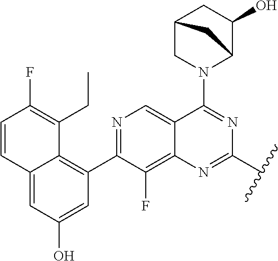 [SMILES: *c1nc(N2C[C@@H]3C[C@@H](O)[C@H]2C3)c2cnc(-c3cc(O)cc4ccc(F)c(CC)c34)c(F)c2n1] |
| 28 a | 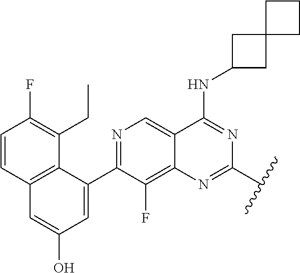 [SMILES: *c1nc(NC2CC3(CCC3)C2)c2cnc(-c3cc(O)cc4ccc(F)c(CC)c34)c(F)c2n1] |
| 29 a | 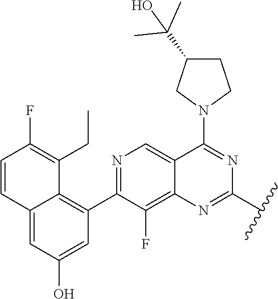 [SMILES: *c1nc(N2CC[C@@H](C(C)(C)O)C2)c2cnc(-c3cc(O)cc4ccc(F)c(CC)c34)c(F)c2n1] |
| 30 a | 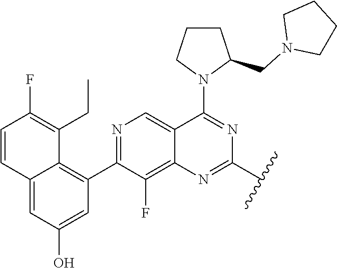 [SMILES: *c1nc(N2CCC[C@H]2CN2CCCC2)c2cnc(-c3cc(O)cc4ccc(F)c(CC)c34)c(F)c2n1] |
| 31 a | 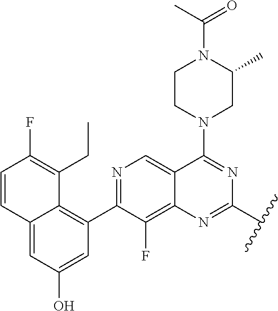 [SMILES: *c1nc(N2CCN(C(C)=O)[C@H](C)C2)c2cnc(-c3cc(O)cc4ccc(F)c(CC)c34)c(F)c2n1] |
| 32 a | 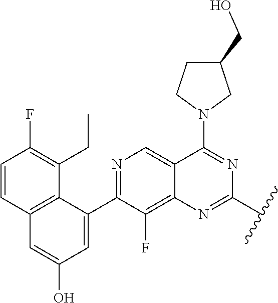 [SMILES: *c1nc(N2CC[C@@H](CO)C2)c2cnc(-c3cc(O)cc4ccc(F)c(CC)c34)c(F)c2n1] |
| 33 a | 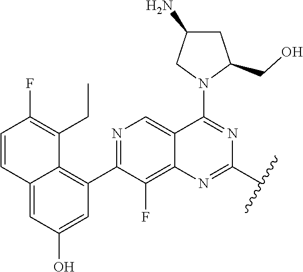 [SMILES: *c1nc(N2C[C@@H](N)C[C@H]2CO)c2cnc(-c3cc(O)cc4ccc(F)c(CC)c34)c(F)c2n1] |
| 34 a | 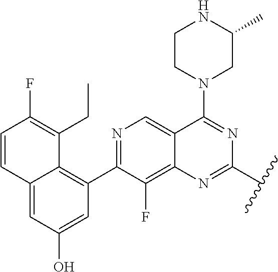 [SMILES: *c1nc(N2CCN[C@H](C)C2)c2cnc(-c3cc(O)cc4ccc(F)c(CC)c34)c(F)c2n1] |
| 35 a | 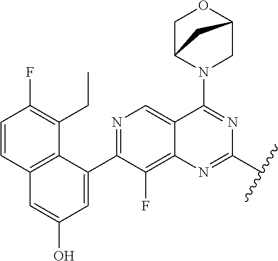 [SMILES: *c1nc(N2C[C@@H]3C[C@H]2CO3)c2cnc(-c3cc(O)cc4ccc(F)c(CC)c34)c(F)c2n1] |
| 36 a | 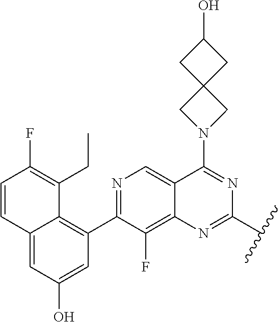 [SMILES: *c1nc(N2CC3(CC(O)C3)C2)c2cnc(-c3cc(O)cc4ccc(F)c(CC)c34)c(F)c2n1] |
| 37 a | 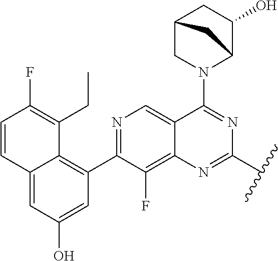 [SMILES: *c1nc(N2C[C@H]3C[C@@H]2[C@@H](O)C3)c2cnc(-c3cc(O)cc4ccc(F)c(CC)c34)c(F)c2n1] |
| 38 a | 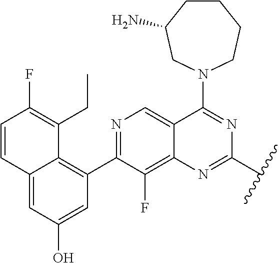 [SMILES: *c1nc(N2CCCC[C@@H](N)C2)c2cnc(-c3cc(O)cc4ccc(F)c(CC)c34)c(F)c2n1] |
| v39 a | 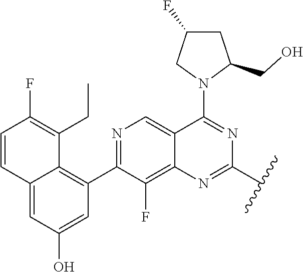 [SMILES: *c1nc(N2C[C@H](F)C[C@H]2CO)c2cnc(-c3cc(O)cc4ccc(F)c(CC)c34)c(F)c2n1] |
| 40 a | 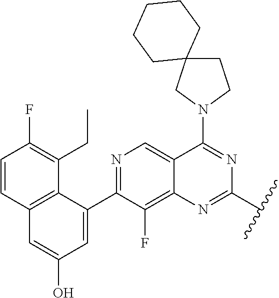 [SMILES: *c1nc(N2CCC3(CCCCC3)C2)c2cnc(-c3cc(O)cc4ccc(F)c(CC)c34)c(F)c2n1] |
| 41 a | 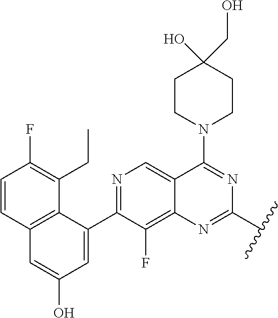 [SMILES: *c1nc(N2CCC(O)(CO)CC2)c2cnc(-c3cc(O)cc4ccc(F)c(CC)c34)c(F)c2n1] |
| 42 a | 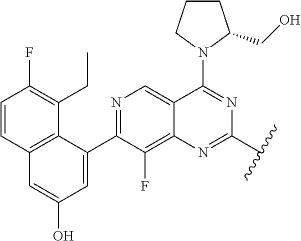 [SMILES: *c1nc(N2CCC[C@@H]2CO)c2cnc(-c3cc(O)cc4ccc(F)c(CC)c34)c(F)c2n1] |
| 43 a | 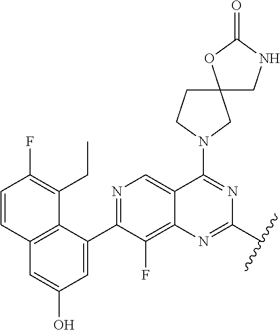 [SMILES: *c1nc(N2CCC3(CNC(=O)O3)C2)c2cnc(-c3cc(O)cc4ccc(F)c(CC)c34)c(F)c2n1] |
| 44 a | 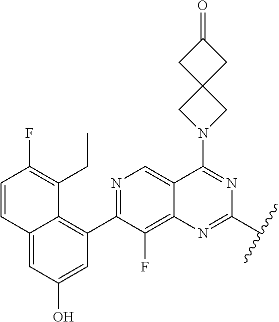 [SMILES: *c1nc(N2CC3(CC(=O)C3)C2)c2cnc(-c3cc(O)cc4ccc(F)c(CC)c34)c(F)c2n1] |
| 45 a | 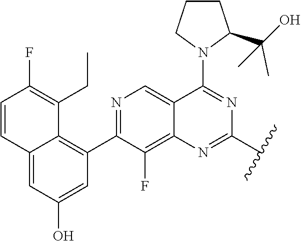 [SMILES: *c1nc(N2CCC[C@H]2C(C)(C)O)c2cnc(-c3cc(O)cc4ccc(F)c(CC)c34)c(F)c2n1] |
| 46 a | 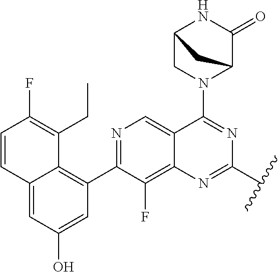 [SMILES: *c1nc(N2C[C@H]3C[C@@H]2C(=O)N3)c2cnc(-c3cc(O)cc4ccc(F)c(CC)c34)c(F)c2n1] |
| v47 a | 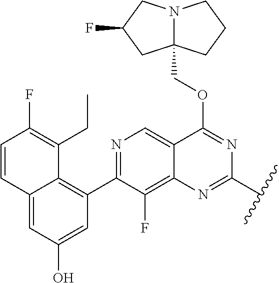 [SMILES: *c1nc(OC[C@@]23CCCN2C[C@H](F)C3)c2cnc(-c3cc(O)cc4ccc(F)c(CC)c34)c(F)c2n1] |
| 48 a | 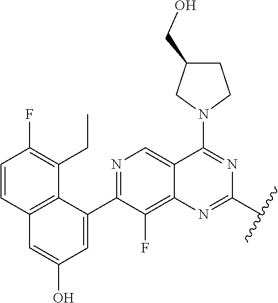 [SMILES: *c1nc(N2CC[C@H](CO)C2)c2cnc(-c3cc(O)cc4ccc(F)c(CC)c34)c(F)c2n1] |
| 49 a | 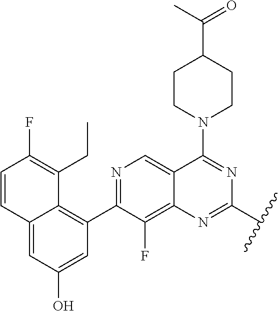 [SMILES: *c1nc(N2CCC(C(C)=O)CC2)c2cnc(-c3cc(O)cc4ccc(F)c(CC)c34)c(F)c2n1] |
| 50 a | 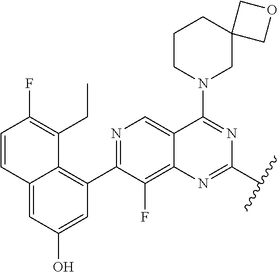 [SMILES: *c1nc(N2CCCC3(COC3)C2)c2cnc(-c3cc(O)cc4ccc(F)c(CC)c34)c(F)c2n1] |
| 51 a | 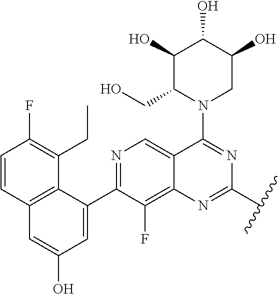 [SMILES: *c1nc(N2C[C@H](O)[C@@H](O)[C@H](O)[C@H]2CO)c2cnc(-c3cc(O)cc4ccc(F)c(CC)c34)c(F)c2n1] |
| 52 a | 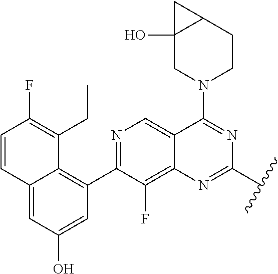 [SMILES: *c1nc(N2CCC3CC3(O)C2)c2cnc(-c3cc(O)cc4ccc(F)c(CC)c34)c(F)c2n1] |
| 53 a | 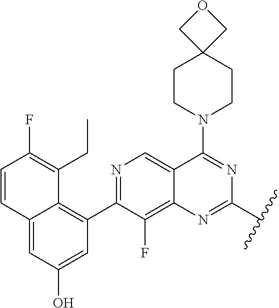 [SMILES: *c1nc(N2CCC3(CC2)COC3)c2cnc(-c3cc(O)cc4ccc(F)c(CC)c34)c(F)c2n1] |
| 54 a | 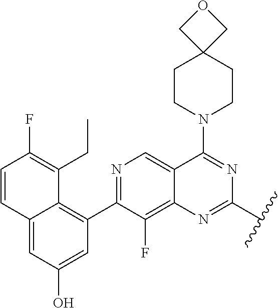 [SMILES: *c1nc(N2CCC3(CC2)COC3)c2cnc(-c3cc(O)cc4ccc(F)c(CC)c34)c(F)c2n1] |
| 55 a | 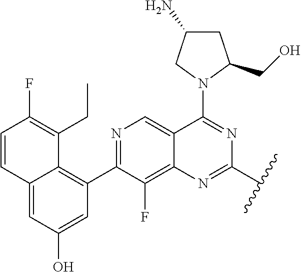 [SMILES: *c1nc(N2C[C@H](N)C[C@H]2CO)c2cnc(-c3cc(O)cc4ccc(F)c(CC)c34)c(F)c2n1] |
| 56 a | 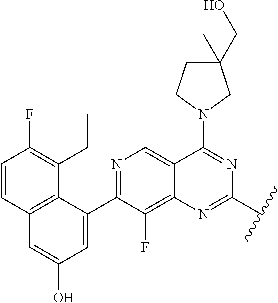 [SMILES: *c1nc(N2CCC(C)(CO)C2)c2cnc(-c3cc(O)cc4ccc(F)c(CC)c34)c(F)c2n1] |
| 57 a | 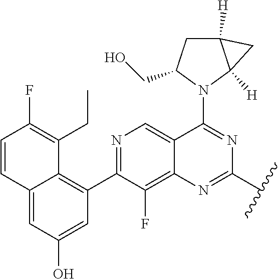 [SMILES: *c1nc(N2[C@H](CO)C[C@H]3C[C@H]32)c2cnc(-c3cc(O)cc4ccc(F)c(CC)c34)c(F)c2n1] |
| 58 a | 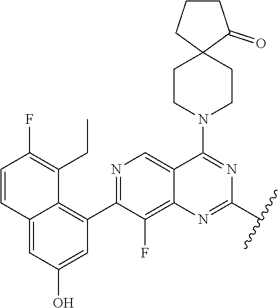 [SMILES: *c1nc(N2CCC3(CCCC3=O)CC2)c2cnc(-c3cc(O)cc4ccc(F)c(CC)c34)c(F)c2n1] |
| 59 a | 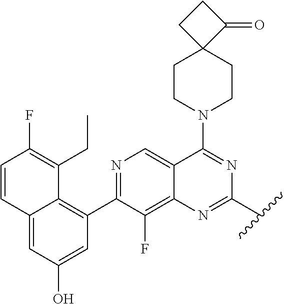 [SMILES: *c1nc(N2CCC3(CCC3=O)CC2)c2cnc(-c3cc(O)cc4ccc(F)c(CC)c34)c(F)c2n1] |
| 60 a | 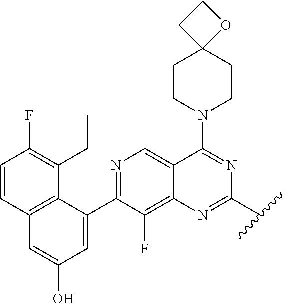 [SMILES: *c1nc(N2CCC3(CCO3)CC2)c2cnc(-c3cc(O)cc4ccc(F)c(CC)c34)c(F)c2n1] |
| 61 a | 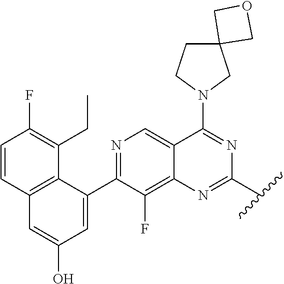 [SMILES: *c1nc(N2CCC3(COC3)C2)c2cnc(-c3cc(O)cc4ccc(F)c(CC)c34)c(F)c2n1] |
| 62 a | 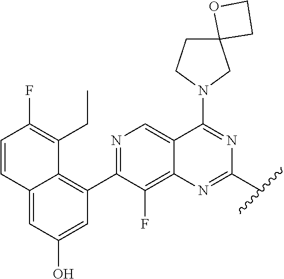 [SMILES: *c1nc(N2CCC3(CCO3)C2)c2cnc(-c3cc(O)cc4ccc(F)c(CC)c34)c(F)c2n1] |
| 63 a | 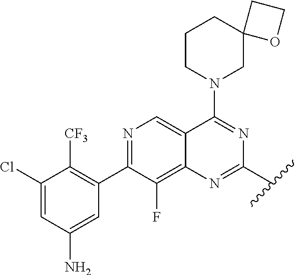 [SMILES: *c1nc(N2CCCC3(CCO3)C2)c2cnc(-c3cc(N)cc(Cl)c3C(F)(F)F)c(F)c2n1] |
| 64 a | 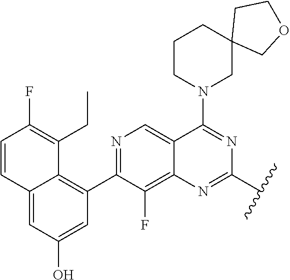 [SMILES: *c1nc(N2CCCC3(CCOC3)C2)c2cnc(-c3cc(O)cc4ccc(F)c(CC)c34)c(F)c2n1] |
| 65 a | 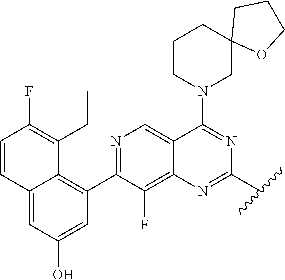 [SMILES: *c1nc(N2CCCC3(CCCO3)C2)c2cnc(-c3cc(O)cc4ccc(F)c(CC)c34)c(F)c2n1] |
| 66 a | 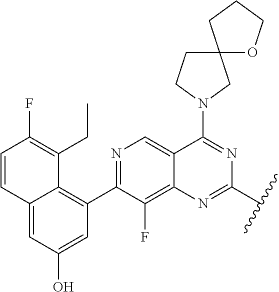 [SMILES: *c1nc(N2CCC3(CCCO3)C2)c2cnc(-c3cc(O)cc4ccc(F)c(CC)c34)c(F)c2n1] |
| 67 a | 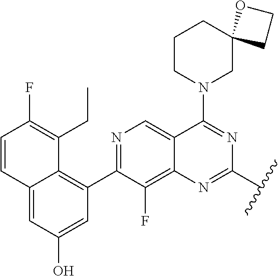 [SMILES: *c1nc(N2CCC[C@@]3(CCO3)C2)c2cnc(-c3cc(O)cc4ccc(F)c(CC)c34)c(F)c2n1] |
| 68 a | 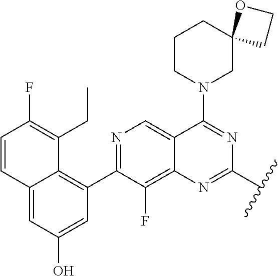 [SMILES: *c1nc(N2CCC[C@]3(CCO3)C2)c2cnc(-c3cc(O)cc4ccc(F)c(CC)c34)c(F)c2n1] |
| 69 a | 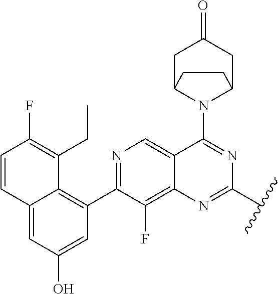 [SMILES: *c1nc(N2C3CCC2CC(=O)C3)c2cnc(-c3cc(O)cc4ccc(F)c(CC)c34)c(F)c2n1] |
| 70 a | 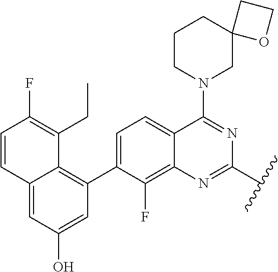 [SMILES: *c1nc(N2CCCC3(CCO3)C2)c2ccc(-c3cc(O)cc4ccc(F)c(CC)c34)c(F)c2n1] |
| 71 a | 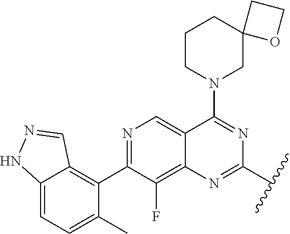 [SMILES: *c1nc(N2CCCC3(CCO3)C2)c2cnc(-c3c(C)ccc4[nH]ncc34)c(F)c2n1] |
| 72 a | 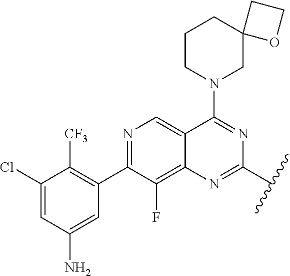 [SMILES: *c1nc(N2CCCC3(CCO3)C2)c2cnc(-c3cc(N)cc(Cl)c3C(F)(F)F)c(F)c2n1] |
| 73 a | 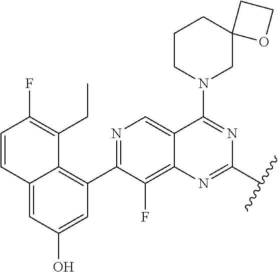 [SMILES: *c1nc(N2CCCC3(CCO3)C2)c2cnc(-c3cc(O)cc4ccc(F)c(CC)c34)c(F)c2n1] |
| 74 a | 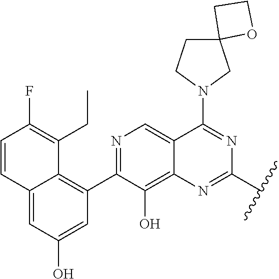 [SMILES: *c1nc(N2CCC3(CCO3)C2)c2cnc(-c3cc(O)cc4ccc(F)c(CC)c34)c(O)c2n1] |
| 75 a | 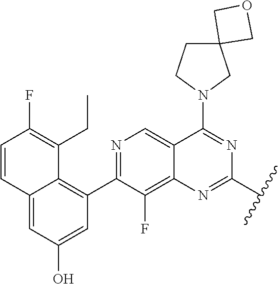 [SMILES: *c1nc(N2CCC3(COC3)C2)c2cnc(-c3cc(O)cc4ccc(F)c(CC)c34)c(F)c2n1] |
| 76 a | 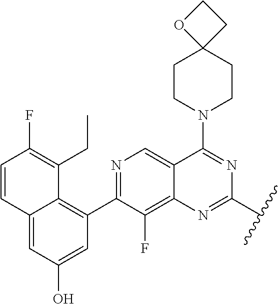 [SMILES: *c1nc(N2CCC3(CCO3)CC2)c2cnc(-c3cc(O)cc4ccc(F)c(CC)c34)c(F)c2n1] |
| 77 a | 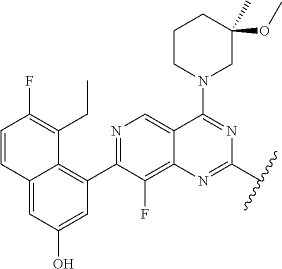 [SMILES: *c1nc(N2CCC[C@@](C)(OC)C2)c2cnc(-c3cc(O)cc4ccc(F)c(CC)c34)c(F)c2n1] |
| 78 a | 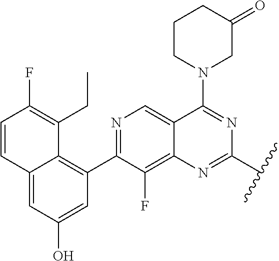 [SMILES: *c1nc(N2CCCC(=O)C2)c2cnc(-c3cc(O)cc4ccc(F)c(CC)c34)c(F)c2n1] |
| 79 a | 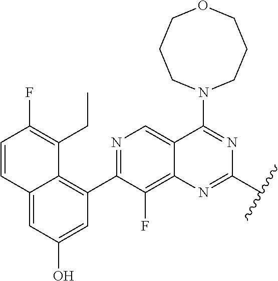 [SMILES: *c1nc(N2CCCOCCC2)c2cnc(-c3cc(O)cc4ccc(F)c(CC)c34)c(F)c2n1] |
| 80 a | 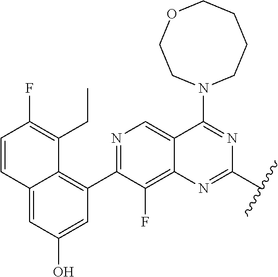 [SMILES: *c1nc(N2CCCCOCC2)c2cnc(-c3cc(O)cc4ccc(F)c(CC)c34)c(F)c2n1] |
| 81 a | 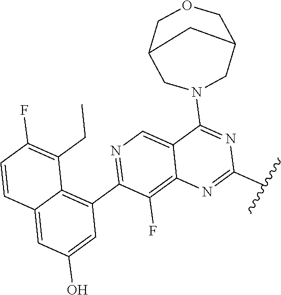 [SMILES: *c1nc(N2CC3COCC(C3)C2)c2cnc(-c3cc(O)cc4ccc(F)c(CC)c34)c(F)c2n1] |
| 82 a | 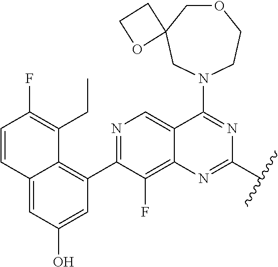 [SMILES: *c1nc(N2CCOCC3(CCO3)C2)c2cnc(-c3cc(O)cc4ccc(F)c(CC)c34)c(F)c2n1] |
| 83 a | 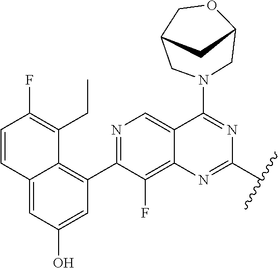 [SMILES: *c1nc(N2C[C@@H]3CO[C@@H](C3)C2)c2cnc(-c3cc(O)cc4ccc(F)c(CC)c34)c(F)c2n1] |
| 84 a | 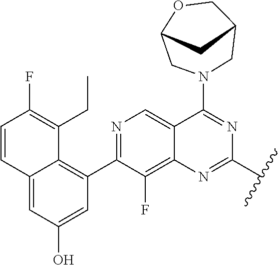 [SMILES: *c1nc(N2C[C@H]3CO[C@H](C3)C2)c2cnc(-c3cc(O)cc4ccc(F)c(CC)c34)c(F)c2n1] |
| 85 a | 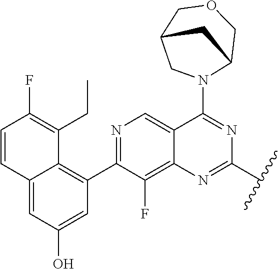 [SMILES: *c1nc(N2C[C@@H]3COC[C@H]2C3)c2cnc(-c3cc(O)cc4ccc(F)c(CC)c34)c(F)c2n1] |
| 86 a | 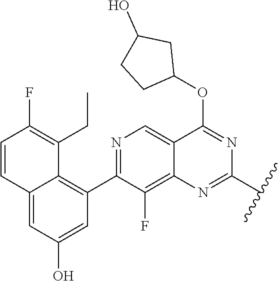 [SMILES: *c1nc(OC2CCC(O)C2)c2cnc(-c3cc(O)cc4ccc(F)c(CC)c34)c(F)c2n1] |
| 87 a | 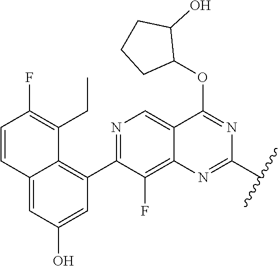 [SMILES: *c1nc(OC2CCCC2O)c2cnc(-c3cc(O)cc4ccc(F)c(CC)c34)c(F)c2n1] |
| 88 a | 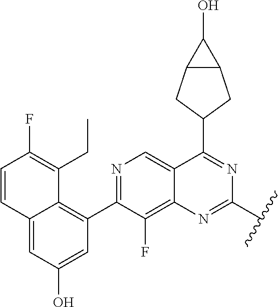 [SMILES: *c1nc(C2CC3C(O)C3C2)c2cnc(-c3cc(O)cc4ccc(F)c(CC)c34)c(F)c2n1] |
| 89 a | 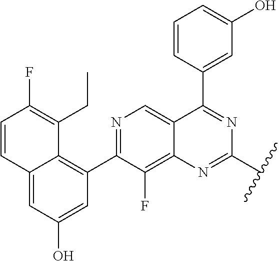 [SMILES: *c1nc(-c2cccc(O)c2)c2cnc(-c3cc(O)cc4ccc(F)c(CC)c34)c(F)c2n1] |
| 90 a | 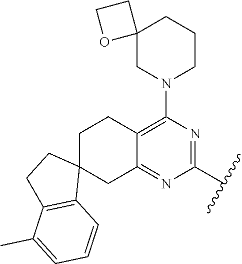 [SMILES: *c1nc2c(c(N3CCCC4(CCO4)C3)n1)CCC1(CCc3c(C)cccc31)C2] |
| 91 a | 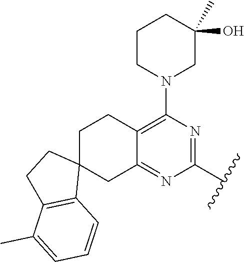 [SMILES: *c1nc2c(c(N3CCC[C@@](C)(O)C3)n1)CCC1(CCc3c(C)cccc31)C2] |
| 92 a | 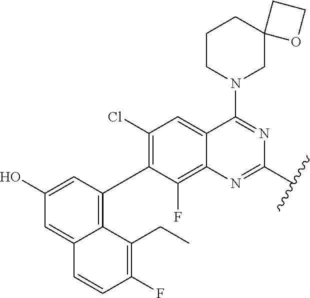 [SMILES: *c1nc(N2CCCC3(CCO3)C2)c2cc(Cl)c(-c3cc(O)cc4ccc(F)c(CC)c34)c(F)c2n1] |
| 93 a | 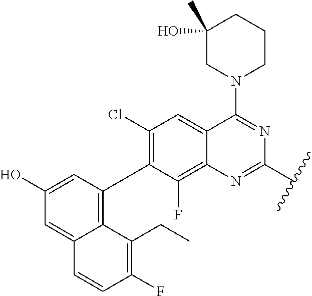 [SMILES: *c1nc(N2CCC[C@@](C)(O)C2)c2cc(Cl)c(-c3cc(O)cc4ccc(F)c(CC)c34)c(F)c2n1] |
| 94 a | 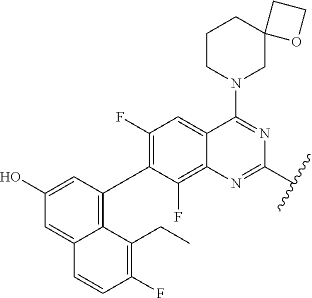 [SMILES: *c1nc(N2CCCC3(CCO3)C2)c2cc(F)c(-c3cc(O)cc4ccc(F)c(CC)c34)c(F)c2n1] |
| 95 a | 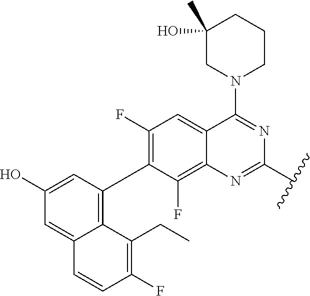 [SMILES: *c1nc(N2CCC[C@@](C)(O)C2)c2cc(F)c(-c3cc(O)cc4ccc(F)c(CC)c34)c(F)c2n1] |
| 96 a | 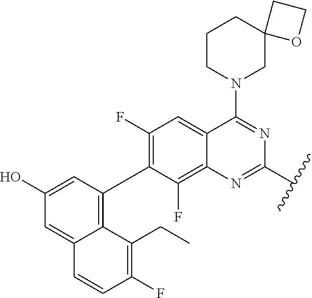 [SMILES: *c1nc(N2CCCC3(CCO3)C2)c2cc(F)c(-c3cc(O)cc4ccc(F)c(CC)c34)c(F)c2n1] |
| 97 a | 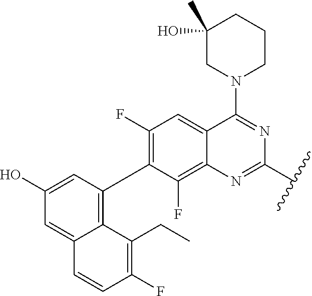 [SMILES: *c1nc(N2CCC[C@@](C)(O)C2)c2cc(F)c(-c3cc(O)cc4ccc(F)c(CC)c34)c(F)c2n1] |
| 98 a | 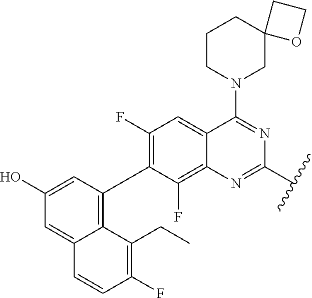 [SMILES: *c1nc(N2CCCC3(CCO3)C2)c2cc(F)c(-c3cc(O)cc4ccc(F)c(CC)c34)c(F)c2n1] |
| 99 a | 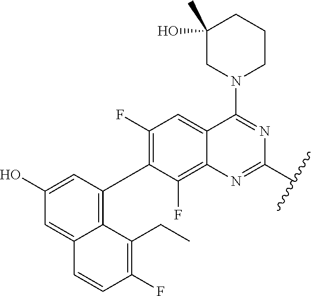 [SMILES: *c1nc(N2CCC[C@@](C)(O)C2)c2cc(F)c(-c3cc(O)cc4ccc(F)c(CC)c34)c(F)c2n1] |
| 100 a | 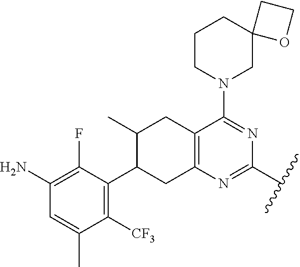 [SMILES: *c1nc2c(c(N3CCCC4(CCO4)C3)n1)CC(C)C(c1c(F)c(N)cc(C)c1C(F)(F)F)C2] |
| 101 a | 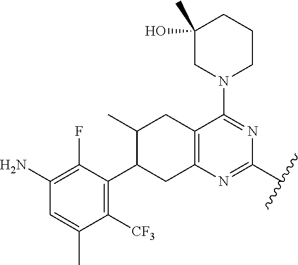 [SMILES: *c1nc2c(c(N3CCC[C@@](C)(O)C3)n1)CC(C)C(c1c(F)c(N)cc(C)c1C(F)(F)F)C2] |
| 102 a | 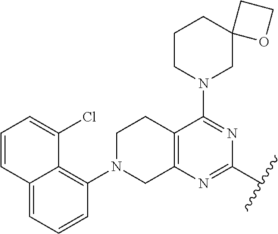 [SMILES: *c1nc2c(c(N3CCCC4(CCO4)C3)n1)CCN(c1cccc3cccc(Cl)c13)C2] |
| 103 a | 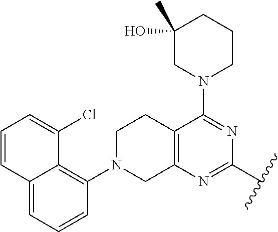 [SMILES: *c1nc2c(c(N3CCC[C@@](C)(O)C3)n1)CCN(c1cccc3cccc(Cl)c13)C2] |
| 104 a | 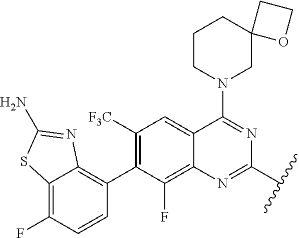 [SMILES: *c1nc(N2CCCC3(CCO3)C2)c2cc(C(F)(F)F)c(-c3ccc(F)c4sc(N)nc34)c(F)c2n1] |
| 105 a | 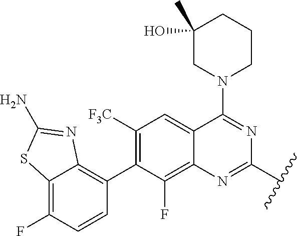 [SMILES: *c1nc(N2CCC[C@@](C)(O)C2)c2cc(C(F)(F)F)c(-c3ccc(F)c4sc(N)nc34)c(F)c2n1] |
| 106 a | 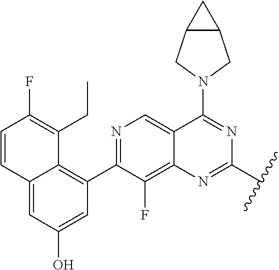 [SMILES: *c1nc(N2CC3CC3C2)c2cnc(-c3cc(O)cc4ccc(F)c(CC)c34)c(F)c2n1] |
| 107 a | 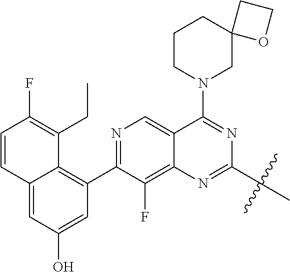 [SMILES: *c1nc(N2CCCC3(CCO3)C2)c2cnc(-c3cc(O)cc4ccc(F)c(CC)c34)c(F)c2n1] |
| 108 a | 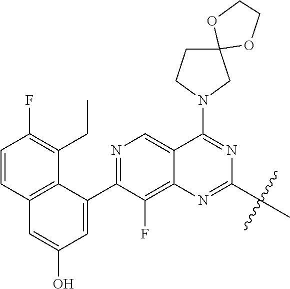 [SMILES: *c1nc(N2CCC3(C2)OCCO3)c2cnc(-c3cc(O)cc4ccc(F)c(CC)c34)c(F)c2n1] |
| 109 a | 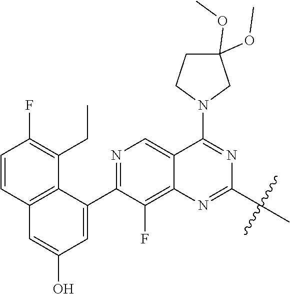 [SMILES: *c1nc(N2CCC(OC)(OC)C2)c2cnc(-c3cc(O)cc4ccc(F)c(CC)c34)c(F)c2n1] |
| 110 a | 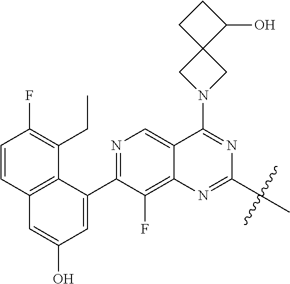 [SMILES: *c1nc(N2CC3(CCC3O)C2)c2cnc(-c3cc(O)cc4ccc(F)c(CC)c34)c(F)c2n1] |
| 111 a | 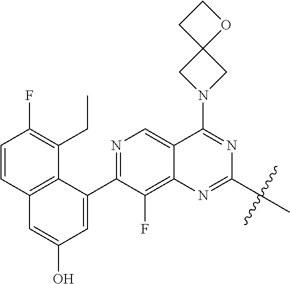 [SMILES: *c1nc(N2CC3(CCO3)C2)c2cnc(-c3cc(O)cc4ccc(F)c(CC)c34)c(F)c2n1] |
| 112 a | 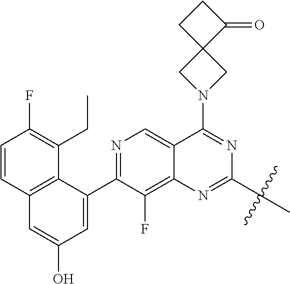 [SMILES: *c1nc(N2CC3(CCC3=O)C2)c2cnc(-c3cc(O)cc4ccc(F)c(CC)c34)c(F)c2n1] |
| 113 a | 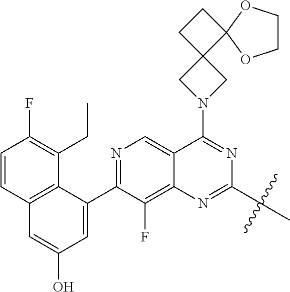 [SMILES: *c1nc(N2CC3(CCC34OCCO4)C2)c2cnc(-c3cc(O)cc4ccc(F)c(CC)c34)c(F)c2n1] |
| 114 a | 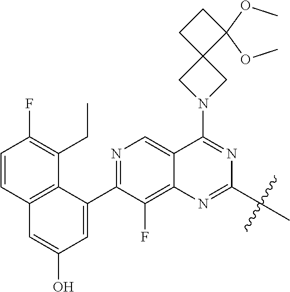 [SMILES: *c1nc(N2CC3(CCC3(OC)OC)C2)c2cnc(-c3cc(O)cc4ccc(F)c(CC)c34)c(F)c2n1] |
| 115 a | 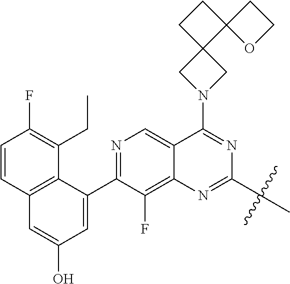 [SMILES: *c1nc(N2CC3(CCC34CCO4)C2)c2cnc(-c3cc(O)cc4ccc(F)c(CC)c34)c(F)c2n1] |
| 116 a | 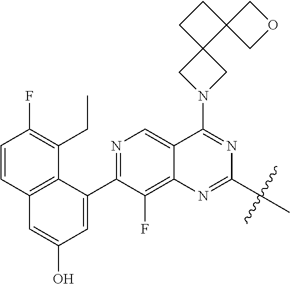 [SMILES: *c1nc(N2CC3(CCC34COC4)C2)c2cnc(-c3cc(O)cc4ccc(F)c(CC)c34)c(F)c2n1] |
| 117 a | 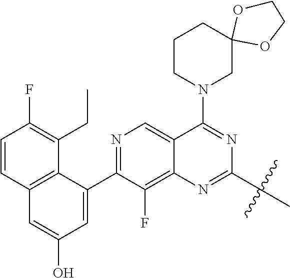 [SMILES: *c1nc(N2CCCC3(C2)OCCO3)c2cnc(-c3cc(O)cc4ccc(F)c(CC)c34)c(F)c2n1] |
| 118 a | 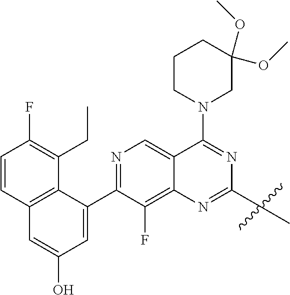 [SMILES: *c1nc(N2CCCC(OC)(OC)C2)c2cnc(-c3cc(O)cc4ccc(F)c(CC)c34)c(F)c2n1] |
| 119 a | 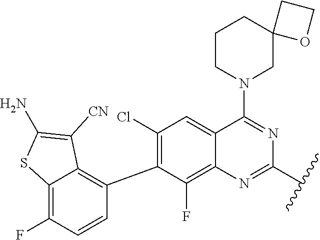 [SMILES: *c1nc(N2CCCC3(CCO3)C2)c2cc(Cl)c(-c3ccc(F)c4sc(N)c(C#N)c34)c(F)c2n1] |
| 120 a | 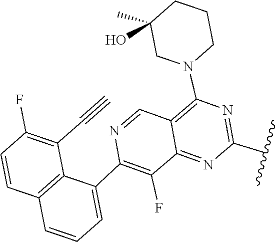 [SMILES: *c1nc(N2CCC[C@](C)(O)C2)c2cnc(-c3cccc4ccc(F)c(C#C)c34)c(F)c2n1] |
| 121 a | 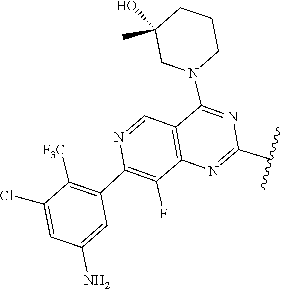 [SMILES: *c1nc(N2CCC[C@@](C)(O)C2)c2cnc(-c3cc(N)cc(Cl)c3C(F)(F)F)c(F)c2n1] |
| 122 a | 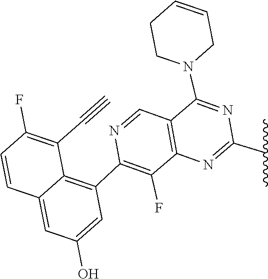 [SMILES: *c1nc(N2CC=CCC2)c2cnc(-c3cc(O)cc4ccc(F)c(C#C)c34)c(F)c2n1] |
| 123 a | 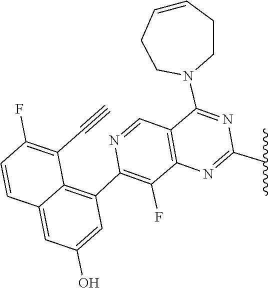 [SMILES: *c1nc(N2CCC=CCC2)c2cnc(-c3cc(O)cc4ccc(F)c(C#C)c34)c(F)c2n1] |
| 124 a | 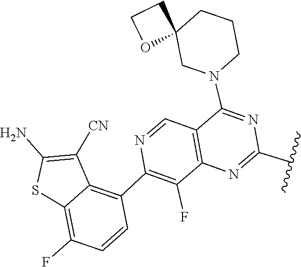 [SMILES: *c1nc(N2CCC[C@]3(CCO3)C2)c2cnc(-c3ccc(F)c4sc(N)c(C#N)c34)c(F)c2n1] |
| 125 a | 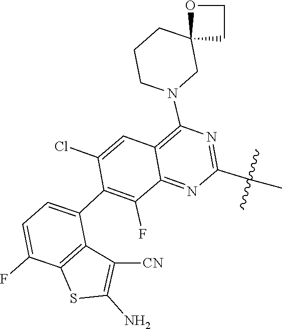 [SMILES: *c1nc(N2CCC[C@]3(CCO3)C2)c2cc(Cl)c(-c3ccc(F)c4sc(N)c(C#N)c34)c(F)c2n1] |
| 126 a | 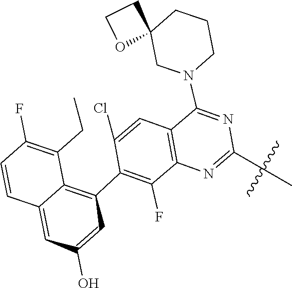 |
| 127 a | 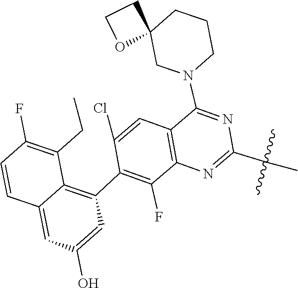 |

In some embodiments of compounds of Formula (A), the targeting group K is directed to a single site on the target protein. In other embodiments of compounds of Formula (A), the targeting group K is directed to multiple sites on the target protein. In some such embodiments, the targeting group K is directed to two, three, or four sites on the target protein.

In certain embodiments of compounds of Formula (A), the E3 ligase binding group (T) comprises a ligand of an E3 ligase (i.e., is a ligand group).

In some embodiments of compounds of Formula (A), the E3 ligase binding group (T) can be linked to a single E3 ubiquitin ligase. In other embodiments of compounds of Formula (A), the ligand group T can be linked to multiple E3 ubiquitin ligases, e.g., two, three, or four ubiquitin ligases.

In some embodiments of compounds of Formula (A), the bivalent linking group L is composed of one or more of L¹, L² and L³. In some such embodiments, the bivalent linking group has a structure of L¹-L²-L³, and the bifunctional compound of the disclosure contains a targeting group K and an E3 ligase binding group T, where K and T are covalently linked to respective positions on the bivalent linking group L¹-L²-L³, forming a compound represented by the following formula: K-L¹-L²-L³-T.

In some embodiments, the bifunctional compound of the disclosure solely comprises a targeting group K and an E3 ligase binding group T, forming a compound represented by the following formula: K-T.

In some embodiments, the bifunctional compound of the disclosure solely comprises a targeting group K, a bivalent linking group L¹, and an E3 ligase binding group T, forming a compound represented by the following formula: K-L¹-T.

In some embodiments, the bifunctional compound of the disclosure solely comprises a targeting group K, a bivalent linking group L¹ and L², and an E3 ligase binding group T, forming a compound represented by the following formula: K-L¹-L²-T.

In some embodiments of compounds of the disclosure, the bivalent linking group L has a structure of L¹-L²-L³, where L¹, L², and L³ are all present, or only one or two of L¹, L², and L³ are present.

In some embodiments of compounds of the disclosure, L¹, L², and L³ (within the bivalent linking group) are independently substituted or unsubstituted hydrocarbyl, hydrocarbyloxy, oxyhydrocarbyl, cyclohydrorocarbyl, heterocyclohydrocarbyl, acylhydrocarbyl, hydrocarbylacyl, carbonylhydrocarbyl, hydrocarbylcarbonyl, amidohydrocarbyl, hydrocarbylamido, aryl, or oligopeptide group, each of L¹, L², and L³ comprising a bivalent connecting site.

In some embodiments, a hydrocarbyl includes, for example and without limitation, saturated hydrocarbyl, unsaturated hydrocarbyl, aromatic hydrocarbyl, oxyhydrocarbyl, azahydrocarbyl, thiahydrocarbyl, phosphahydrocarbyl, and/or mixed heterohydrocarbyl with various heteroatoms. The chain length of the hydrocarbyl or heterohydrocarbyl generally ranges from 1 to 20 atoms. In some embodiments, when the hydrocarbyl is heterohydrocarbyl, it contains 1 to 5 heteroatoms, and the chemical valence of these heteroatoms can be satisfied by hydrogen, oxygen, nitrogen, etc., as needed, through appropriate bonding. In some embodiments, the heterocyclic ring in the heterocyclohydrocarbyl includes, for example and without limitation, substituted or unsubstituted monocyclic, spirocyclic, or fused ring, and the like.

In some embodiments of compounds of the disclosure, L¹ (within the bivalent linking group) is —O— or —NH₂—.

In some embodiments of compounds of the disclosure, L¹ (within the bivalent linking group) is a structure shown in (IIa), (IIb), (IIc), (IId), (IIe), (IIf), (IIg), (IIh), (IIi), (IIj), or (IIk):

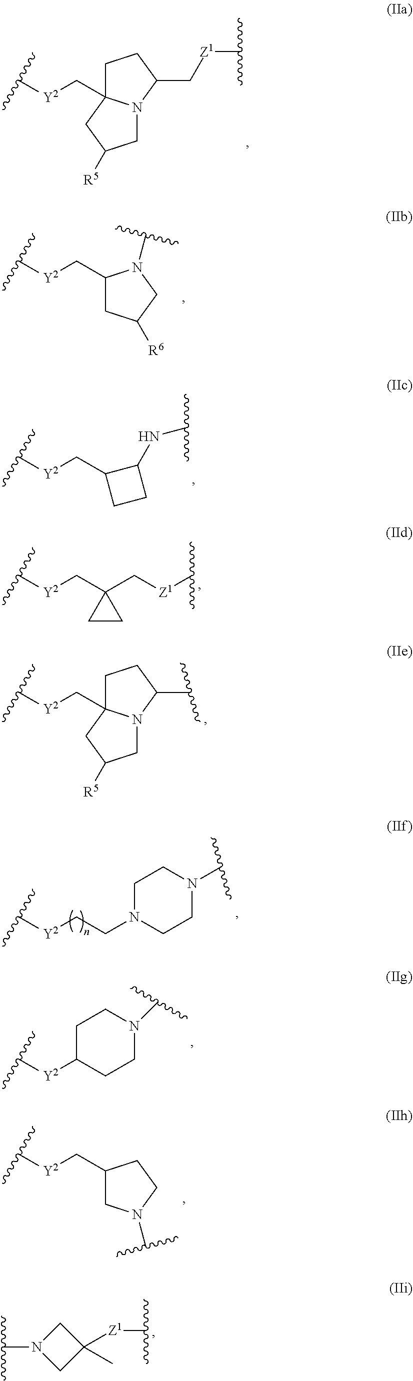

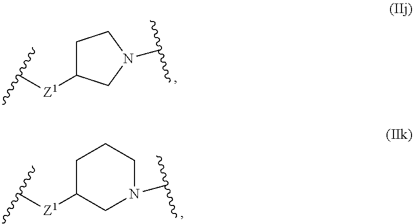

wherein:

Y² and Z¹ are independently oxygen (O), nitrogen (NH), or sulfur (S);

n is an integer from 0 to 20;

R⁵ and R⁶ are independently hydrogen, halogen (e.g., fluorine, chlorine, bromine, or iodine), hydroxyl, alkoxy, amino, or amine; wherein, when the structure contains chiral centers, its stereochemical structures are independently R-configuration, S-configuration, or a mixture of R- and S-configurations.

In some such embodiments, n is an integer from 0 to 5 (i.e., n is 0, 1, 2, 3, 4, 5).

In some such embodiments, Z¹ is a six-membered nitrogen-containing heterocycle.

In some embodiments of compounds of the disclosure, L¹ (within the bivalent linking group) is selected from:

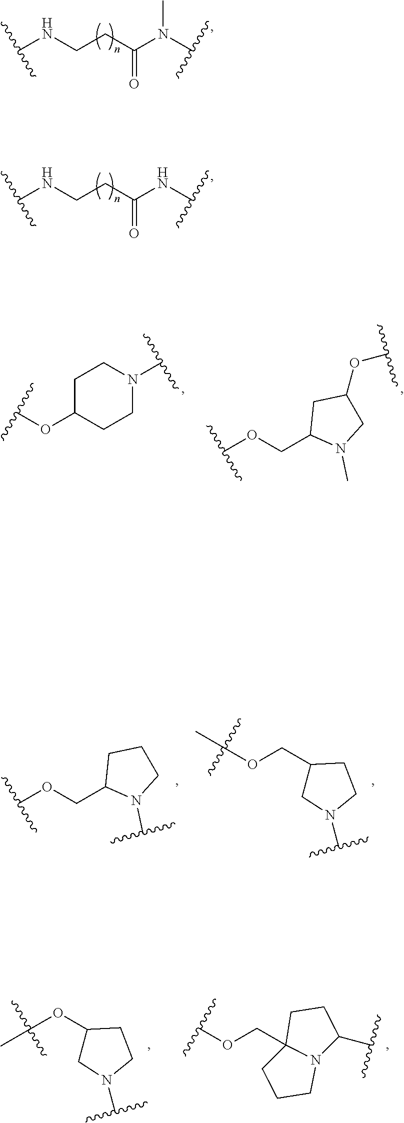

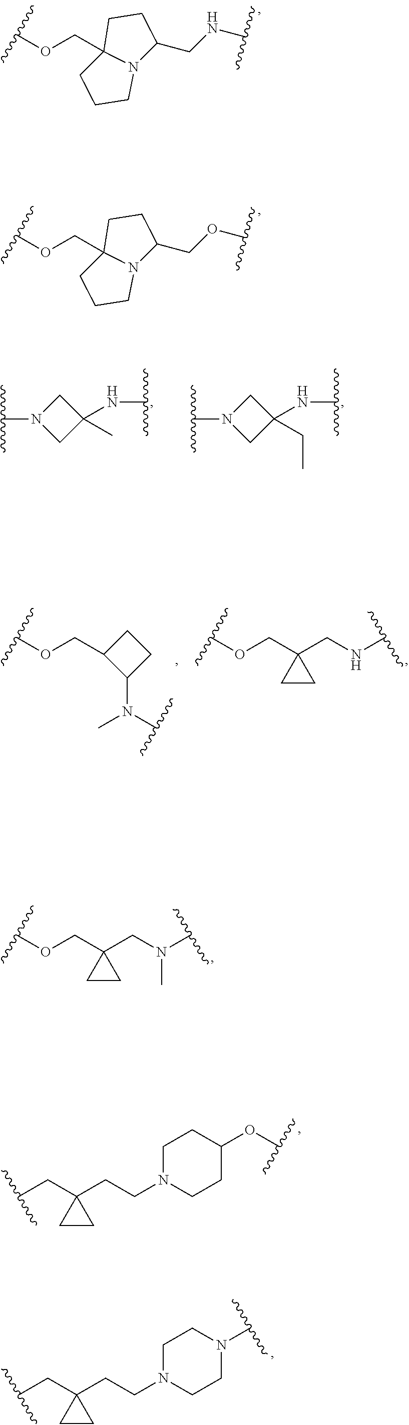

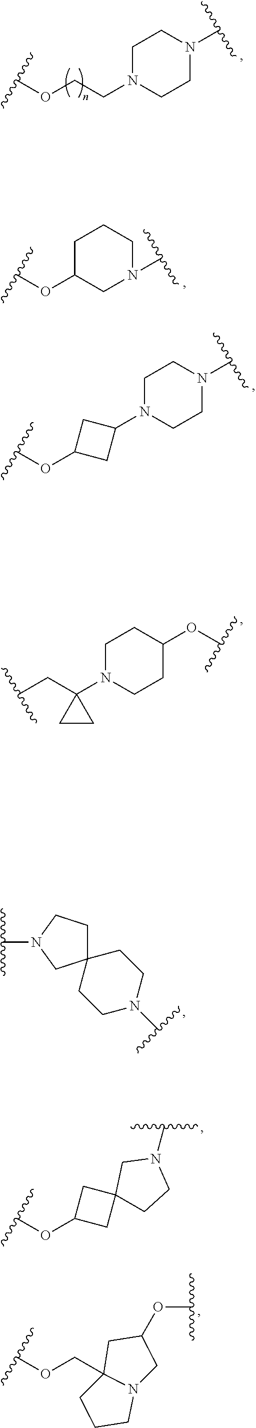

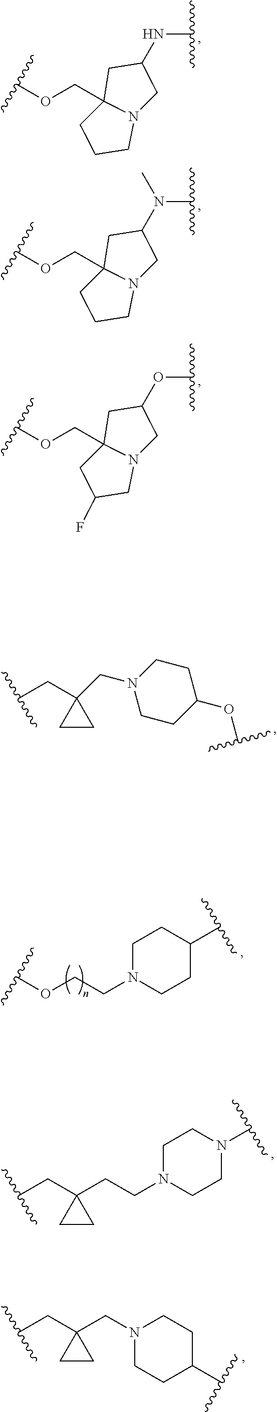

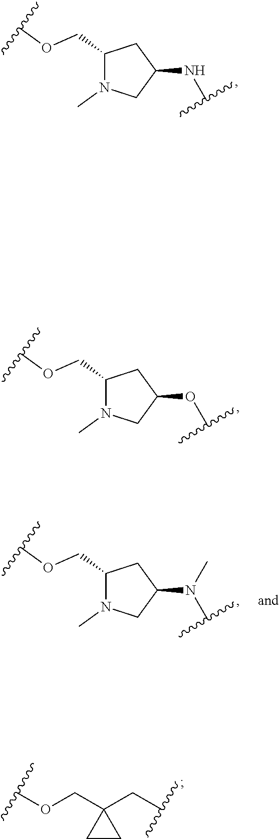

wherein, n is an integer from 0 to 20. In some embodiments, n is an integer from 0 to 5. In some embodiments, n is an integer from 1 to 2. In some embodiments, n is 1. In some embodiments, n is 2.

In some embodiments of compounds of the disclosure, L² and L³ (within the bivalent linking group) are independently selected from:

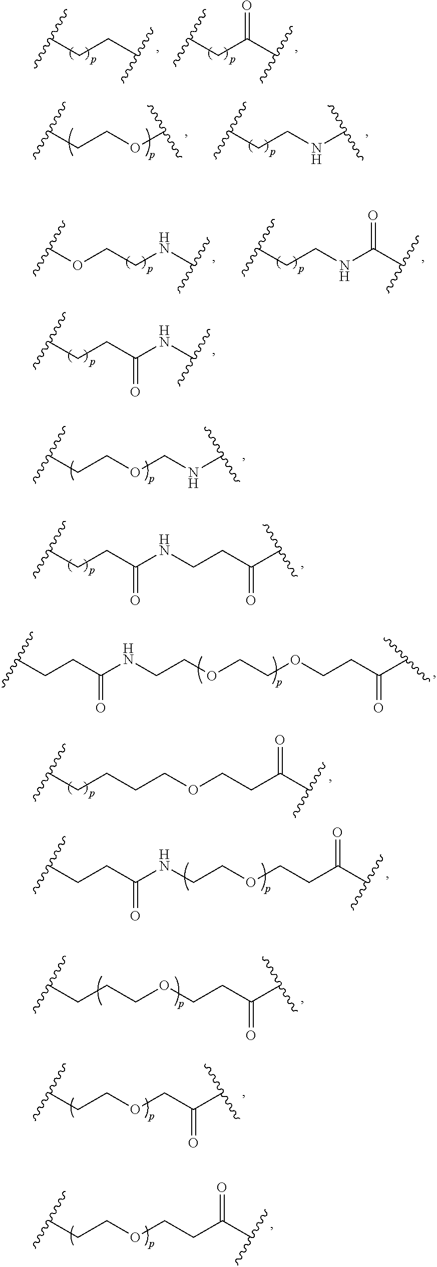

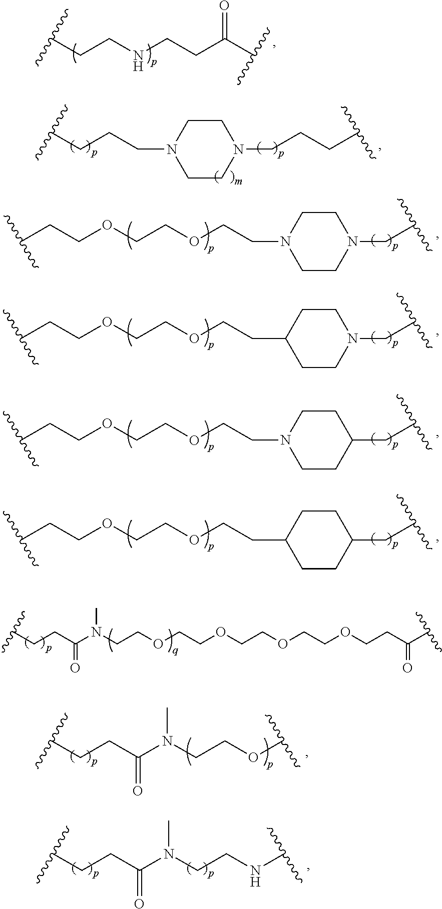

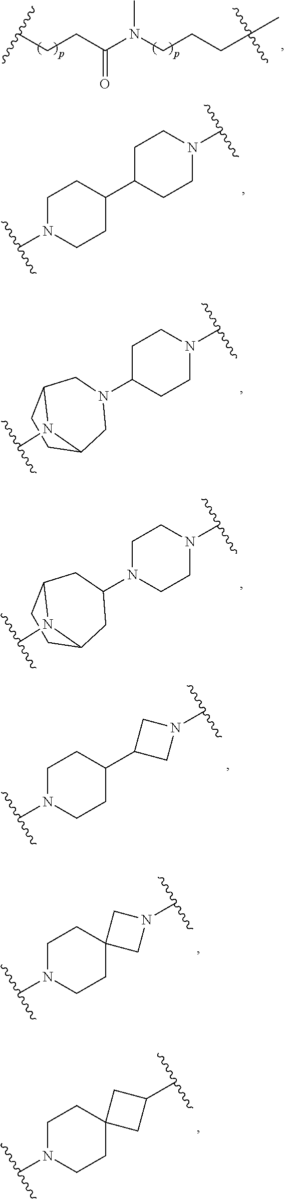

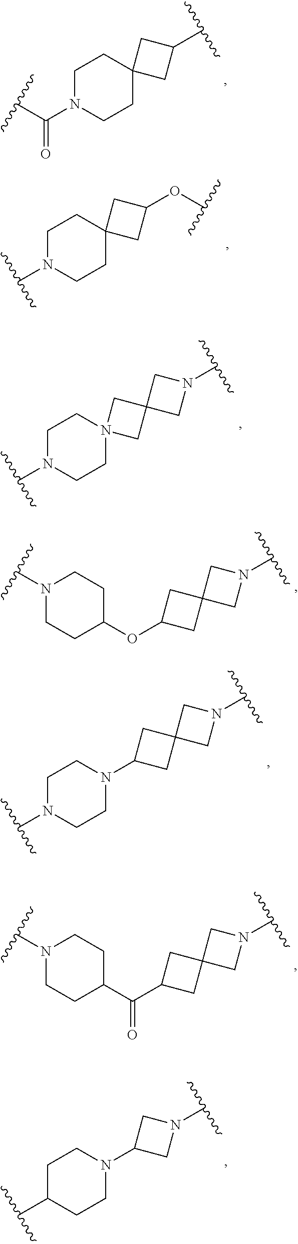

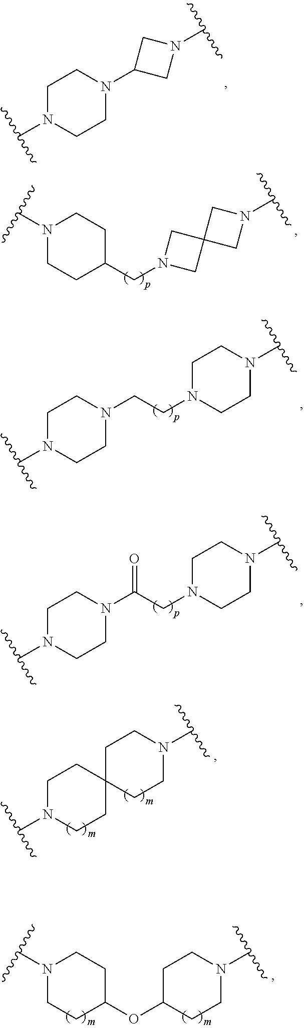

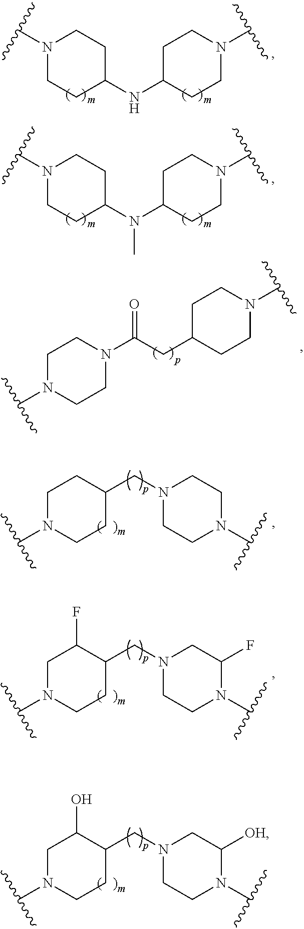

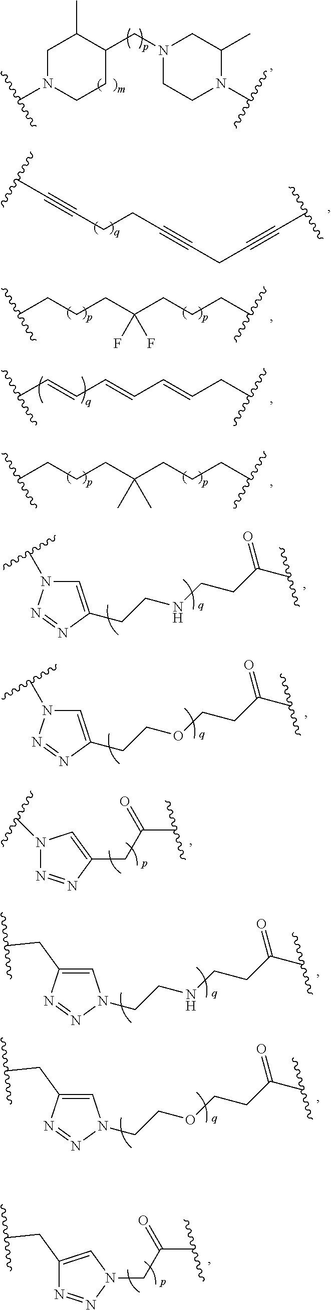

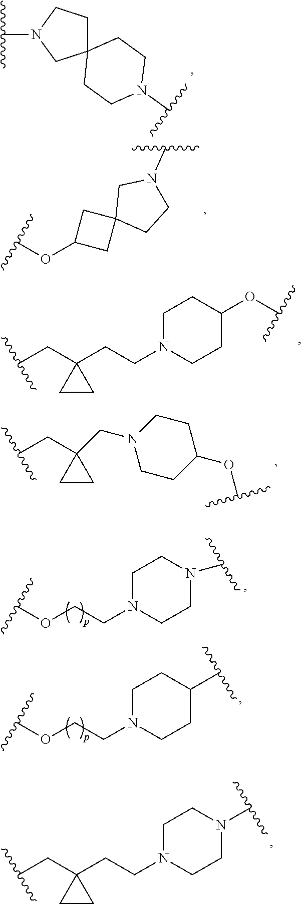

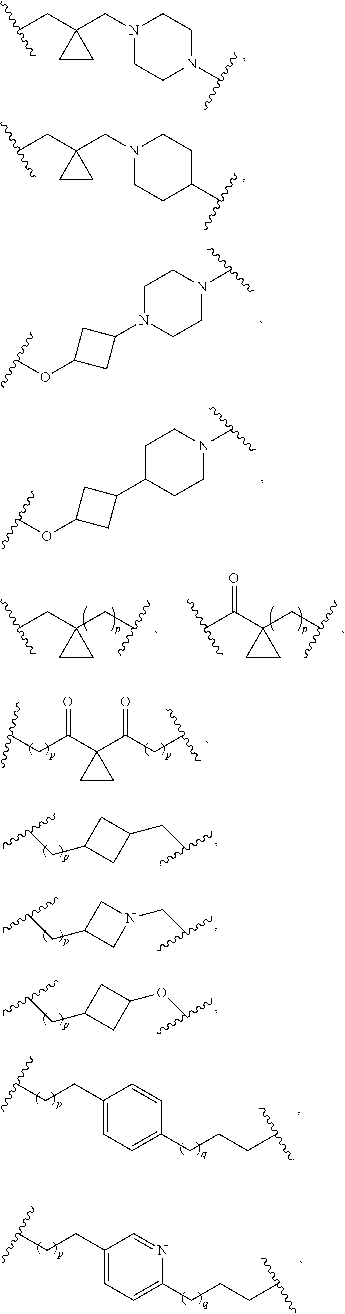

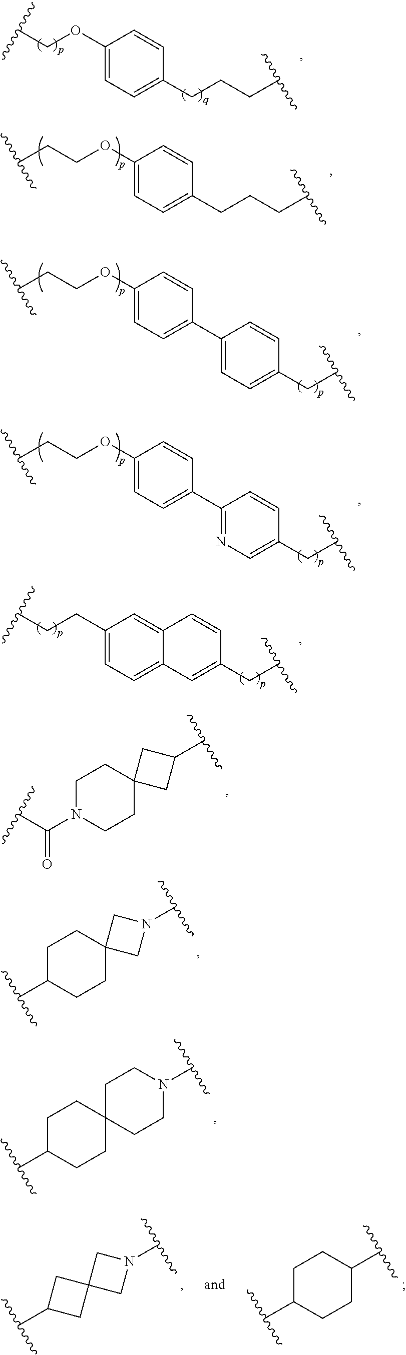

wherein, p is from 0-20, m is from 0-8, and q is from 0-10. In some embodiments, p is from 0-10 (i.e., p is 0, 1, 2, 3, 4, 5, 6, 7, 8, 9 or 10). In some embodiments, q is from 0-5 (i.e., q is 0, 1, 2, 3, 4 or 5).

In some embodiments of compounds of the disclosure, only one of L² and L³ is present (i.e., L² and L³ are not both present) within the bivalent linking group.

In some embodiments of compounds of the disclosure, L² and L³ together form one of the following structures.

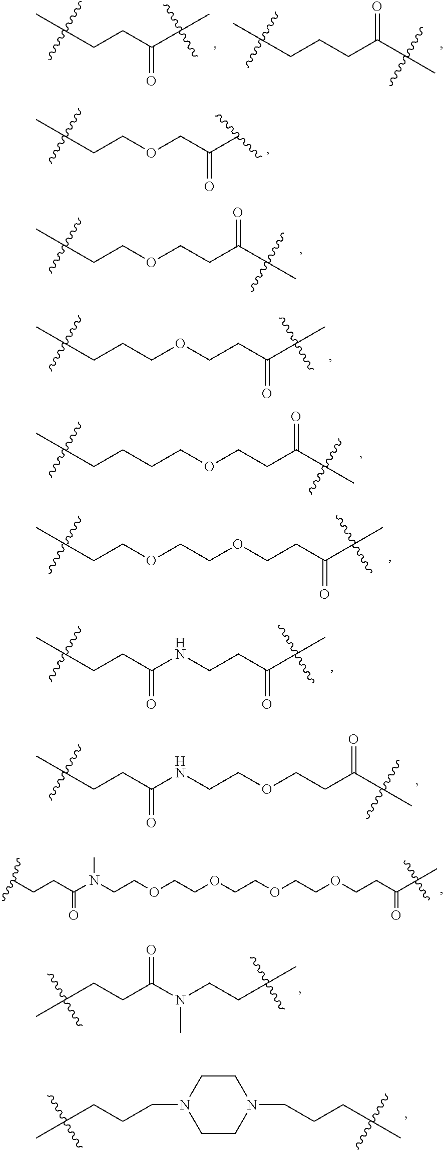

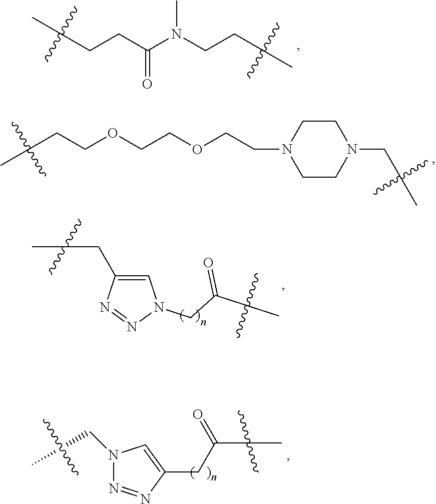

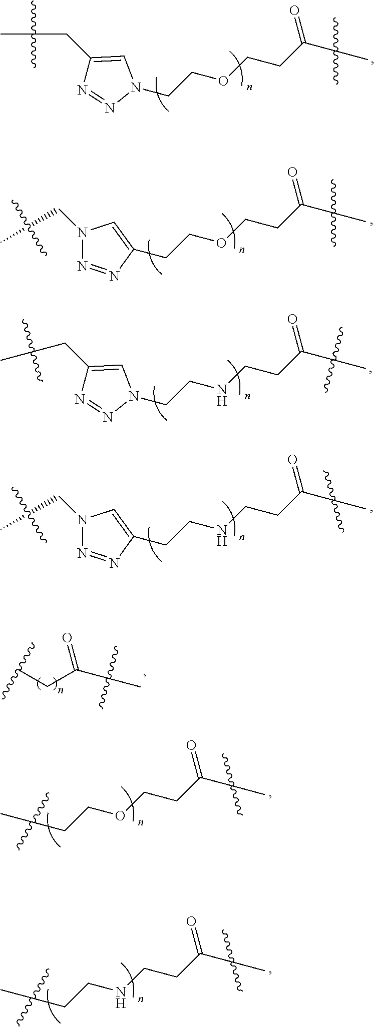

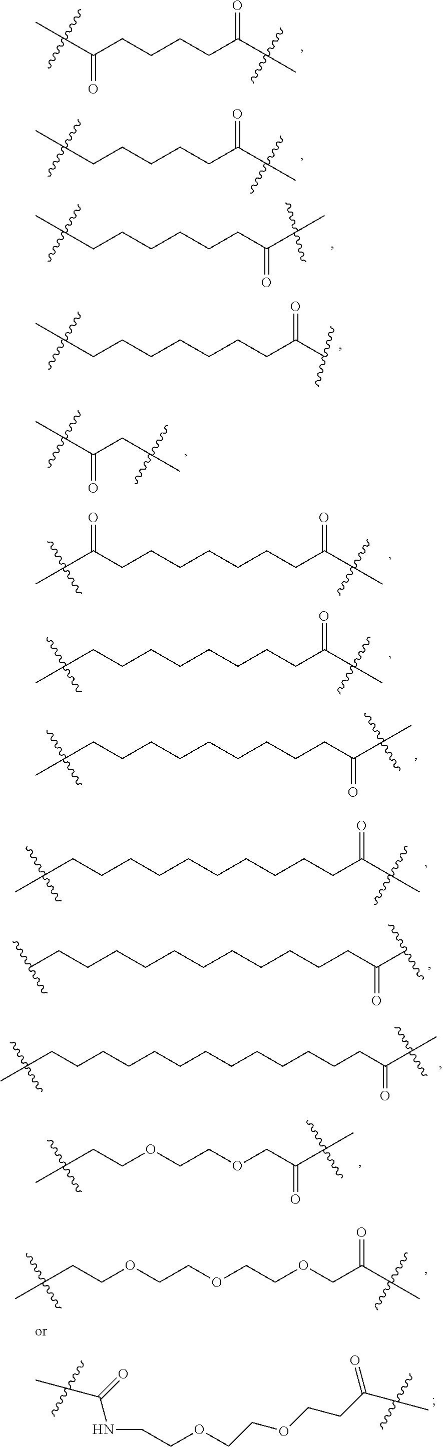

wherein n is a integer from 0 to 5 (i.e., n is 0, 1, 2, 3, 4 or 5).

In some embodiments of compounds of the disclosure, L¹, L² and L³ together form one of the following structures:

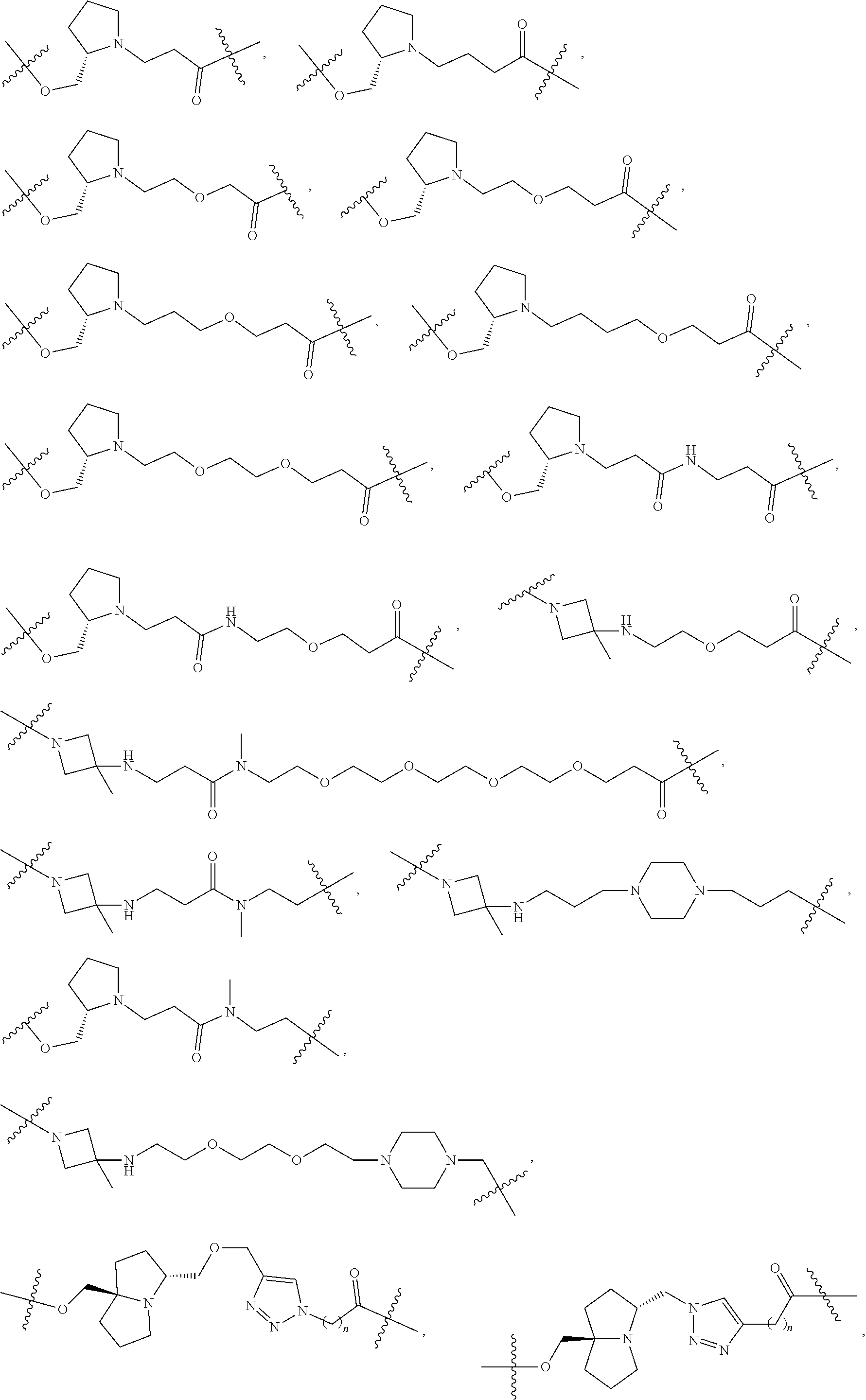

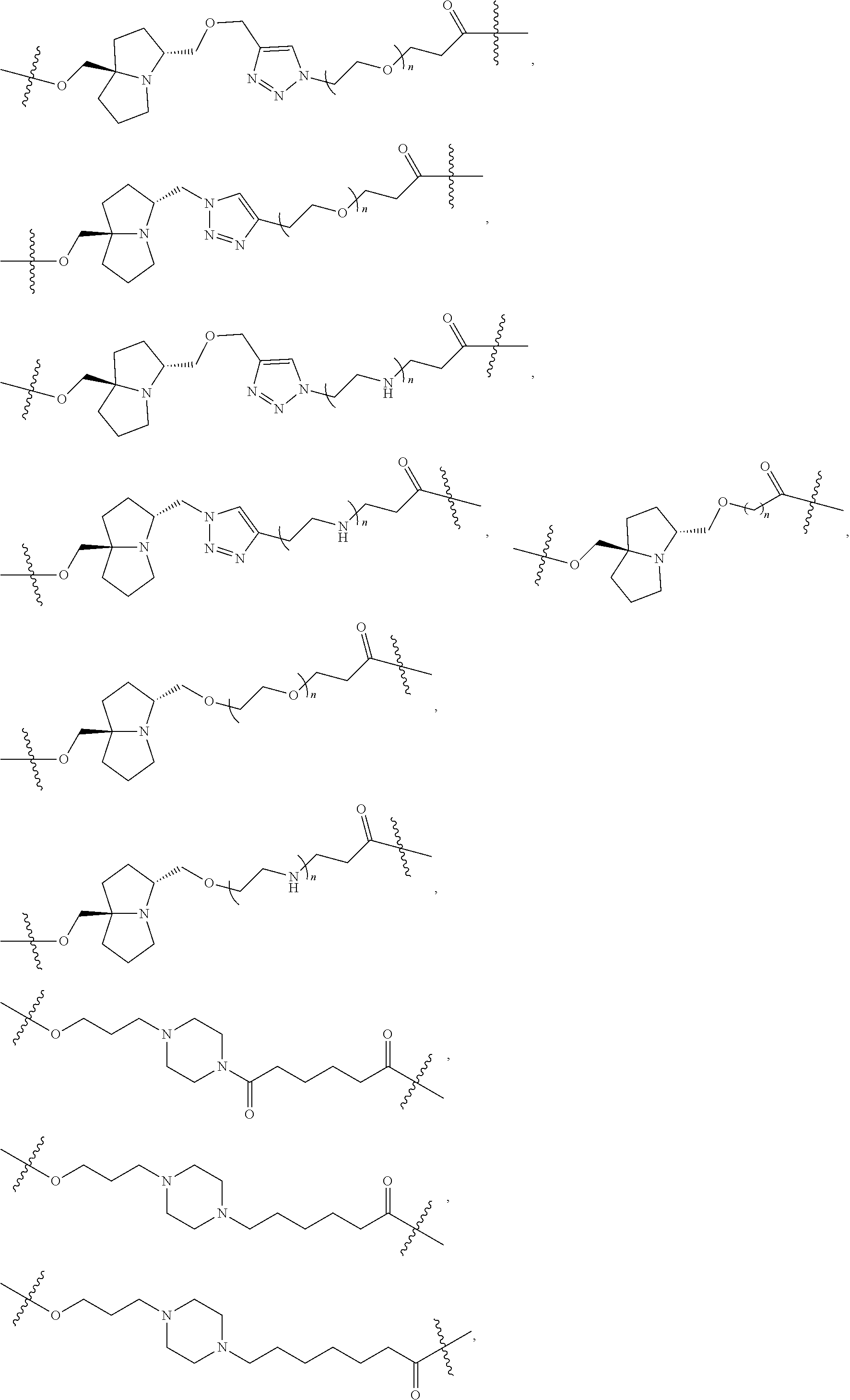

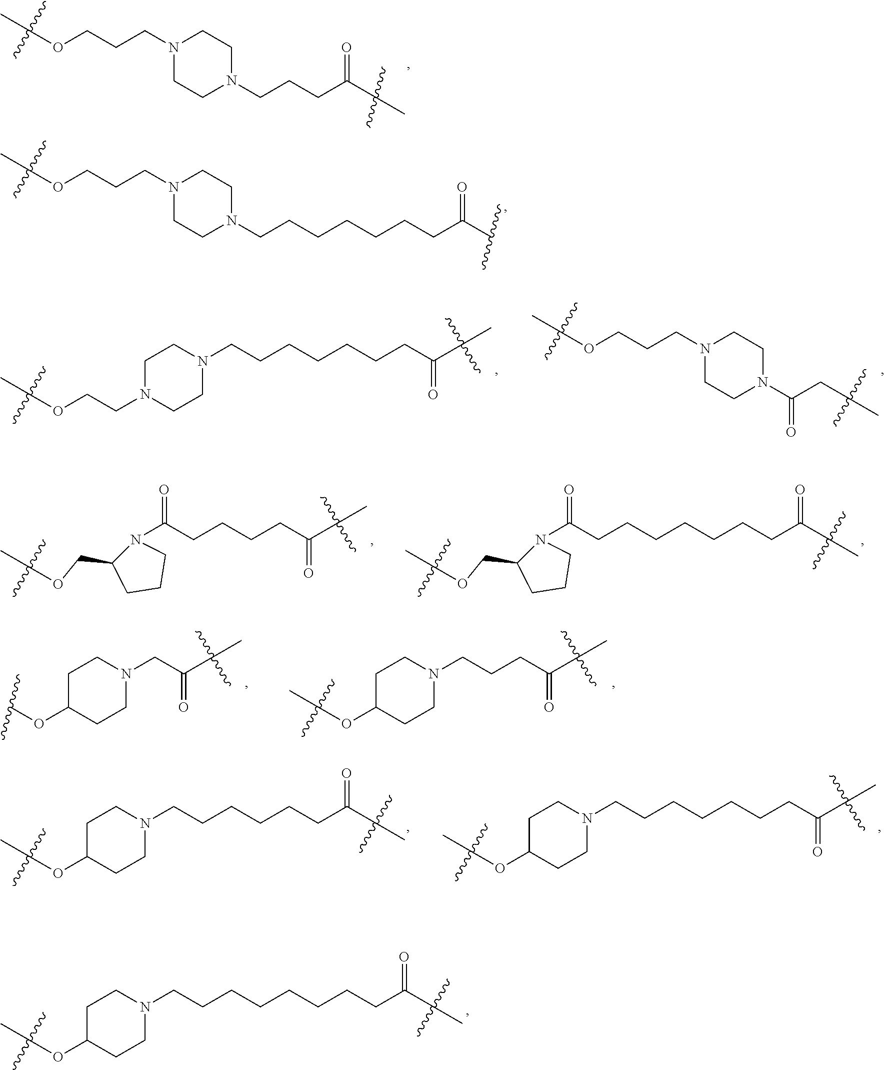

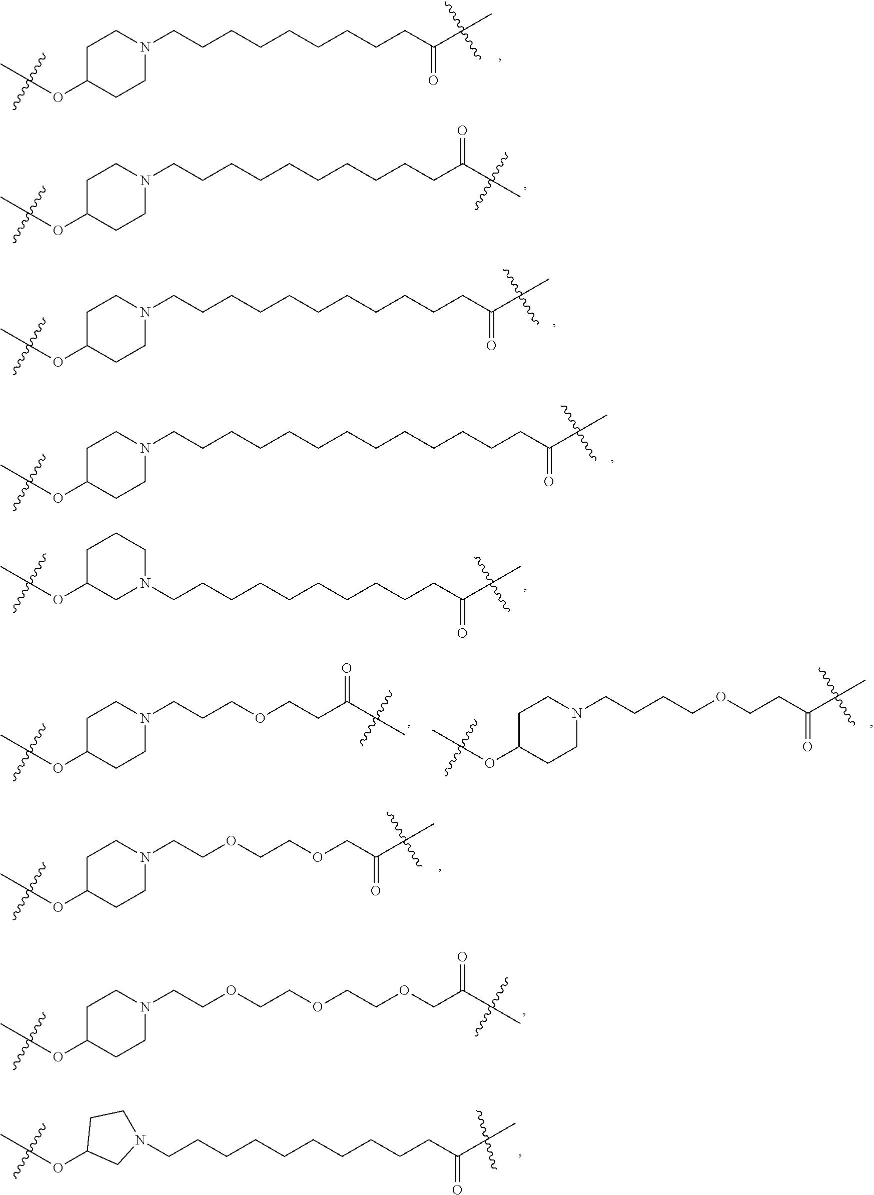

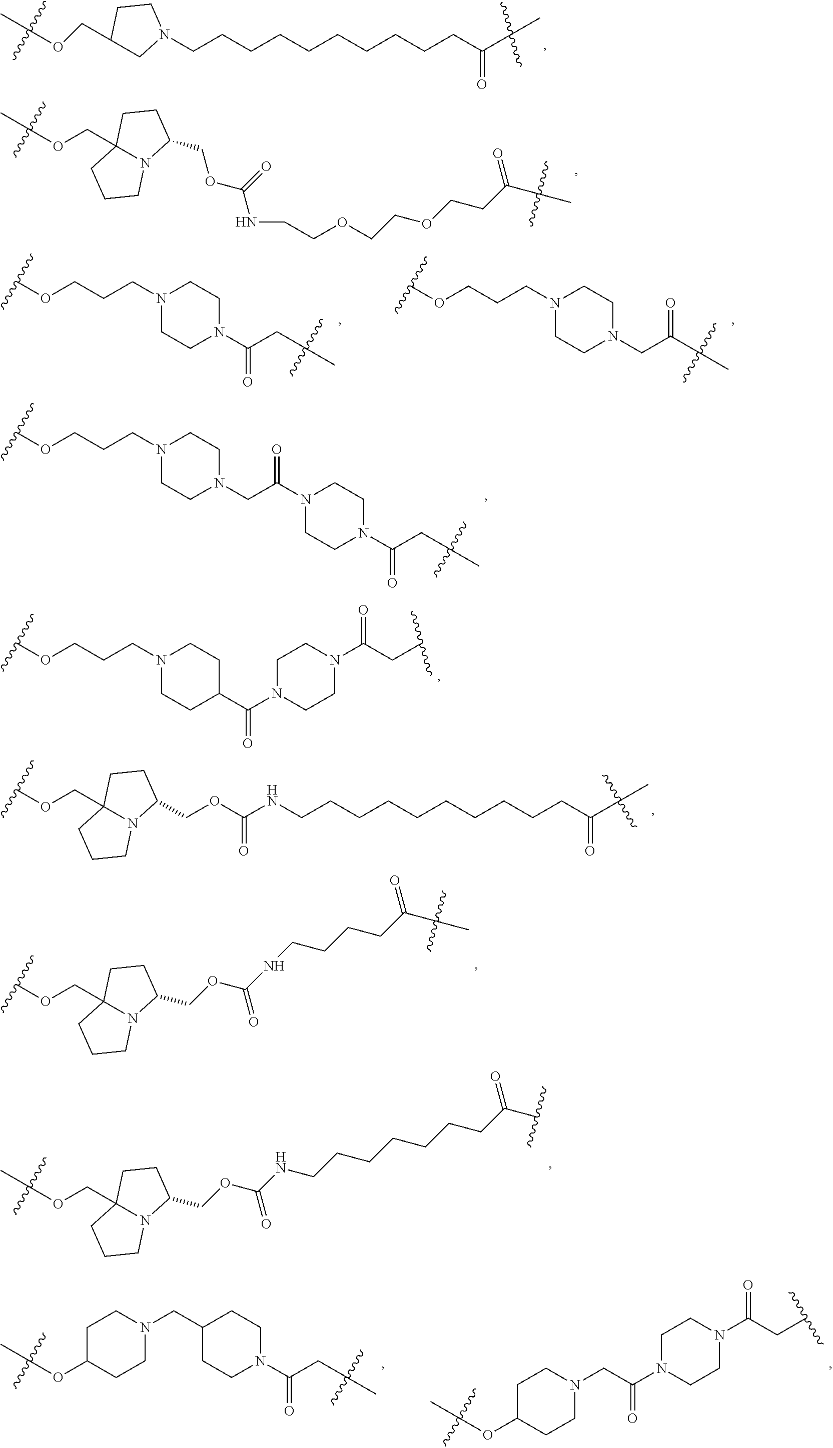

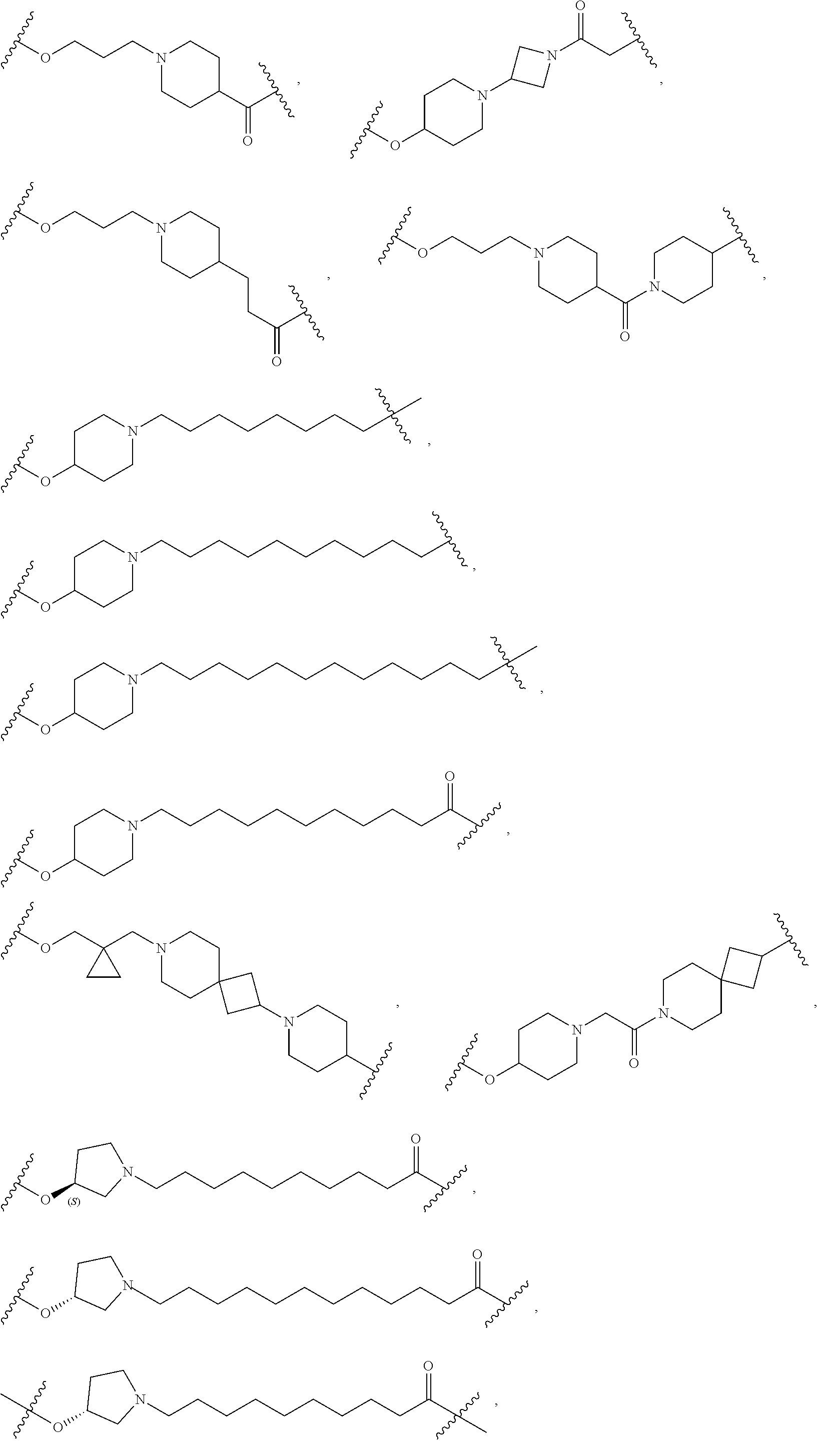

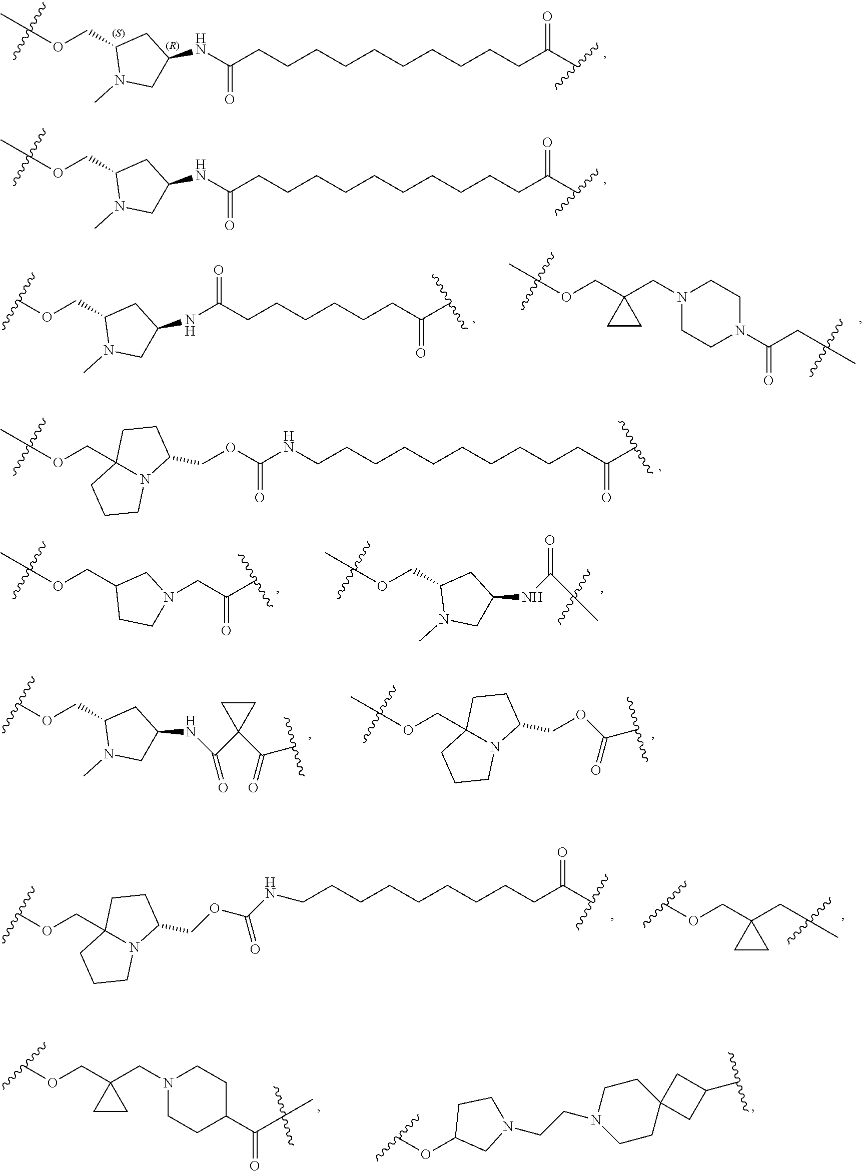

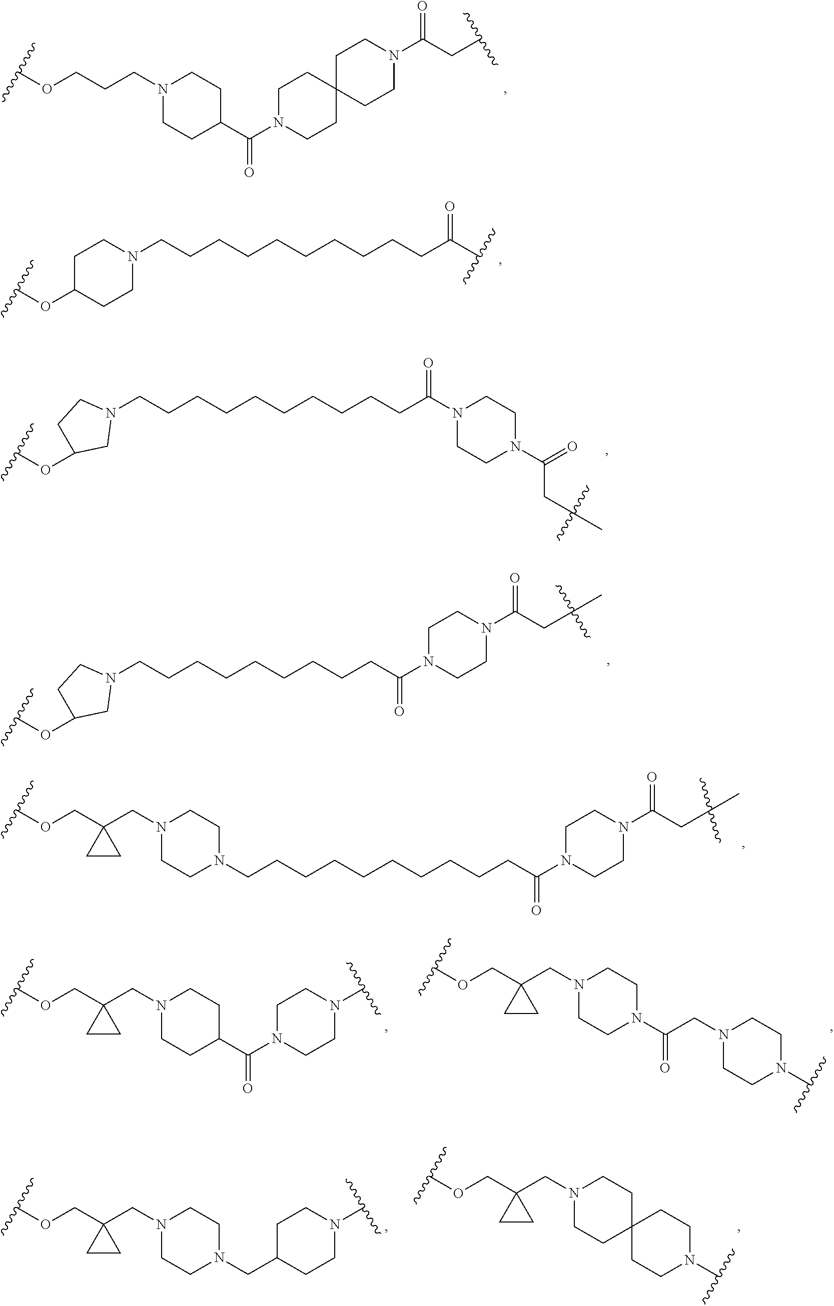

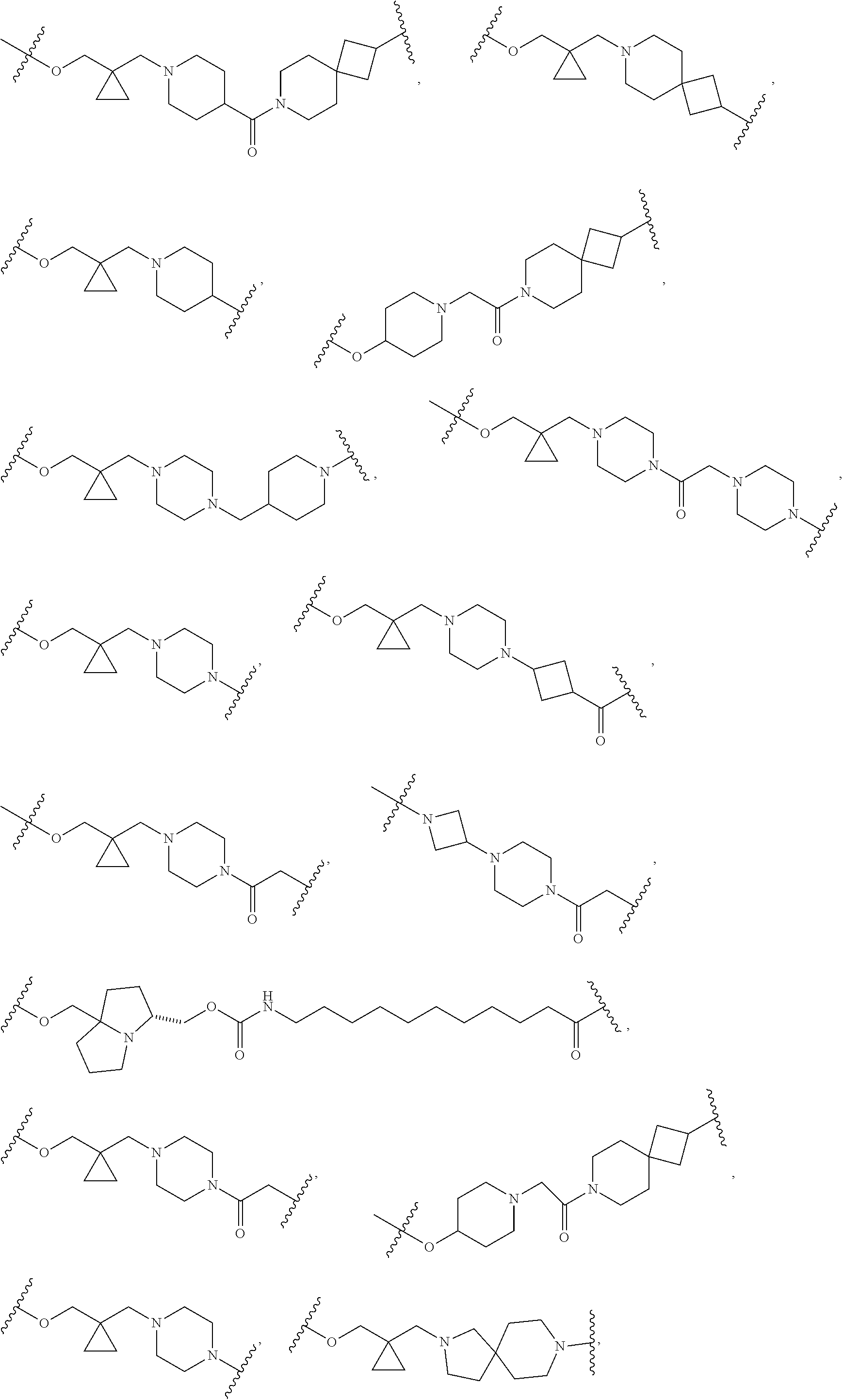

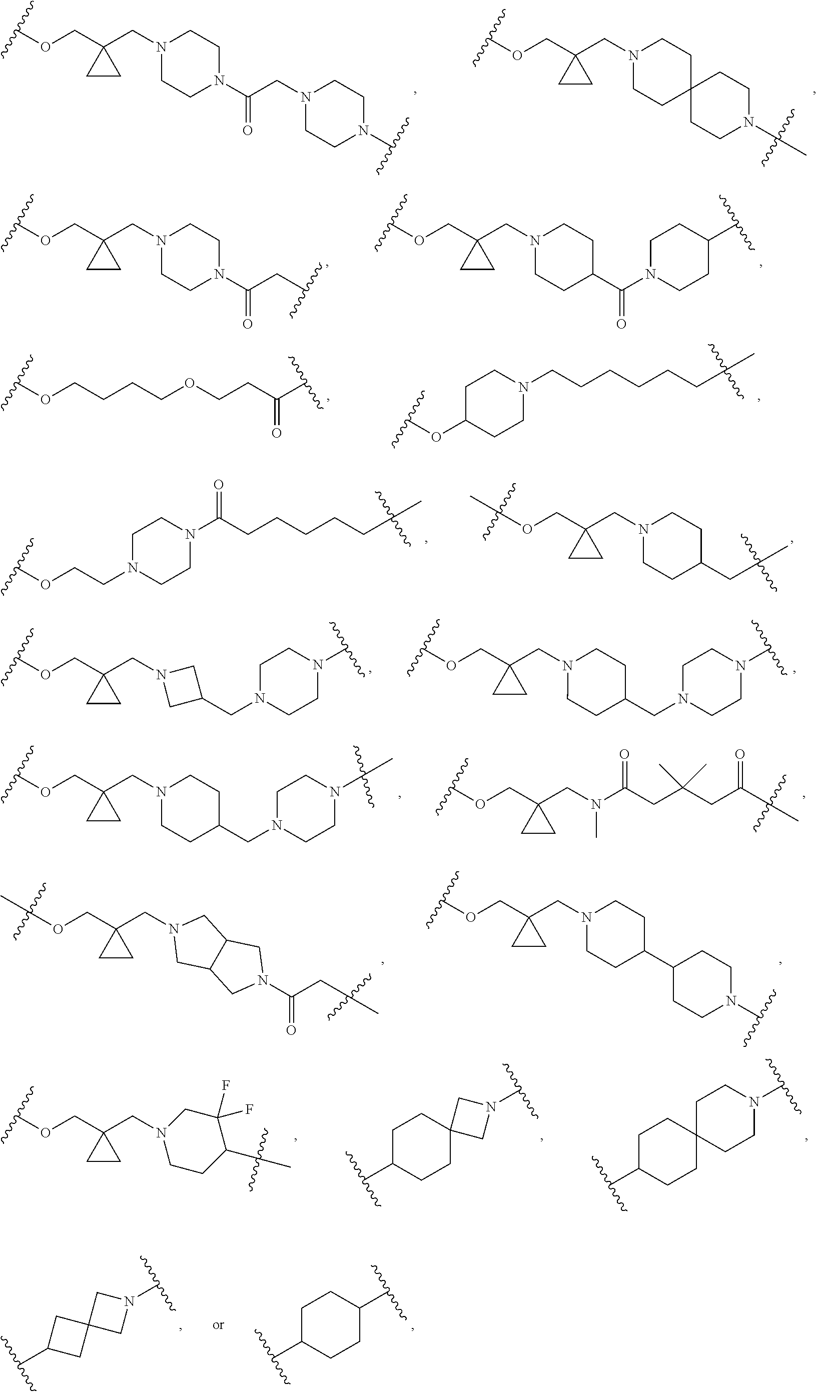

wherein, when the structure contains chiral centers, its stereochemical structures are independently the R-configuration, S-configuration, or a mixture of R and S configurations.

In certain embodiments of compounds of the disclosure, the E3 ligase binding group (T) binds specifically to an E3 ligase which is VHL (Von Hippel-Lindau), CRBN (Cereblon), MDM2, c-IAP1, AhR, Nimbolide, CCW16, KB02, KEAP1, beta-TrCP1, DCAF15, DCAF16, RNF114, or another E3 ligase. In some embodiments, the E3 ligase binding group (T) binds specifically to an E3 ligase which is VHL (Von Hippel-Lindau), CRBN (Cereblon), MDM2, c-IAP1, AhR, Nimbolide, CCW16, KB02 or KEAP1. In some embodiments, the E3 ligase binding group (T) binds specifically to an E3 ligase which is VHL. In some embodiments, the E3 ligase binding group (T) binds specifically to an E3 ligase which is CRBN.

In certain embodiments of compounds of the disclosure, the E3 ligase binding group T is selected from:

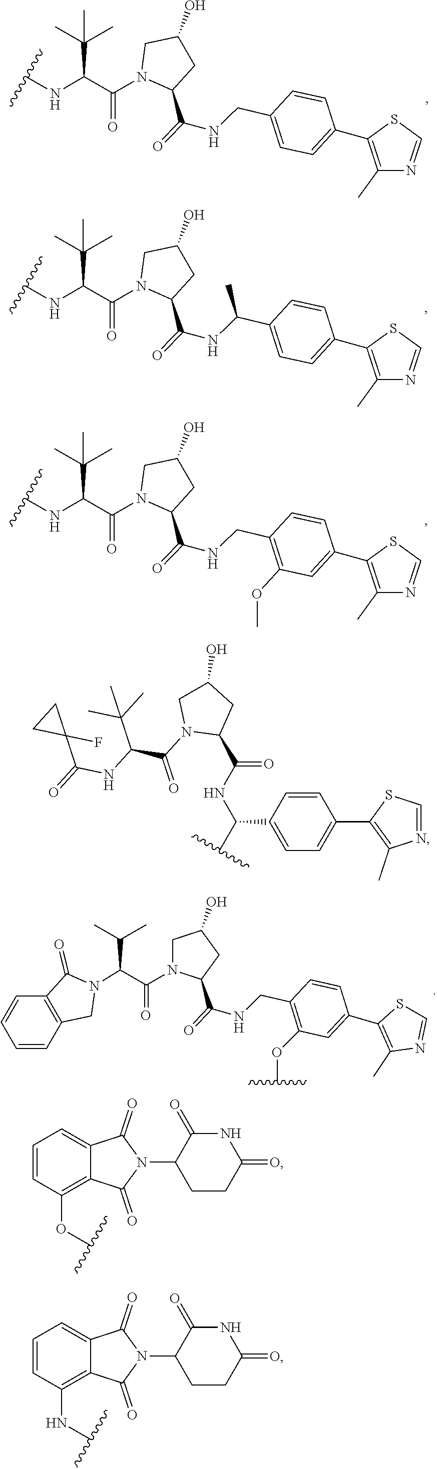

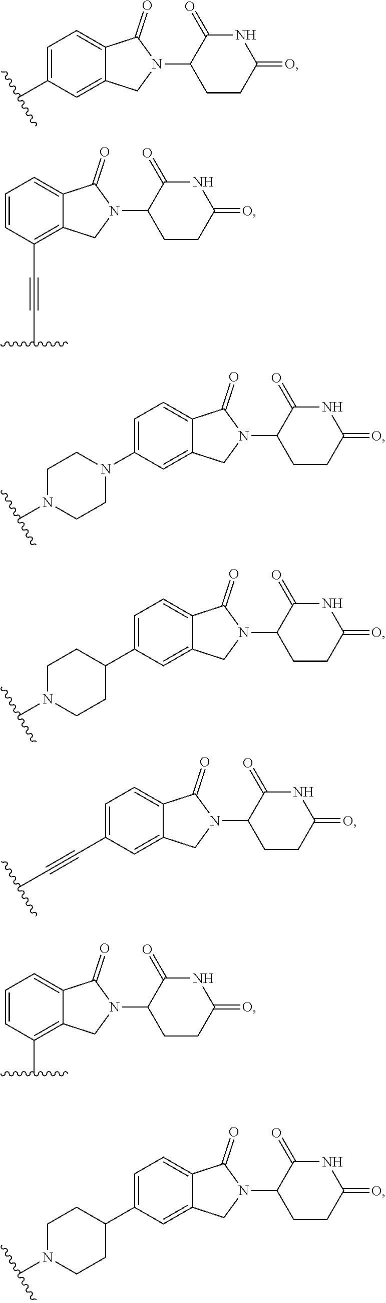

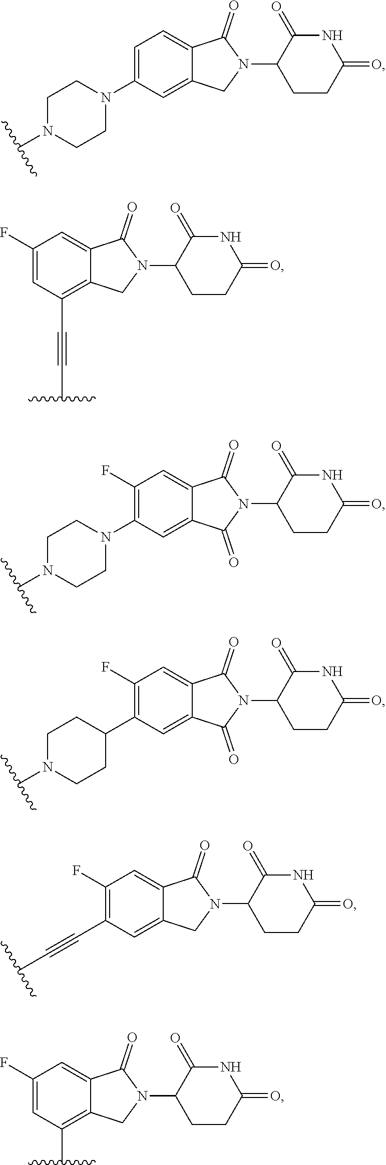

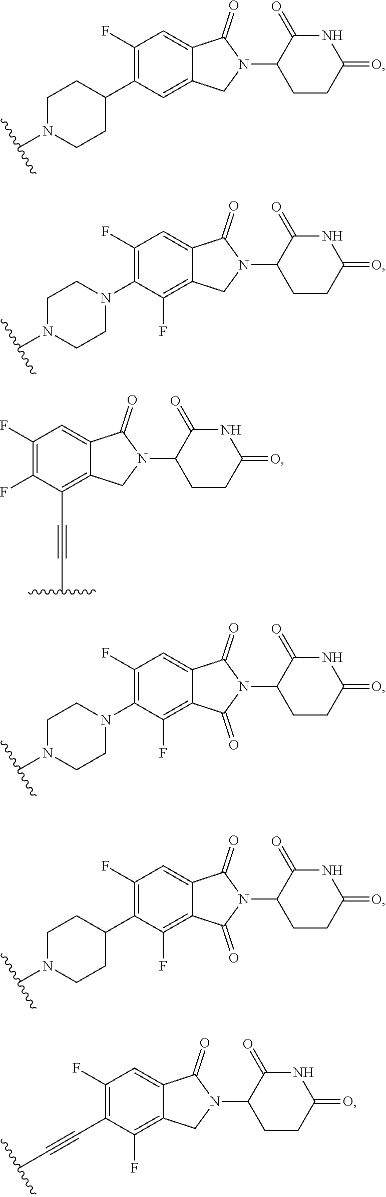

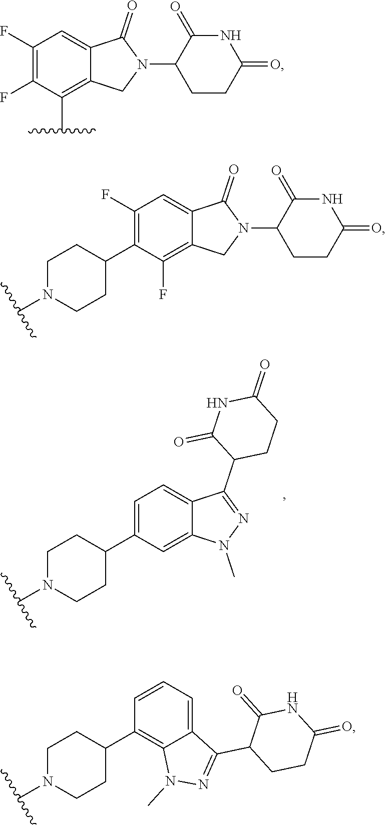

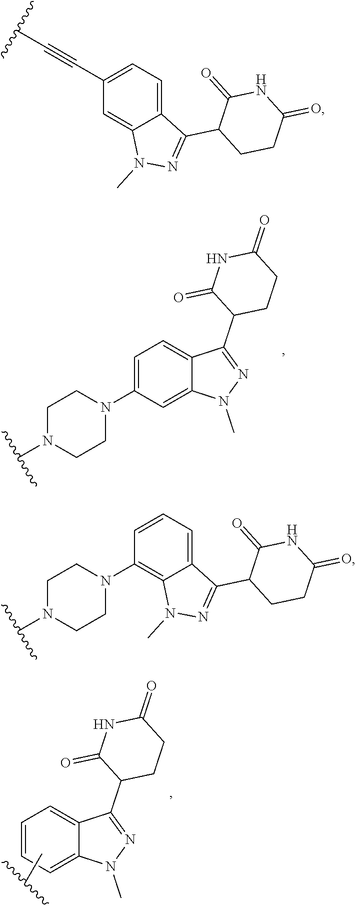

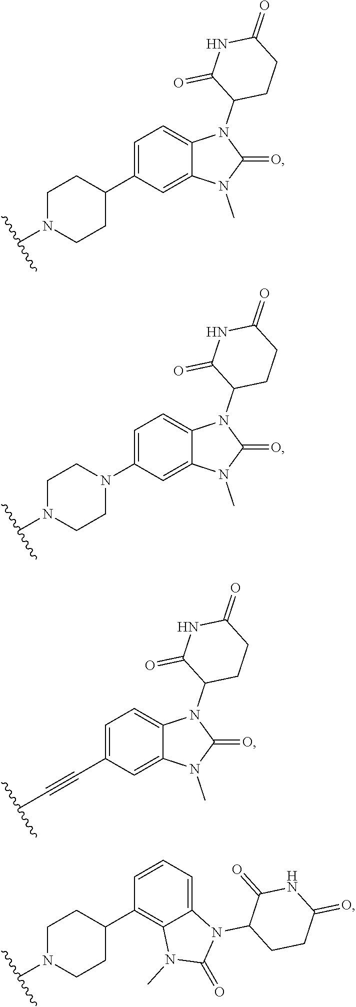

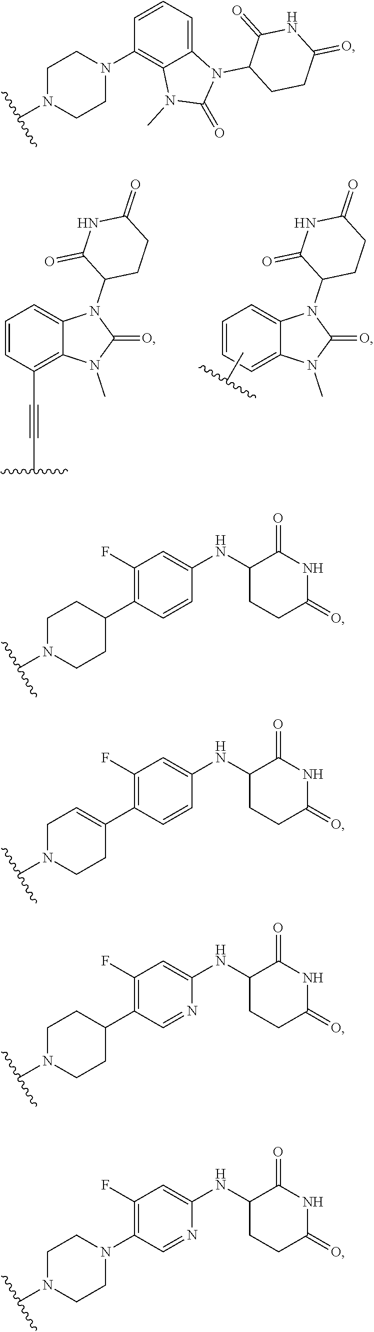

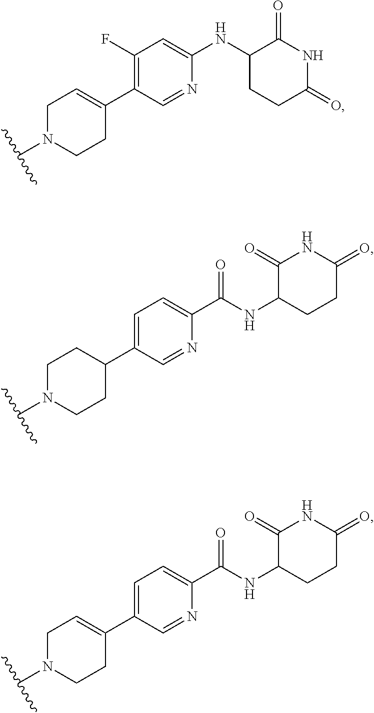

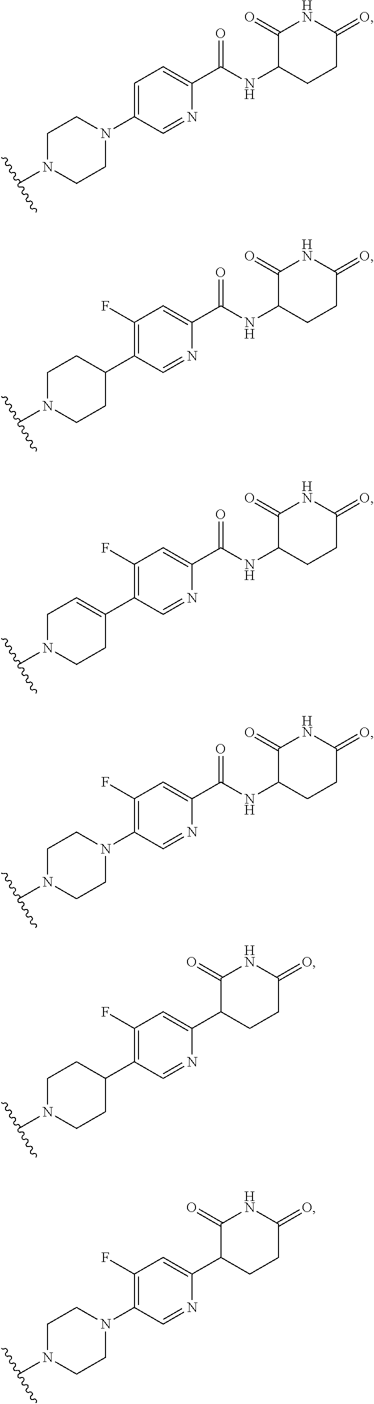

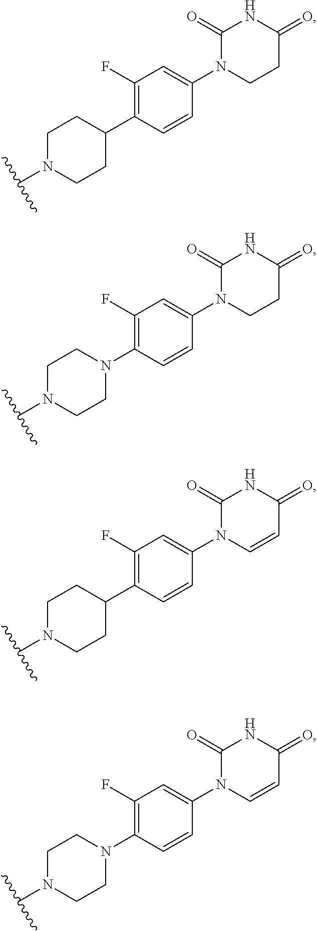

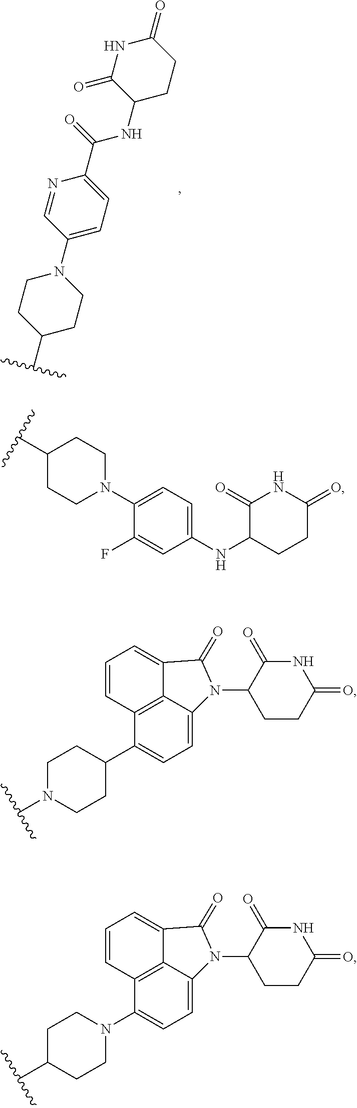

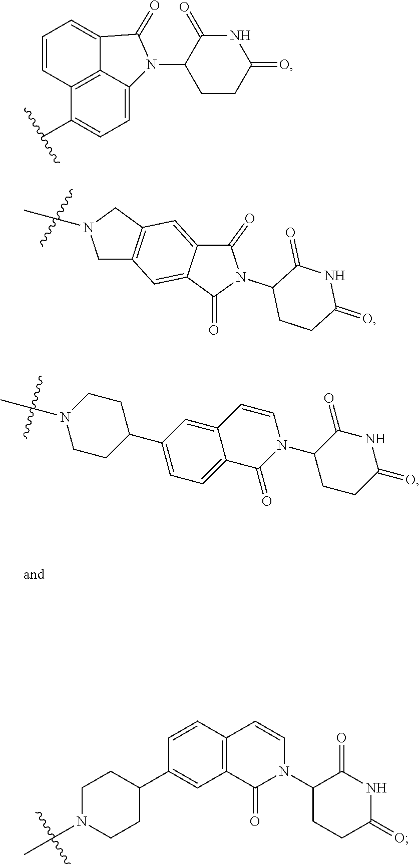

wherein, when the structure contains chiral centers, its stereochemical structures are independently the R-configuration, S-configuration, or a mixture of R and S configurations.

In certain embodiments of compounds of the disclosure, the E3 ligase binding group T is selected from:

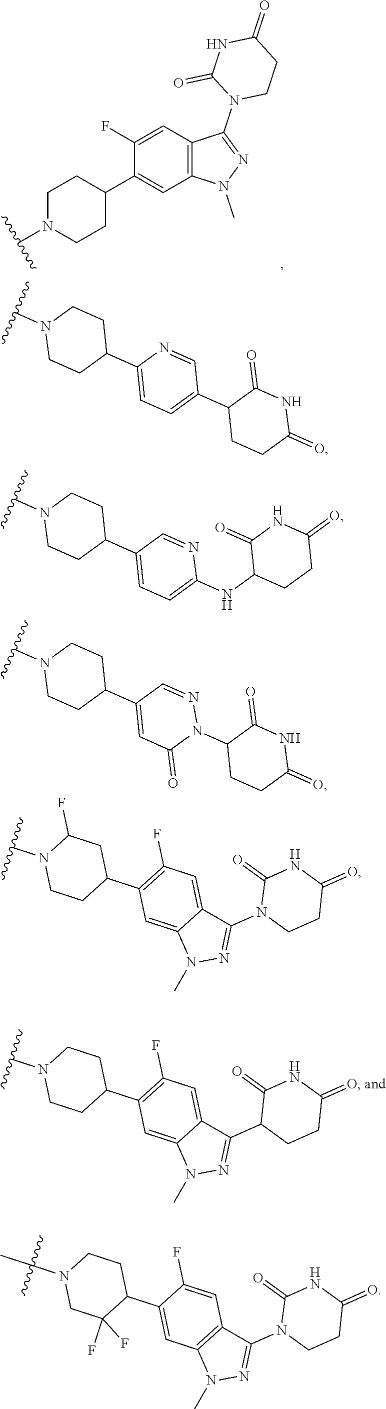

In some embodiments of bifunctional compounds of the disclosure, the compound is a compound of Formula (A-2), or a pharmaceutically acceptable salt, ester, stereoisomer, hydrate or solvate thereof:

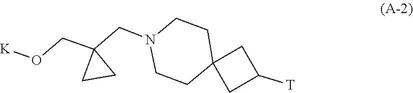

wherein:

K is

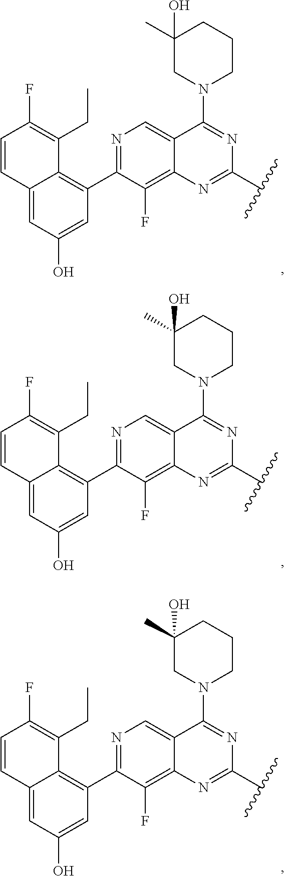 [SMILES: *c1nc(N2CCCC(C)(O)C2)c2cnc(-c3cc(O)cc4ccc(F)c(CC)c34)c(F)c2n1 ; *c1nc(N2CCC[C@](C)(O)C2)c2cnc(-c3cc(O)cc4ccc(F)c(CC)c34)c(F)c2n1 ; *c1nc(N2CCC[C@@](C)(O)C2)c2cnc(-c3cc(O)cc4ccc(F)c(CC)c34)c(F)c2n1]

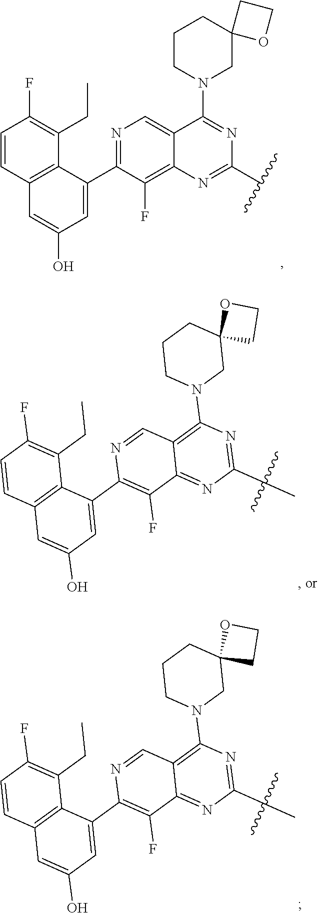 [SMILES: *c1nc(N2CCCC3(CCO3)C2)c2cnc(-c3cc(O)cc4ccc(F)c(CC)c34)c(F)c2n1 ; *c1nc(N2CCC[C@]3(CCO3)C2)c2cnc(-c3cc(O)cc4ccc(F)c(CC)c34)c(F)c2n1 ; *c1nc(N2CCC[C@@]3(CCO3)C2)c2cnc(-c3cc(O)cc4ccc(F)c(CC)c34)c(F)c2n1]

and

T is

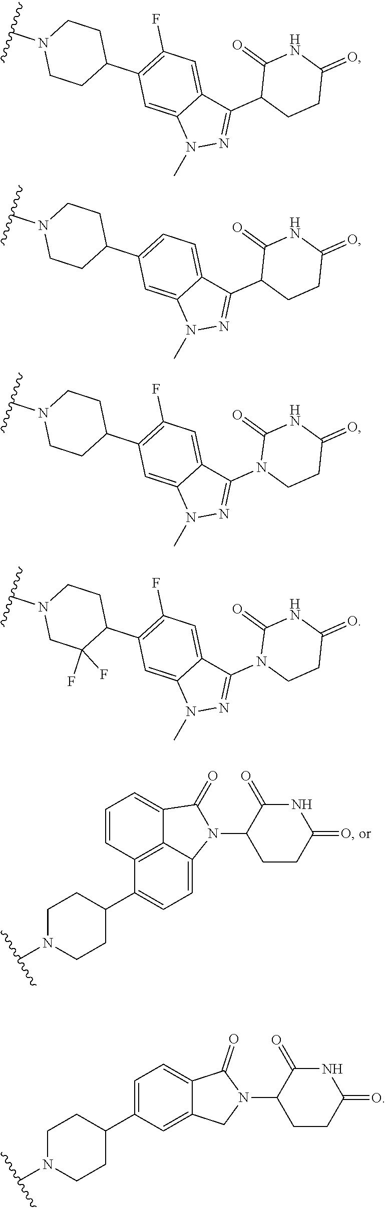

In some embodiments of bifunctional compounds of the disclosure, the compound is a compound shown in Table 2, or a pharmaceutical acceptable salt, ester, stereoisomer, hydrate or solvate thereof, wherein n is an integer from 0 to 5 (i.e., n is 0, 1, 2, 3, 4, or 5).

In some embodiments of bifunctional compounds of the disclosure, the compound is a compound shown in Table 3, or a pharmaceutical acceptable salt, ester, stereoisomer, hydrate or solvate thereof, wherein n is an integer from 0 to 5 (i.e., n is 0, 1, 2, 3, 4, or 5).

TABLE 2
Exemplary bifunctional compounds of the disclosure in accordance with certain embodiments.

| Com- | |
|---|---|
| pound | |
| No. | Structure |
| 1 | 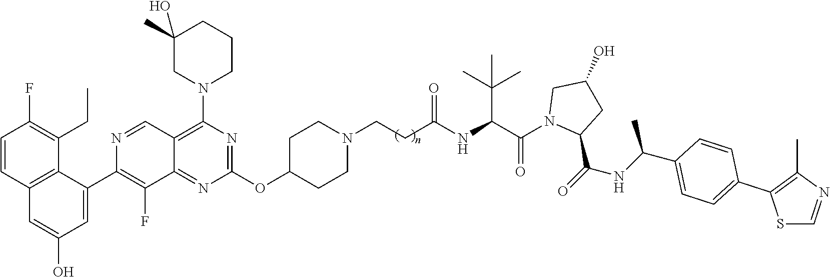 |
| 2 | 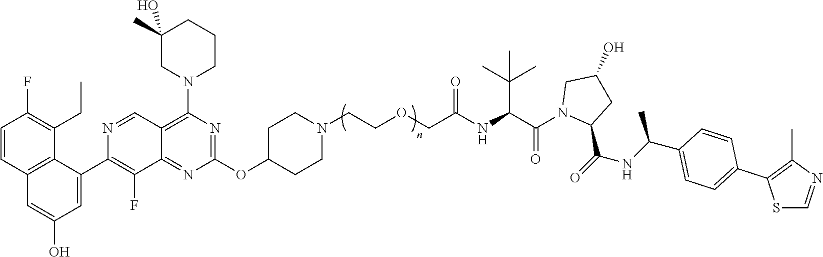 |
| 3 | 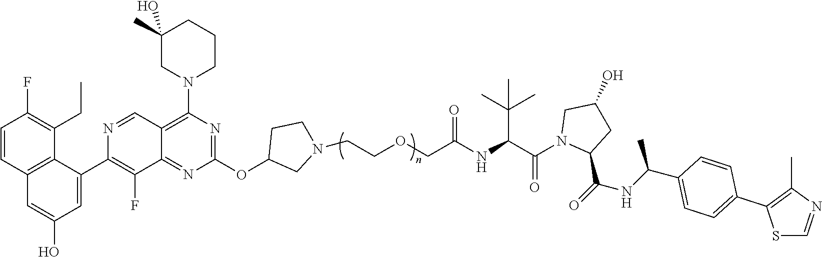 |
| 4 | 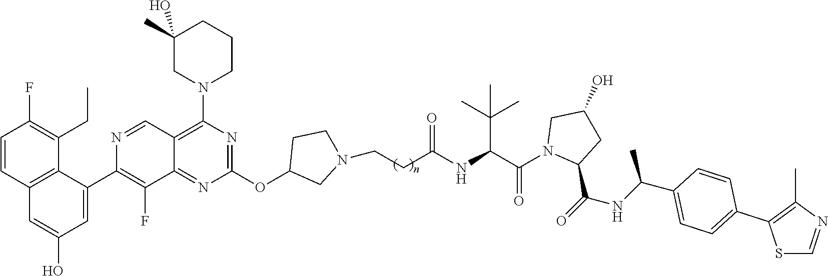 |
| 5 | 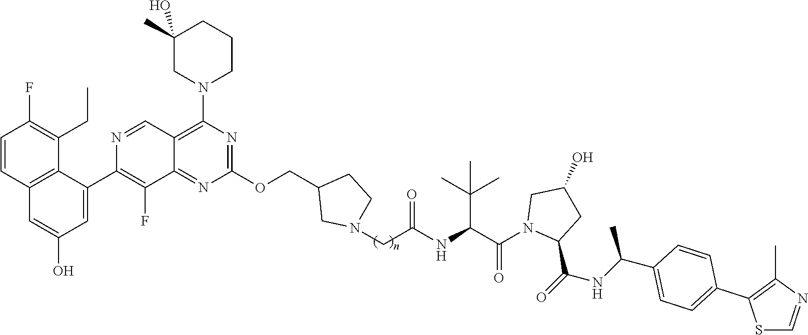 |
| 6 | 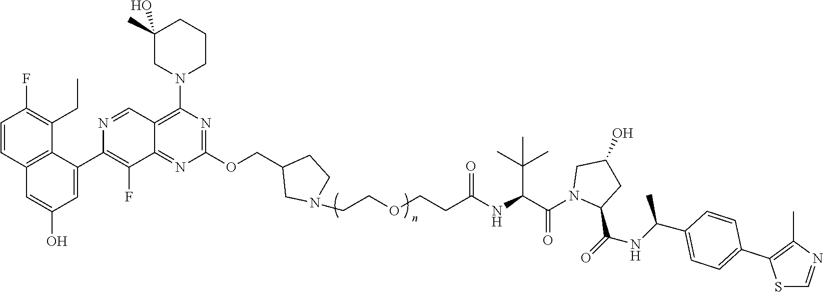 |
| 7 | 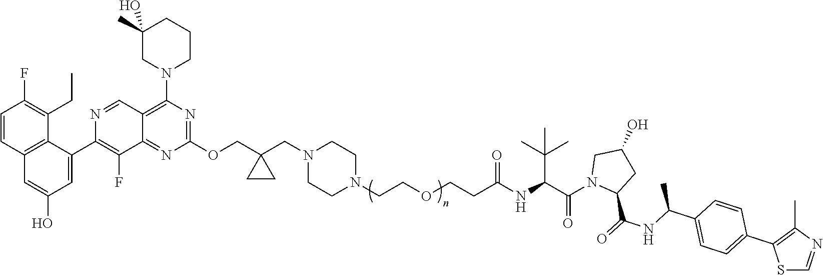 |
| 8 | 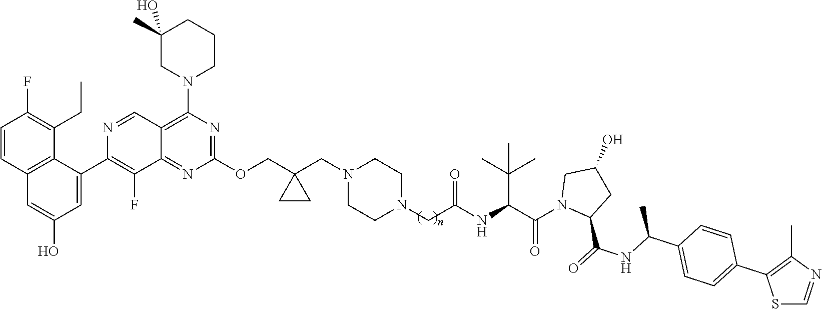 |
| 9 | 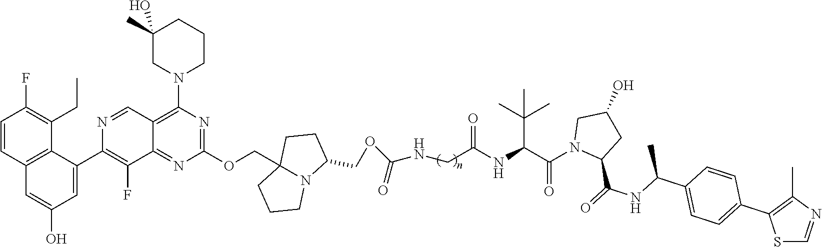 |
| 10 | 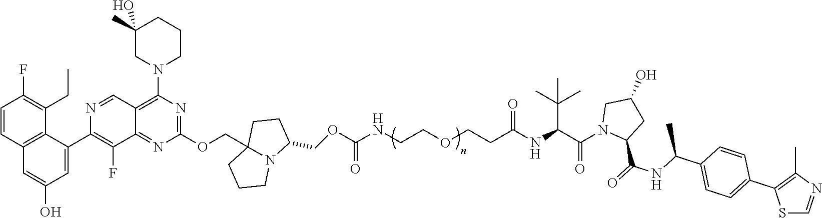 |
| 11 | 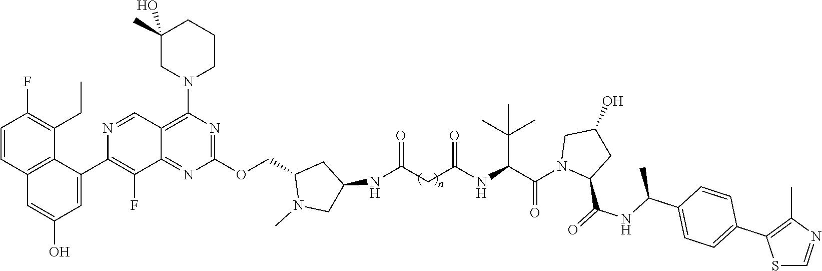 |
| 12 | 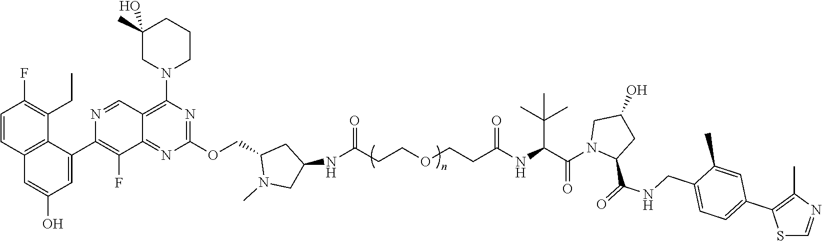 |
| 13 | 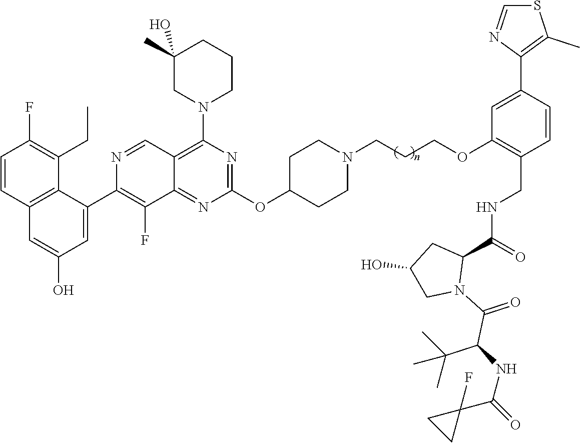 |
| 14 | 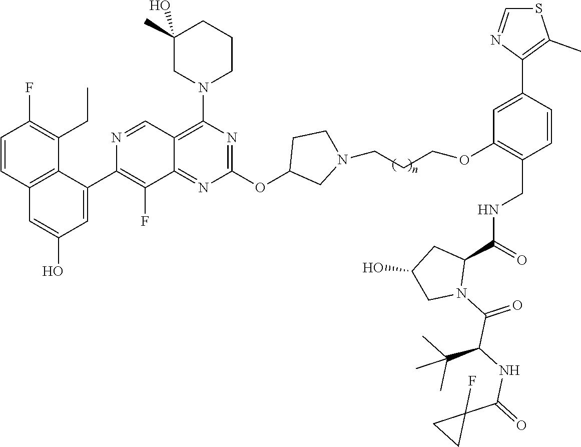 |
| 15 | 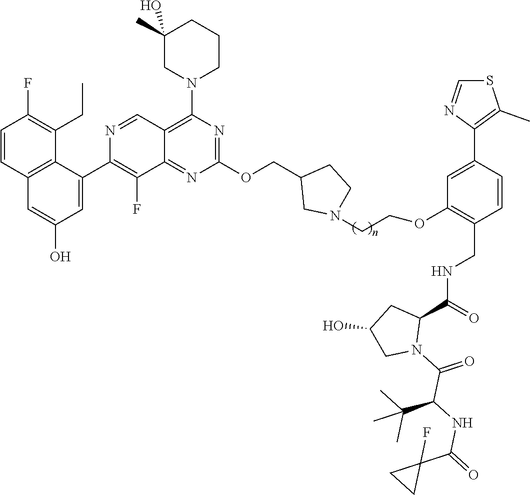 |
| 16 | 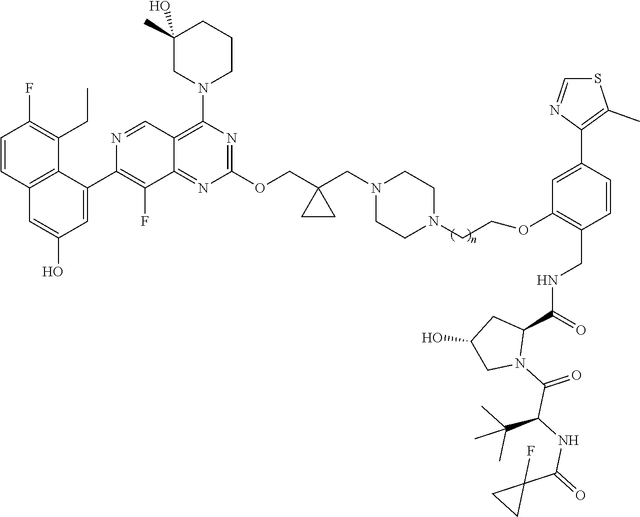 |
| 17 | 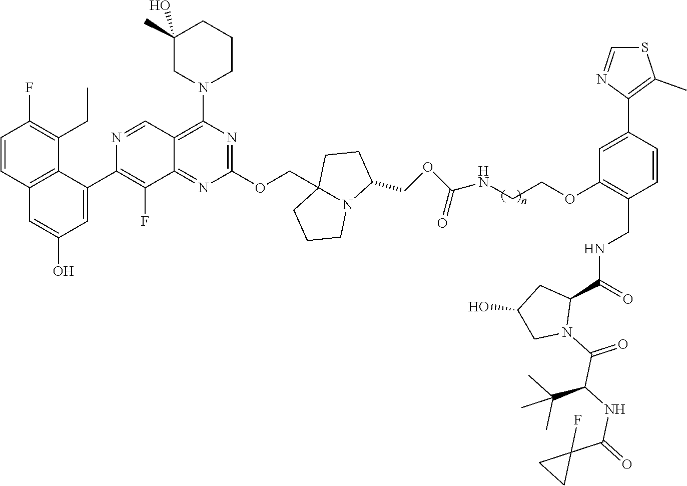 |
| 18 | 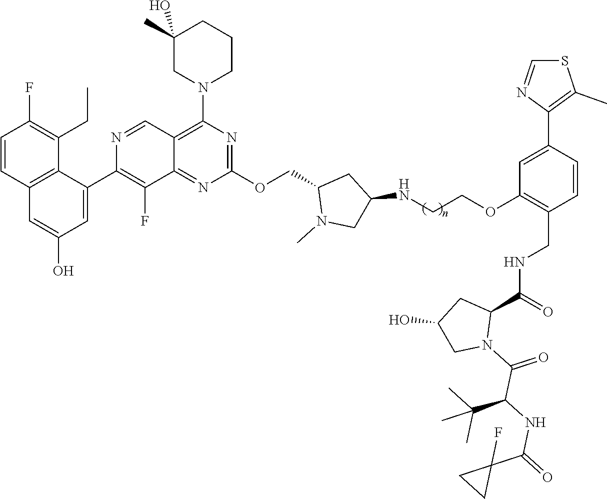 |
| 19 | 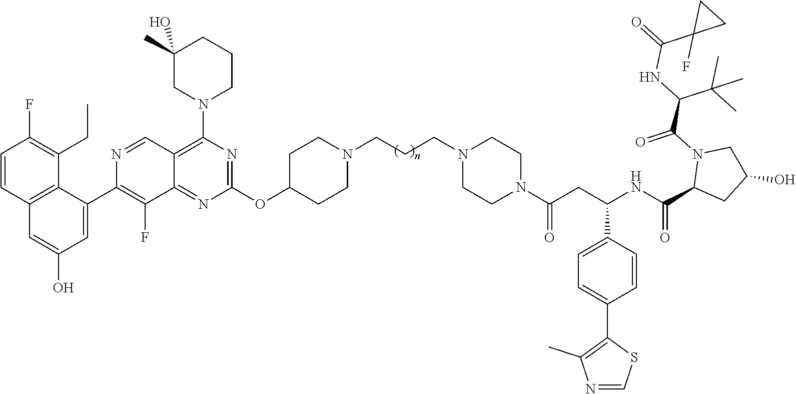 |
| 20 | 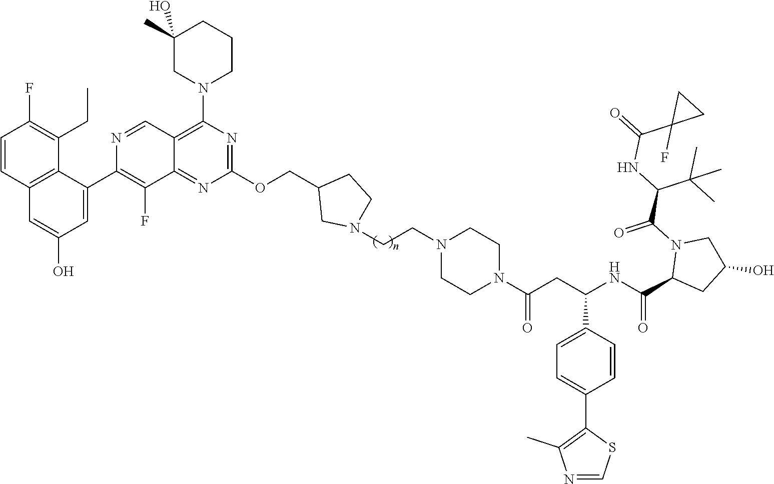 |
| 21 | 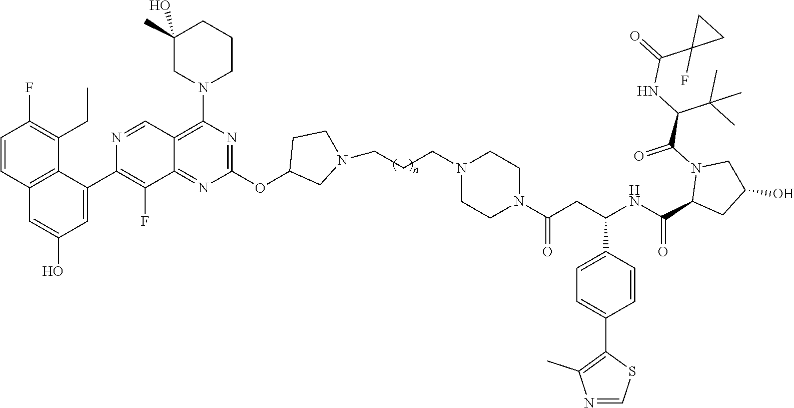 |
| 22 | 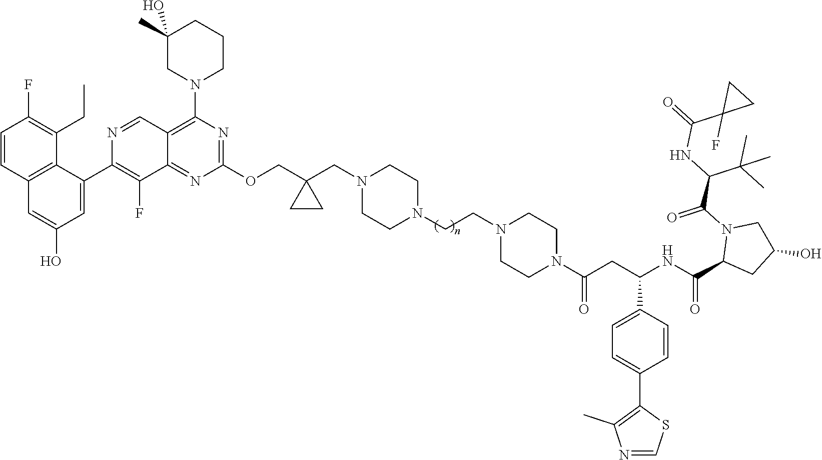 |
| 23 | 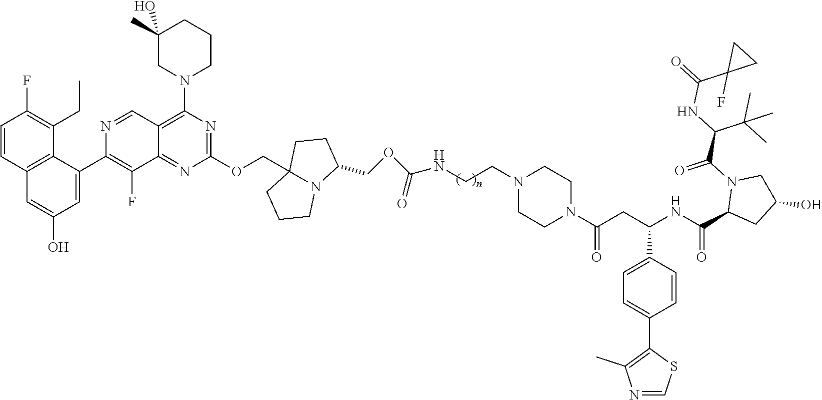 |
| 24 | 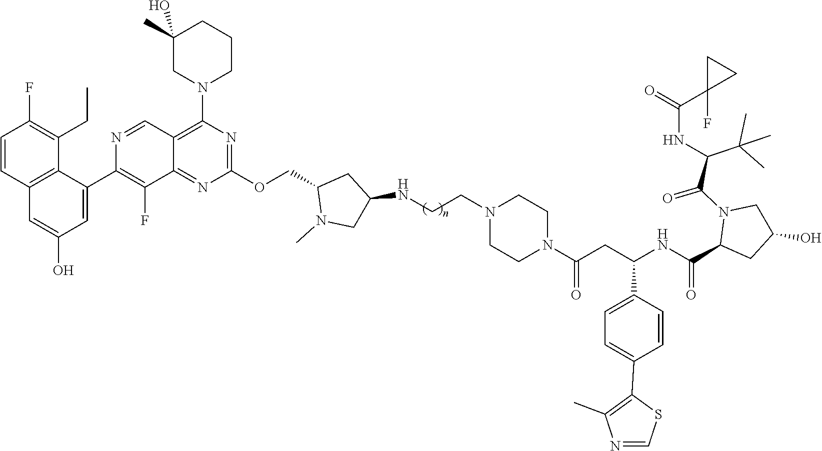 |
| 25 | 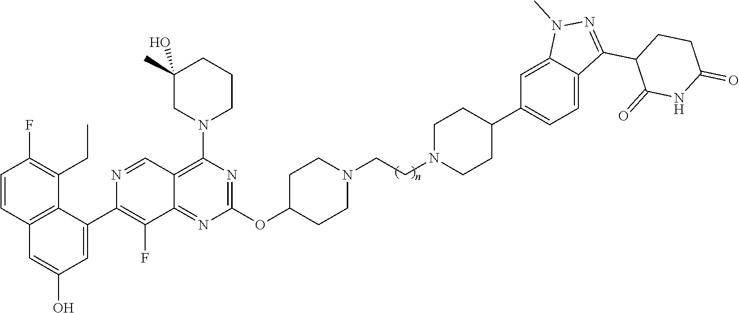 |
| 26 | 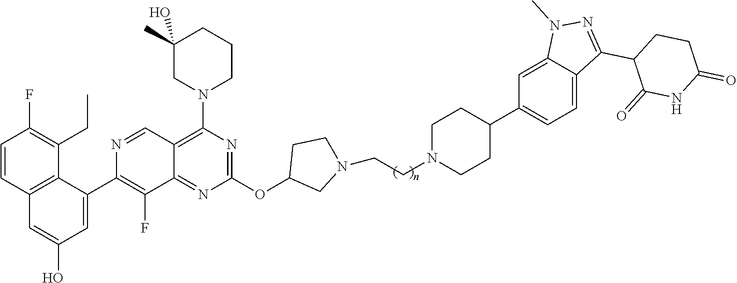 |
| 27 | 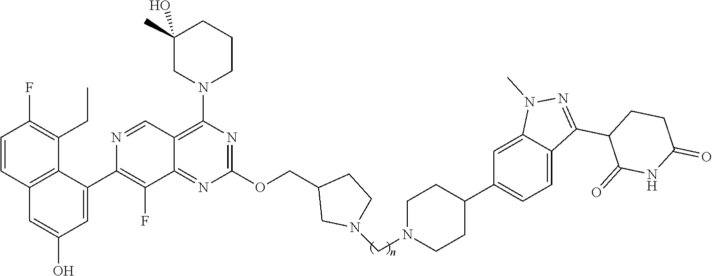 |
| 28 | 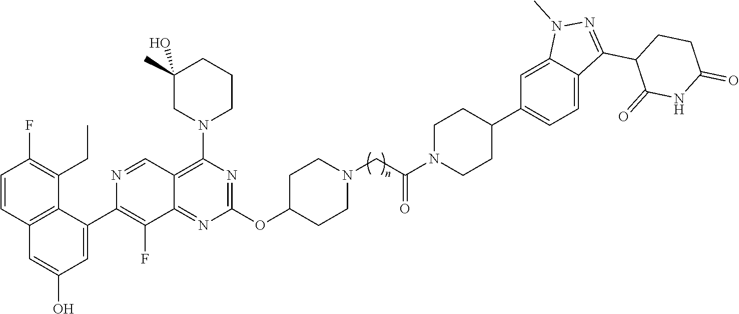 |
| 29 | 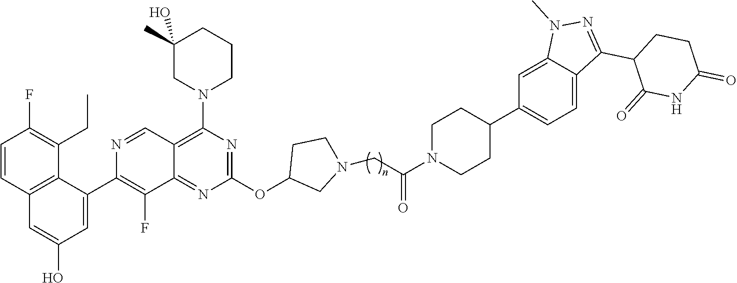 |
| 30 | 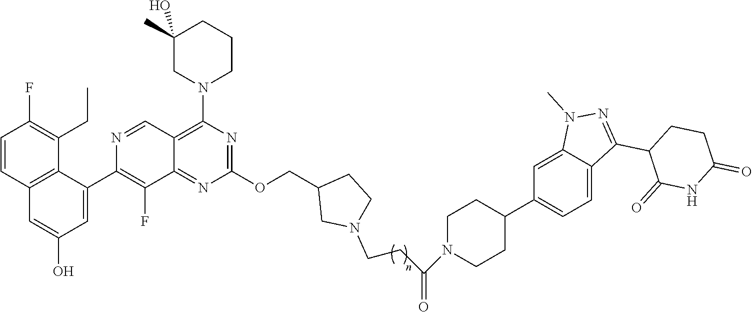 |
| 31 | 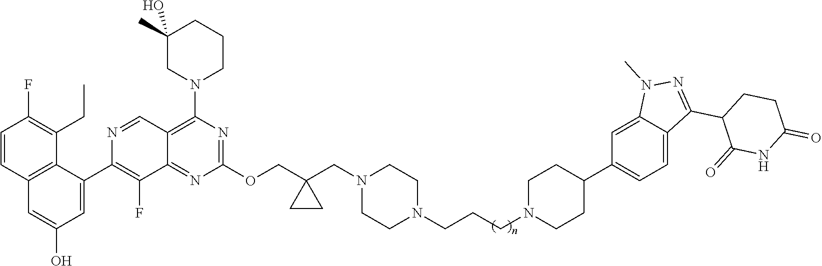 |
| 32 | 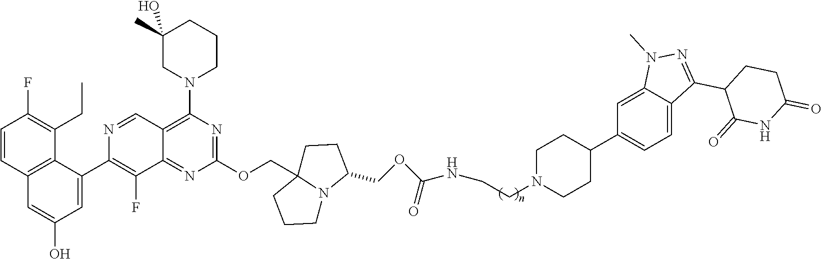 |
| 33 | 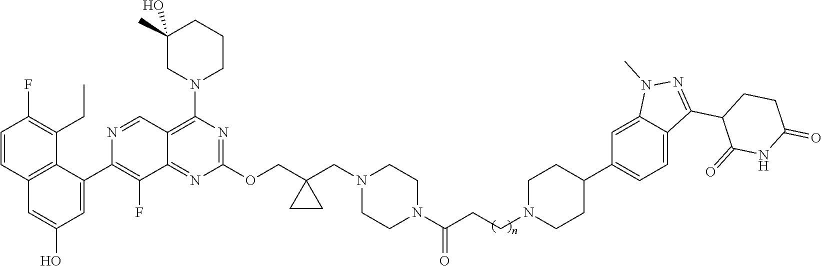 |
| 34 | 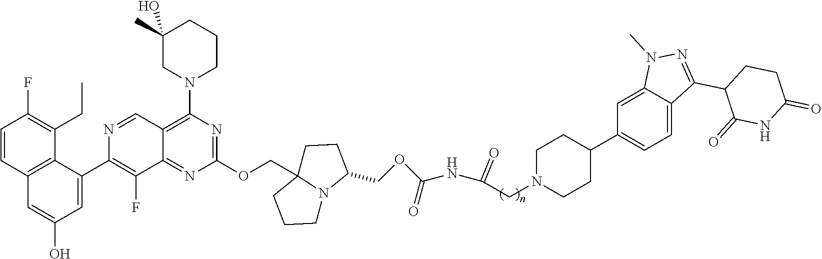 |
| 35 | 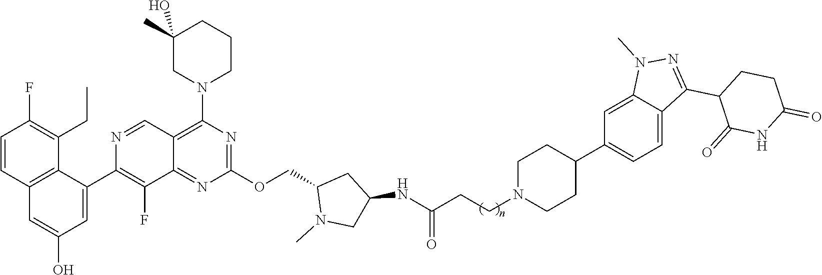 |
| 36 | 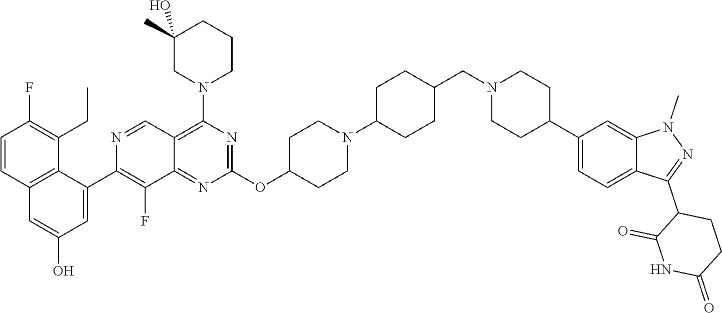 [SMILES: CCc1c(F)ccc2cc(O)cc(-c3ncc4c(N5CCC[C@@](C)(O)C5)nc(OC5CCN(C6CCC(CN7CCC(c8ccc9c(C%10CCC(=O)NC%10=O)nn(C)c9c8)CC7)CC6)CC5)nc4c3F)c12] |
| 37 | 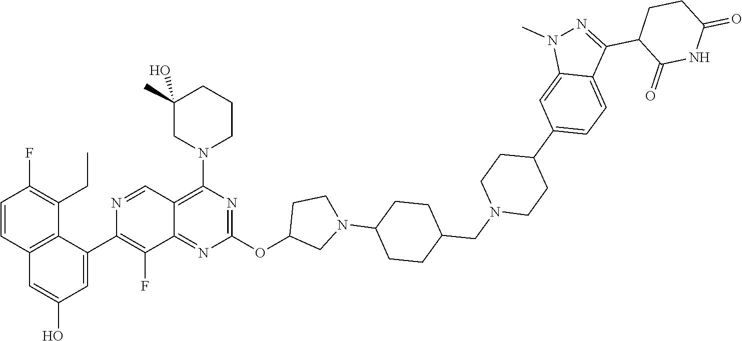 [SMILES: CCc1c(F)ccc2cc(O)cc(-c3ncc4c(N5CCC[C@@](C)(O)C5)nc(OC5CCN(C6CCC(CN7CCC(c8ccc9c(C%10CCC(=O)NC%10=O)nn(C)c9c8)CC7)CC6)C5)nc4c3F)c12] |
| 38 | 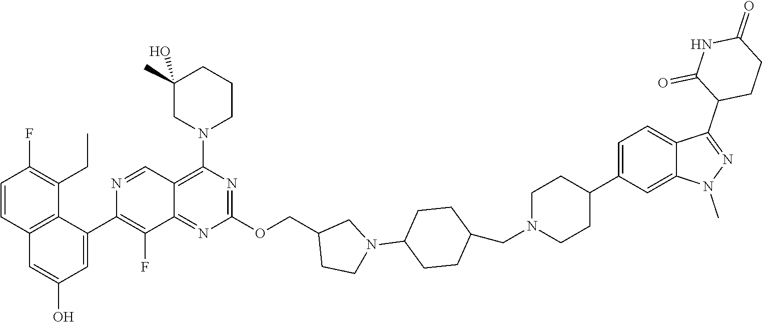 [SMILES: CCc1c(F)ccc2cc(O)cc(-c3ncc4c(N5CCC[C@@](C)(O)C5)nc(OCC5CCN(C6CCC(CN7CCC(c8ccc9c(C%10CCC(=O)NC%10=O)nn(C)c9c8)CC7)CC6)C5)nc4c3F)c12] |
| 39 | 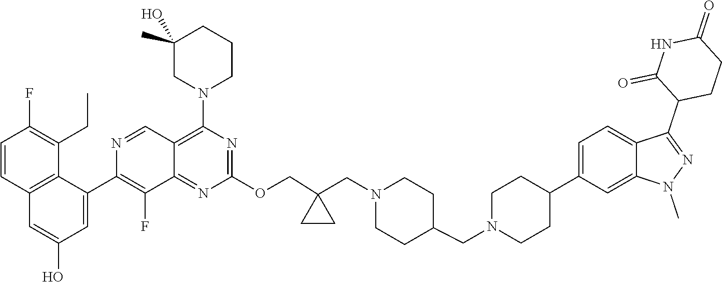 [SMILES: CCc1c(F)ccc2cc(O)cc(-c3ncc4c(N5CCC[C@@](C)(O)C5)nc(OCC5(CN6CCC(CN7CCC(c8ccc9c(C%10CCC(=O)NC%10=O)nn(C)c9c8)CC7)CC6)CC5)nc4c3F)c12] |
| 40 | 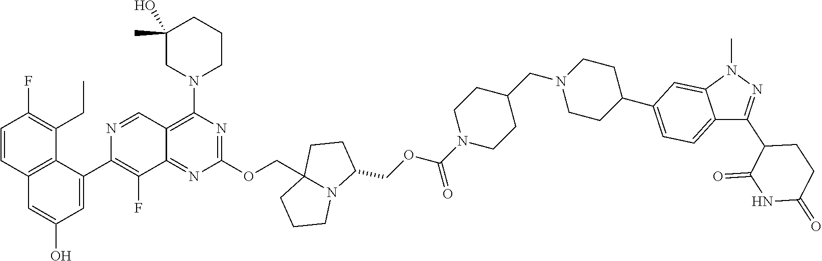 [SMILES: CCc1c(F)ccc2cc(O)cc(-c3ncc4c(N5CCC[C@@](C)(O)C5)nc(OCC56CCCN5[C@@H](COC(=O)N5CCC(CN7CCC(c8ccc9c(C%10CCC(=O)NC%10=O)nn(C)c9c8)CC7)CC5)CC6)nc4c3F)c12] |
| 41 | 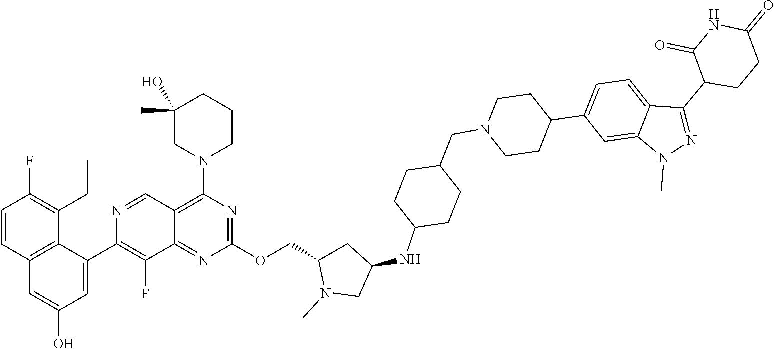 [SMILES: CCc1c(F)ccc2cc(O)cc(-c3ncc4c(N5CCC[C@@](C)(O)C5)nc(OC[C@@H]5C[C@@H](NC6CCC(CN7CCC(c8ccc9c(C%10CCC(=O)NC%10=O)nn(C)c9c8)CC7)CC6)CN5C)nc4c3F)c12] |
| 42 | 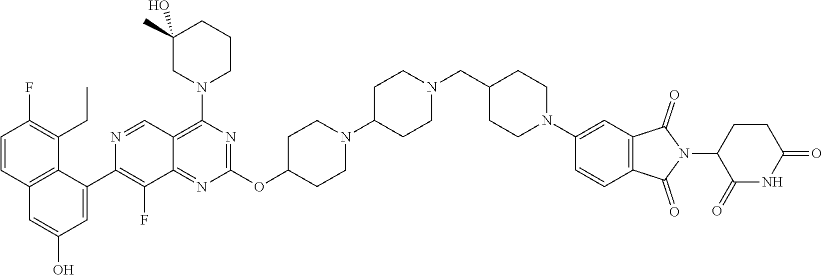 [SMILES: CCc1c(F)ccc2cc(O)cc(-c3ncc4c(N5CCC[C@@](C)(O)C5)nc(OC5CCN(C6CCN(CC7CCN(c8ccc9c(c8)C(=O)N(C8CCC(=O)NC8=O)C9=O)CC7)CC6)CC5)nc4c3F)c12] |
| 43 | 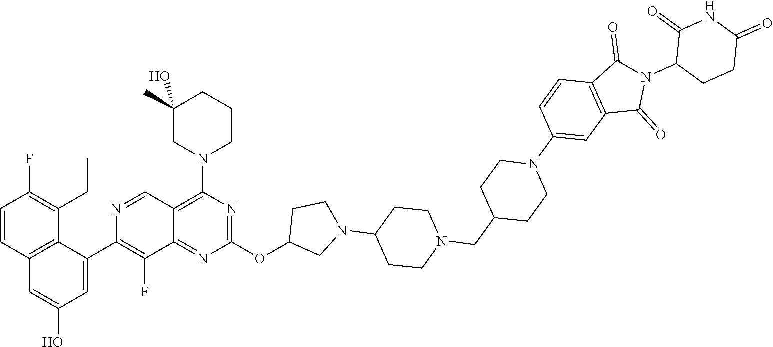 [SMILES: CCc1c(F)ccc2cc(O)cc(-c3ncc4c(N5CCC[C@@](C)(O)C5)nc(OC5CCN(C6CCN(CC7CCN(c8ccc9c(c8)C(=O)N(C8CCC(=O)NC8=O)C9=O)CC7)CC6)C5)nc4c3F)c12] |
| 44 | 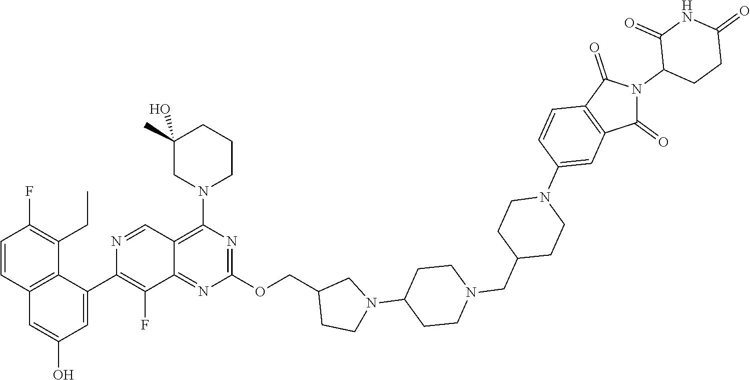 [SMILES: CCc1c(F)ccc2cc(O)cc(-c3ncc4c(N5CCC[C@@](C)(O)C5)nc(OCC5CCN(C6CCN(CC7CCN(c8ccc9c(c8)C(=O)N(C8CCC(=O)NC8=O)C9=O)CC7)CC6)C5)nc4c3F)c12] |
| 45 | 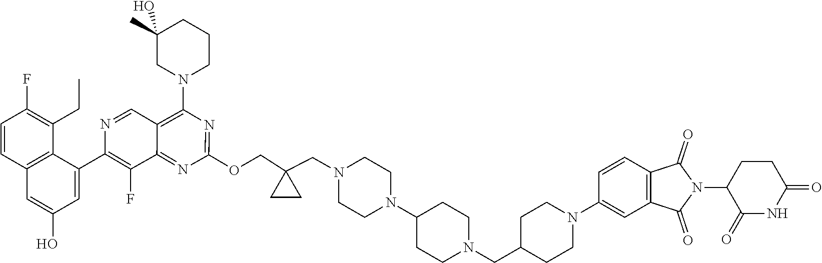 [SMILES: CCc1c(F)ccc2cc(O)cc(-c3ncc4c(N5CCC[C@@](C)(O)C5)nc(OCC5(CN6CCN(C7CCN(CC8CCN(c9ccc%10c(c9)C(=O)N(C9CCC(=O)NC9=O)C%10=O)CC8)CC7)CC6)CC5)nc4c3F)c12] |
| 46 | 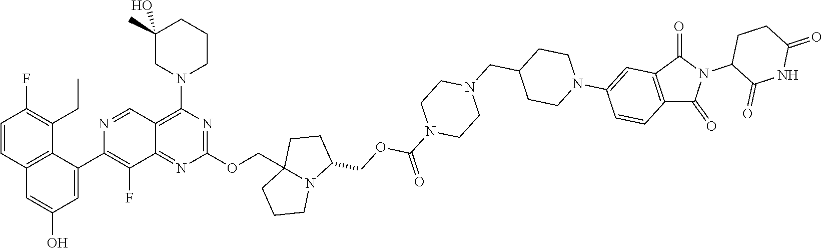 [SMILES: CCc1c(F)ccc2cc(O)cc(-c3ncc4c(N5CCC[C@@](C)(O)C5)nc(OCC56CCCN5[C@@H](COC(=O)N5CCN(CC7CCN(c8ccc9c(c8)C(=O)N(C8CCC(=O)NC8=O)C9=O)CC7)CC5)CC6)nc4c3F)c12] |
| 47 | 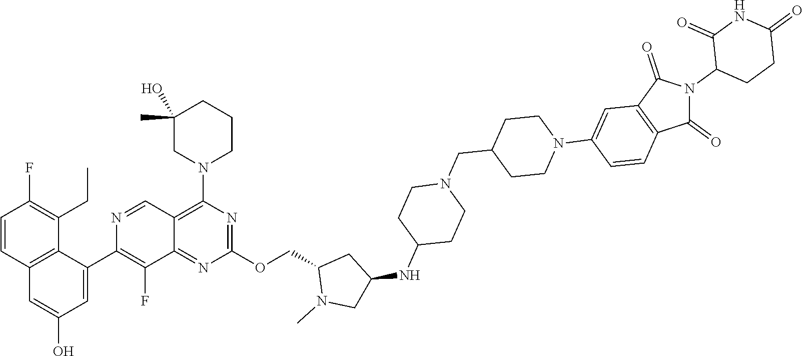 [SMILES: CCc1c(F)ccc2cc(O)cc(-c3ncc4c(N5CCC[C@@](C)(O)C5)nc(OC[C@@H]5C[C@@H](NC6CCN(CC7CCN(c8ccc9c(c8)C(=O)N(C8CCC(=O)NC8=O)C9=O)CC7)CC6)CN5C)nc4c3F)c12] |
| 48 | 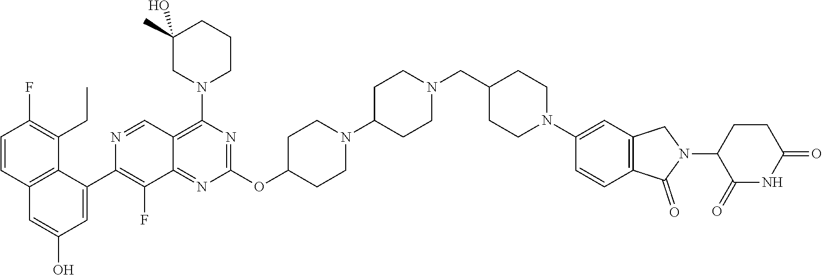 [SMILES: CCc1c(F)ccc2cc(O)cc(-c3ncc4c(N5CCC[C@@](C)(O)C5)nc(OC5CCN(C6CCN(CC7CCN(c8ccc9c(c8)CN(C8CCC(=O)NC8=O)C9=O)CC7)CC6)CC5)nc4c3F)c12] |
| 49 | 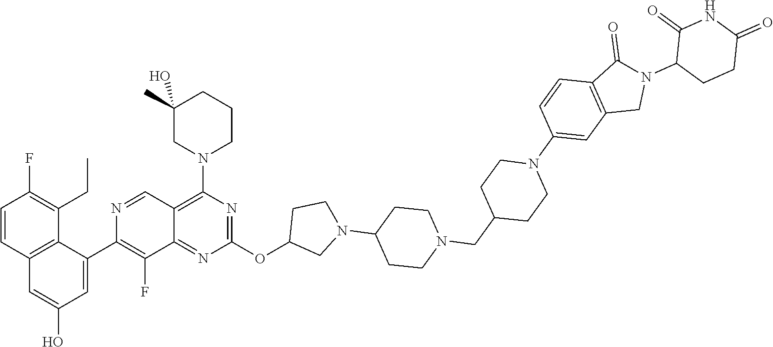 [SMILES: CCc1c(F)ccc2cc(O)cc(-c3ncc4c(N5CCC[C@@](C)(O)C5)nc(OC5CCN(C6CCN(CC7CCN(c8ccc9c(c8)CN(C8CCC(=O)NC8=O)C9=O)CC7)CC6)C5)nc4c3F)c12] |
| 50 | 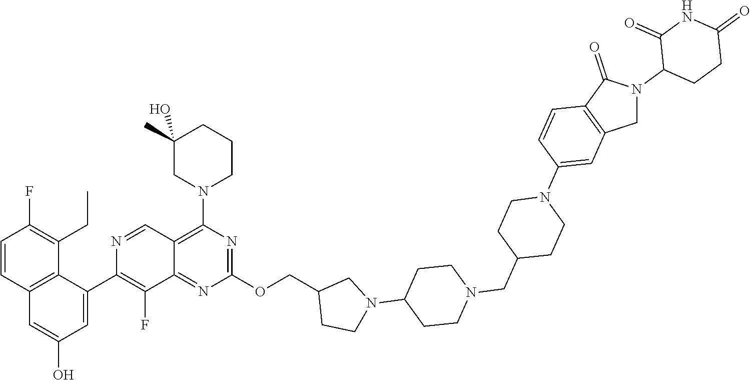 [SMILES: CCc1c(F)ccc2cc(O)cc(-c3ncc4c(N5CCC[C@@](C)(O)C5)nc(OCC5CCN(C6CCN(CC7CCN(c8ccc9c(c8)CN(C8CCC(=O)NC8=O)C9=O)CC7)CC6)C5)nc4c3F)c12] |
| 51 | 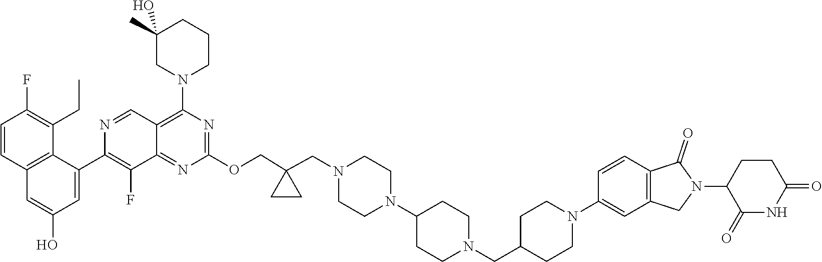 [SMILES: CCc1c(F)ccc2cc(O)cc(-c3ncc4c(N5CCC[C@@](C)(O)C5)nc(OCC5(CN6CCN(C7CCN(CC8CCN(c9ccc%10c(c9)CN(C9CCC(=O)NC9=O)C%10=O)CC8)CC7)CC6)CC5)nc4c3F)c12] |
| 52 | 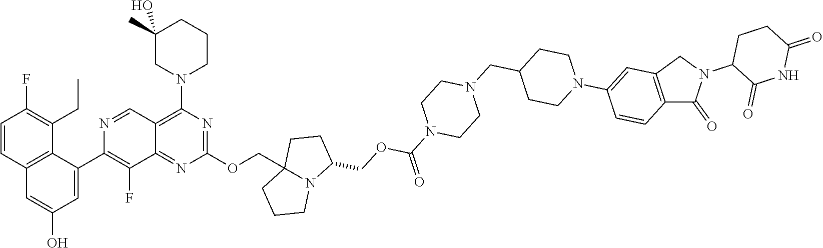 [SMILES: CCc1c(F)ccc2cc(O)cc(-c3ncc4c(N5CCC[C@@](C)(O)C5)nc(OCC56CCCN5[C@@H](COC(=O)N5CCN(CC7CCN(c8ccc9c(c8)CN(C8CCC(=O)NC8=O)C9=O)CC7)CC5)CC6)nc4c3F)c12] |
| 53 | 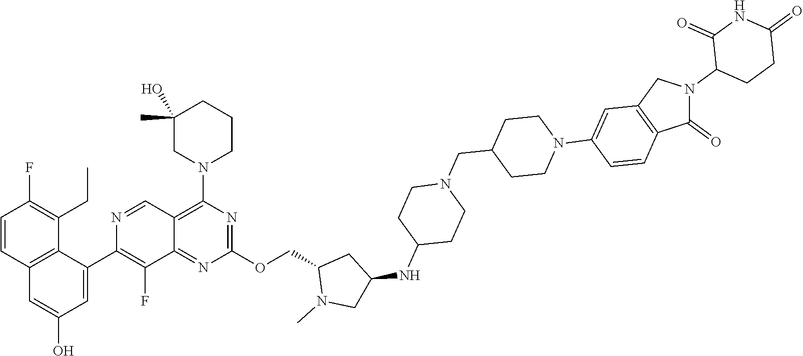 [SMILES: CCc1c(F)ccc2cc(O)cc(-c3ncc4c(N5CCC[C@@](C)(O)C5)nc(OC[C@@H]5C[C@@H](NC6CCN(CC7CCN(c8ccc9c(c8)CN(C8CCC(=O)NC8=O)C9=O)CC7)CC6)CN5C)nc4c3F)c12] |
| 54 | 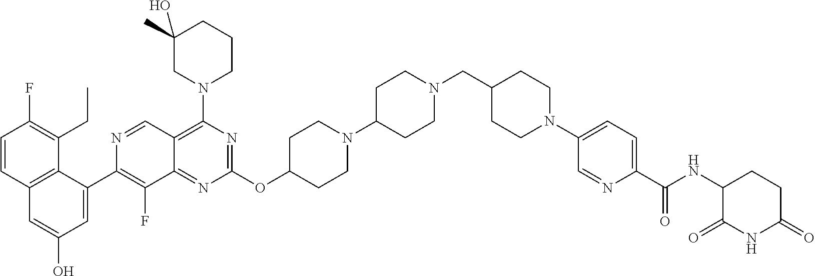 [SMILES: CCc1c(F)ccc2cc(O)cc(-c3ncc4c(N5CCC[C@@](C)(O)C5)nc(OC5CCN(C6CCN(CC7CCN(c8ccc(C(=O)NC9CCC(=O)NC9=O)nc8)CC7)CC6)CC5)nc4c3F)c12] |
| 55 | 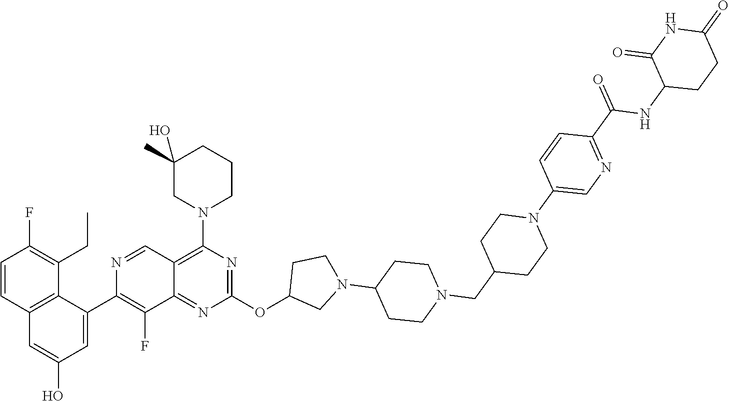 [SMILES: CCc1c(F)ccc2cc(O)cc(-c3ncc4c(N5CCC[C@@](C)(O)C5)nc(OC5CCN(C6CCN(CC7CCN(c8ccc(C(=O)NC9CCC(=O)NC9=O)nc8)CC7)CC6)C5)nc4c3F)c12] |
| 56 | 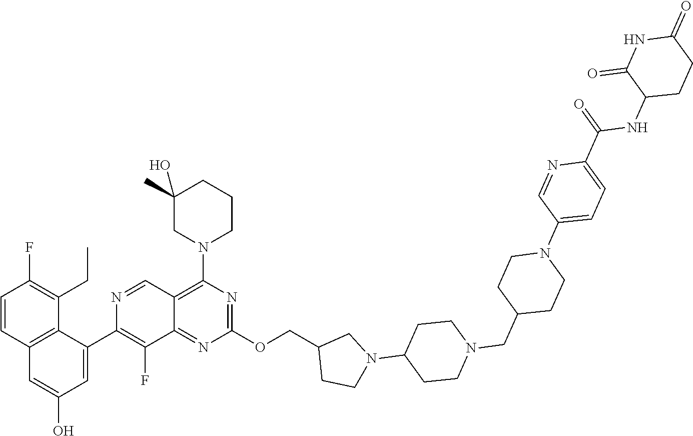 [SMILES: CCc1c(F)ccc2cc(O)cc(-c3ncc4c(N5CCC[C@@](C)(O)C5)nc(OCC5CCN(C6CCN(CC7CCN(c8ccc(C(=O)NC9CCC(=O)NC9=O)nc8)CC7)CC6)C5)nc4c3F)c12] |
| 57 | 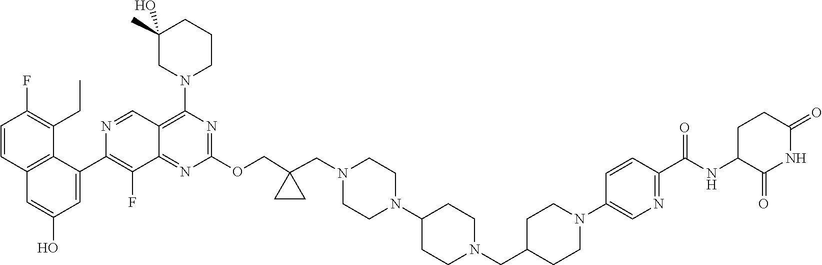 [SMILES: CCc1c(F)ccc2cc(O)cc(-c3ncc4c(N5CCC[C@@](C)(O)C5)nc(OCC5(CN6CCN(C7CCN(CC8CCN(c9ccc(C(=O)NC%10CCC(=O)NC%10=O)nc9)CC8)CC7)CC6)CC5)nc4c3F)c12] |
| 58 | 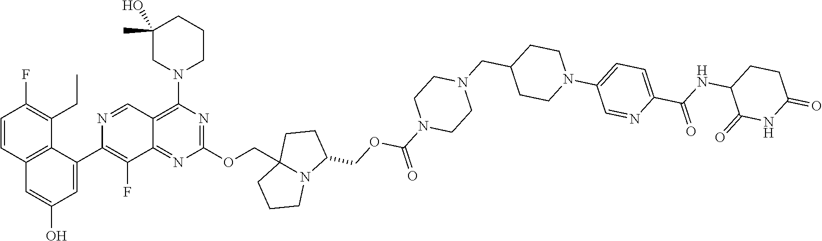 [SMILES: CCc1c(F)ccc2cc(O)cc(-c3ncc4c(N5CCC[C@@](C)(O)C5)nc(OCC56CCCN5[C@@H](COC(=O)N5CCN(CC7CCN(c8ccc(C(=O)NC9CCC(=O)NC9=O)nc8)CC7)CC5)CC6)nc4c3F)c12] |
| 59 | 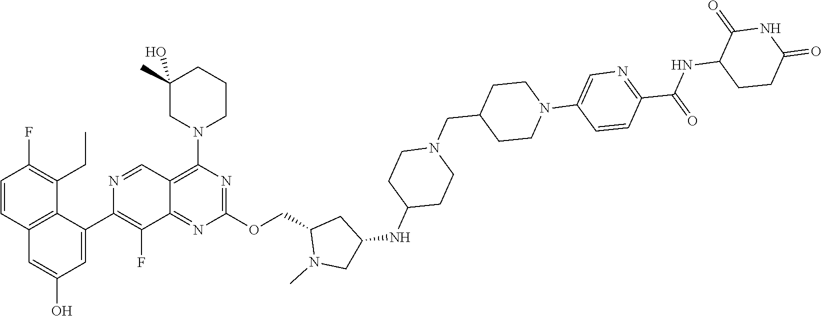 [SMILES: CCc1c(F)ccc2cc(O)cc(-c3ncc4c(N5CCC[C@@](C)(O)C5)nc(OC[C@@H]5C[C@H](NC6CCN(CC7CCN(c8ccc(C(=O)NC9CCC(=O)NC9=O)nc8)CC7)CC6)CN5C)nc4c3F)c12] |
| 60 | 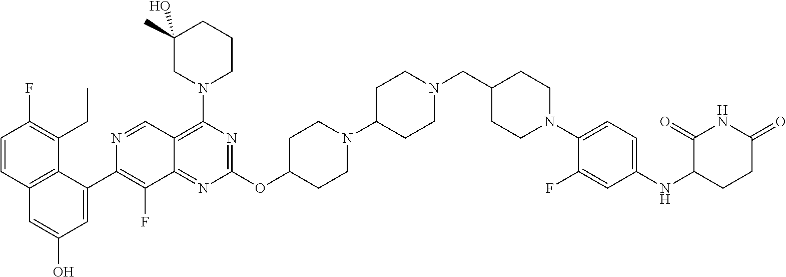 [SMILES: CCc1c(F)ccc2cc(O)cc(-c3ncc4c(N5CCC[C@@](C)(O)C5)nc(OC5CCN(C6CCN(CC7CCN(c8ccc(NC9CCC(=O)NC9=O)cc8F)CC7)CC6)CC5)nc4c3F)c12] |
| 61 | 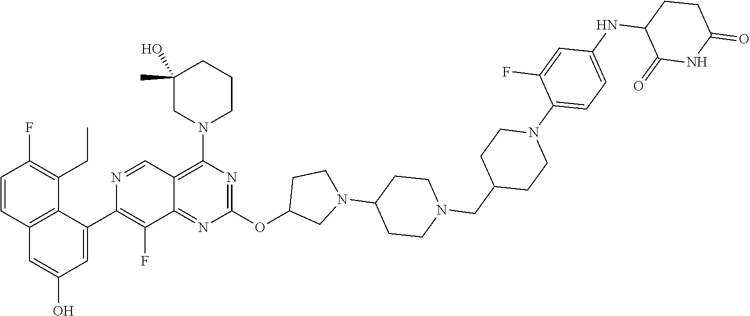 [SMILES: CCc1c(F)ccc2cc(O)cc(-c3ncc4c(N5CCC[C@@](C)(O)C5)nc(OC5CCN(C6CCN(CC7CCN(c8ccc(NC9CCC(=O)NC9=O)cc8F)CC7)CC6)C5)nc4c3F)c12] |
| 62 | 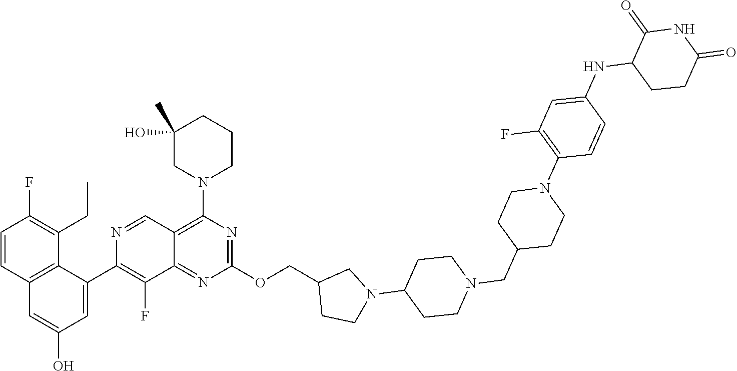 [SMILES: CCc1c(F)ccc2cc(O)cc(-c3ncc4c(N5CCC[C@@](C)(O)C5)nc(OCC5CCN(C6CCN(CC7CCN(c8ccc(NC9CCC(=O)NC9=O)cc8F)CC7)CC6)C5)nc4c3F)c12] |
| 63 | 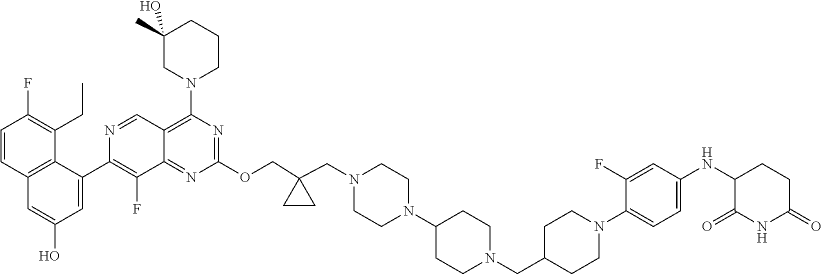 [SMILES: CCc1c(F)ccc2cc(O)cc(-c3ncc4c(N5CCC[C@@](C)(O)C5)nc(OCC5(CN6CCN(C7CCN(CC8CCN(c9ccc(NC%10CCC(=O)NC%10=O)cc9F)CC8)CC7)CC6)CC5)nc4c3F)c12] |
| 64 | 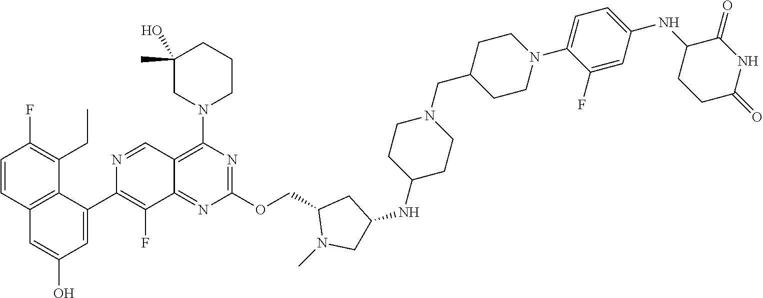 [SMILES: CCc1c(F)ccc2cc(O)cc(-c3ncc4c(N5CCC[C@@](C)(O)C5)nc(OC[C@@H]5C[C@H](NC6CCN(CC7CCN(c8ccc(NC9CCC(=O)NC9=O)cc8F)CC7)CC6)CN5C)nc4c3F)c12] |
| 65 | 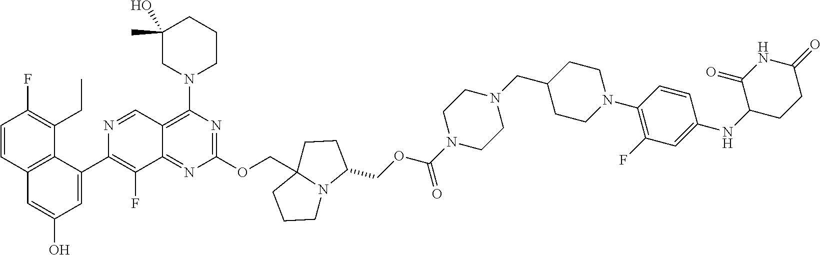 |
| 66 | 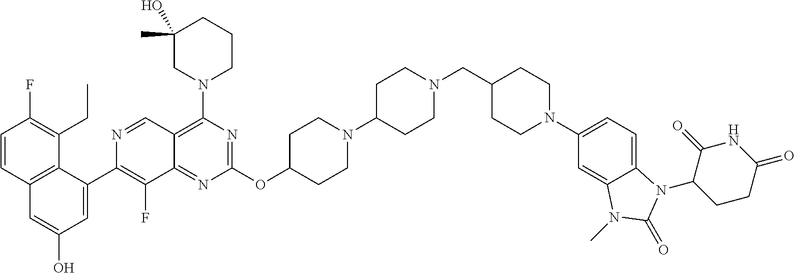 [SMILES: CCc1c(F)ccc2cc(O)cc(-c3ncc4c(N5CCC[C@@](C)(O)C5)nc(OC5CCN(C6CCN(CC7CCN(c8ccc9c(c8)n(C)c(=O)n9C8CCC(=O)NC8=O)CC7)CC6)CC5)nc4c3F)c12] |
| 67 | 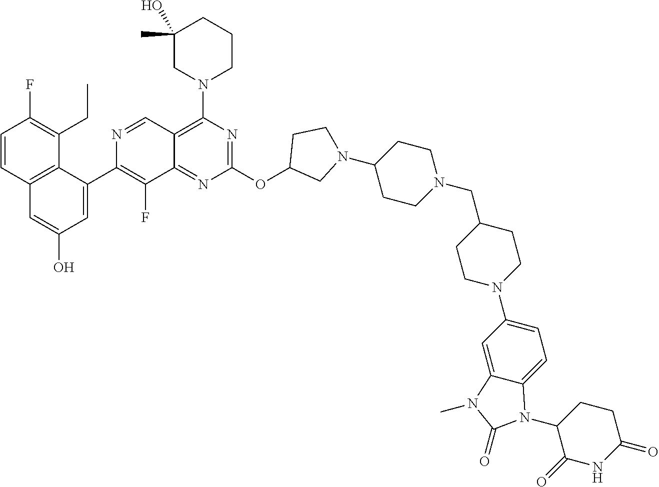 [SMILES: CCc1c(F)ccc2cc(O)cc(-c3ncc4c(N5CCC[C@@](C)(O)C5)nc(OC5CCN(C6CCN(CC7CCN(c8ccc9c(c8)n(C)c(=O)n9C8CCC(=O)NC8=O)CC7)CC6)C5)nc4c3F)c12] |
| 68 | 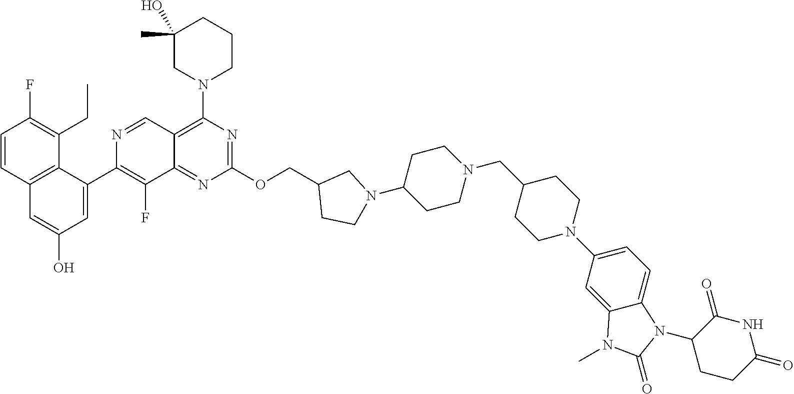 [SMILES: CCc1c(F)ccc2cc(O)cc(-c3ncc4c(N5CCC[C@@](C)(O)C5)nc(OCC5CCN(C6CCN(CC7CCN(c8ccc9c(c8)n(C)c(=O)n9C8CCC(=O)NC8=O)CC7)CC6)C5)nc4c3F)c12] |
| 69 | 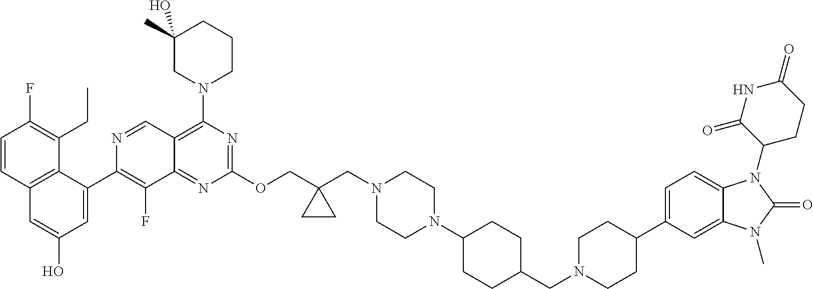 [SMILES: CCc1c(F)ccc2cc(O)cc(-c3ncc4c(N5CCC[C@@](C)(O)C5)nc(OCC5(CN6CCN(C7CCC(CN8CCC(c9ccc%10c(c9)n(C)c(=O)n%10C9CCC(=O)NC9=O)CC8)CC7)CC6)CC5)nc4c3F)c12] |
| 70 | 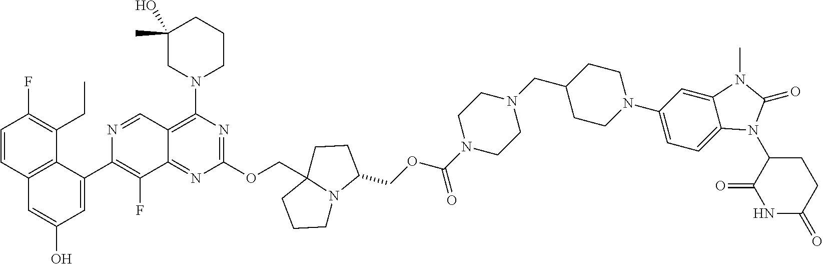 |
| 71 | 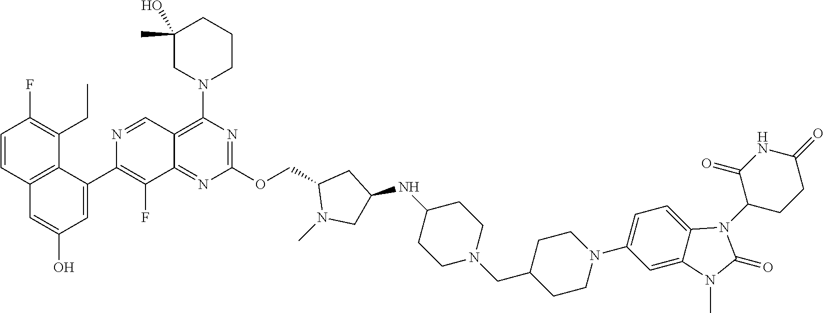 [SMILES: CCc1c(F)ccc2cc(O)cc(-c3ncc4c(N5CCC[C@@](C)(O)C5)nc(OC[C@@H]5C[C@@H](NC6CCN(CC7CCN(c8ccc9c(c8)n(C)c(=O)n9C8CCC(=O)NC8=O)CC7)CC6)CN5C)nc4c3F)c12] |
| 72 | 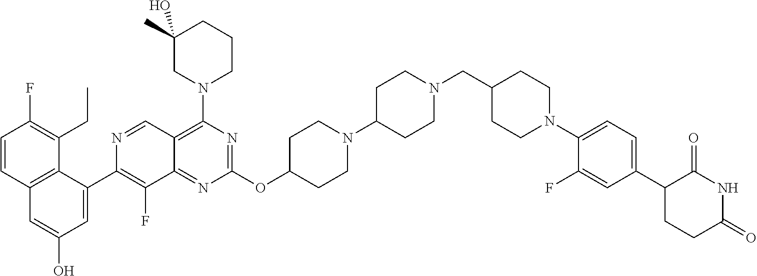 [SMILES: CCc1c(F)ccc2cc(O)cc(-c3ncc4c(N5CCC[C@@](C)(O)C5)nc(OC5CCN(C6CCN(CC7CCN(c8ccc(C9CCC(=O)NC9=O)cc8F)CC7)CC6)CC5)nc4c3F)c12] |
| 73 | 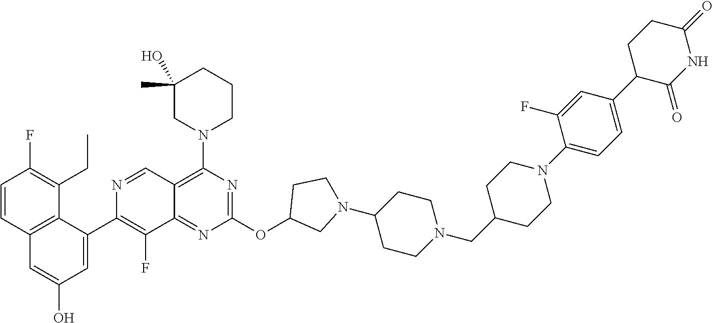 [SMILES: CCc1c(F)ccc2cc(O)cc(-c3ncc4c(N5CCC[C@@](C)(O)C5)nc(OC5CCN(C6CCN(CC7CCN(c8ccc(C9CCC(=O)NC9=O)cc8F)CC7)CC6)C5)nc4c3F)c12] |
| 74 | 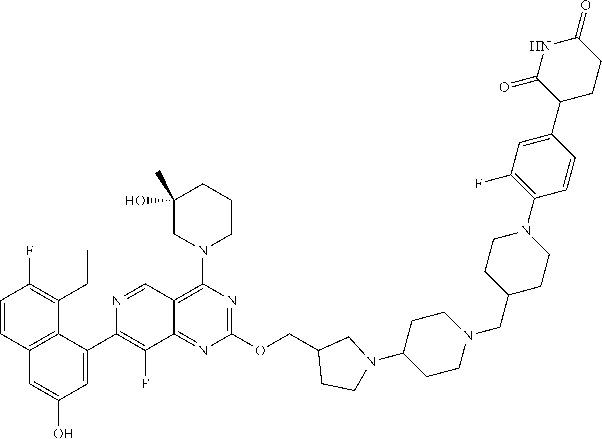 [SMILES: CCc1c(F)ccc2cc(O)cc(-c3ncc4c(N5CCC[C@@](C)(O)C5)nc(OCC5CCN(C6CCN(CC7CCN(c8ccc(C9CCC(=O)NC9=O)cc8F)CC7)CC6)C5)nc4c3F)c12] |
| 75 | 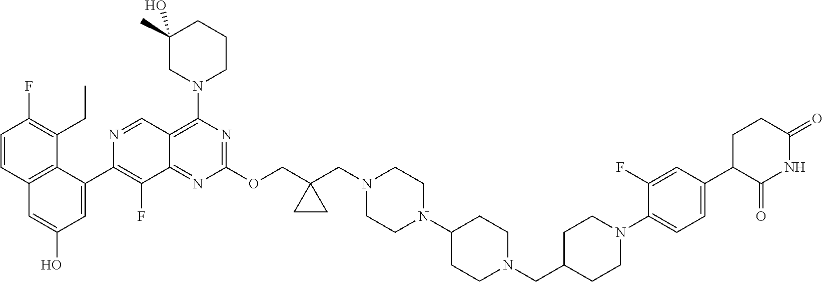 [SMILES: CCc1c(F)ccc2cc(O)cc(-c3ncc4c(N5CCC[C@@](C)(O)C5)nc(OCC5(CN6CCN(C7CCN(CC8CCN(c9ccc(C%10CCC(=O)NC%10=O)cc9F)CC8)CC7)CC6)CC5)nc4c3F)c12] |
| 76 | 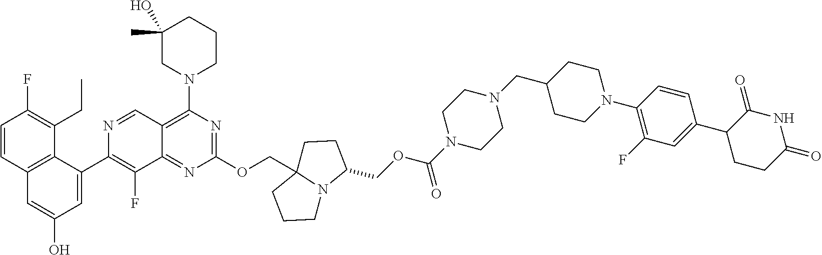 |
| 77 | 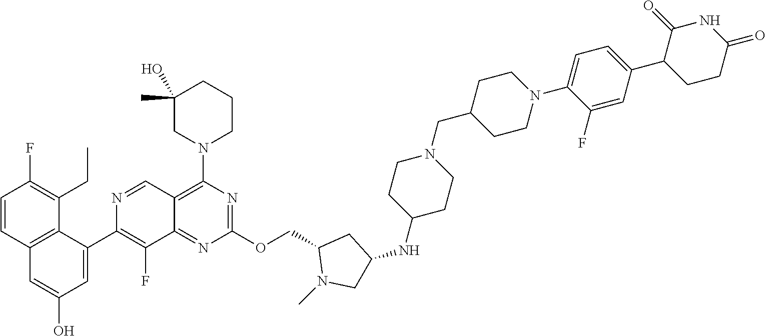 [SMILES: CCc1c(F)ccc2cc(O)cc(-c3ncc4c(N5CCC[C@@](C)(O)C5)nc(OC[C@@H]5C[C@H](NC6CCN(CC7CCN(c8ccc(C9CCC(=O)NC9=O)cc8F)CC7)CC6)CN5C)nc4c3F)c12] |
| 78 | 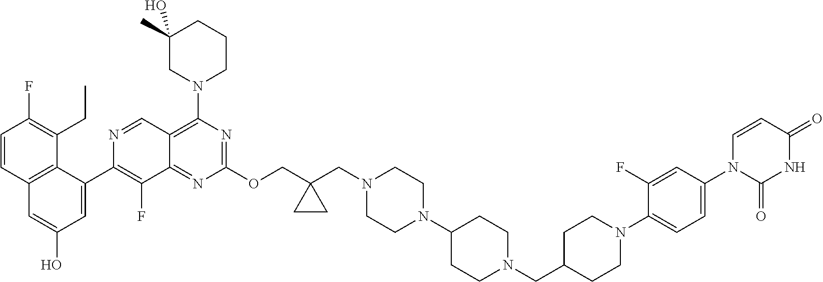 [SMILES: CCc1c(F)ccc2cc(O)cc(-c3ncc4c(N5CCC[C@@](C)(O)C5)nc(OCC5(CN6CCN(C7CCN(CC8CCN(c9ccc(-n%10ccc(=O)[nH]c%10=O)cc9F)CC8)CC7)CC6)CC5)nc4c3F)c12] |
| 79 | 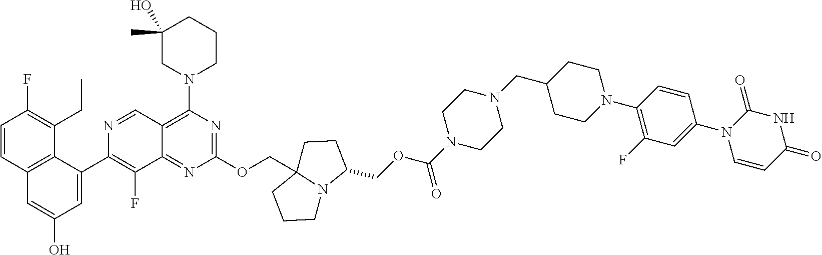 |
| 80 | 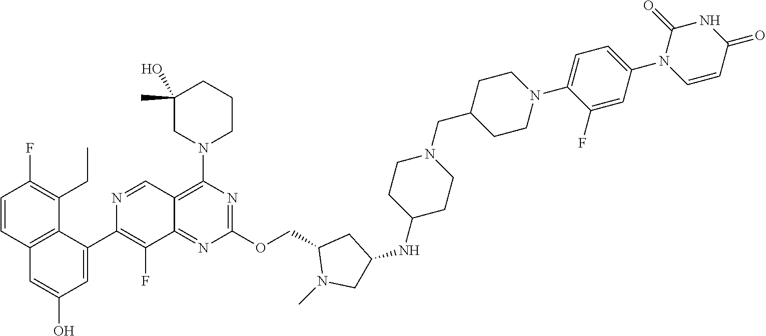 [SMILES: CCc1c(F)ccc2cc(O)cc(-c3ncc4c(N5CCC[C@@](C)(O)C5)nc(OC[C@@H]5C[C@H](NC6CCN(CC7CCN(c8ccc(-n9ccc(=O)[nH]c9=O)cc8F)CC7)CC6)CN5C)nc4c3F)c12] |
| 81 | 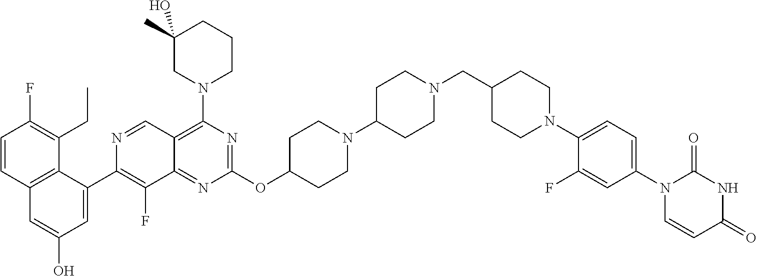 [SMILES: CCc1c(F)ccc2cc(O)cc(-c3ncc4c(N5CCC[C@@](C)(O)C5)nc(OC5CCN(C6CCN(CC7CCN(c8ccc(-n9ccc(=O)[nH]c9=O)cc8F)CC7)CC6)CC5)nc4c3F)c12] |
| 82 | 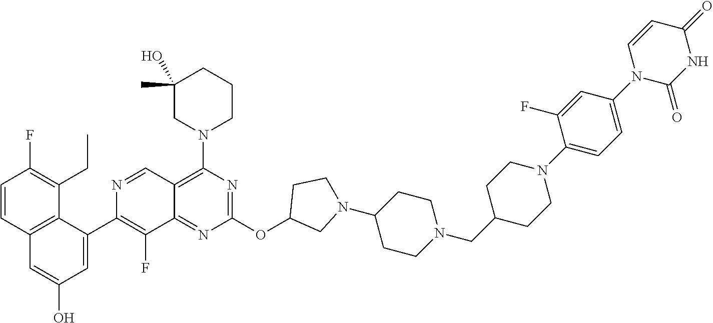 [SMILES: CCc1c(F)ccc2cc(O)cc(-c3ncc4c(N5CCC[C@@](C)(O)C5)nc(OC5CCN(C6CCN(CC7CCN(c8ccc(-n9ccc(=O)[nH]c9=O)cc8F)CC7)CC6)C5)nc4c3F)c12] |
| 83 | 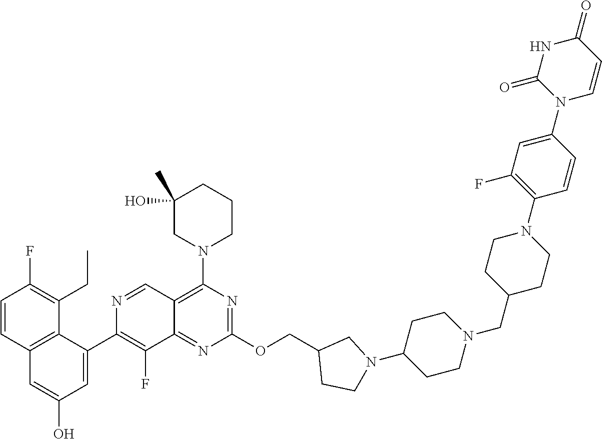 [SMILES: CCc1c(F)ccc2cc(O)cc(-c3ncc4c(N5CCC[C@@](C)(O)C5)nc(OCC5CCN(C6CCN(CC7CCN(c8ccc(-n9ccc(=O)[nH]c9=O)cc8F)CC7)CC6)C5)nc4c3F)c12] |
| 84 | 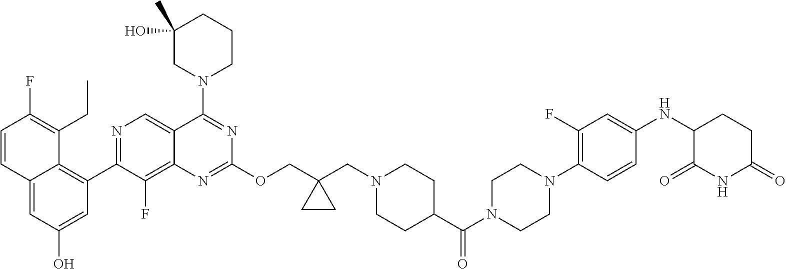 [SMILES: CCc1c(F)ccc2cc(O)cc(-c3ncc4c(N5CCC[C@@](C)(O)C5)nc(OCC5(CN6CCC(C(=O)N7CCN(c8ccc(NC9CCC(=O)NC9=O)cc8F)CC7)CC6)CC5)nc4c3F)c12] |
| 85 | 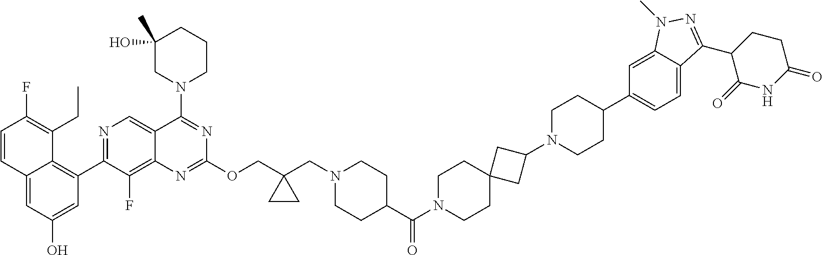 [SMILES: CCc1c(F)ccc2cc(O)cc(-c3ncc4c(N5CCC[C@@](C)(O)C5)nc(OCC5(CN6CCC(C(=O)N7CCC8(CC7)CC(N7CCC(c9ccc%10c(C%11CCC(=O)NC%11=O)nn(C)c%10c9)CC7)C8)CC6)CC5)nc4c3F)c12] |
| 86 | 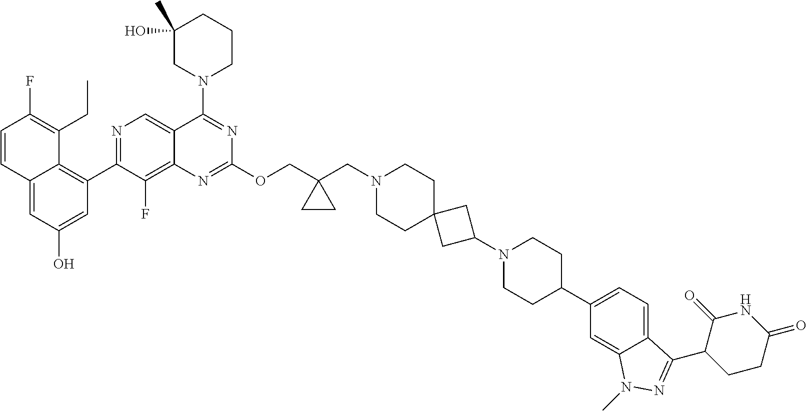 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 87 | 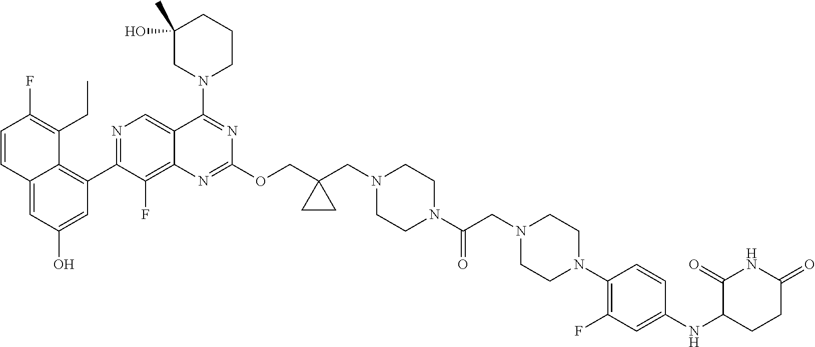 [SMILES: CCc1c(F)ccc2cc(O)cc(-c3ncc4c(N5CCC[C@@](C)(O)C5)nc(OCC5(CN6CCN(C(=O)CN7CCN(c8ccc(NC9CCC(=O)NC9=O)cc8F)CC7)CC6)CC5)nc4c3F)c12] |
| 88 | 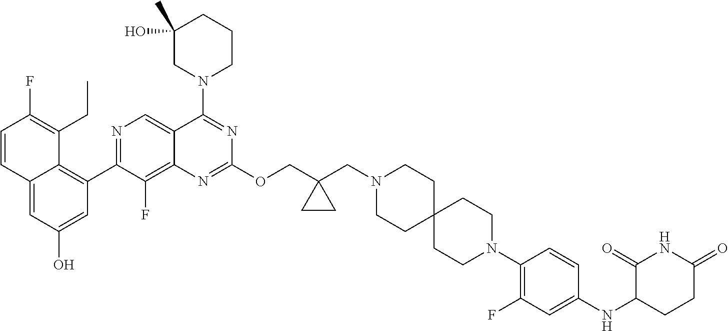 [SMILES: CCc1c(F)ccc2cc(O)cc(-c3ncc4c(N5CCC[C@@](C)(O)C5)nc(OCC5(CN6CCC7(CC6)CCN(c6ccc(NC8CCC(=O)NC8=O)cc6F)CC7)CC5)nc4c3F)c12] |
| 89 | 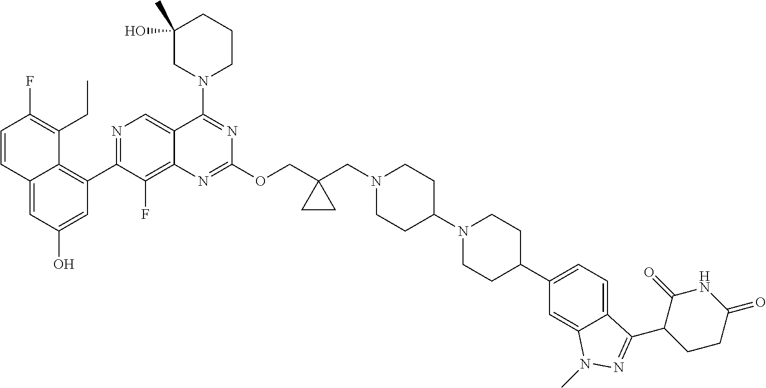 [SMILES: CCc1c(F)ccc2cc(O)cc(-c3ncc4c(N5CCC[C@@](C)(O)C5)nc(OCC5(CN6CCC(N7CCC(c8ccc9c(C%10CCC(=O)NC%10=O)nn(C)c9c8)CC7)CC6)CC5)nc4c3F)c12] |
| 90 | 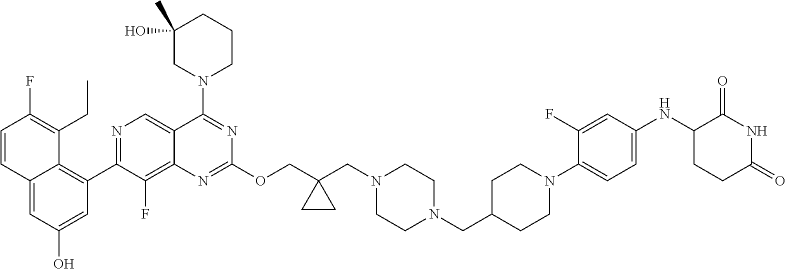 [SMILES: CCc1c(F)ccc2cc(O)cc(-c3ncc4c(N5CCC[C@@](C)(O)C5)nc(OCC5(CN6CCN(CC7CCN(c8ccc(NC9CCC(=O)NC9=O)cc8F)CC7)CC6)CC5)nc4c3F)c12] |
| 91 | 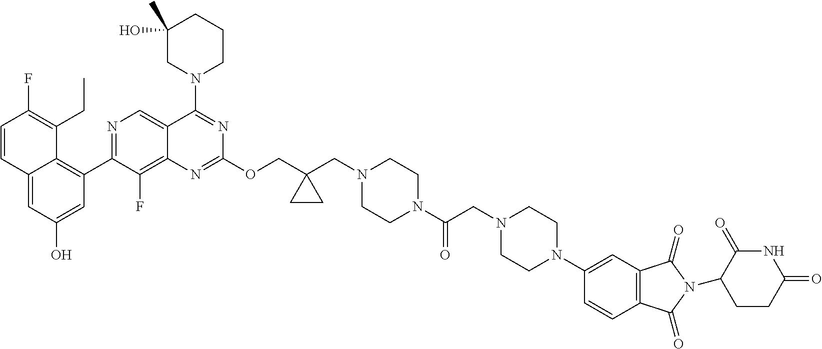 [SMILES: CCc1c(F)ccc2cc(O)cc(-c3ncc4c(N5CCC[C@@](C)(O)C5)nc(OCC5(CN6CCN(C(=O)CN7CCN(c8ccc9c(c8)C(=O)N(C8CCC(=O)NC8=O)C9=O)CC7)CC6)CC5)nc4c3F)c12] |
| 92 | 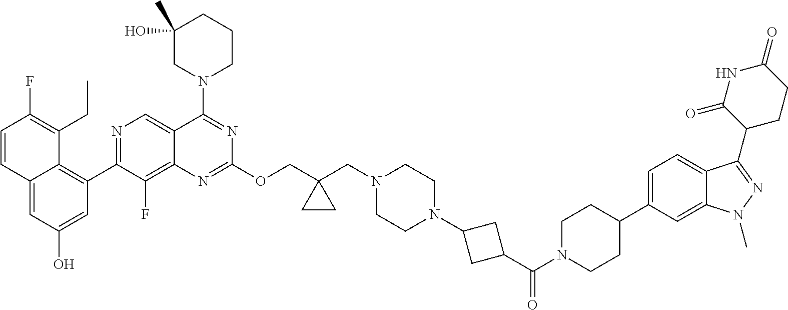 [SMILES: CCc1c(F)ccc2cc(O)cc(-c3ncc4c(N5CCC[C@@](C)(O)C5)nc(OCC5(CN6CCN(C7CC(C(=O)N8CCC(c9ccc%10c(C%11CCC(=O)NC%11=O)nn(C)c%10c9)CC8)C7)CC6)CC5)nc4c3F)c12] |
| 93 | 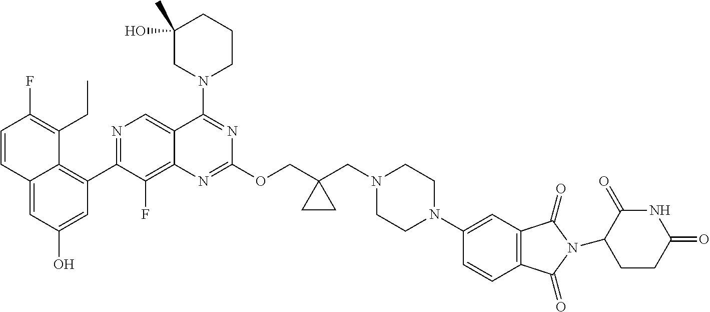 [SMILES: CCc1c(F)ccc2cc(O)cc(-c3ncc4c(N5CCC[C@@](C)(O)C5)nc(OCC5(CN6CCN(c7ccc8c(c7)C(=O)N(C7CCC(=O)NC7=O)C8=O)CC6)CC5)nc4c3F)c12] |
| 94 | 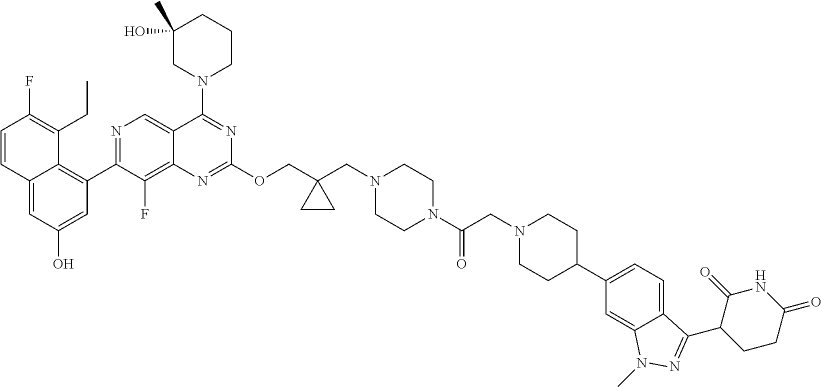 [SMILES: CCc1c(F)ccc2cc(O)cc(-c3ncc4c(N5CCC[C@@](C)(O)C5)nc(OCC5(CN6CCN(C(=O)CN7CCC(c8ccc9c(C%10CCC(=O)NC%10=O)nn(C)c9c8)CC7)CC6)CC5)nc4c3F)c12] |
| 95 | 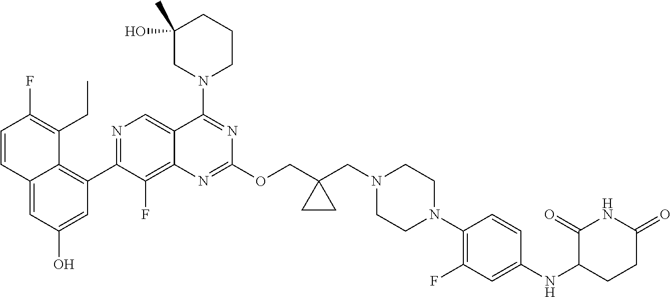 [SMILES: CCc1c(F)ccc2cc(O)cc(-c3ncc4c(N5CCC[C@@](C)(O)C5)nc(OCC5(CN6CCN(c7ccc(NC8CCC(=O)NC8=O)cc7F)CC6)CC5)nc4c3F)c12] |
| 96 | 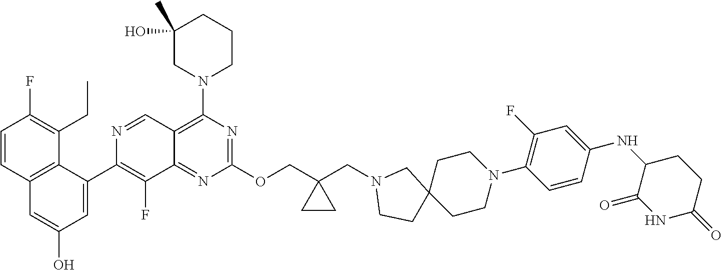 [SMILES: CCc1c(F)ccc2cc(O)cc(-c3ncc4c(N5CCC[C@@](C)(O)C5)nc(OCC5(CN6CCC7(CCN(c8ccc(NC9CCC(=O)NC9=O)cc8F)CC7)C6)CC5)nc4c3F)c12] |
| 97 | 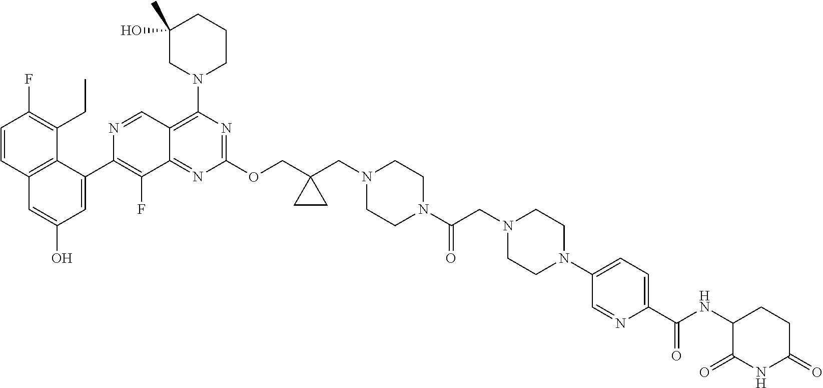 [SMILES: CCc1c(F)ccc2cc(O)cc(-c3ncc4c(N5CCC[C@@](C)(O)C5)nc(OCC5(CN6CCN(C(=O)CN7CCN(c8ccc(C(=O)NC9CCC(=O)NC9=O)nc8)CC7)CC6)CC5)nc4c3F)c12] |
| 98 | 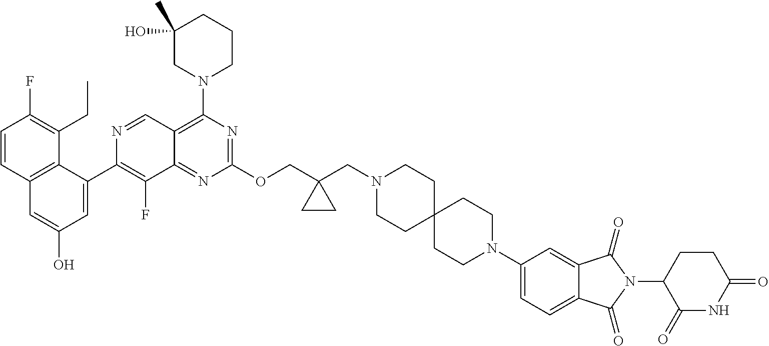 [SMILES: CCc1c(F)ccc2cc(O)cc(-c3ncc4c(N5CCC[C@@](C)(O)C5)nc(OCC5(CN6CCC7(CC6)CCN(c6ccc8c(c6)C(=O)N(C6CCC(=O)NC6=O)C8=O)CC7)CC5)nc4c3F)c12] |
| 99 | 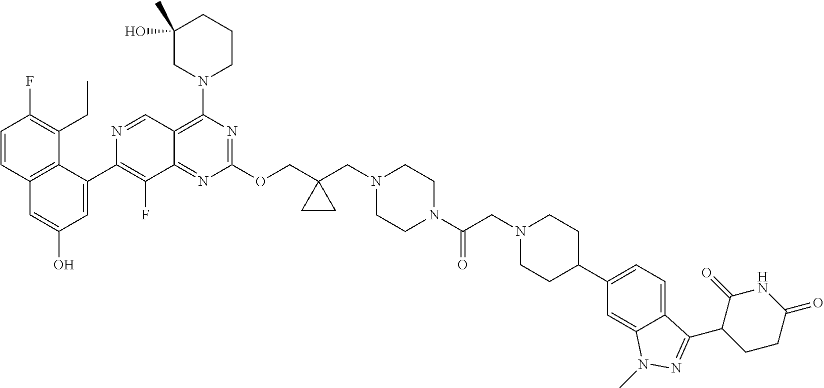 [SMILES: CCc1c(F)ccc2cc(O)cc(-c3ncc4c(N5CCC[C@@](C)(O)C5)nc(OCC5(CN6CCN(C(=O)CN7CCC(c8ccc9c(C%10CCC(=O)NC%10=O)nn(C)c9c8)CC7)CC6)CC5)nc4c3F)c12] |
| 100 | 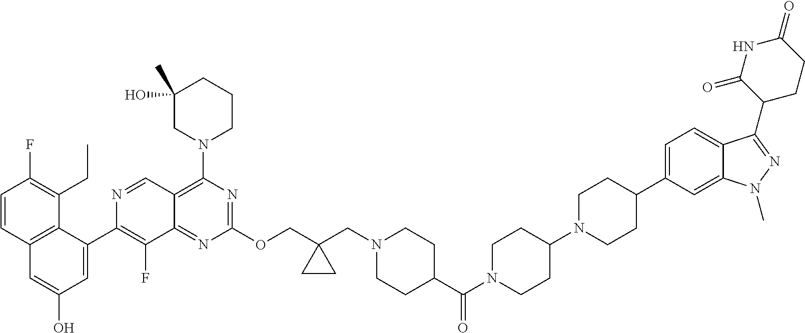 [SMILES: CCc1c(F)ccc2cc(O)cc(-c3ncc4c(N5CCC[C@@](C)(O)C5)nc(OCC5(CN6CCC(C(=O)N7CCC(N8CCC(c9ccc%10c(C%11CCC(=O)NC%11=O)nn(C)c%10c9)CC8)CC7)CC6)CC5)nc4c3F)c12] |
| 101 | 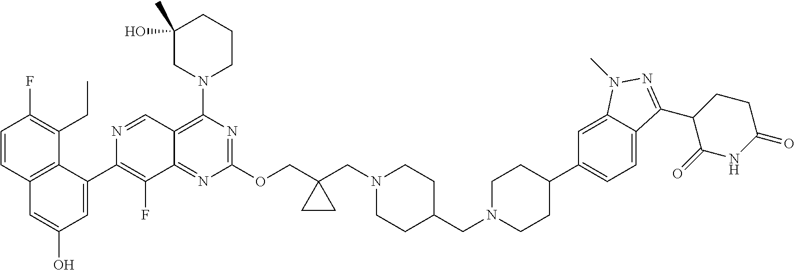 [SMILES: CCc1c(F)ccc2cc(O)cc(-c3ncc4c(N5CCC[C@@](C)(O)C5)nc(OCC5(CN6CCC(CN7CCC(c8ccc9c(C%10CCC(=O)NC%10=O)nn(C)c9c8)CC7)CC6)CC5)nc4c3F)c12] |
| 102 | 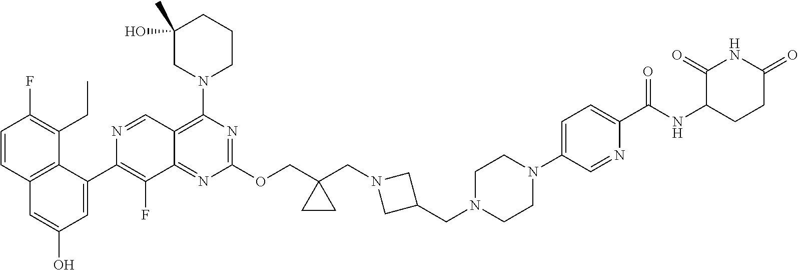 [SMILES: CCc1c(F)ccc2cc(O)cc(-c3ncc4c(N5CCC[C@@](C)(O)C5)nc(OCC5(CN6CC(CN7CCN(c8ccc(C(=O)NC9CCC(=O)NC9=O)nc8)CC7)C6)CC5)nc4c3F)c12] |
| 103 | 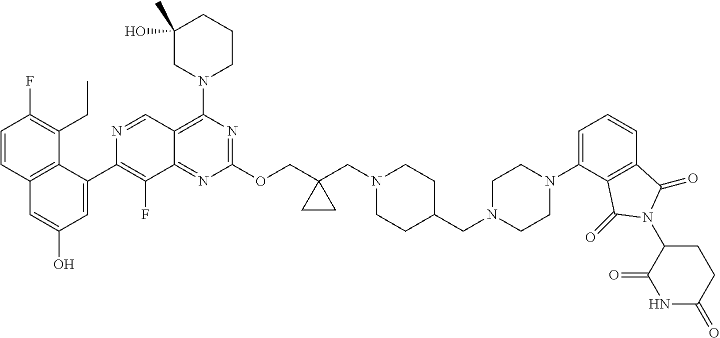 [SMILES: CCc1c(F)ccc2cc(O)cc(-c3ncc4c(N5CCC[C@@](C)(O)C5)nc(OCC5(CN6CCC(CN7CCN(c8cccc9c8C(=O)N(C8CCC(=O)NC8=O)C9=O)CC7)CC6)CC5)nc4c3F)c12] |
| 104 | 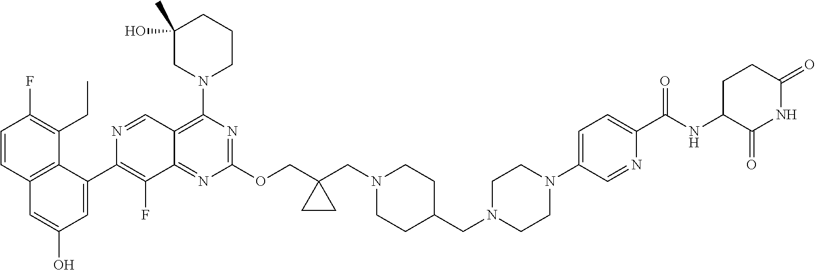 [SMILES: CCc1c(F)ccc2cc(O)cc(-c3ncc4c(N5CCC[C@@](C)(O)C5)nc(OCC5(CN6CCC(CN7CCN(c8ccc(C(=O)NC9CCC(=O)NC9=O)nc8)CC7)CC6)CC5)nc4c3F)c12] |
| 105 | 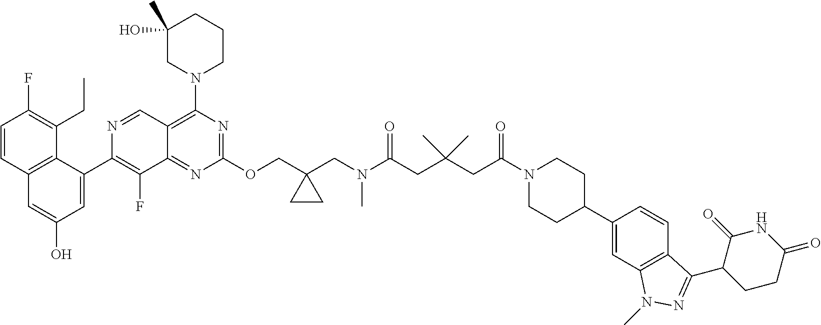 [SMILES: CCc1c(F)ccc2cc(O)cc(-c3ncc4c(N5CCC[C@@](C)(O)C5)nc(OCC5(CN(C)C(=O)CC(C)(C)CC(=O)N6CCC(c7ccc8c(C9CCC(=O)NC9=O)nn(C)c8c7)CC6)CC5)nc4c3F)c12] |
| 106 | 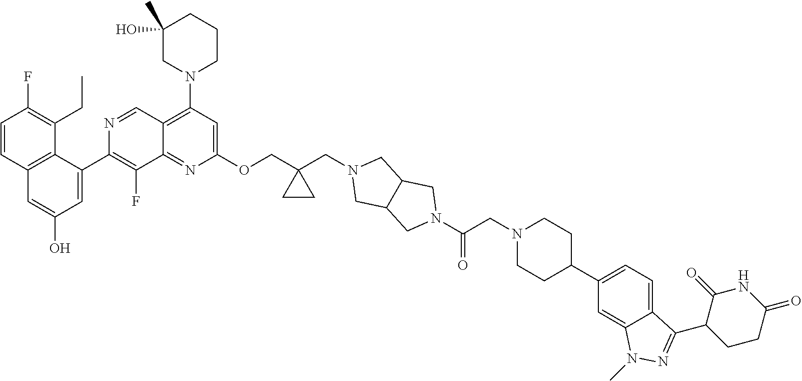 [SMILES: CCc1c(F)ccc2cc(O)cc(-c3ncc4c(N5CCC[C@@](C)(O)C5)cc(OCC5(CN6CC7CN(C(=O)CN8CCC(c9ccc%10c(C%11CCC(=O)NC%11=O)nn(C)c%10c9)CC8)CC7C6)CC5)nc4c3F)c12] |
| 107 | 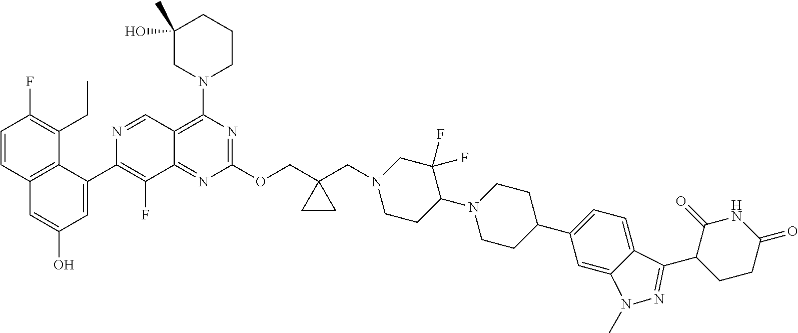 [SMILES: CCc1c(F)ccc2cc(O)cc(-c3ncc4c(N5CCC[C@@](C)(O)C5)nc(OCC5(CN6CCC(N7CCC(c8ccc9c(C%10CCC(=O)NC%10=O)nn(C)c9c8)CC7)C(F)(F)C6)CC5)nc4c3F)c12] |
| 108 | 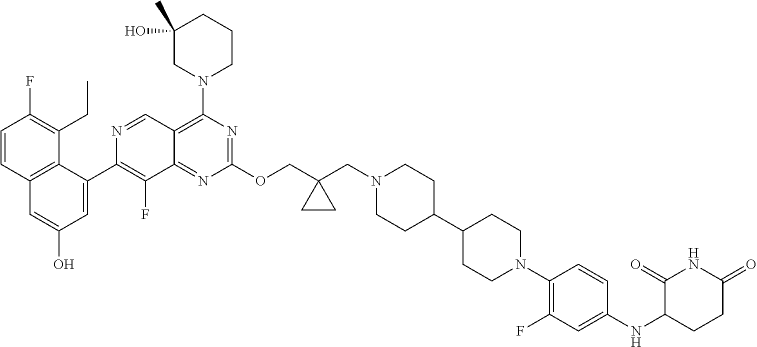 [SMILES: CCc1c(F)ccc2cc(O)cc(-c3ncc4c(N5CCC[C@@](C)(O)C5)nc(OCC5(CN6CCC(C7CCN(c8ccc(NC9CCC(=O)NC9=O)cc8F)CC7)CC6)CC5)nc4c3F)c12] |
| 109 | 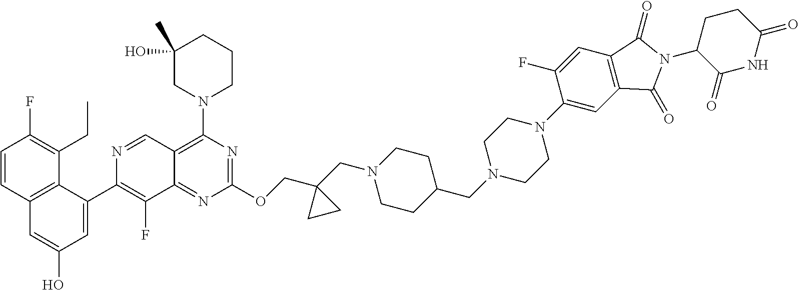 [SMILES: CCc1c(F)ccc2cc(O)cc(-c3ncc4c(N5CCC[C@@](C)(O)C5)nc(OCC5(CN6CCC(CN7CCN(c8cc9c(cc8F)C(=O)N(C8CCC(=O)NC8=O)C9=O)CC7)CC6)CC5)nc4c3F)c12] |
| 110 | 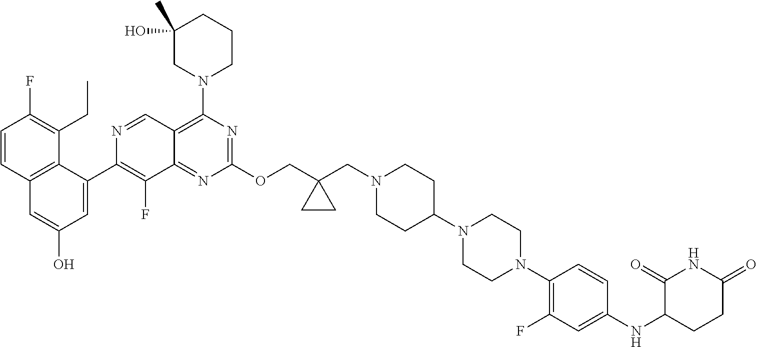 [SMILES: CCc1c(F)ccc2cc(O)cc(-c3ncc4c(N5CCC[C@@](C)(O)C5)nc(OCC5(CN6CCC(N7CCN(c8ccc(NC9CCC(=O)NC9=O)cc8F)CC7)CC6)CC5)nc4c3F)c12] |
| 111 | 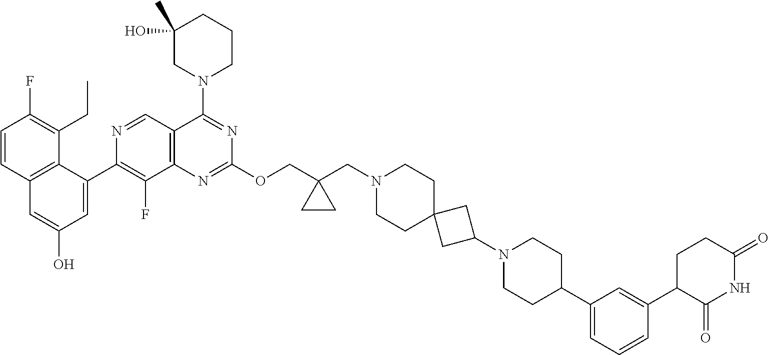 [SMILES: CCc1c(F)ccc2cc(O)cc(-c3ncc4c(N5CCC[C@@](C)(O)C5)nc(OCC5(CN6CCC7(CC6)CC(N6CCC(c8cccc(C9CCC(=O)NC9=O)c8)CC6)C7)CC5)nc4c3F)c12] |
| 112 | 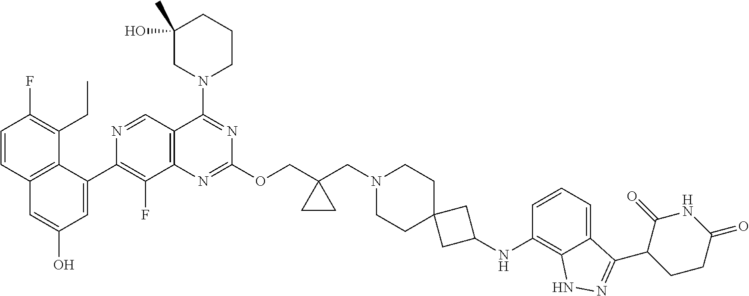 [SMILES: CCc1c(F)ccc2cc(O)cc(-c3ncc4c(N5CCC[C@@](C)(O)C5)nc(OCC5(CN6CCC7(CC6)CC(Nc6cccc8c(C9CCC(=O)NC9=O)n[nH]c68)C7)CC5)nc4c3F)c12] |
| 113 | 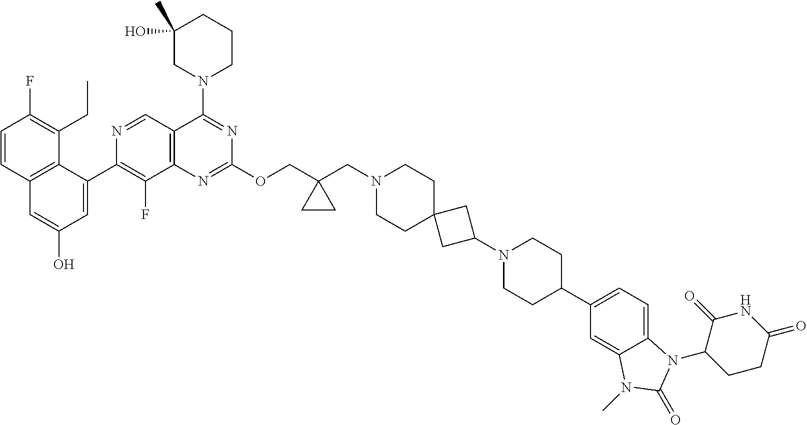 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c(c8)n(C)c(=O)n9C8CCC(=O)NC8=O)CC6)C7)CC5)nc4c3F)c12] |
| 114 | 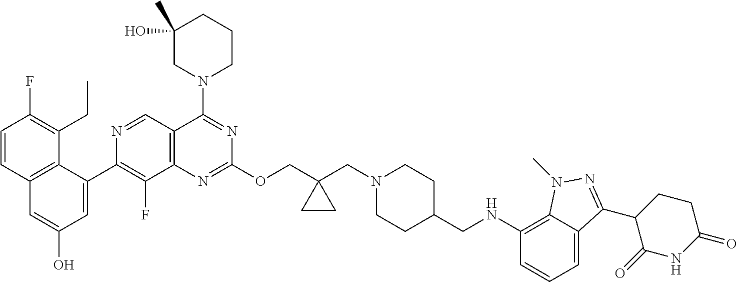 [SMILES: CCc1c(F)ccc2cc(O)cc(-c3ncc4c(N5CCC[C@@](C)(O)C5)nc(OCC5(CN6CCC(CNc7cccc8c(C9CCC(=O)NC9=O)nn(C)c78)CC6)CC5)nc4c3F)c12] |
| 115 | 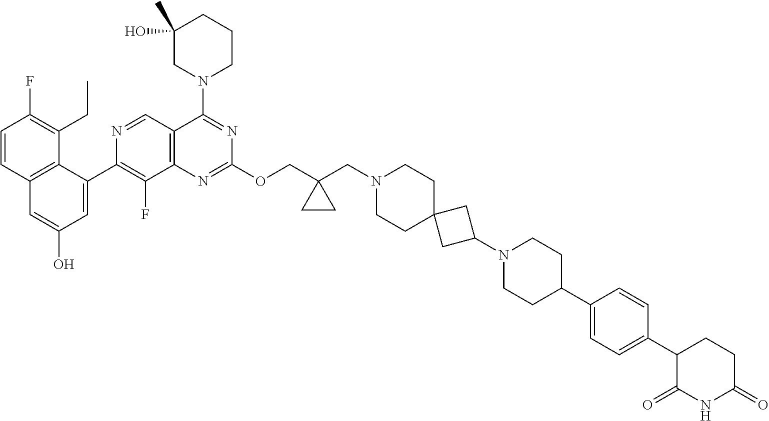 [SMILES: CCc1c(F)ccc2cc(O)cc(-c3ncc4c(N5CCC[C@@](C)(O)C5)nc(OCC5(CN6CCC7(CC6)CC(N6CCC(c8ccc(C9CCC(=O)NC9=O)cc8)CC6)C7)CC5)nc4c3F)c12] |
| 116 | 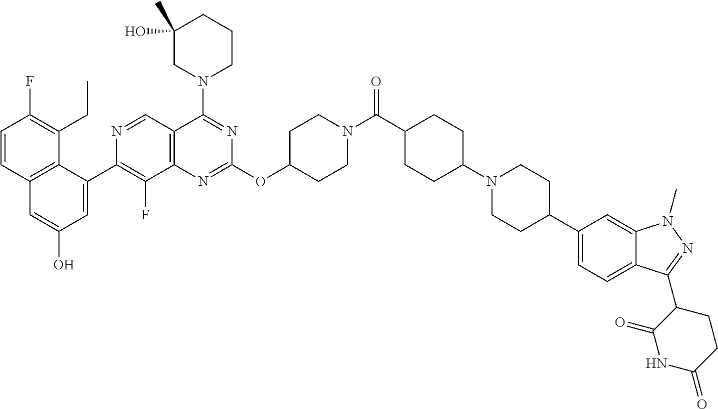 [SMILES: CCc1c(F)ccc2cc(O)cc(-c3ncc4c(N5CCC[C@@](C)(O)C5)nc(OC5CCN(C(=O)C6CCC(N7CCC(c8ccc9c(C%10CCC(=O)NC%10=O)nn(C)c9c8)CC7)CC6)CC5)nc4c3F)c12] |
| 117 | 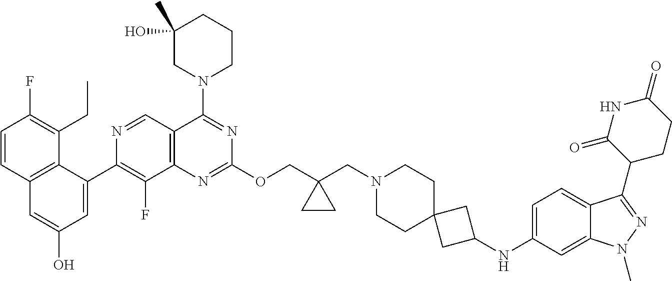 [SMILES: CCc1c(F)ccc2cc(O)cc(-c3ncc4c(N5CCC[C@@](C)(O)C5)nc(OCC5(CN6CCC7(CC6)CC(Nc6ccc8c(C9CCC(=O)NC9=O)nn(C)c8c6)C7)CC5)nc4c3F)c12] |
| 118 | 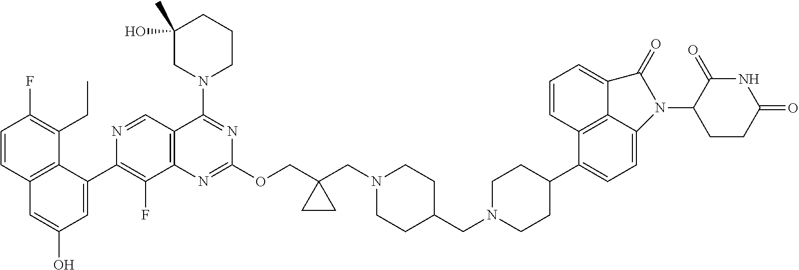 [SMILES: CCc1c(F)ccc2cc(O)cc(-c3ncc4c(N5CCC[C@@](C)(O)C5)nc(OCC5(CN6CCC(CN7CCC(c8ccc9c%10c(cccc8%10)C(=O)N9C8CCC(=O)NC8=O)CC7)CC6)CC5)nc4c3F)c12] |
| 119 | 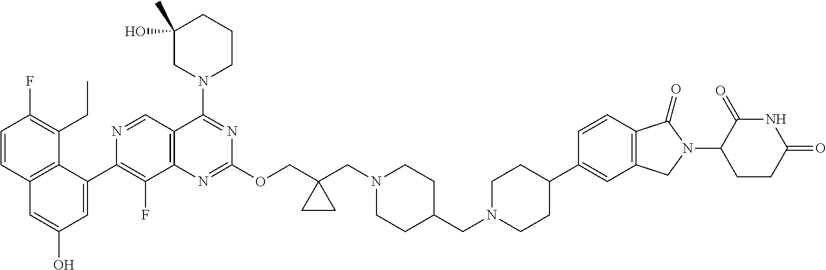 [SMILES: CCc1c(F)ccc2cc(O)cc(-c3ncc4c(N5CCC[C@@](C)(O)C5)nc(OCC5(CN6CCC(CN7CCC(c8ccc9c(c8)CN(C8CCC(=O)NC8=O)C9=O)CC7)CC6)CC5)nc4c3F)c12] |
| 120 | 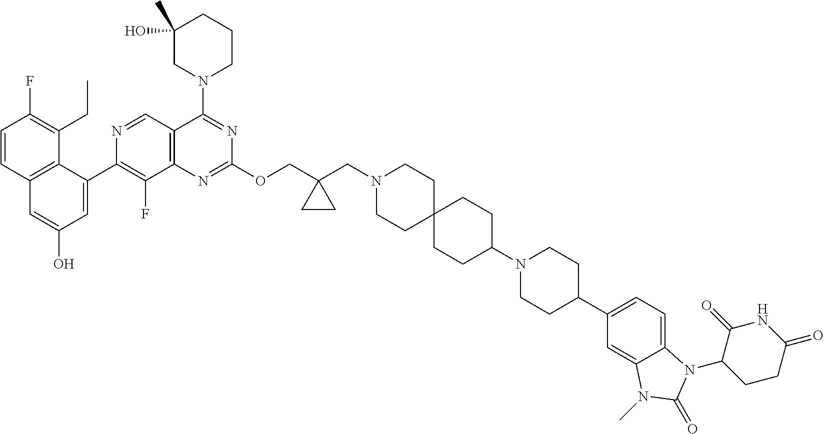 [SMILES: CCc1c(F)ccc2cc(O)cc(-c3ncc4c(N5CCC[C@@](C)(O)C5)nc(OCC5(CN6CCC7(CCC(N8CCC(c9ccc%10c(c9)n(C)c(=O)n%10C9CCC(=O)NC9=O)CC8)CC7)CC6)CC5)nc4c3F)c12] |
| 121 | 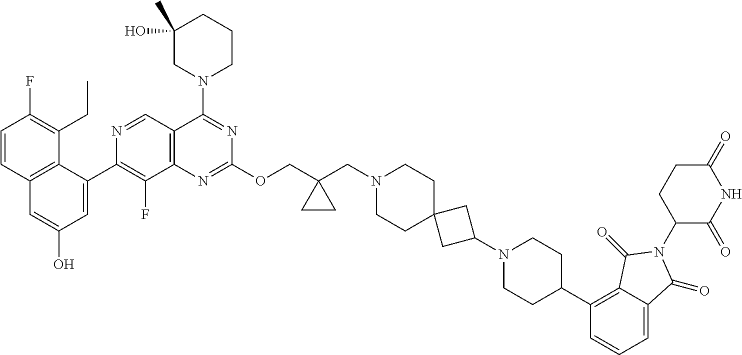 [SMILES: CCc1c(F)ccc2cc(O)cc(-c3ncc4c(N5CCC[C@@](C)(O)C5)nc(OCC5(CN6CCC7(CC6)CC(N6CCC(c8cccc9c8C(=O)N(C8CCC(=O)NC8=O)C9=O)CC6)C7)CC5)nc4c3F)c12] |
| 122 | 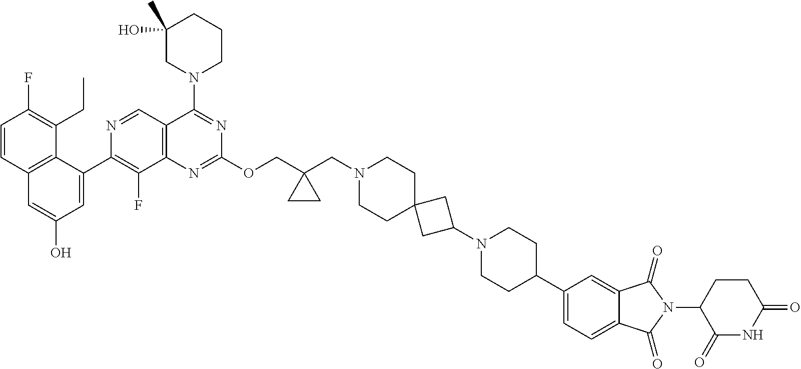 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c(c8)C(=O)N(C8CCC(=O)NC8=O)C9=O)CC6)C7)CC5)nc4c3F)c12] |
| 123 | 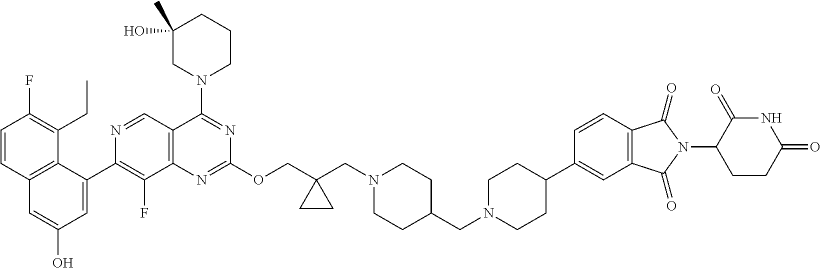 [SMILES: CCc1c(F)ccc2cc(O)cc(-c3ncc4c(N5CCC[C@@](C)(O)C5)nc(OCC5(CN6CCC(CN7CCC(c8ccc9c(c8)C(=O)N(C8CCC(=O)NC8=O)C9=O)CC7)CC6)CC5)nc4c3F)c12] |
| 124 | 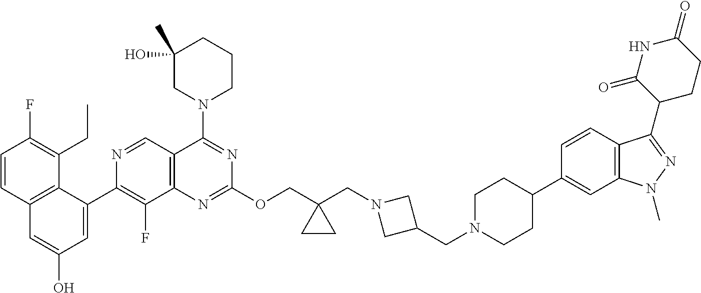 [SMILES: CCc1c(F)ccc2cc(O)cc(-c3ncc4c(N5CCC[C@@](C)(O)C5)nc(OCC5(CN6CC(CN7CCC(c8ccc9c(C%10CCC(=O)NC%10=O)nn(C)c9c8)CC7)C6)CC5)nc4c3F)c12] |
| 125 | 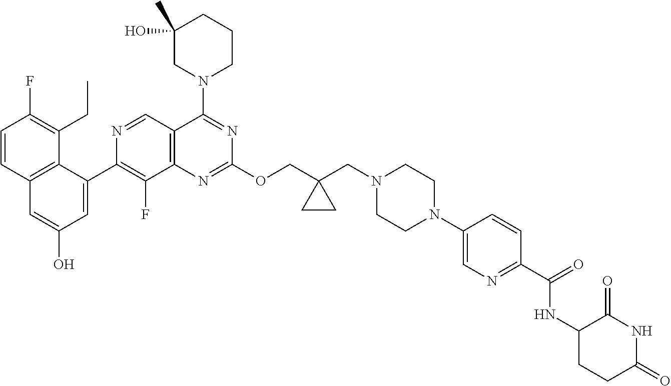 [SMILES: CCc1c(F)ccc2cc(O)cc(-c3ncc4c(N5CCC[C@@](C)(O)C5)nc(OCC5(CN6CCN(c7ccc(C(=O)NC8CCC(=O)NC8=O)nc7)CC6)CC5)nc4c3F)c12] |
| 126 | 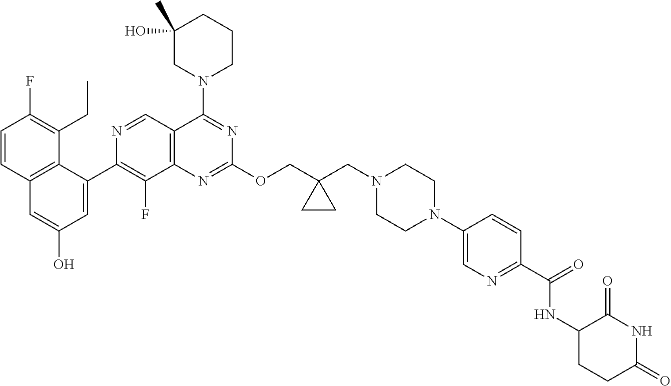 [SMILES: CCc1c(F)ccc2cc(O)cc(-c3ncc4c(N5CCC[C@@](C)(O)C5)nc(OCC5(CN6CCN(c7ccc(C(=O)NC8CCC(=O)NC8=O)nc7)CC6)CC5)nc4c3F)c12] |
| 127 | 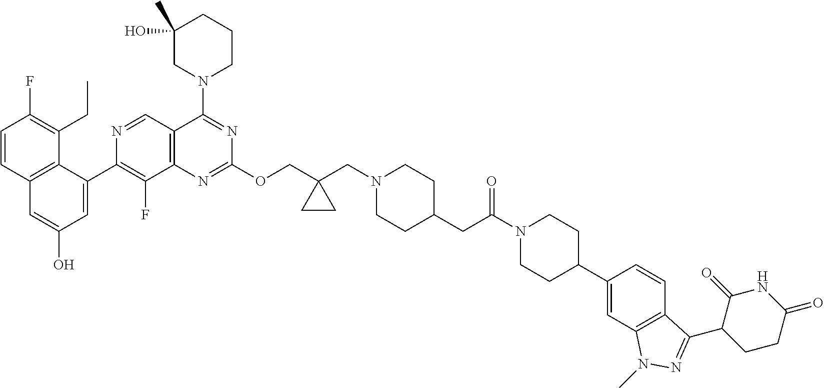 [SMILES: CCc1c(F)ccc2cc(O)cc(-c3ncc4c(N5CCC[C@@](C)(O)C5)nc(OCC5(CN6CCC(CC(=O)N7CCC(c8ccc9c(C%10CCC(=O)NC%10=O)nn(C)c9c8)CC7)CC6)CC5)nc4c3F)c12] |
| 128 | 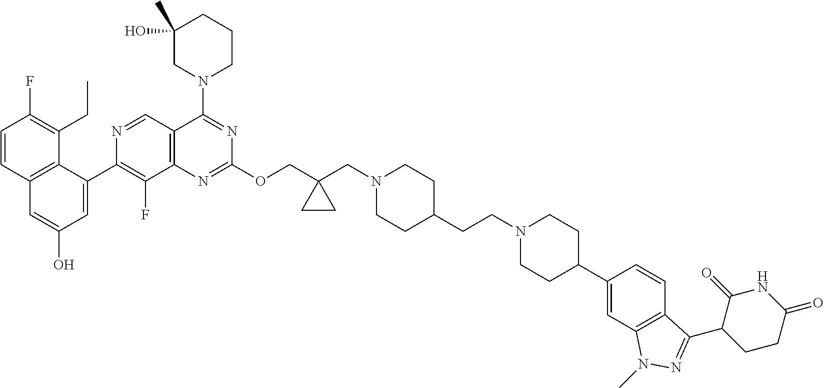 [SMILES: CCc1c(F)ccc2cc(O)cc(-c3ncc4c(N5CCC[C@@](C)(O)C5)nc(OCC5(CN6CCC(CCN7CCC(c8ccc9c(C%10CCC(=O)NC%10=O)nn(C)c9c8)CC7)CC6)CC5)nc4c3F)c12] |
| 129 | 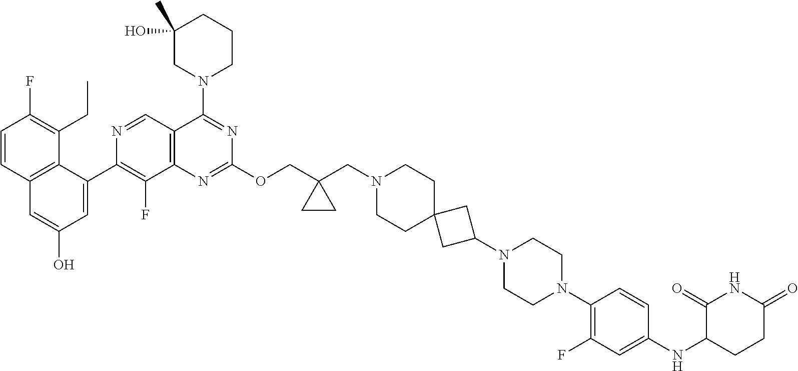 [SMILES: CCc1c(F)ccc2cc(O)cc(-c3ncc4c(N5CCC[C@@](C)(O)C5)nc(OCC5(CN6CCC7(CC6)CC(N6CCN(c8ccc(NC9CCC(=O)NC9=O)cc8F)CC6)C7)CC5)nc4c3F)c12] |
| 130 | 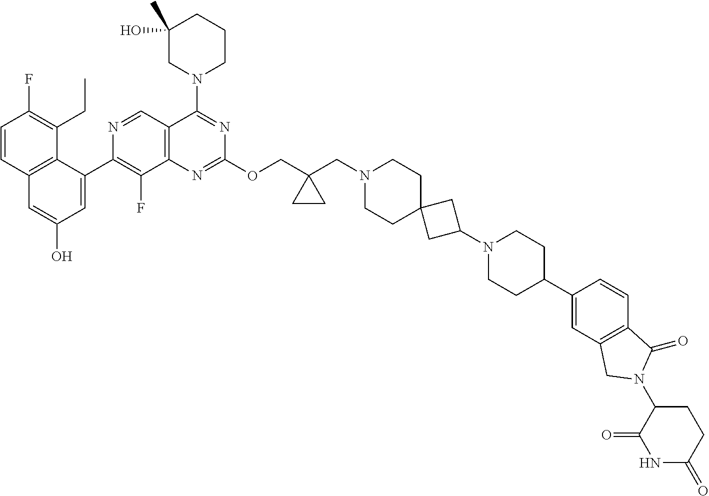 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c(c8)CN(C8CCC(=O)NC8=O)C9=O)CC6)C7)CC5)nc4c3F)c12] |
| 131 | 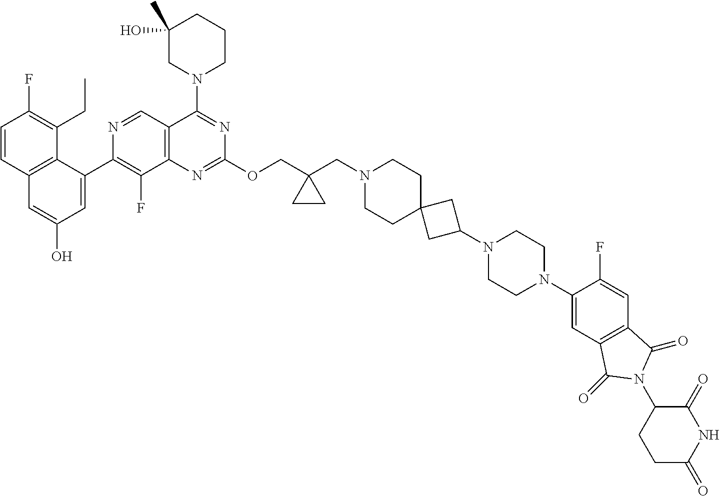 [SMILES: CCc1c(F)ccc2cc(O)cc(-c3ncc4c(N5CCC[C@@](C)(O)C5)nc(OCC5(CN6CCC7(CC6)CC(N6CCN(c8cc9c(cc8F)C(=O)N(C8CCC(=O)NC8=O)C9=O)CC6)C7)CC5)nc4c3F)c12] |
| 132 | 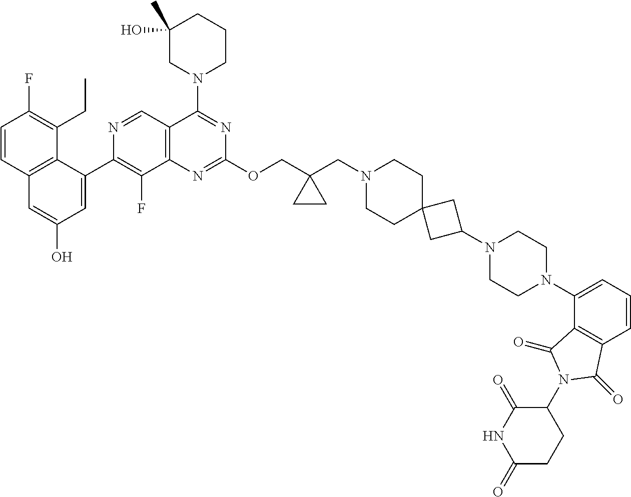 [SMILES: CCc1c(F)ccc2cc(O)cc(-c3ncc4c(N5CCC[C@@](C)(O)C5)nc(OCC5(CN6CCC7(CC6)CC(N6CCN(c8cccc9c8C(=O)N(C8CCC(=O)NC8=O)C9=O)CC6)C7)CC5)nc4c3F)c12] |
| 133 | 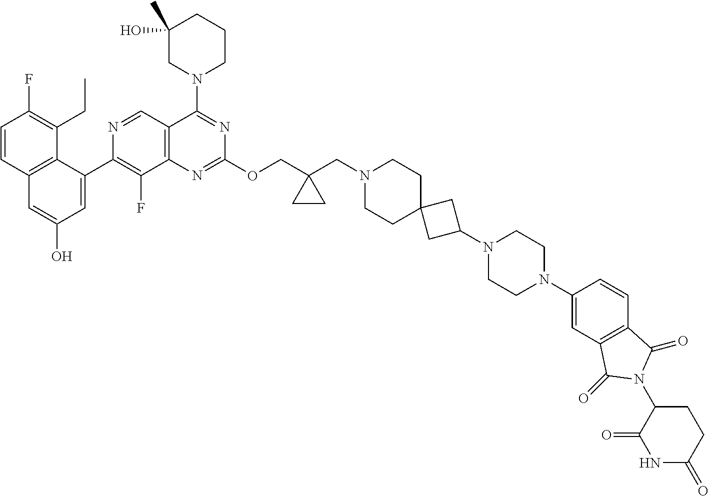 [SMILES: CCc1c(F)ccc2cc(O)cc(-c3ncc4c(N5CCC[C@@](C)(O)C5)nc(OCC5(CN6CCC7(CC6)CC(N6CCN(c8ccc9c(c8)C(=O)N(C8CCC(=O)NC8=O)C9=O)CC6)C7)CC5)nc4c3F)c12] |
| 134 | 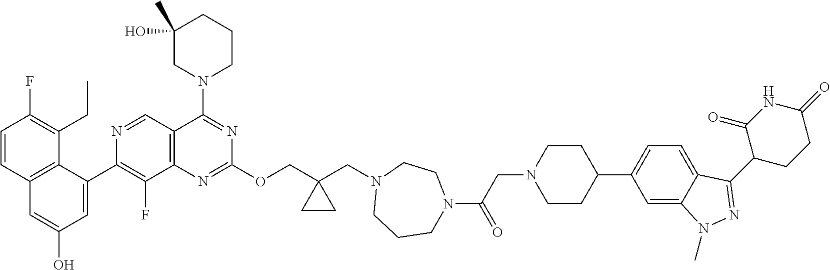 [SMILES: CCc1c(F)ccc2cc(O)cc(-c3ncc4c(N5CCC[C@@](C)(O)C5)nc(OCC5(CN6CCCN(C(=O)CN7CCC(c8ccc9c(C%10CCC(=O)NC%10=O)nn(C)c9c8)CC7)CC6)CC5)nc4c3F)c12] |
| 135 | 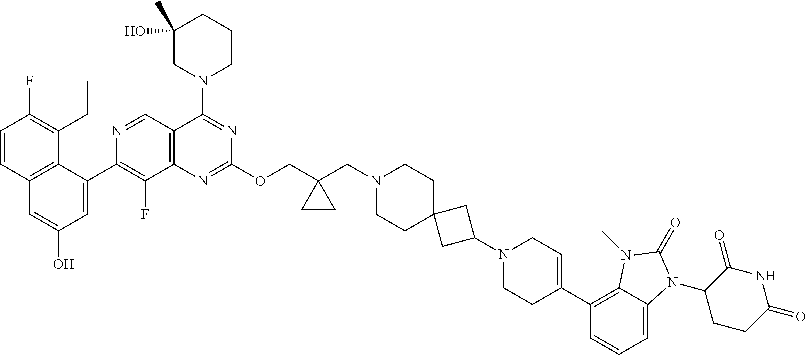 [SMILES: CCc1c(F)ccc2cc(O)cc(-c3ncc4c(N5CCC[C@@](C)(O)C5)nc(OCC5(CN6CCC7(CC6)CC(N6CC=C(c8cccc9c8n(C)c(=O)n9C8CCC(=O)NC8=O)CC6)C7)CC5)nc4c3F)c12] |
| 136 | 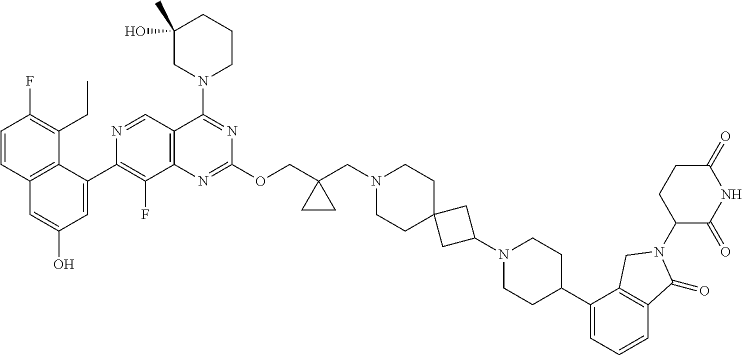 [SMILES: CCc1c(F)ccc2cc(O)cc(-c3ncc4c(N5CCC[C@@](C)(O)C5)nc(OCC5(CN6CCC7(CC6)CC(N6CCC(c8cccc9c8CN(C8CCC(=O)NC8=O)C9=O)CC6)C7)CC5)nc4c3F)c12] |
| 137 | 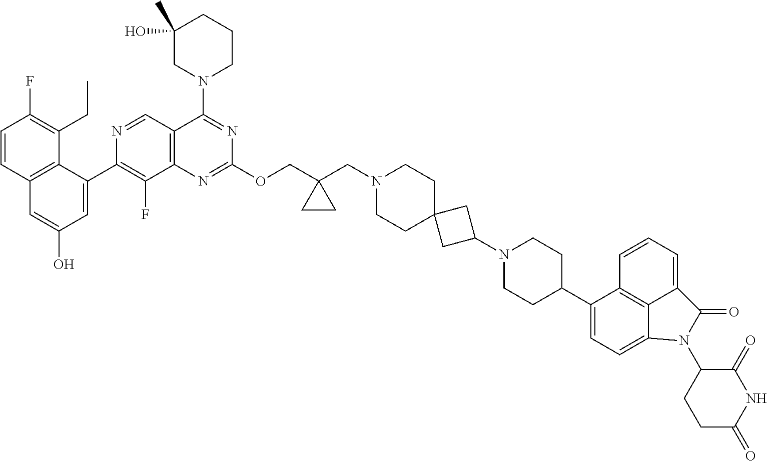 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |
| 138 | 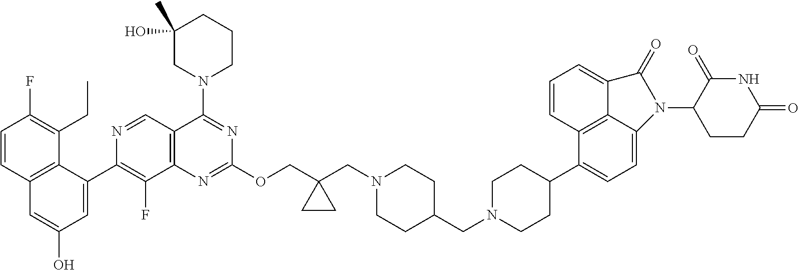 [SMILES: CCc1c(F)ccc2cc(O)cc(-c3ncc4c(N5CCC[C@@](C)(O)C5)nc(OCC5(CN6CCC(CN7CCC(c8ccc9c%10c(cccc8%10)C(=O)N9C8CCC(=O)NC8=O)CC7)CC6)CC5)nc4c3F)c12] |

TABLE 3
Exemplary bifunctional compounds of the disclosure in accordance with certain
embodiments.

| Compound | |
|---|---|
| No. | Structure |
| 139 | 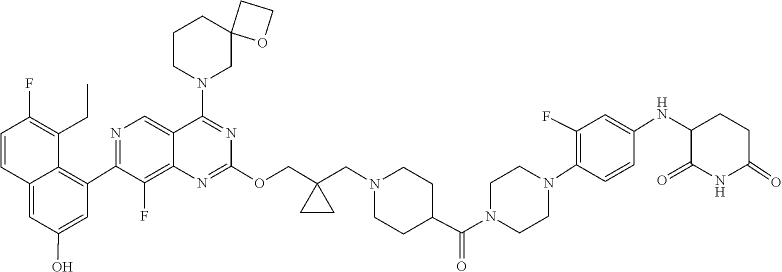 [SMILES: CCc1c(F)ccc2cc(O)cc(-c3ncc4c(N5CCCC6(CCO6)C5)nc(OCC5(CN6CCC(C(=O)N7CCN(c8ccc(NC9CCC(=O)NC9=O)cc8F)CC7)CC6)CC5)nc4c3F)c12] |
| 140 | 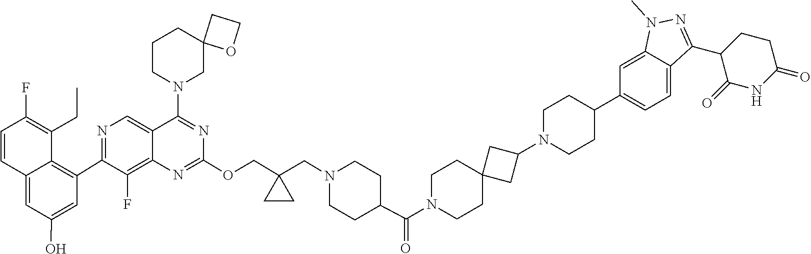 [SMILES: CCc1c(F)ccc2cc(O)cc(-c3ncc4c(N5CCCC6(CCO6)C5)nc(OCC5(CN6CCC(C(=O)N7CCC8(CC7)CC(N7CCC(c9ccc%10c(C%11CCC(=O)NC%11=O)nn(C)c%10c9)CC7)C8)CC6)CC5)nc4c3F)c12] |
| 141 | 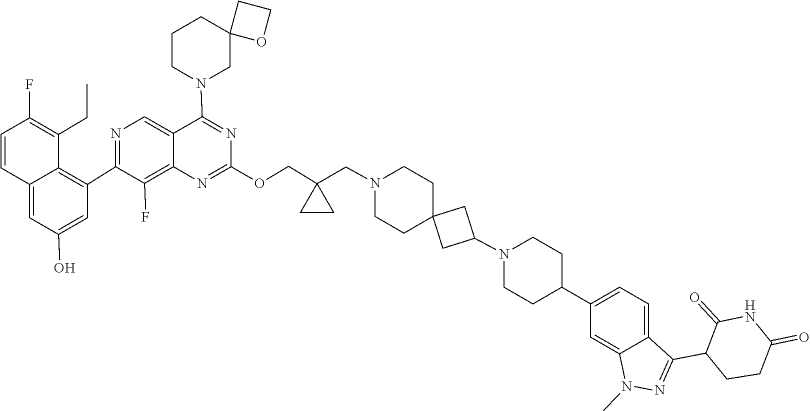 [SMILES: CCc1c(F)ccc2cc(O)cc(-c3ncc4c(N5CCCC6(CCO6)C5)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 142 | 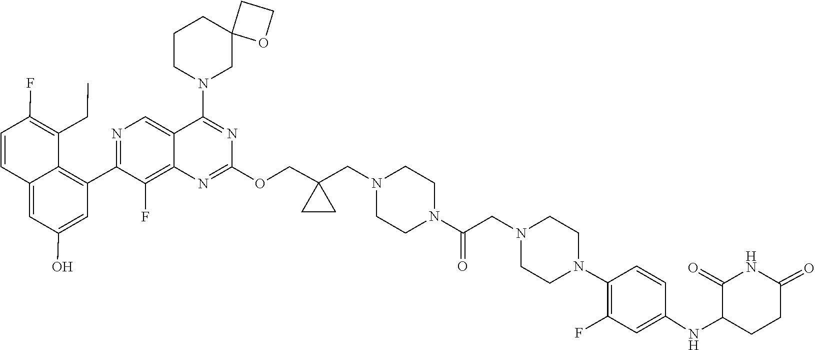 [SMILES: CCc1c(F)ccc2cc(O)cc(-c3ncc4c(N5CCCC6(CCO6)C5)nc(OCC5(CN6CCN(C(=O)CN7CCN(c8ccc(NC9CCC(=O)NC9=O)cc8F)CC7)CC6)CC5)nc4c3F)c12] |
| 143 | 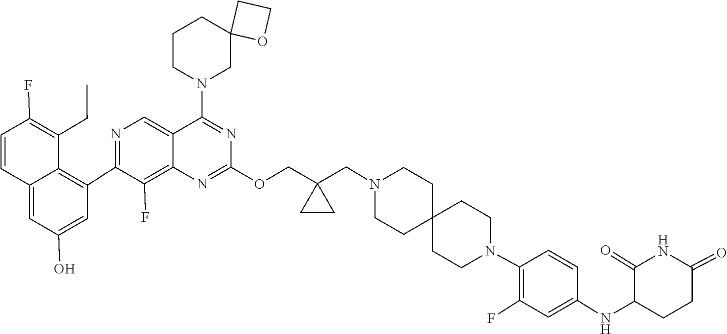 [SMILES: CCc1c(F)ccc2cc(O)cc(-c3ncc4c(N5CCCC6(CCO6)C5)nc(OCC5(CN6CCC7(CC6)CCN(c6ccc(NC8CCC(=O)NC8=O)cc6F)CC7)CC5)nc4c3F)c12] |
| 144 | 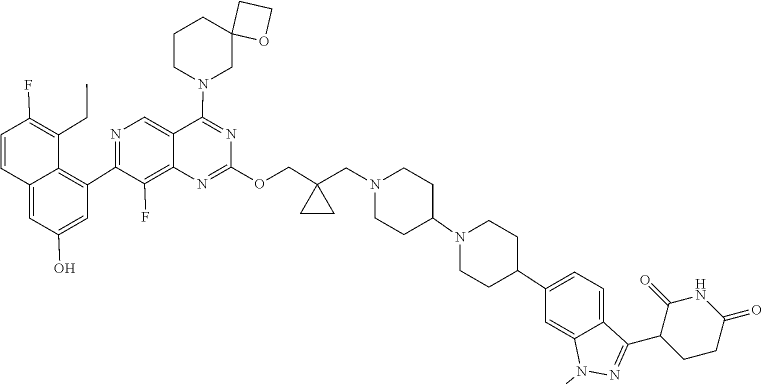 [SMILES: CCc1c(F)ccc2cc(O)cc(-c3ncc4c(N5CCCC6(CCO6)C5)nc(OCC5(CN6CCC(N7CCC(c8ccc9c(C%10CCC(=O)NC%10=O)nn(C)c9c8)CC7)CC6)CC5)nc4c3F)c12] |
| 145 | 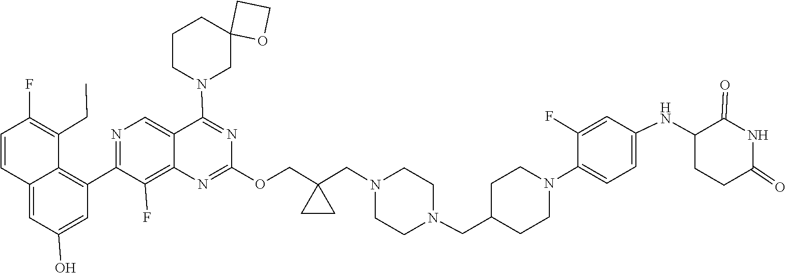 [SMILES: CCc1c(F)ccc2cc(O)cc(-c3ncc4c(N5CCCC6(CCO6)C5)nc(OCC5(CN6CCN(CC7CCN(c8ccc(NC9CCC(=O)NC9=O)cc8F)CC7)CC6)CC5)nc4c3F)c12] |
| 146 | 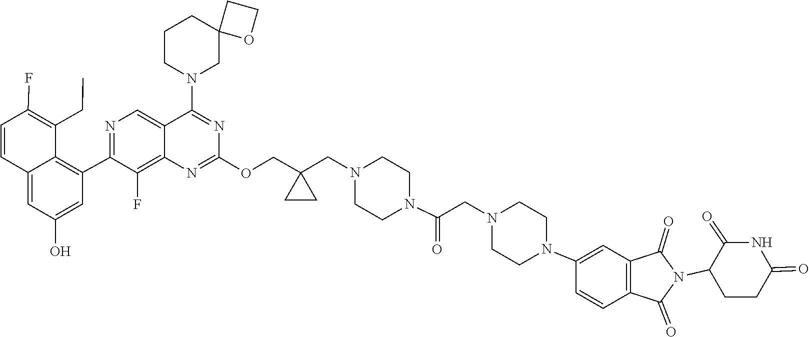 [SMILES: CCc1c(F)ccc2cc(O)cc(-c3ncc4c(N5CCCC6(CCO6)C5)nc(OCC5(CN6CCN(C(=O)CN7CCN(c8ccc9c(c8)C(=O)N(C8CCC(=O)NC8=O)C9=O)CC7)CC6)CC5)nc4c3F)c12] |
| 147 | 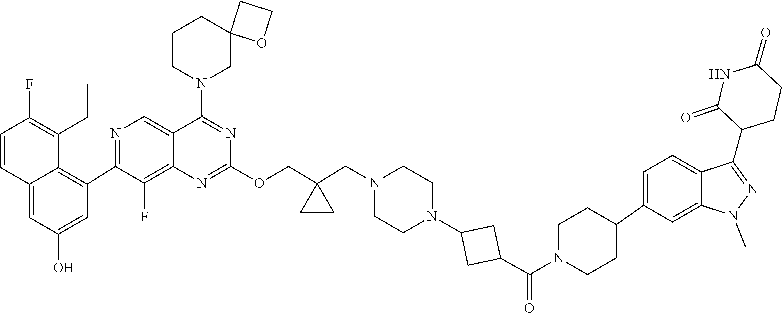 [SMILES: CCc1c(F)ccc2cc(O)cc(-c3ncc4c(N5CCCC6(CCO6)C5)nc(OCC5(CN6CCN(C7CC(C(=O)N8CCC(c9ccc%10c(C%11CCC(=O)NC%11=O)nn(C)c%10c9)CC8)C7)CC6)CC5)nc4c3F)c12] |
| 148 | 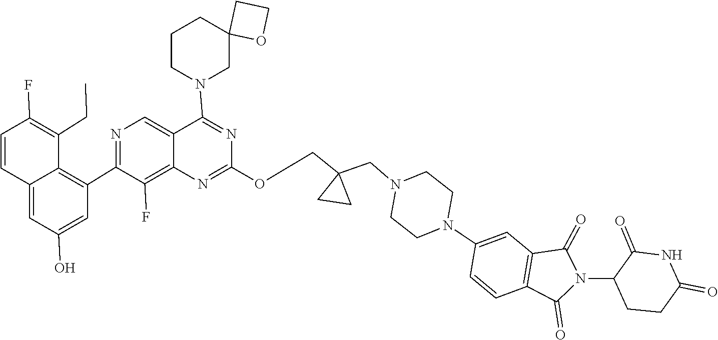 [SMILES: CCc1c(F)ccc2cc(O)cc(-c3ncc4c(N5CCCC6(CCO6)C5)nc(OCC5(CN6CCN(c7ccc8c(c7)C(=O)N(C7CCC(=O)NC7=O)C8=O)CC6)CC5)nc4c3F)c12] |
| 149 | 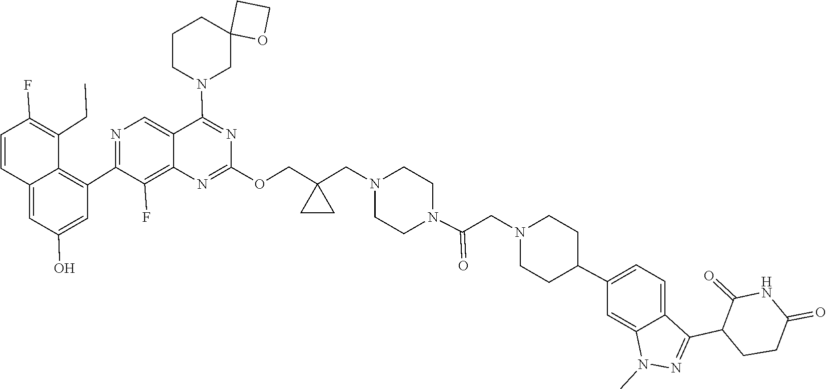 [SMILES: CCc1c(F)ccc2cc(O)cc(-c3ncc4c(N5CCCC6(CCO6)C5)nc(OCC5(CN6CCN(C(=O)CN7CCC(c8ccc9c(C%10CCC(=O)NC%10=O)nn(C)c9c8)CC7)CC6)CC5)nc4c3F)c12] |
| 150 | 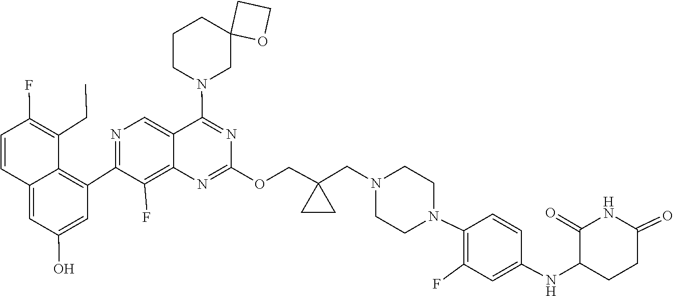 [SMILES: CCc1c(F)ccc2cc(O)cc(-c3ncc4c(N5CCCC6(CCO6)C5)nc(OCC5(CN6CCN(c7ccc(NC8CCC(=O)NC8=O)cc7F)CC6)CC5)nc4c3F)c12] |
| 151 | 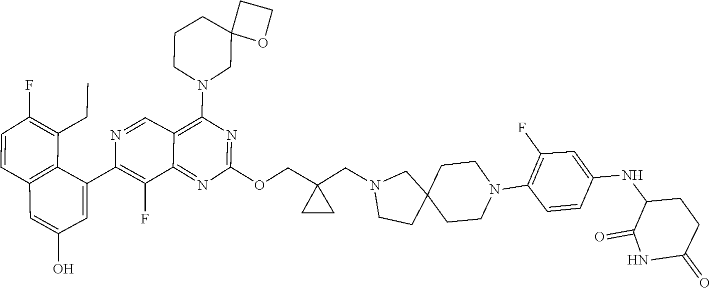 [SMILES: CCc1c(F)ccc2cc(O)cc(-c3ncc4c(N5CCCC6(CCO6)C5)nc(OCC5(CN6CCC7(CCN(c8ccc(NC9CCC(=O)NC9=O)cc8F)CC7)C6)CC5)nc4c3F)c12] |
| 152 | 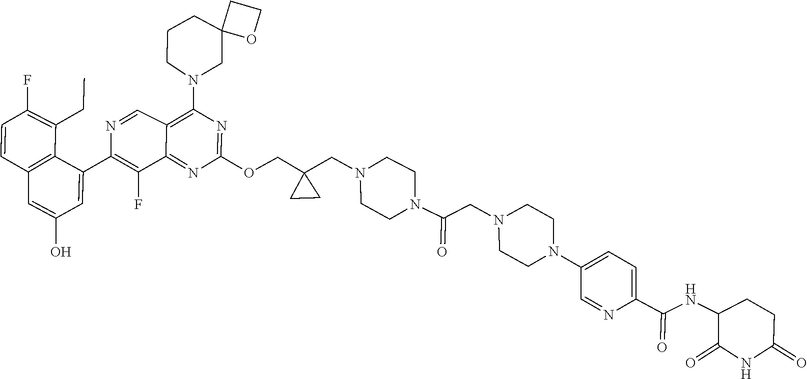 [SMILES: CCc1c(F)ccc2cc(O)cc(-c3ncc4c(N5CCCC6(CCO6)C5)nc(OCC5(CN6CCN(C(=O)CN7CCN(c8ccc(C(=O)NC9CCC(=O)NC9=O)nc8)CC7)CC6)CC5)nc4c3F)c12] |
| 153 | 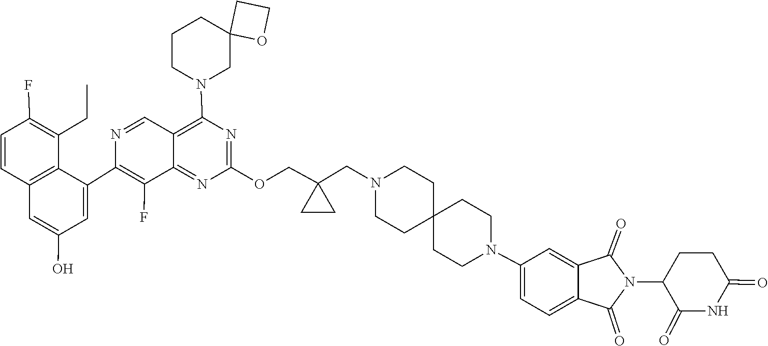 [SMILES: CCc1c(F)ccc2cc(O)cc(-c3ncc4c(N5CCCC6(CCO6)C5)nc(OCC5(CN6CCC7(CC6)CCN(c6ccc8c(c6)C(=O)N(C6CCC(=O)NC6=O)C8=O)CC7)CC5)nc4c3F)c12] |
| 154 | 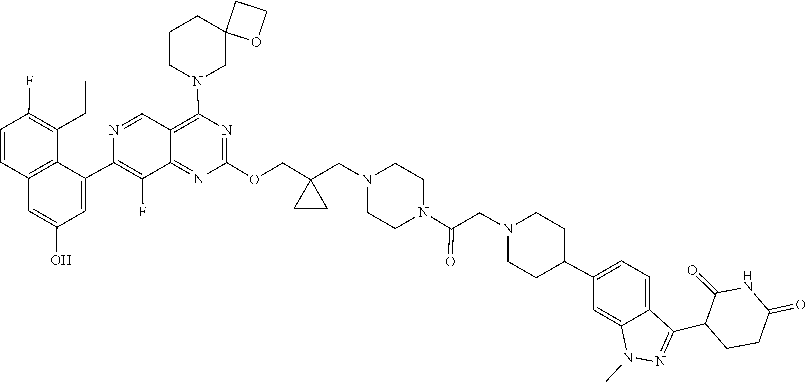 [SMILES: CCc1c(F)ccc2cc(O)cc(-c3ncc4c(N5CCCC6(CCO6)C5)nc(OCC5(CN6CCN(C(=O)CN7CCC(c8ccc9c(C%10CCC(=O)NC%10=O)nn(C)c9c8)CC7)CC6)CC5)nc4c3F)c12] |
| 155 | 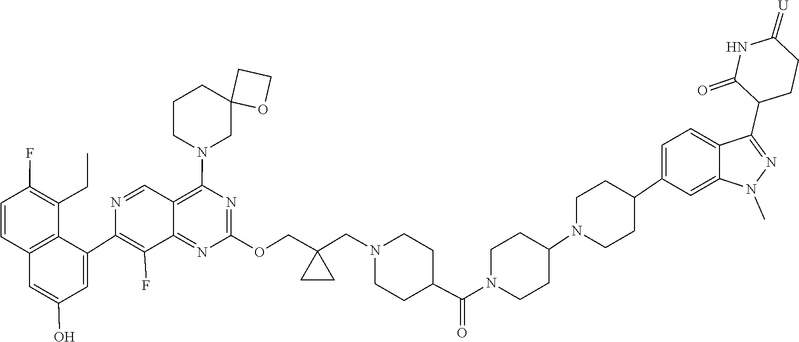 |
| 156 | 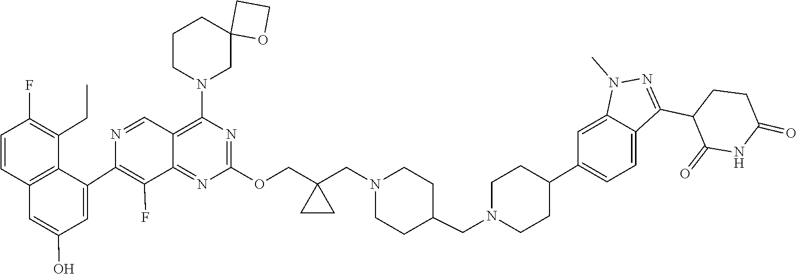 [SMILES: CCc1c(F)ccc2cc(O)cc(-c3ncc4c(N5CCCC6(CCO6)C5)nc(OCC5(CN6CCC(CN7CCC(c8ccc9c(C%10CCC(=O)NC%10=O)nn(C)c9c8)CC7)CC6)CC5)nc4c3F)c12] |
| 157 | 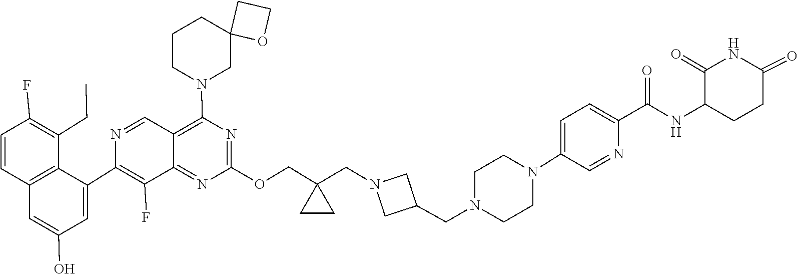 [SMILES: CCc1c(F)ccc2cc(O)cc(-c3ncc4c(N5CCCC6(CCO6)C5)nc(OCC5(CN6CC(CN7CCN(c8ccc(C(=O)NC9CCC(=O)NC9=O)nc8)CC7)C6)CC5)nc4c3F)c12] |
| 158 | 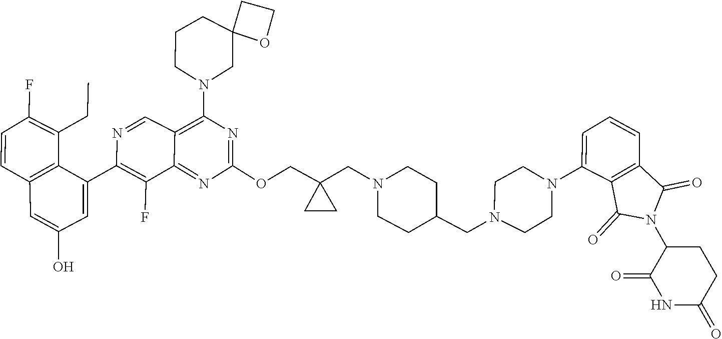 [SMILES: CCc1c(F)ccc2cc(O)cc(-c3ncc4c(N5CCCC6(CCO6)C5)nc(OCC5(CN6CCC(CN7CCN(c8cccc9c8C(=O)N(C8CCC(=O)NC8=O)C9=O)CC7)CC6)CC5)nc4c3F)c12] |
| 159 | 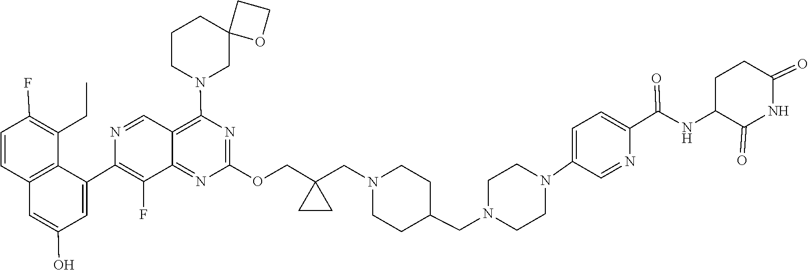 [SMILES: CCc1c(F)ccc2cc(O)cc(-c3ncc4c(N5CCCC6(CCO6)C5)nc(OCC5(CN6CCC(CN7CCN(c8ccc(C(=O)NC9CCC(=O)NC9=O)nc8)CC7)CC6)CC5)nc4c3F)c12] |
| 160 | 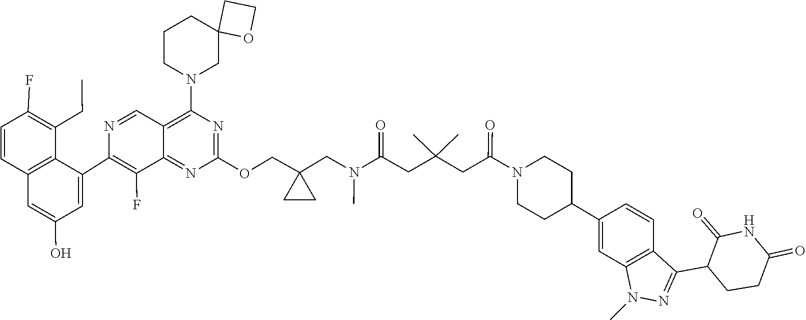 [SMILES: CCc1c(F)ccc2cc(O)cc(-c3ncc4c(N5CCCC6(CCO6)C5)nc(OCC5(CN(C)C(=O)CC(C)(C)CC(=O)N6CCC(c7ccc8c(C9CCC(=O)NC9=O)nn(C)c8c7)CC6)CC5)nc4c3F)c12] |
| 161 | 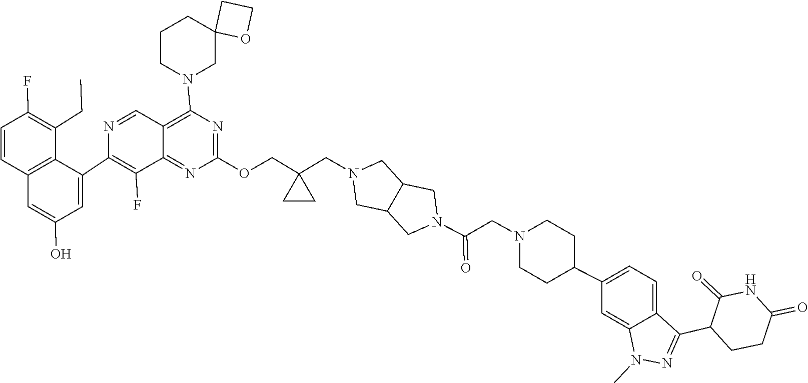 [SMILES: CCc1c(F)ccc2cc(O)cc(-c3ncc4c(N5CCCC6(CCO6)C5)nc(OCC5(CN6CC7CN(C(=O)CN8CCC(c9ccc%10c(C%11CCC(=O)NC%11=O)nn(C)c%10c9)CC8)CC7C6)CC5)nc4c3F)c12] |
| 162 | 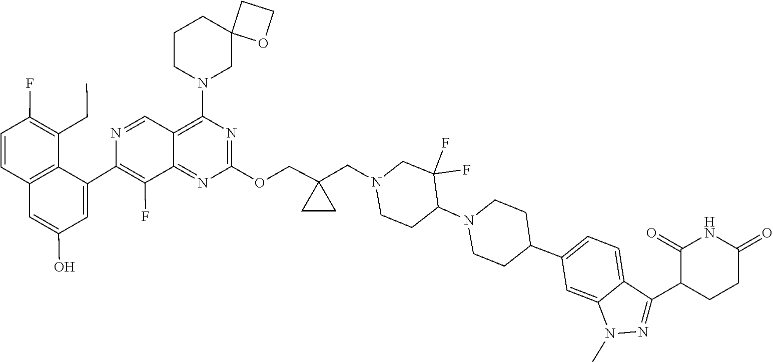 [SMILES: CCc1c(F)ccc2cc(O)cc(-c3ncc4c(N5CCCC6(CCO6)C5)nc(OCC5(CN6CCC(N7CCC(c8ccc9c(C%10CCC(=O)NC%10=O)nn(C)c9c8)CC7)C(F)(F)C6)CC5)nc4c3F)c12] |
| 163 | 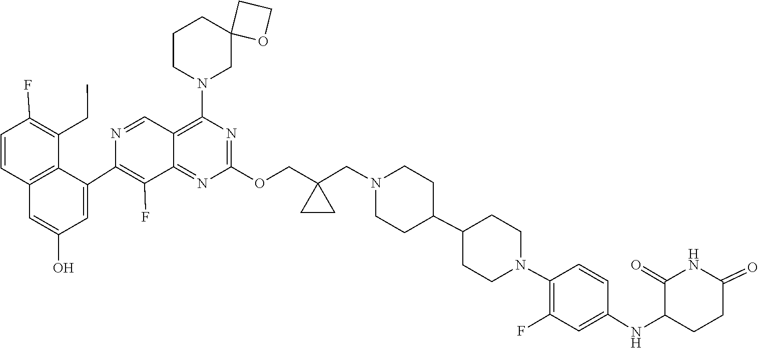 [SMILES: CCc1c(F)ccc2cc(O)cc(-c3ncc4c(N5CCCC6(CCO6)C5)nc(OCC5(CN6CCC(C7CCN(c8ccc(NC9CCC(=O)NC9=O)cc8F)CC7)CC6)CC5)nc4c3F)c12] |
| 164 | 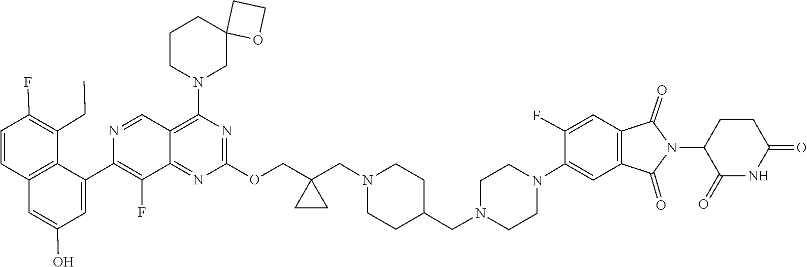 [SMILES: CCc1c(F)ccc2cc(O)cc(-c3ncc4c(N5CCCC6(CCO6)C5)nc(OCC5(CN6CCC(CN7CCN(c8cc9c(cc8F)C(=O)N(C8CCC(=O)NC8=O)C9=O)CC7)CC6)CC5)nc4c3F)c12] |
| 165 | 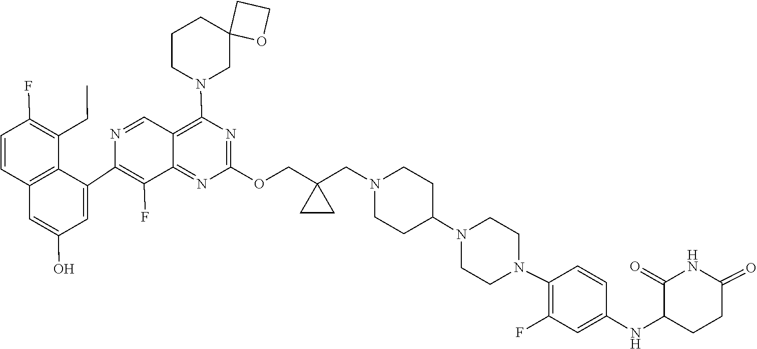 [SMILES: CCc1c(F)ccc2cc(O)cc(-c3ncc4c(N5CCCC6(CCO6)C5)nc(OCC5(CN6CCC(N7CCN(c8ccc(NC9CCC(=O)NC9=O)cc8F)CC7)CC6)CC5)nc4c3F)c12] |
| 166 | 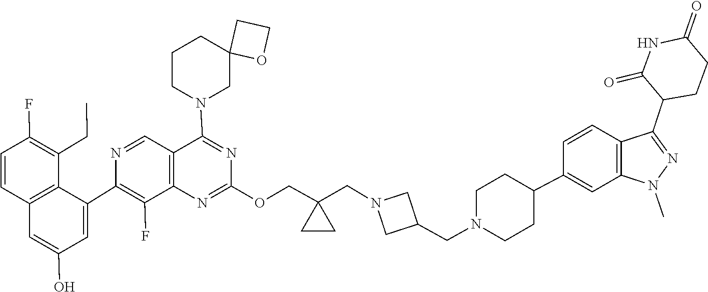 [SMILES: CCc1c(F)ccc2cc(O)cc(-c3ncc4c(N5CCCC6(CCO6)C5)nc(OCC5(CN6CC(CN7CCC(c8ccc9c(C%10CCC(=O)NC%10=O)nn(C)c9c8)CC7)C6)CC5)nc4c3F)c12] |
| 167 | 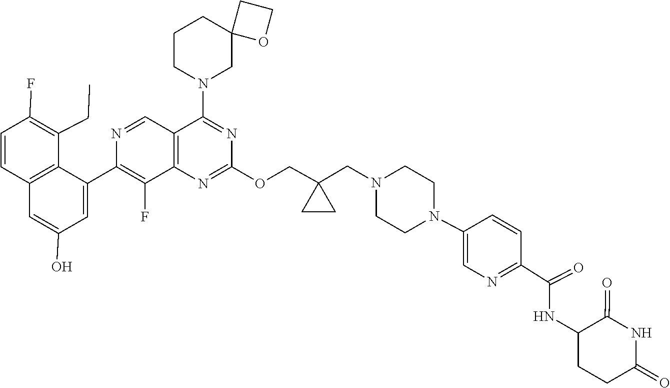 [SMILES: CCc1c(F)ccc2cc(O)cc(-c3ncc4c(N5CCCC6(CCO6)C5)nc(OCC5(CN6CCN(c7ccc(C(=O)NC8CCC(=O)NC8=O)nc7)CC6)CC5)nc4c3F)c12] |
| 168 | 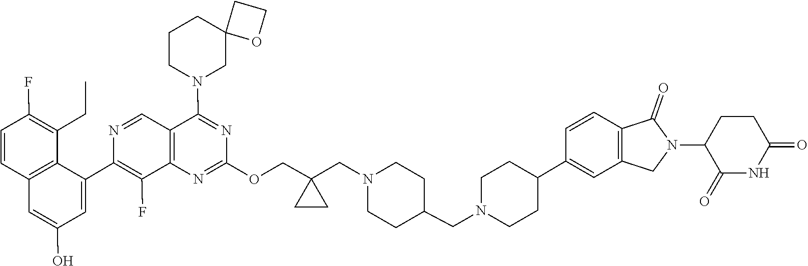 [SMILES: CCc1c(F)ccc2cc(O)cc(-c3ncc4c(N5CCCC6(CCO6)C5)nc(OCC5(CN6CCC(CN7CCC(c8ccc9c(c8)CN(C8CCC(=O)NC8=O)C9=O)CC7)CC6)CC5)nc4c3F)c12] |
| 169 | 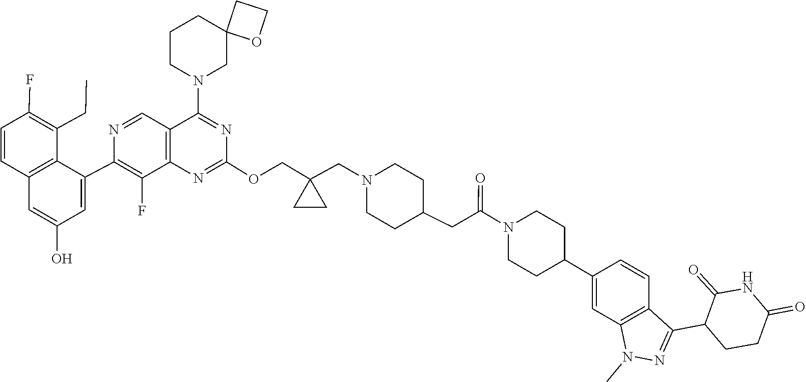 [SMILES: CCc1c(F)ccc2cc(O)cc(-c3ncc4c(N5CCCC6(CCO6)C5)nc(OCC5(CN6CCC(CC(=O)N7CCC(c8ccc9c(C%10CCC(=O)NC%10=O)nn(C)c9c8)CC7)CC6)CC5)nc4c3F)c12] |
| 170 | 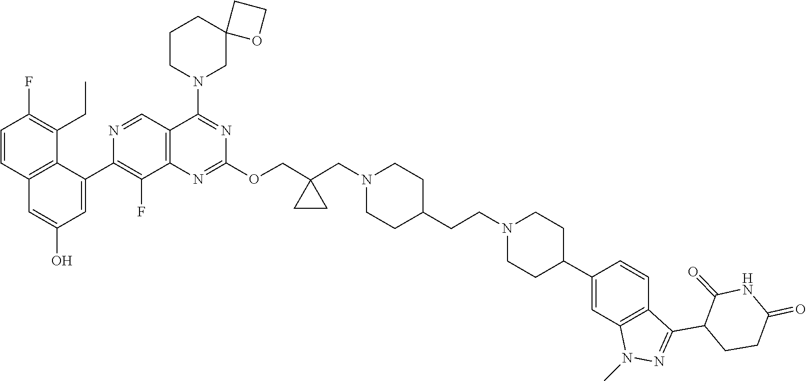 [SMILES: CCc1c(F)ccc2cc(O)cc(-c3ncc4c(N5CCCC6(CCO6)C5)nc(OCC5(CN6CCC(CCN7CCC(c8ccc9c(C%10CCC(=O)NC%10=O)nn(C)c9c8)CC7)CC6)CC5)nc4c3F)c12] |
| 171 | 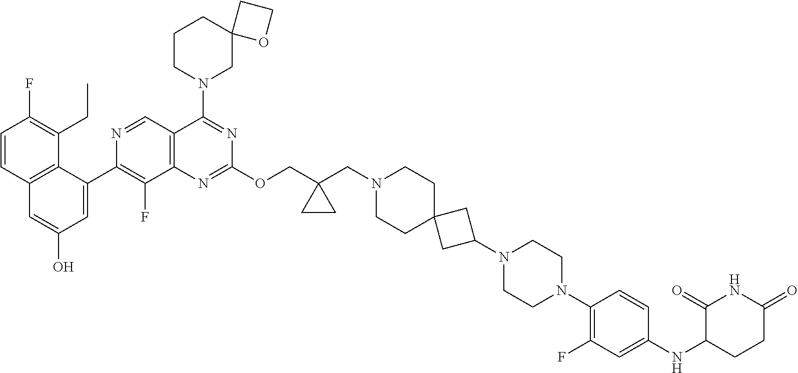 [SMILES: CCc1c(F)ccc2cc(O)cc(-c3ncc4c(N5CCCC6(CCO6)C5)nc(OCC5(CN6CCC7(CC6)CC(N6CCN(c8ccc(NC9CCC(=O)NC9=O)cc8F)CC6)C7)CC5)nc4c3F)c12] |
| 172 | 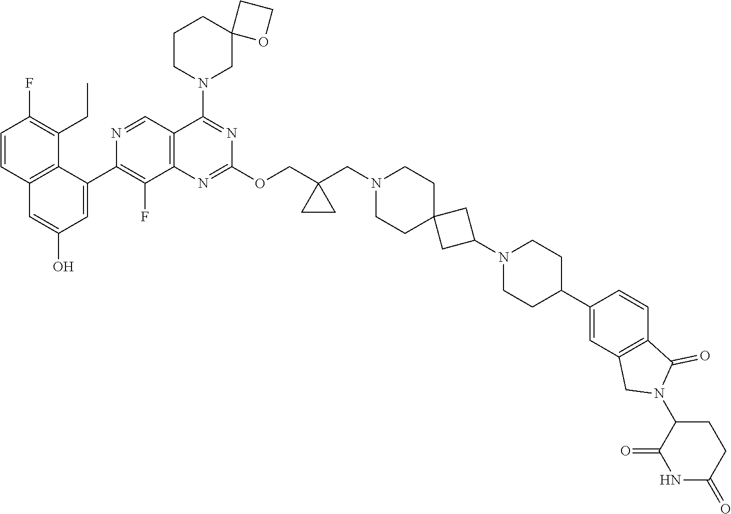 [SMILES: CCc1c(F)ccc2cc(O)cc(-c3ncc4c(N5CCCC6(CCO6)C5)nc(OCC5(CN6CCC7(CC6)CC(N6CCC(c8ccc9c(c8)CN(C8CCC(=O)NC8=O)C9=O)CC6)C7)CC5)nc4c3F)c12] |
| 173 | 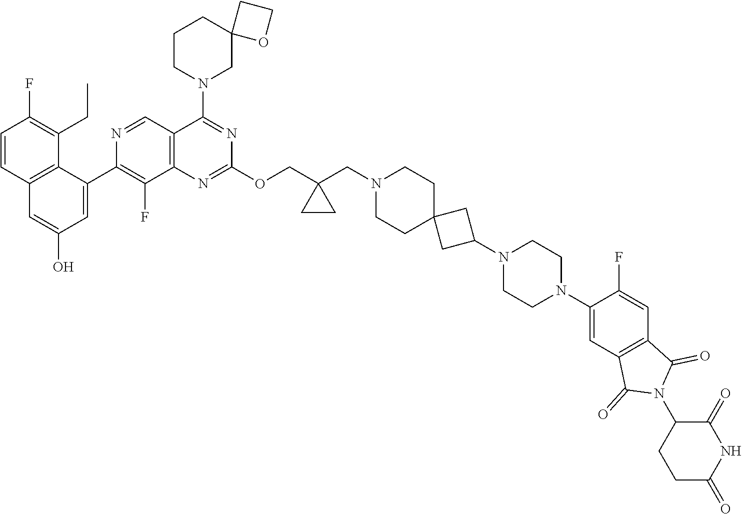 [SMILES: CCc1c(F)ccc2cc(O)cc(-c3ncc4c(N5CCCC6(CCO6)C5)nc(OCC5(CN6CCC7(CC6)CC(N6CCN(c8cc9c(cc8F)C(=O)N(C8CCC(=O)NC8=O)C9=O)CC6)C7)CC5)nc4c3F)c12] |
| 174 | 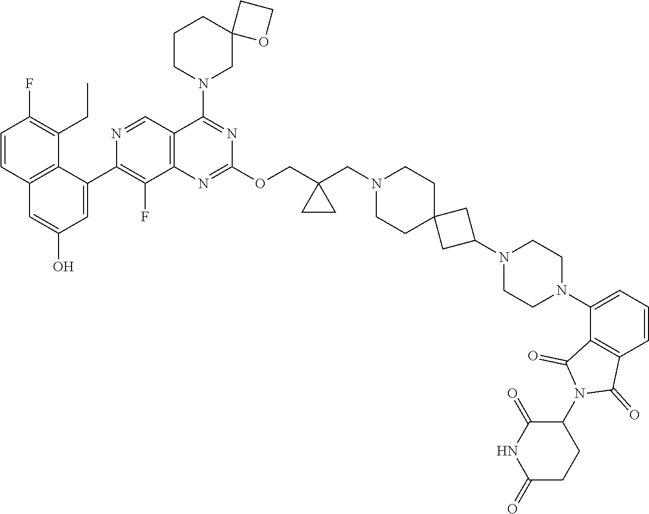 [SMILES: CCc1c(F)ccc2cc(O)cc(-c3ncc4c(N5CCCC6(CCO6)C5)nc(OCC5(CN6CCC7(CC6)CC(N6CCN(c8cccc9c8C(=O)N(C8CCC(=O)NC8=O)C9=O)CC6)C7)CC5)nc4c3F)c12] |
| 175 | 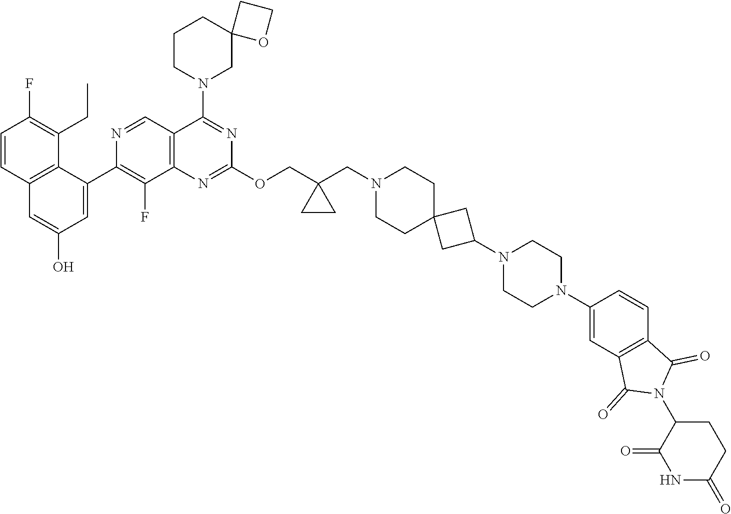 [SMILES: CCc1c(F)ccc2cc(O)cc(-c3ncc4c(N5CCCC6(CCO6)C5)nc(OCC5(CN6CCC7(CC6)CC(N6CCN(c8ccc9c(c8)C(=O)N(C8CCC(=O)NC8=O)C9=O)CC6)C7)CC5)nc4c3F)c12] |
| 176 | 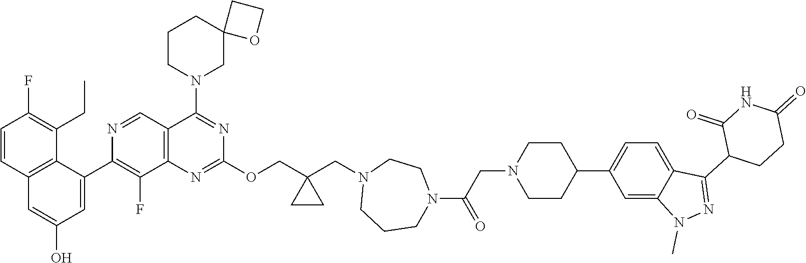 [SMILES: CCc1c(F)ccc2cc(O)cc(-c3ncc4c(N5CCCC6(CCO6)C5)nc(OCC5(CN6CCCN(C(=O)CN7CCC(c8ccc9c(C%10CCC(=O)NC%10=O)nn(C)c9c8)CC7)CC6)CC5)nc4c3F)c12] |
| 177 | 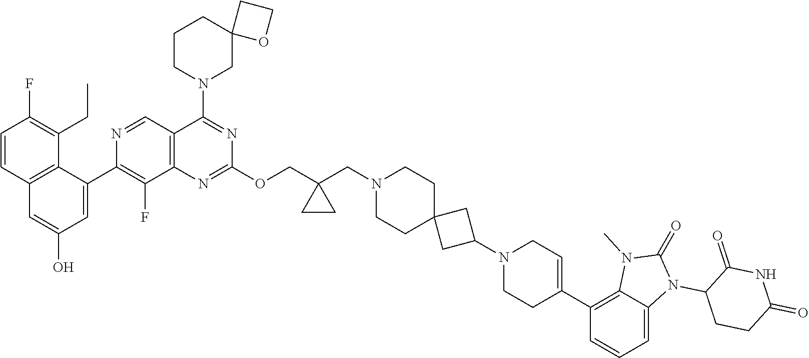 [SMILES: CCc1c(F)ccc2cc(O)cc(-c3ncc4c(N5CCCC6(CCO6)C5)nc(OCC5(CN6CCC7(CC6)CC(N6CC=C(c8cccc9c8n(C)c(=O)n9C8CCC(=O)NC8=O)CC6)C7)CC5)nc4c3F)c12] |
| 178 | 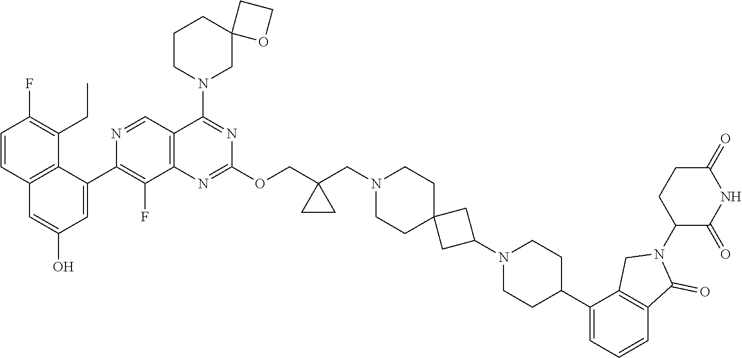 [SMILES: CCc1c(F)ccc2cc(O)cc(-c3ncc4c(N5CCCC6(CCO6)C5)nc(OCC5(CN6CCC7(CC6)CC(N6CCC(c8cccc9c8CN(C8CCC(=O)NC8=O)C9=O)CC6)C7)CC5)nc4c3F)c12] |
| 179 | 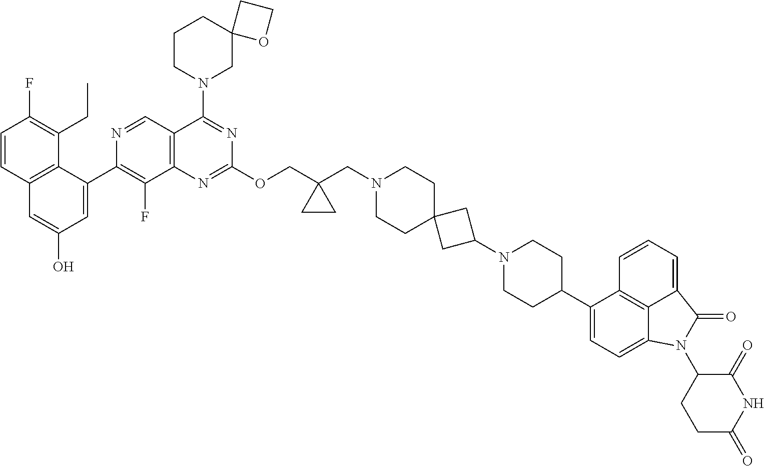 [SMILES: CCc1c(F)ccc2cc(O)cc(-c3ncc4c(N5CCCC6(CCO6)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |
| 180 | 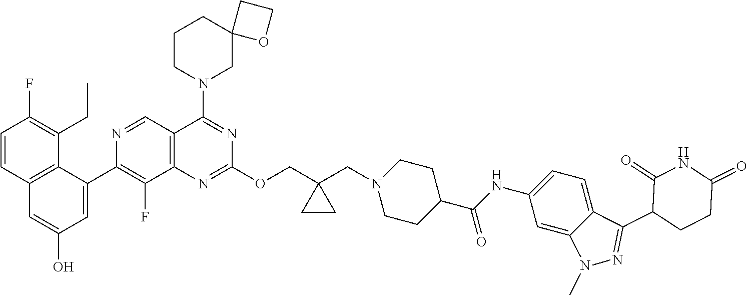 [SMILES: CCc1c(F)ccc2cc(O)cc(-c3ncc4c(N5CCCC6(CCO6)C5)nc(OCC5(CN6CCC(C(=O)Nc7ccc8c(C9CCC(=O)NC9=O)nn(C)c8c7)CC6)CC5)nc4c3F)c12] |
| 181 | 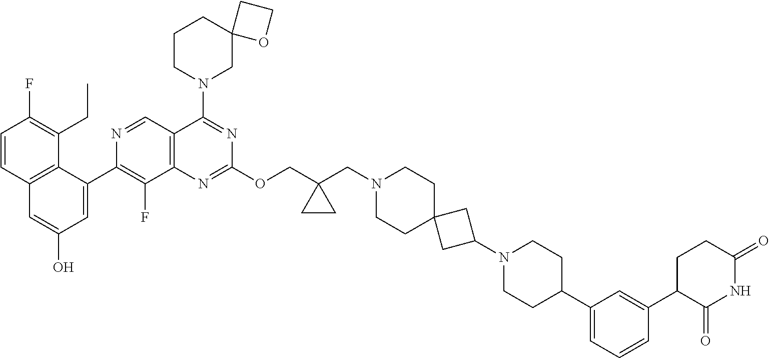 [SMILES: CCc1c(F)ccc2cc(O)cc(-c3ncc4c(N5CCCC6(CCO6)C5)nc(OCC5(CN6CCC7(CC6)CC(N6CCC(c8cccc(C9CCC(=O)NC9=O)c8)CC6)C7)CC5)nc4c3F)c12] |
| 182 | 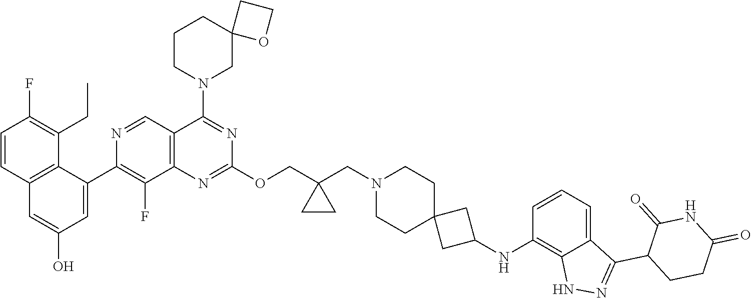 [SMILES: CCc1c(F)ccc2cc(O)cc(-c3ncc4c(N5CCCC6(CCO6)C5)nc(OCC5(CN6CCC7(CC6)CC(Nc6cccc8c(C9CCC(=O)NC9=O)n[nH]c68)C7)CC5)nc4c3F)c12] |
| 183 | 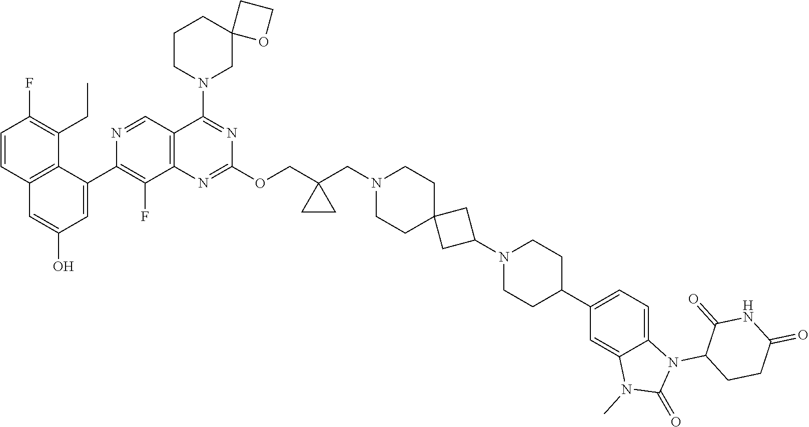 [SMILES: CCc1c(F)ccc2cc(O)cc(-c3ncc4c(N5CCCC6(CCO6)C5)nc(OCC5(CN6CCC7(CC6)CC(N6CCC(c8ccc9c(c8)n(C)c(=O)n9C8CCC(=O)NC8=O)CC6)C7)CC5)nc4c3F)c12] |
| 184 | 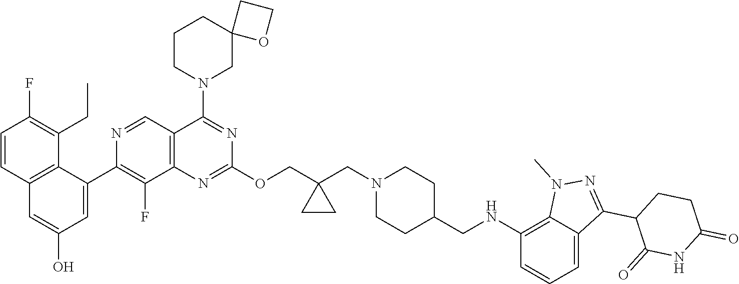 [SMILES: CCc1c(F)ccc2cc(O)cc(-c3ncc4c(N5CCCC6(CCO6)C5)nc(OCC5(CN6CCC(CNc7cccc8c(C9CCC(=O)NC9=O)nn(C)c78)CC6)CC5)nc4c3F)c12] |
| 185 | 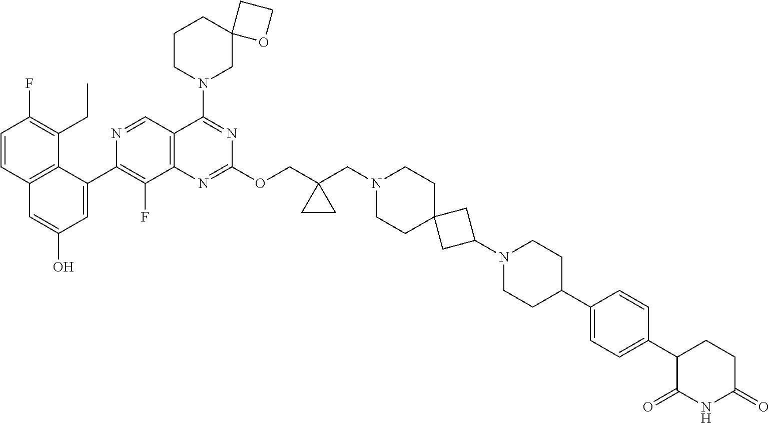 [SMILES: CCc1c(F)ccc2cc(O)cc(-c3ncc4c(N5CCCC6(CCO6)C5)nc(OCC5(CN6CCC7(CC6)CC(N6CCC(c8ccc(C9CCC(=O)NC9=O)cc8)CC6)C7)CC5)nc4c3F)c12] |
| 186 | 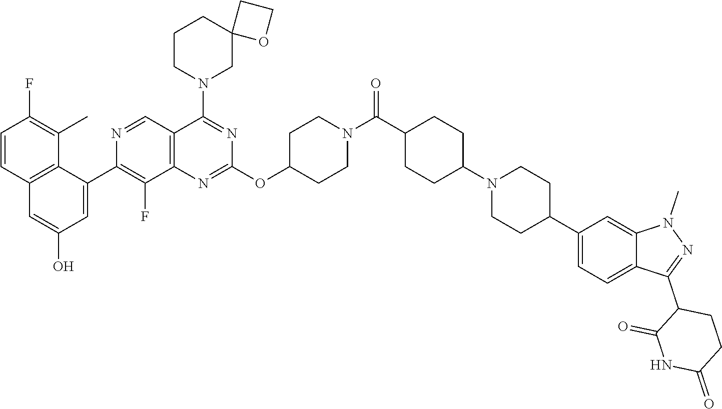 [SMILES: Cc1c(F)ccc2cc(O)cc(-c3ncc4c(N5CCCC6(CCO6)C5)nc(OC5CCN(C(=O)C6CCC(N7CCC(c8ccc9c(C%10CCC(=O)NC%10=O)nn(C)c9c8)CC7)CC6)CC5)nc4c3F)c12] |
| 187 | 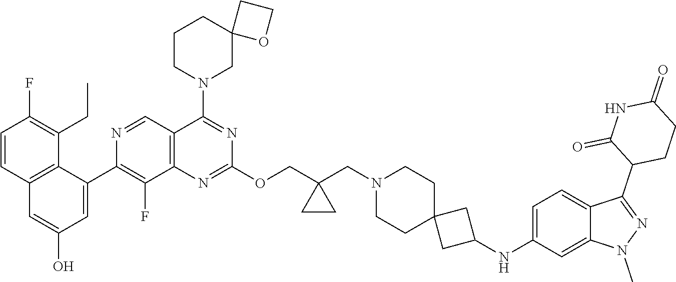 [SMILES: CCc1c(F)ccc2cc(O)cc(-c3ncc4c(N5CCCC6(CCO6)C5)nc(OCC5(CN6CCC7(CC6)CC(Nc6ccc8c(C9CCC(=O)NC9=O)nn(C)c8c6)C7)CC5)nc4c3F)c12] |
| 188 | 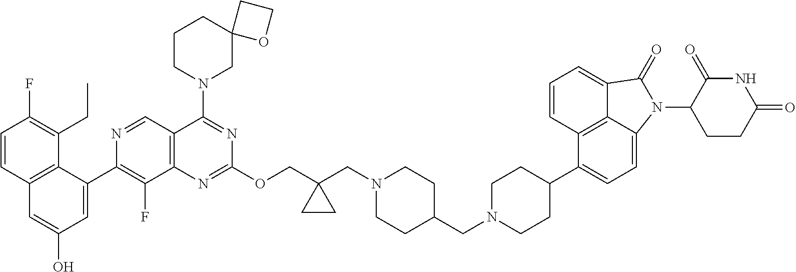 [SMILES: CCc1c(F)ccc2cc(O)cc(-c3ncc4c(N5CCCC6(CCO6)C5)nc(OCC5(CN6CCC(CN7CCC(c8ccc9c%10c(cccc8%10)C(=O)N9C8CCC(=O)NC8=O)CC7)CC6)CC5)nc4c3F)c12] |
| 189 | 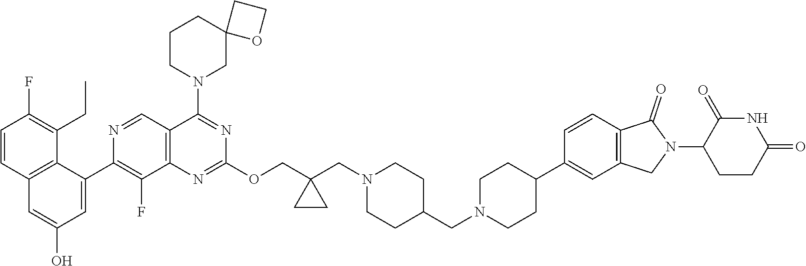 [SMILES: CCc1c(F)ccc2cc(O)cc(-c3ncc4c(N5CCCC6(CCO6)C5)nc(OCC5(CN6CCC(CN7CCC(c8ccc9c(c8)CN(C8CCC(=O)NC8=O)C9=O)CC7)CC6)CC5)nc4c3F)c12] |
| 190 | 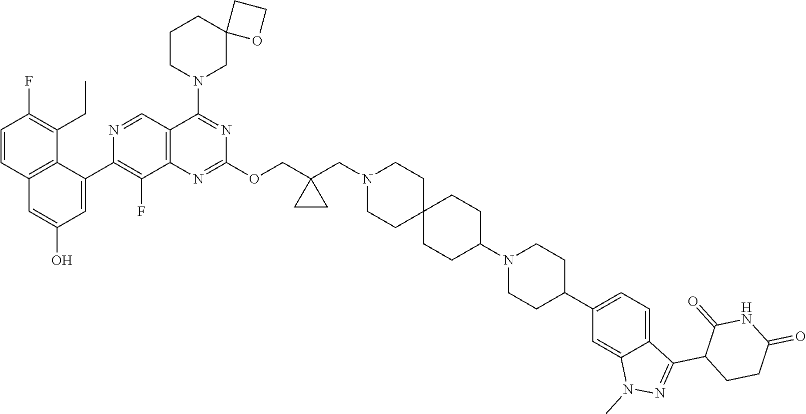 |
| 191 | 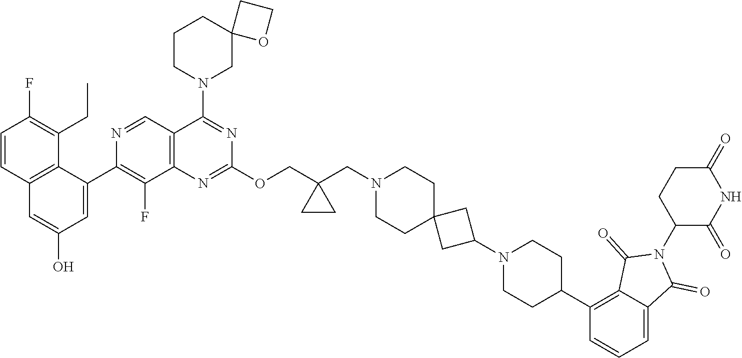 [SMILES: CCc1c(F)ccc2cc(O)cc(-c3ncc4c(N5CCCC6(CCO6)C5)nc(OCC5(CN6CCC7(CC6)CC(N6CCC(c8cccc9c8C(=O)N(C8CCC(=O)NC8=O)C9=O)CC6)C7)CC5)nc4c3F)c12] |
| 192 | 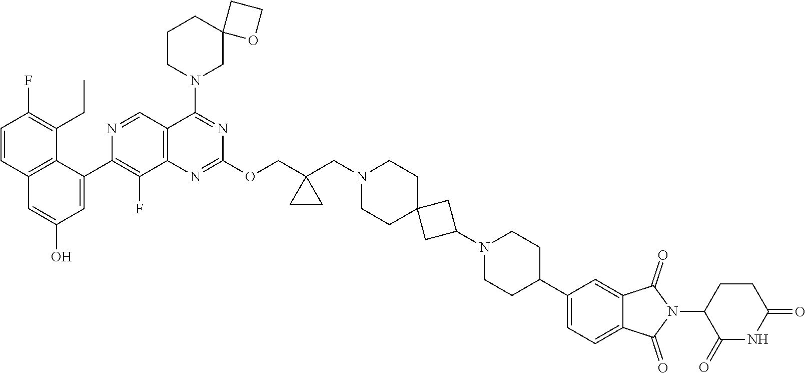 [SMILES: CCc1c(F)ccc2cc(O)cc(-c3ncc4c(N5CCCC6(CCO6)C5)nc(OCC5(CN6CCC7(CC6)CC(N6CCC(c8ccc9c(c8)C(=O)N(C8CCC(=O)NC8=O)C9=O)CC6)C7)CC5)nc4c3F)c12] |
| 193 | 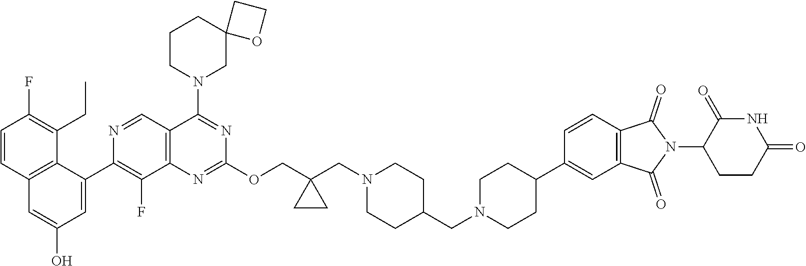 [SMILES: CCc1c(F)ccc2cc(O)cc(-c3ncc4c(N5CCCC6(CCO6)C5)nc(OCC5(CN6CCC(CN7CCC(c8ccc9c(c8)C(=O)N(C8CCC(=O)NC8=O)C9=O)CC7)CC6)CC5)nc4c3F)c12] |
| 194 | 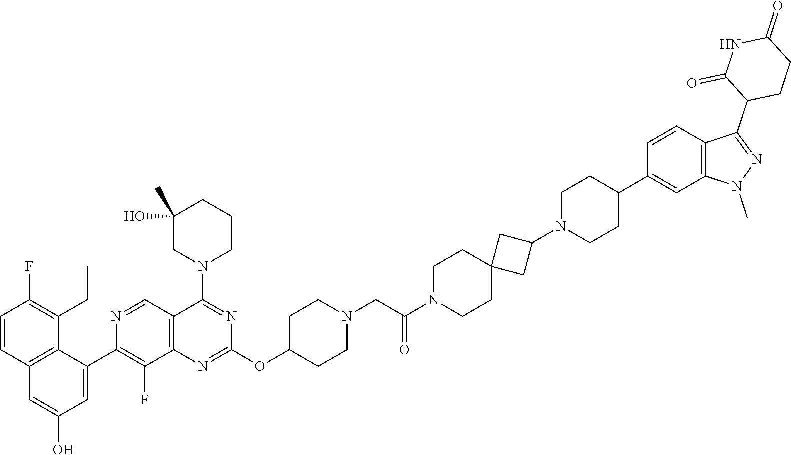 [SMILES: CCc1c(F)ccc2cc(O)cc(-c3ncc4c(N5CCC[C@@](C)(O)C5)nc(OC5CCN(CC(=O)N6CCC7(CC6)CC(N6CCC(c8ccc9c(C%10CCC(=O)NC%10=O)nn(C)c9c8)CC6)C7)CC5)nc4c3F)c12] |
| 195 | 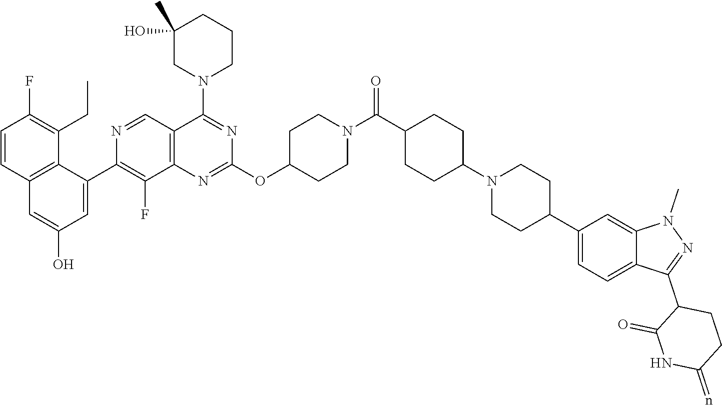 |
| 196 | 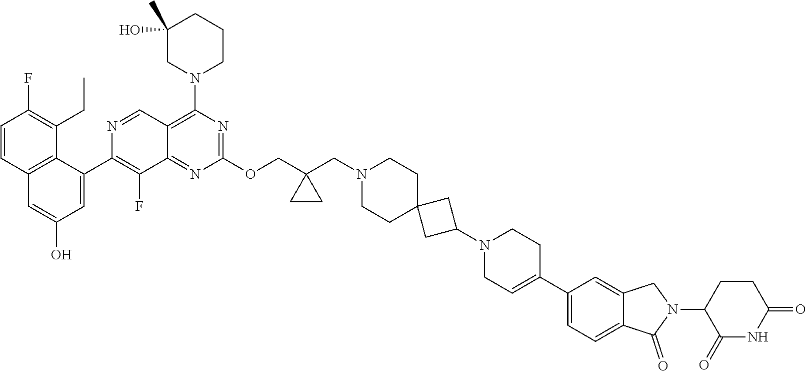 [SMILES: CCc1c(F)ccc2cc(O)cc(-c3ncc4c(N5CCC[C@@](C)(O)C5)nc(OCC5(CN6CCC7(CC6)CC(N6CC=C(c8ccc9c(c8)CN(C8CCC(=O)NC8=O)C9=O)CC6)C7)CC5)nc4c3F)c12] |
| 197 | 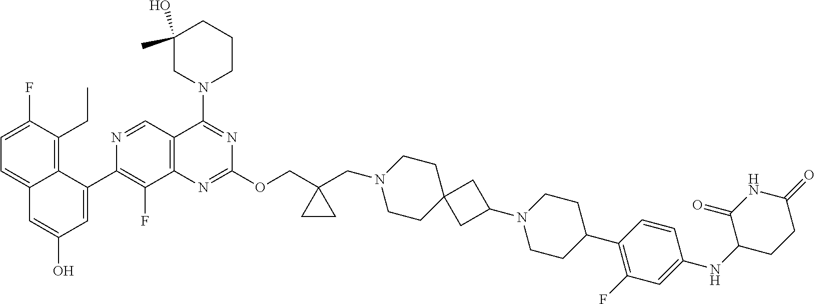 [SMILES: CCc1c(F)ccc2cc(O)cc(-c3ncc4c(N5CCC[C@@](C)(O)C5)nc(OCC5(CN6CCC7(CC6)CC(N6CCC(c8ccc(NC9CCC(=O)NC9=O)cc8F)CC6)C7)CC5)nc4c3F)c12] |
| 198 | 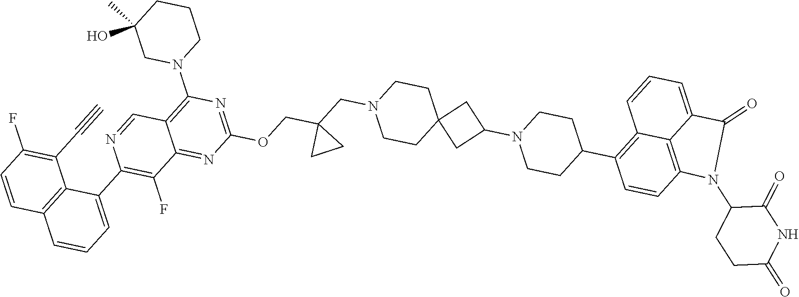 [SMILES: C#Cc1c(F)ccc2cccc(-c3ncc4c(N5CCC[C@](C)(O)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |
| 199 | 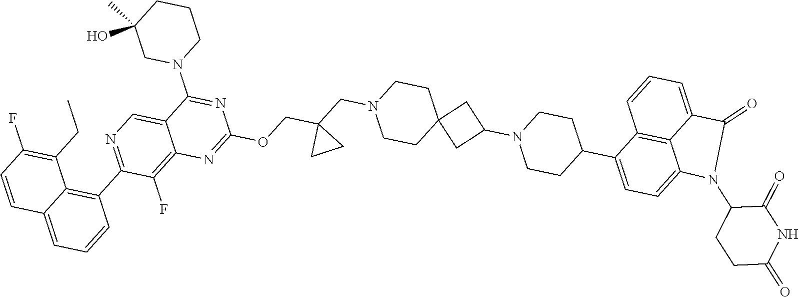 [SMILES: CCc1c(F)ccc2cccc(-c3ncc4c(N5CCC[C@](C)(O)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |
| 200 | 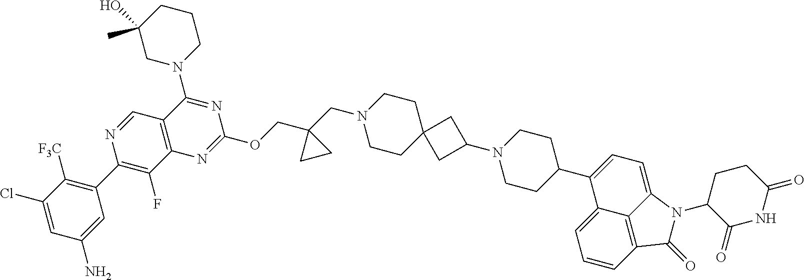 [SMILES: C[C@@]1(O)CCCN(c2nc(OCC3(CN4CCC5(CC4)CC(N4CCC(c6ccc7c8c(cccc68)C(=O)N7C6CCC(=O)NC6=O)CC4)C5)CC3)nc3c(F)c(-c4cc(N)cc(Cl)c4C(F)(F)F)ncc23)C1] |
| 201 | 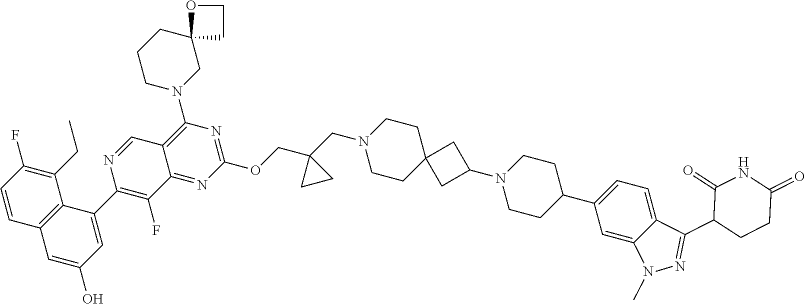 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 202 | 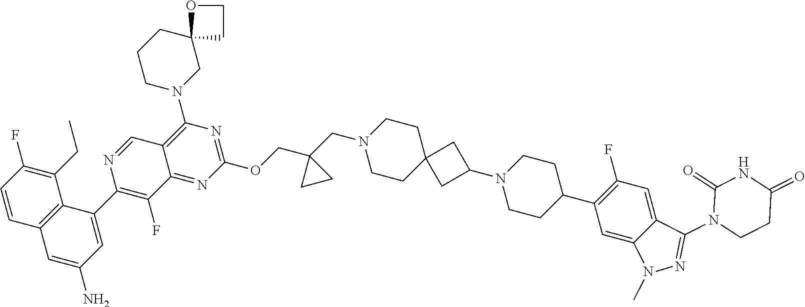 [SMILES: CCc1c(F)ccc2cc(N)cc(-c3ncc4c(N5CCC[C@]6(CCO6)C5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 203 | 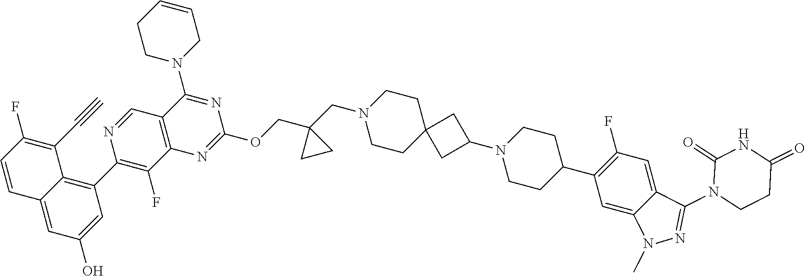 [SMILES: C#Cc1c(F)ccc2cc(O)cc(-c3ncc4c(N5CC=CCC5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 204 | 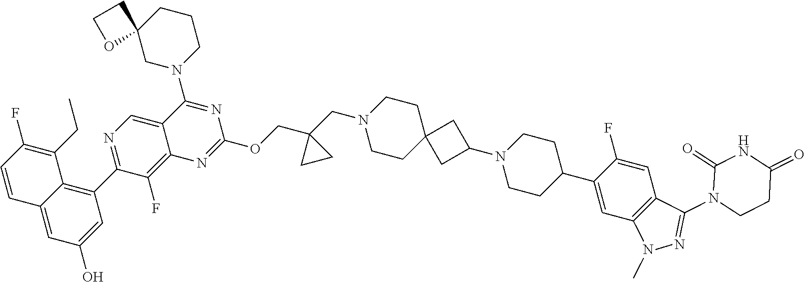 [SMILES: CCc1c(F)ccc2cc(O)cc(-c3ncc4c(N5CCC[C@]6(CCO6)C5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 205 | 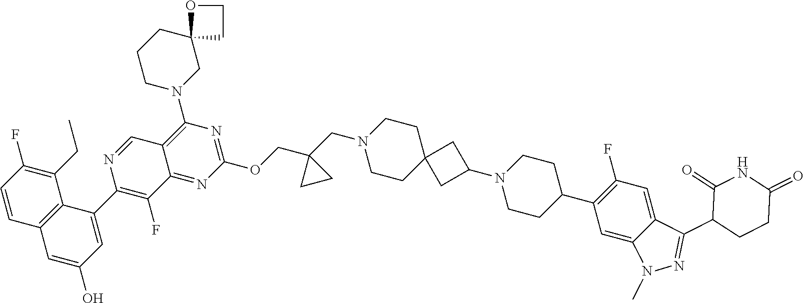 [SMILES: CCc1c(F)ccc2cc(O)cc(-c3ncc4c(N5CCC[C@]6(CCO6)C5)nc(OCC5(CN6CCC7(CC6)CC(N6CCC(c8cc9c(cc8F)c(C8CCC(=O)NC8=O)nn9C)CC6)C7)CC5)nc4c3F)c12] |
| 206 | 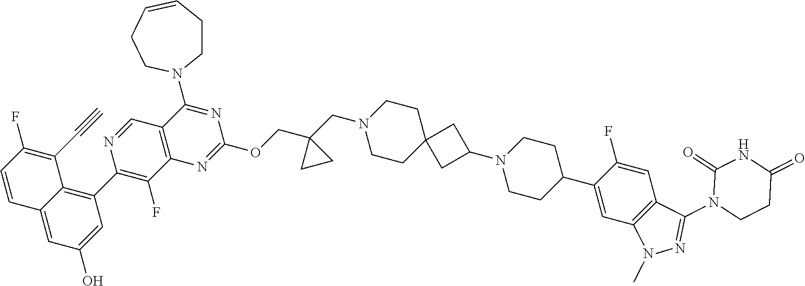 [SMILES: C#Cc1c(F)ccc2cc(O)cc(-c3ncc4c(N5CCC=CCC5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 207 | 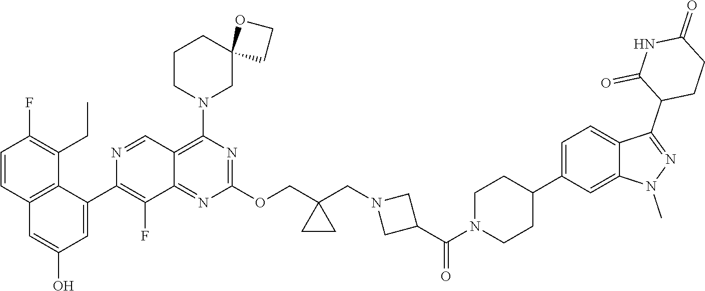 [SMILES: CCc1c(F)ccc2cc(O)cc(-c3ncc4c(N5CCC[C@]6(CCO6)C5)nc(OCC5(CN6CC(C(=O)N7CCC(c8ccc9c(C%10CCC(=O)NC%10=O)nn(C)c9c8)CC7)C6)CC5)nc4c3F)c12] |
| 208 | 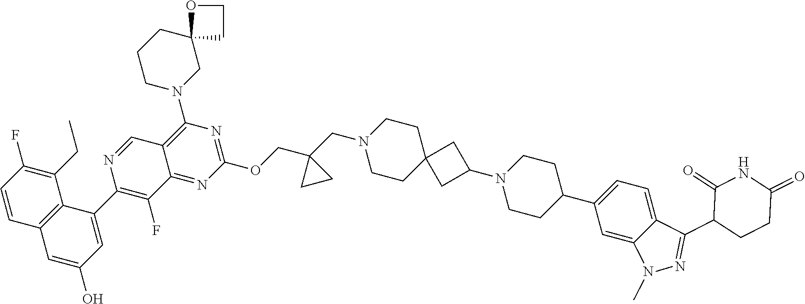 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 209 | 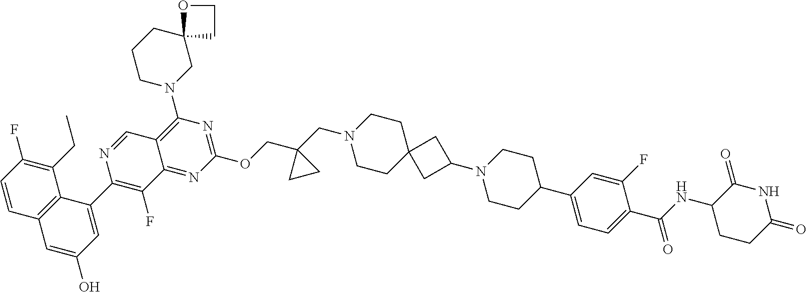 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(C(=O)NC9CCC(=O)NC9=O)c(F)c8)CC6)C7)CC5)nc4c3F)c12] |
| 210 | 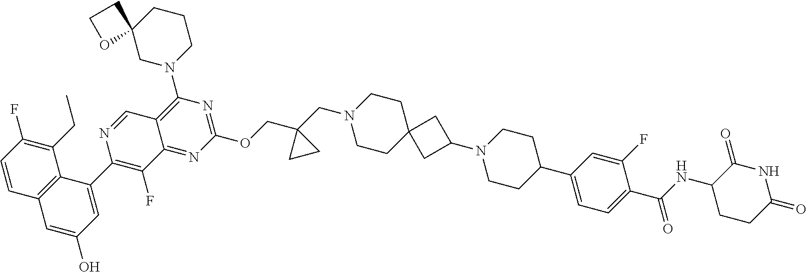 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(C(=O)NC9CCC(=O)NC9=O)c(F)c8)CC6)C7)CC5)nc4c3F)c12] |
| 211 | 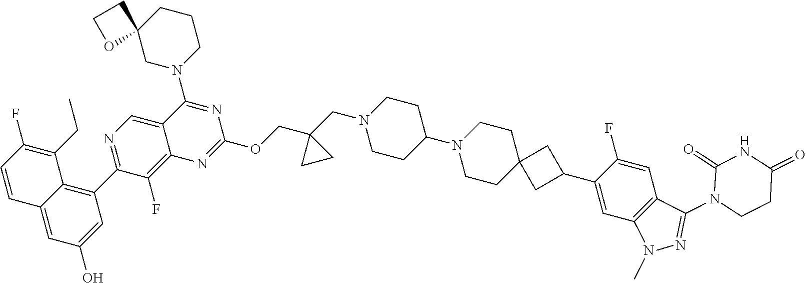 [SMILES: CCc1c(F)ccc2cc(O)cc(-c3ncc4c(N5CCC[C@]6(CCO6)C5)nc(OCC5(CN6CCC(N7CCC8(CC7)CC(c7cc9c(cc7F)c(N7CCC(=O)NC7=O)nn9C)C8)CC6)CC5)nc4c3F)c12] |
| 212 | 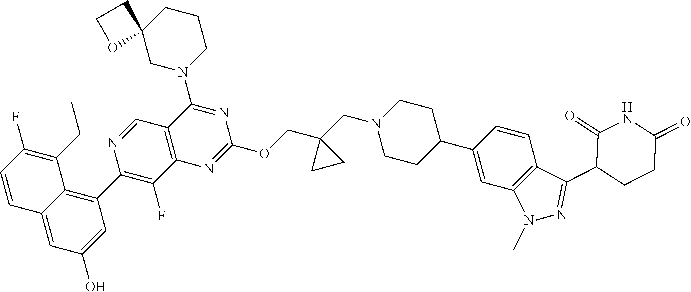 [SMILES: CCc1c(F)ccc2cc(O)cc(-c3ncc4c(N5CCC[C@]6(CCO6)C5)nc(OCC5(CN6CCC(c7ccc8c(C9CCC(=O)NC9=O)nn(C)c8c7)CC6)CC5)nc4c3F)c12] |
| 213 | 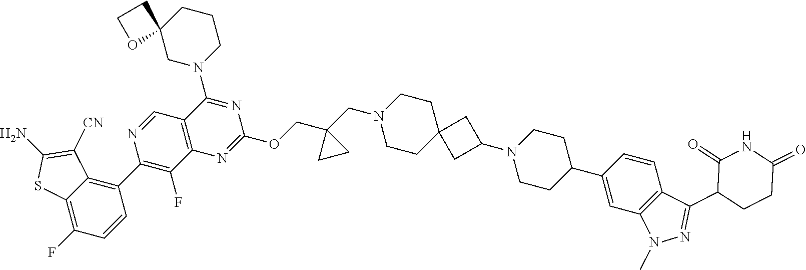 [SMILES: Cn1nc(C2CCC(=O)NC2=O)c2ccc(C3CCN(C4CC5(CCN(CC6(COc7nc(N8CCC[C@]9(CCO9)C8)c8cnc(-c9ccc(F)c%10sc(N)c(C#N)c9%10)c(F)c8n7)CC6)CC5)C4)CC3)cc21] |
| 214 | 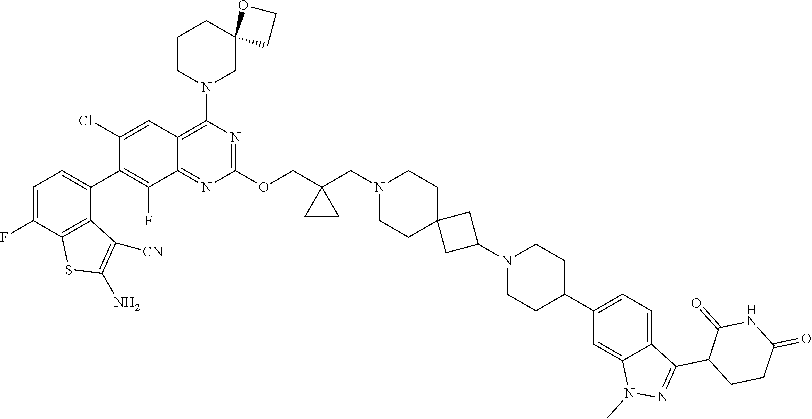 [SMILES: Cn1nc(C2CCC(=O)NC2=O)c2ccc(C3CCN(C4CC5(CCN(CC6(COc7nc(N8CCC[C@]9(CCO9)C8)c8cc(Cl)c(-c9ccc(F)c%10sc(N)c(C#N)c9%10)c(F)c8n7)CC6)CC5)C4)CC3)cc21] |
| 215 | 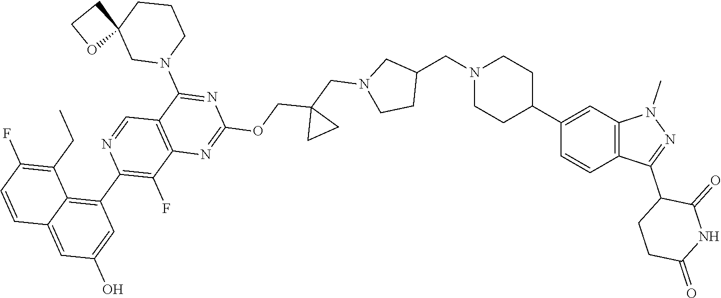 [SMILES: CCc1c(F)ccc2cc(O)cc(-c3ncc4c(N5CCC[C@]6(CCO6)C5)nc(OCC5(CN6CCC(CN7CCC(c8ccc9c(C%10CCC(=O)NC%10=O)nn(C)c9c8)CC7)C6)CC5)nc4c3F)c12] |
| 216 | 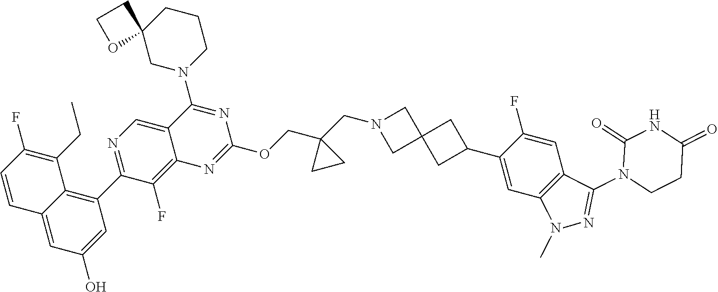 [SMILES: CCc1c(F)ccc2cc(O)cc(-c3ncc4c(N5CCC[C@]6(CCO6)C5)nc(OCC5(CN6CC7(CC(c8cc9c(cc8F)c(N8CCC(=O)NC8=O)nn9C)C7)C6)CC5)nc4c3F)c12] |
| 217 | 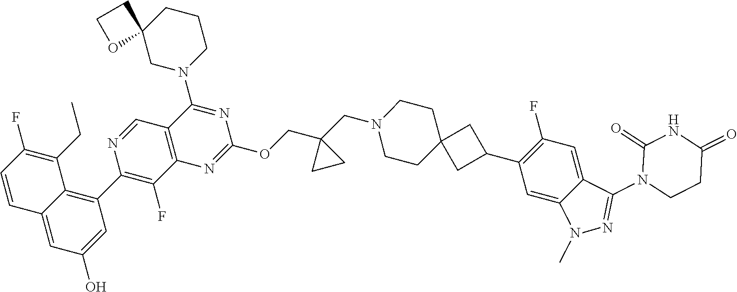 [SMILES: CCc1c(F)ccc2cc(O)cc(-c3ncc4c(N5CCC[C@]6(CCO6)C5)nc(OCC5(CN6CCC7(CC6)CC(c6cc8c(cc6F)c(N6CCC(=O)NC6=O)nn8C)C7)CC5)nc4c3F)c12] |
| 218 | 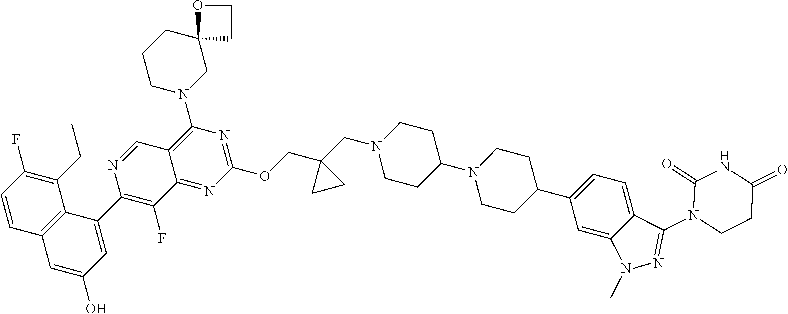 [SMILES: CCc1c(F)ccc2cc(O)cc(-c3ncc4c(N5CCC[C@]6(CCO6)C5)nc(OCC5(CN6CCC(N7CCC(c8ccc9c(N%10CCC(=O)NC%10=O)nn(C)c9c8)CC7)CC6)CC5)nc4c3F)c12] |
| 219 | 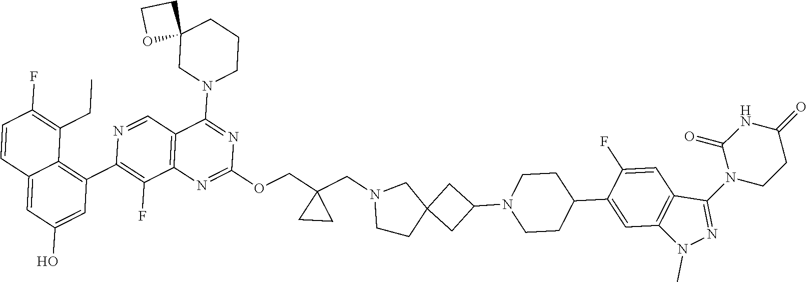 [SMILES: CCc1c(F)ccc2cc(O)cc(-c3ncc4c(N5CCC[C@]6(CCO6)C5)nc(OCC5(CN6CCC7(CC(N8CCC(c9cc%10c(cc9F)c(N9CCC(=O)NC9=O)nn%10C)CC8)C7)C6)CC5)nc4c3F)c12] |
| 220 | 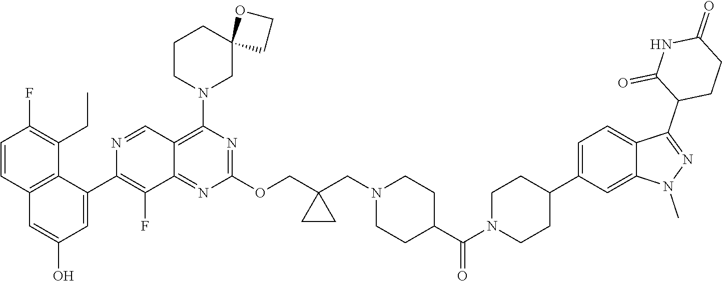 [SMILES: CCc1c(F)ccc2cc(O)cc(-c3ncc4c(N5CCC[C@]6(CCO6)C5)nc(OCC5(CN6CCC(C(=O)N7CCC(c8ccc9c(C%10CCC(=O)NC%10=O)nn(C)c9c8)CC7)CC6)CC5)nc4c3F)c12] |
| 221 | 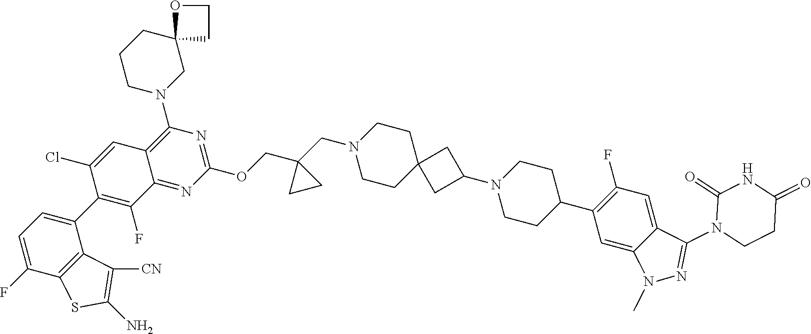 [SMILES: Cn1nc(N2CCC(=O)NC2=O)c2cc(F)c(C3CCN(C4CC5(CCN(CC6(COc7nc(N8CCC[C@]9(CCO9)C8)c8cc(Cl)c(-c9ccc(F)c%10sc(N)c(C#N)c9%10)c(F)c8n7)CC6)CC5)C4)CC3)cc21] |
| 222 | 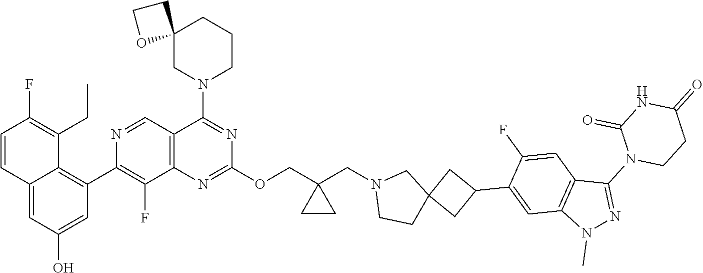 [SMILES: CCc1c(F)ccc2cc(O)cc(-c3ncc4c(N5CCC[C@]6(CCO6)C5)nc(OCC5(CN6CCC7(CC(c8cc9c(cc8F)c(N8CCC(=O)NC8=O)nn9C)C7)C6)CC5)nc4c3F)c12] |
| 223 | 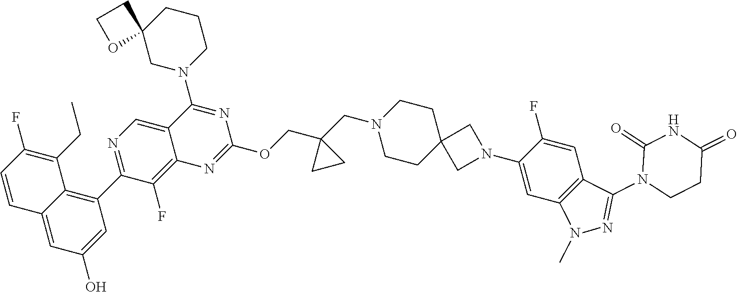 [SMILES: CCc1c(F)ccc2cc(O)cc(-c3ncc4c(N5CCC[C@]6(CCO6)C5)nc(OCC5(CN6CCC7(CC6)CN(c6cc8c(cc6F)c(N6CCC(=O)NC6=O)nn8C)C7)CC5)nc4c3F)c12] |
| 224 | 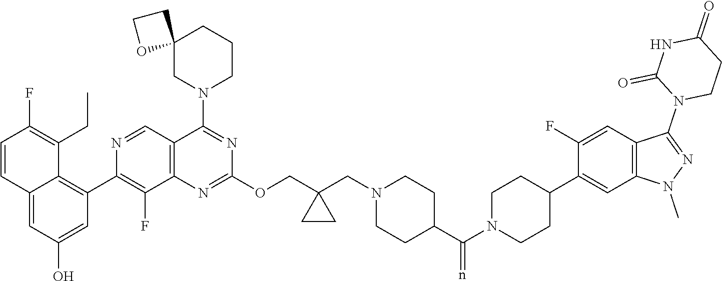 |
| 225 | 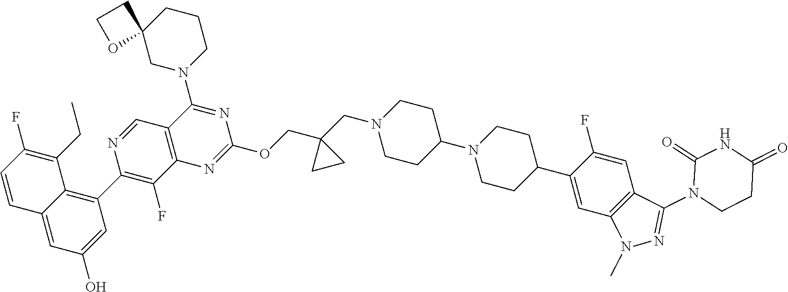 [SMILES: CCc1c(F)ccc2cc(O)cc(-c3ncc4c(N5CCC[C@]6(CCO6)C5)nc(OCC5(CN6CCC(N7CCC(c8cc9c(cc8F)c(N8CCC(=O)NC8=O)nn9C)CC7)CC6)CC5)nc4c3F)c12] |
| 226 | 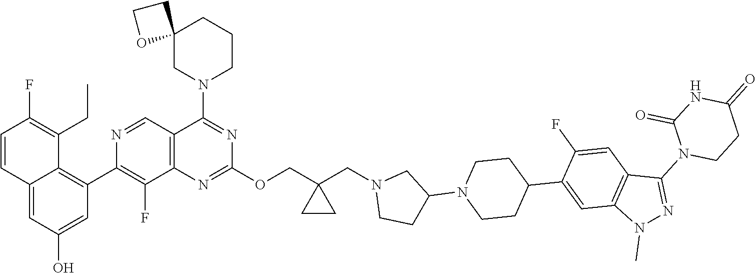 [SMILES: CCc1c(F)ccc2cc(O)cc(-c3ncc4c(N5CCC[C@]6(CCO6)C5)nc(OCC5(CN6CCC(N7CCC(c8cc9c(cc8F)c(N8CCC(=O)NC8=O)nn9C)CC7)C6)CC5)nc4c3F)c12] |
| 227 | 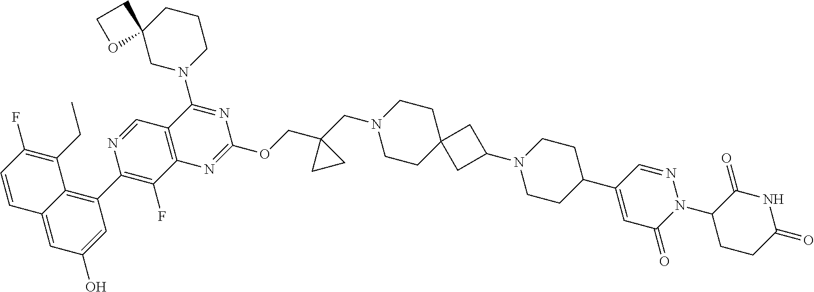 [SMILES: CCc1c(F)ccc2cc(O)cc(-c3ncc4c(N5CCC[C@]6(CCO6)C5)nc(OCC5(CN6CCC7(CC6)CC(N6CCC(c8cnn(C9CCC(=O)NC9=O)c(=O)c8)CC6)C7)CC5)nc4c3F)c12] |
| 228 | 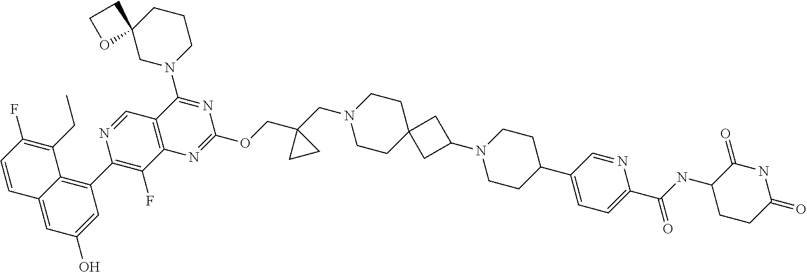 |
| 229 | 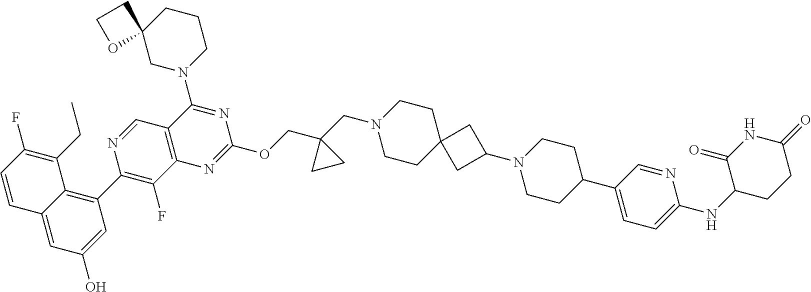 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(NC9CCC(=O)NC9=O)nc8)CC6)C7)CC5)nc4c3F)c12] |
| 230 | 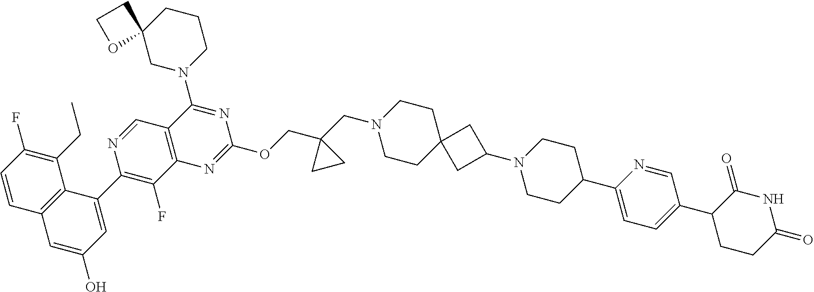 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(C9CCC(=O)NC9=O)cn8)CC6)C7)CC5)nc4c3F)c12] |
| 231 | 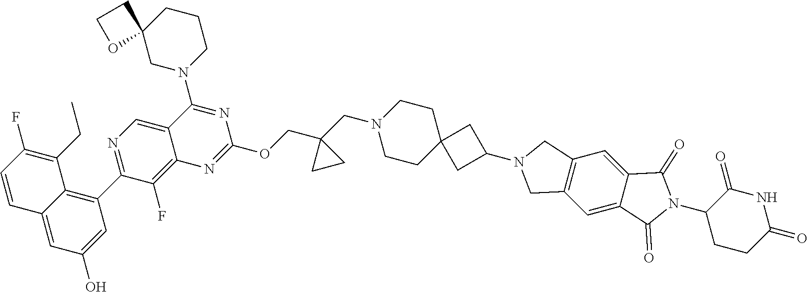 [SMILES: CCc1c(F)ccc2cc(O)cc(-c3ncc4c(N5CCC[C@]6(CCO6)C5)nc(OCC5(CN6CCC7(CC6)CC(N6Cc8cc9c(cc8C6)C(=O)N(C6CCC(=O)NC6=O)C9=O)C7)CC5)nc4c3F)c12] |
| 232 | 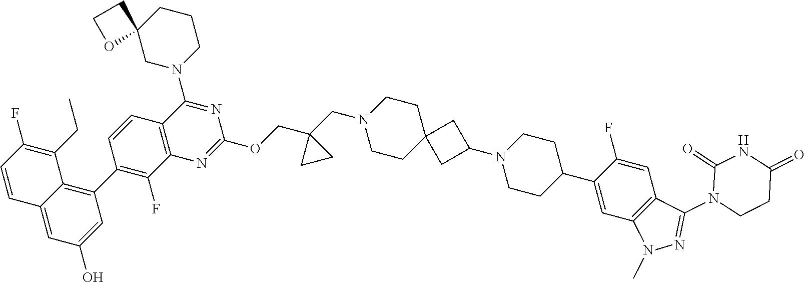 [SMILES: CCc1c(F)ccc2cc(O)cc(-c3ccc4c(N5CCC[C@]6(CCO6)C5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 233 | 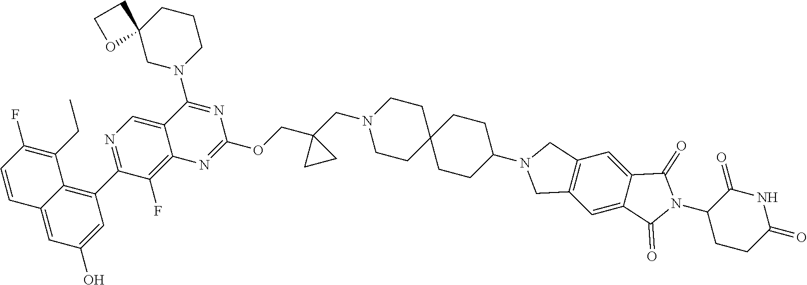 [SMILES: CCc1c(F)ccc2cc(O)cc(-c3ncc4c(N5CCC[C@]6(CCO6)C5)nc(OCC5(CN6CCC7(CCC(N8Cc9cc%10c(cc9C8)C(=O)N(C8CCC(=O)NC8=O)C%10=O)CC7)CC6)CC5)nc4c3F)c12] |
| 234 | 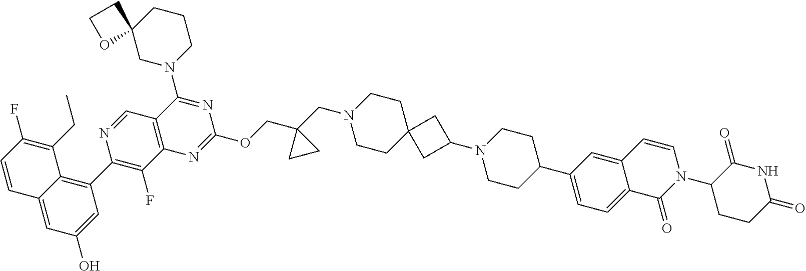 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c(=O)n(C%10CCC(=O)NC%10=O)ccc9c8)CC6)C7)CC5)nc4c3F)c12] |
| 235 | 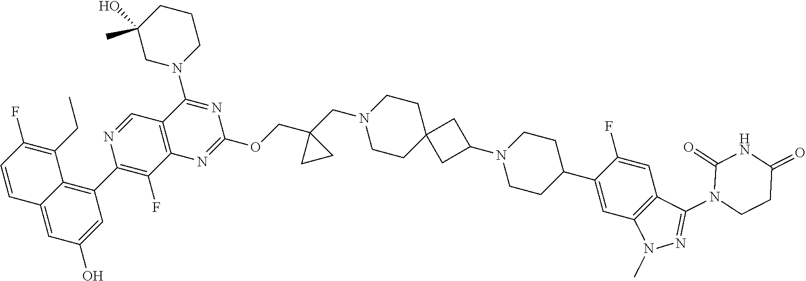 [SMILES: CCc1c(F)ccc2cc(O)cc(-c3ncc4c(N5CCC[C@@](C)(O)C5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 236 | 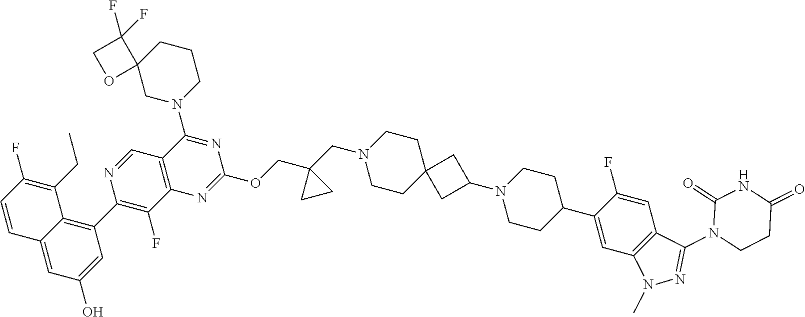 [SMILES: CCc1c(F)ccc2cc(O)cc(-c3ncc4c(N5CCCC6(C5)OCC6(F)F)nc(OCC5(CN6CCC7(CC6)CC(N6CCC(c8cc9c(cc8F)c(N8CCC(=O)NC8=O)nn9C)CC6)C7)CC5)nc4c3F)c12] |
| 237 | 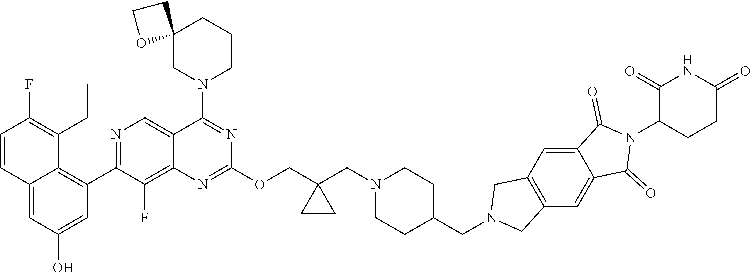 [SMILES: CCc1c(F)ccc2cc(O)cc(-c3ncc4c(N5CCC[C@]6(CCO6)C5)nc(OCC5(CN6CCC(CN7Cc8cc9c(cc8C7)C(=O)N(C7CCC(=O)NC7=O)C9=O)CC6)CC5)nc4c3F)c12] |
| 238 | 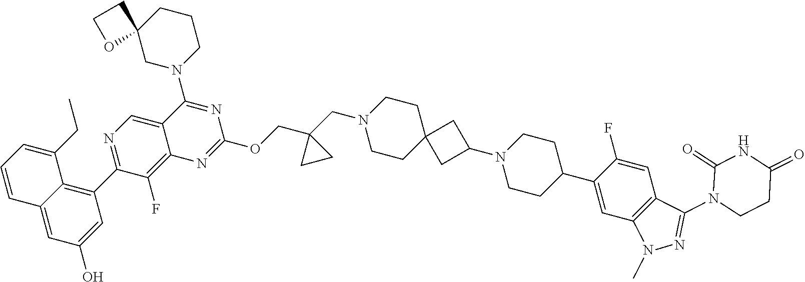 [SMILES: CCc1cccc2cc(O)cc(-c3ncc4c(N5CCC[C@]6(CCO6)C5)nc(OCC5(CN6CCC7(CC6)CC(N6CCC(c8cc9c(cc8F)c(N8CCC(=O)NC8=O)nn9C)CC6)C7)CC5)nc4c3F)c12] |
| 239 | 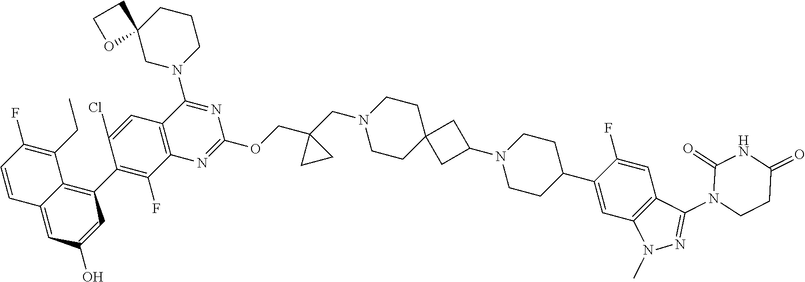 |
| 240 | 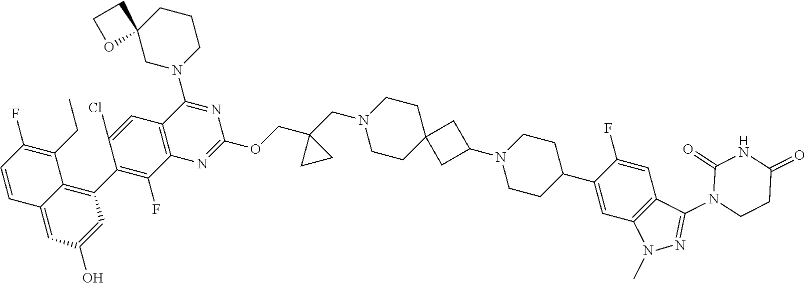 |
| 241 | 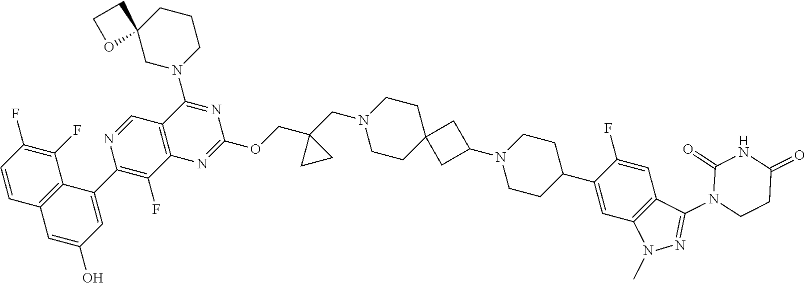 [SMILES: Cn1nc(N2CCC(=O)NC2=O)c2cc(F)c(C3CCN(C4CC5(CCN(CC6(COc7nc(N8CCC[C@]9(CCO9)C8)c8cnc(-c9cc(O)cc%10ccc(F)c(F)c9%10)c(F)c8n7)CC6)CC5)C4)CC3)cc21] |
| 242 | 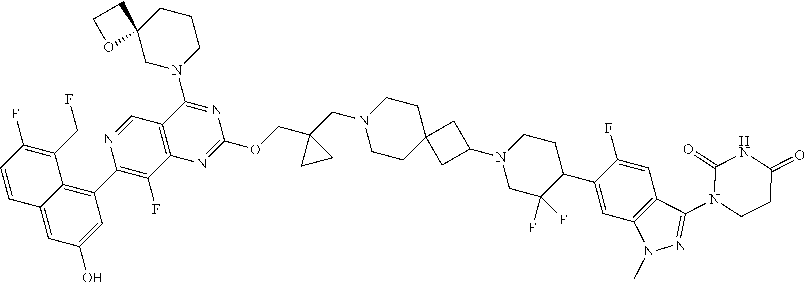 [SMILES: Cn1nc(N2CCC(=O)NC2=O)c2cc(F)c(C3CCN(C4CC5(CCN(CC6(COc7nc(N8CCC[C@]9(CCO9)C8)c8cnc(-c9cc(O)cc%10ccc(F)c(CF)c9%10)c(F)c8n7)CC6)CC5)C4)CC3(F)F)cc21] |
| 243 | 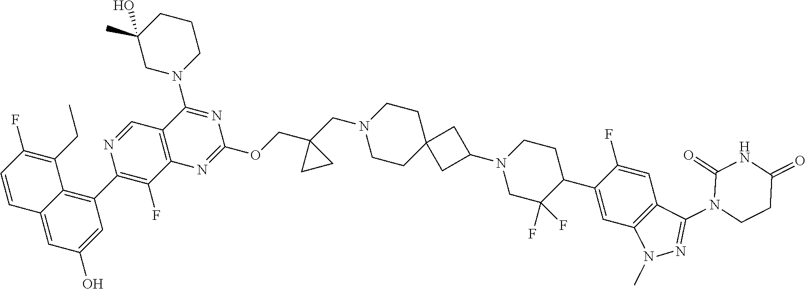 [SMILES: CCc1c(F)ccc2cc(O)cc(-c3ncc4c(N5CCC[C@@](C)(O)C5)nc(OCC5(CN6CCC7(CC6)CC(N6CCC(c8cc9c(cc8F)c(N8CCC(=O)NC8=O)nn9C)C(F)(F)C6)C7)CC5)nc4c3F)c12] |
| 244 | 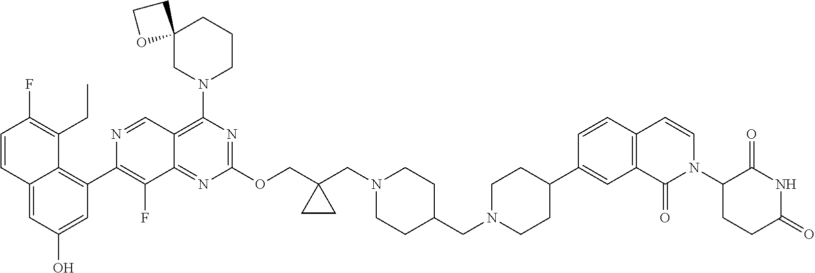 [SMILES: CCc1c(F)ccc2cc(O)cc(-c3ncc4c(N5CCC[C@]6(CCO6)C5)nc(OCC5(CN6CCC(CN7CCC(c8ccc9ccn(C%10CCC(=O)NC%10=O)c(=O)c9c8)CC7)CC6)CC5)nc4c3F)c12] |
| 179a | 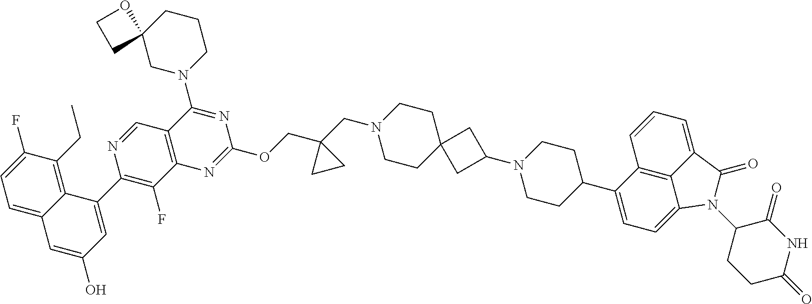 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |

In some embodiments, there is provided a compound of Table 2, or a pharmaceutically acceptable salt, ester, stereoisomer, hydrate, or solvate thereof.

In some embodiments, there is provided a compound of Table 3, or a pharmaceutically acceptable salt, ester, stereoisomer, hydrate, or solvate thereof.

For compounds of the disclosure, when a chiral center is present, it should be understood that the configuration of the stereoisomer is not limited. Thus, when a chiral center is present, the configuration of the stereoisomer may be R-configuration, S-configuration, or a mixture of R- and S-configurations. All isomeric forms, including stereosiomers, diastereoisomers, and the like are intended to be included.

In some embodiments, there is provided a compound as described herein wherein the C, H, O, and N atoms in the compound are each independently selected from atoms of natural abundance and isotope-enriched atoms. Examples of isotopes of natural abundance include ¹²C, ¹H, ¹⁶O and ¹⁴N. Examples of isotope-enriched atoms include, without limitation, ¹³C and ¹⁴C for carbon; ²H (D) and ³H (T) for hydrogen; ¹⁷O and ¹⁸O for oxygen; and ¹⁵N for nitrogen. In some embodiments of compounds of the disclosure, all the elements or atoms in a compound are isotopes of natural abundance. In other embodiments, one or more elements or atoms in a compound are isotope-enriched.

In another broad aspect, there are provided pharmaceutical compositions comprising a compound described herein, or a pharmaceutically acceptable salt, ester, hydrate, solvate or stereoisomer thereof, and a pharmaceutically acceptable excipient, carrier or diluent. In some embodiments, there are provided pharmaceutical compositions comprising a compound of Formula (A), or a pharmaceutically acceptable salt, ester, hydrate, solvate or stereoisomer thereof, and a pharmaceutically acceptable carrier. In some embodiments, there are provided pharmaceutical compositions comprising a compound of Table 2, or a pharmaceutically acceptable salt, ester, hydrate, solvate or stereoisomer thereof, and a pharmaceutically acceptable carrier. In some embodiments, there are provided pharmaceutical compositions comprising a compound of Table 3, or a pharmaceutically acceptable salt, ester, hydrate, solvate or stereoisomer thereof, and a pharmaceutically acceptable carrier.

In some such embodiments, the composition comprises a pharmaceutically acceptable excipient comprising one or more adhesive, filler, disintegrant, lubricant, and/or dispersant. In some embodiments, the pharmaceutically acceptable carrier comprises a cream, an emulsion, a gel, a liposome, or a nanoparticle.

In some embodiments, the pharmaceutical composition is suitable for oral administration. In some such embodiments, the composition is in the form of a hard-shell gelatin capsule, a soft-shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In some embodiments, the pharmaceutical composition is injectable.

In some embodiments, the pharmaceutically acceptable carrier further comprises at least one additional therapeutic agent, such as, without limitation, a chemotherapeutic agent or another anti-cancer agent. In an embodiment, the at least one additional therapeutic agent is an immune checkpoint inhibitor. Non-limiting examples of immune checkpoint inhibitors include ipulimumab, nivolumab and lambrolizumab.

In another broad aspect, there are provided methods of inhibiting KRAS activity in a subject in need thereof, comprising administering to the subject an effective amount of a compound and/or a pharmaceutical composition described herein.

In certain embodiments, there are provided methods of treating or preventing a KRAS-associated disease, disorder or condition in a subject in need thereof, comprising administering an effective amount of a compound and/or a pharmaceutical composition described herein, such that the KRAS-associated disease, disorder or condition is treated or prevented in the subject.

In particular embodiments, the compounds described herein act to inhibit KRAS (mutant protein or wild-type, e.g., KRAS G12A, KRAS G12C, KRAS G12D, KRAS G12R, KRAS G12S, KRAS G12V, KRAS G13D, KRAS Q61H, and/or wild-type KRAS) and are useful as therapeutic or prophylactic therapy when such inhibition is desired, e.g., for the prevention or treatment of KRAS-associated diseases, conditions and/or disorders. Unless otherwise indicated, when uses of the compounds of the present disclosure are described herein, it is to be understood that such compounds may be in the form of a composition (e.g., a pharmaceutical composition). As used herein, the terms “KRAS inhibitor”, “KRAS degrader” and “bifunctional compound” are used interchangeably to refer to a compound of the disclosure capable of inhibiting and/or degrading a KRAS protein (mutant or wild-type) in a cellular assay, an in vivo model, and/or other assay means indicative of KRAS inhibition and potential therapeutic or prophylactic efficacy. “KRAS inhibition” includes inter alia modulation or promotion of degradation of a KRAS protein (mutant or wild-type), e.g., via a Protacs-type mechanism. The terms also refer to compounds that exhibit at least some therapeutic or prophylactic benefit in a human subject. Although the compounds of the present invention are believed to have effect by promoting degradation of KRAS protein (mutant or wild-type) in a cell, a precise understanding of the compounds' underlying mechanism of action is not required to practice the invention.

In some embodiments, there are provided methods for preventing or treating a KRAS-associated disease, disorder or condition in a subject in need thereof. The KRAS-associated disease, disorder or condition may be, for example and without limitation, a cancer or tumor or hyperplastic or hyperproliferative disease or disorder related to or associated with the KRAS protein, e.g., with a KRAS mutant protein, e.g., KRAS G12A, KRAS G12C, KRAS G12D, KRAS G12R, KRAS G12S, KRAS G12V, KRAS G13D or KRAS Q61H. In some embodiments, the KRAS-associated disease, disorder or condition is a hyperplastic disorder. In some embodiments, the KRAS-associated disease, disorder or condition is a malignant cancer or tumor. In some embodiments, the KRAS-associated disease, disorder or condition is a cardiac, lung, gastrointestinal, genitourinary tract, liver, bone, nervous system, gynecological, hematologic, skin, or adrenal gland cancer or tumor. In some embodiments, the KRAS-associated disease, disorder or condition is a non-small-cell lung cancer (NSCLC), a small cell lung cancer, a pancreatic cancer, a biliary tract cancer, a colorectal cancer, a colon cancer, a bile duct cancer, a cervical cancer, a bladder cancer, a liver cancer or a breast cancer.

In some embodiments, there are provided methods for preventing or treating a KRAS-G12A associated disease, disorder or condition in a subject in need thereof.

In some embodiments, there are provided methods for preventing or treating a KRAS-G12C associated disease, disorder or condition in a subject in need thereof.

In some embodiments, there are provided methods for preventing or treating a KRAS-G12D associated disease, disorder or condition in a subject in need thereof.

In some embodiments, there are provided methods for preventing or treating a KRAS-G12R associated disease, disorder or condition in a subject in need thereof.

In some embodiments, there are provided methods for preventing or treating a KRAS-G12S associated disease, disorder or condition in a subject in need thereof.

In some embodiments, there are provided methods for preventing or treating a KRAS-G12V associated disease, disorder or condition in a subject in need thereof.

In some embodiments, there are provided methods for preventing or treating a KRAS-G13D associated disease, disorder or condition in a subject in need thereof.

In some embodiments, there are provided methods for preventing or treating a KRAS-Q61H associated disease, disorder or condition in a subject in need thereof.

In some embodiments, there are provided methods of inhibiting one or more protein selected from wild-type KRAS, KRAS G12A, KRAS G12C, KRAS G12D, KRAS G12R, KRAS G12S, KRAS G12V, KRAS G13D and/or KRAS Q61H.

In some embodiments, there are provided methods of inhibiting two or more proteins selected from wild-type KRAS, KRAS G12A, KRAS G12C, KRAS G12D, KRAS G12R, KRAS G12S, KRAS G12V, KRAS G13D and KRAS Q61H.

In some embodiments, there are provided methods for treating or preventing cancer in a subject (e.g., a human) comprising administering to the subject a therapeutically effective amount of at least one KRAS inhibitor compound or composition described herein. In some embodiments of such methods, the subject is administered at least one KRAS inhibitor compound or composition in an amount effective to reverse, slow or stop the progression of a KRAS-associated disease, disorder or condition.

The type of cancer or tumor that can be treated or prevented using the compounds and compositions described herein is not meant to be particularly limited. Examples of cancers and tumors that can be treated or prevented using the compounds and compositions described herein include, but are not limited to, cancers and tumors of the: (i) cardiac tissue or heart (including sarcoma, angiosarcoma, fibrosarcoma, rhabdomyosarcoma, liposarcoma, myxoma, rhabdomyoma, fibroma, lipoma, teratoma); (ii) lung (including bronchogenic carcinoma, squamous cell carcinoma, undifferentiated small cell carcinoma, undifferentiated large cell carcinoma, adenocarcinoma, alveolar (bronchiolar) carcinoma, bronchial adenoma, bronchoalveolar carcinoma, sarcoma, lymphoma, chondroma, chondromatous hamartoma, mesothelioma); (iii) gastrointestinal system (including esophagus (squamous, cell carcinoma, adenocarcinoma, leiomyoma, leiomyosarcoma, lymphoma), stomach (carcinoma, lymphoma, leiomyosarcoma, leiomyoma), pancreas (ductal adenocarcinoma, insulinoma, glucagonoma, gastrinoma, carcinoid tumors, somatostatinoma, Kaposi's sarcoma, leiomyoma, hemangioma, lipoma, neurofibroma, fibroma, small intestine (adenocarcinoma, lymphoma, carcinoid tumor, Kaposi's sarcoma, leiomyoma, hemangioma, lipoma, neurofibroma, fibroma), large intestine or bowel (adenocarcinoma, tubular adenoma, villous adenoma, hematoma, leiomyoma)); (iv) genitourinary tract (including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embroyonal carcinoma, teratocarcinoma, choriocarcinoma, sarcoma, interstitial cell carcinoma, fibroma, fibroadenoma, adenomatoid tumors, lipoma); (v) liver (including hepatoma (hepatocellular carcinoma), bile duct cancer (cholangiocarcinoma), hepatoblastoma, angiosarcoma, hepatocellular adenoma, hemangioma, hemangiosarcoma); (vi) bone (including osteogenic sarcoma (osteosarcoma, bone sarcoma), fibrosarcoma, malignant fibrous histocytoma, chondrosarcoma, Ewing's sarcoma, malignant lymphoma (reticulum cell sarcoma), multiple myeloma, malignant giant cell tumor chordoma, osteochrondroma (osteoenchondroma, osteocartilaginous exostoses), benign chondroma, chondroblastoma, chondromyxoid fibroma, osteoid osteoma and giant cell tumors); (vii) nervous system or neurological (including cranium or skull (osteoma, hemangioma, granuloma, xanthoma, osteitis deformans, meningioma, scleromalacia), meninges (meningioma, meningiosarcoma, gliomatosis), brain (astrocytoma, myeloblastoma, medulloblastoma, glioma, ependymoma, germinoma (pinealoma), glioblastoma multiform, oligodendroglioma, schwannoma, glioma, retinoblastoma, congenitial tumors), spinal cord (neurofibroma, meningioma, glioma, sarcoma)); (viii) gynecological tissues (including uterus (endometrial carcinoma, serous carcinoma, mucinous carcinoma, unclassified carcinoma, granulosa cell tumor, serum stromal tumor, dysgerminoma, malignant teratoma), cervix (cervical carcinoma, pre-tumor cervical dsplasia), ovaries (ovarian carcinoma [serous cystadenocarcinoma, mucinous cystadenocarcinoma], granulose-thecal cell tumors, Sertoli-Leydig cell tumors, dysgerminoma, malignant teratoma), vulva (squamous cell carcinoma, intraepithelial neoplasia, adenocarcinoma, fibrosarcoma, melanoma) vagina (clear cell carcinoma, squamous cell carcinoma, botryoid sarcoma (embryonal rhabdomyosarcoma), fallopian tubes carcinoma)); (ix) hematologic system (including blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkins's lymphoma, malignant lymphoma); (x) skin (including malignant melanoma, basal cell carcinoma, squamous cell carcinoma, Kaposi's sarcoma, moles dysplastic nevi, lipoma, hemangioma, dermatofibroma, keloids, psoriasis); (xi) adrenal glands (including neuroblastoma); (xii) biliary tract (gallbladder cancer, ampullary cancer, bile duct cancer); and (xiii) sarcomas (hemangiosarcoma, fibrosarcoma, rhabdomyosarcoma and liposarcoma), myxoma, rhabdomyoma, fibroma, lipoma and teratoma.

In some embodiments of methods of the present disclosure, the cancer is non-small cell lung cancer (NSCLC), small cell lung cancer, pancreatic cancer, colorectal cancer, colon cancer, bile duct cancer, cervical cancer, biliary tract cancer, bladder cancer, liver cancer or breast cancer.

In certain embodiments, there are provided methods for treating or preventing a hyperplastic or hyperproliferative disease or disorder (e.g., a cancer or a tumor) in a subject (e.g., a human) comprising administering to the subject a therapeutically effective amount of at least one KRAS inhibitor compound or composition provided herein. In some embodiments, the hyperplastic or hyperproliferative disease or disorder is a cancer or a tumor, such as without limitation non-small cell lung cancer (NSCLC), pancreatic cancer, biliary tract cancer, colorectal cancer, colon cancer, bile duct cancer, cervical cancer, bladder cancer, liver cancer or breast cancer. In some embodiments, the hyperplastic or hyperproliferative disease or disorder is a malignant tumor or cancer associated with wild-type KRAS or a mutated KRAS, e.g., KRAS-G12D, G12A, G12C, G12R, G12S, G12V, G13D and/or Q61H.

In other embodiments, there are provided methods for inhibiting, treating, and/or preventing immune-related diseases, disorders, and conditions, as well as diseases with inflammatory components, and dysregulations associated with the foregoing, using at least one bifunctional compound or its compositions provided by the present disclosure.

Other diseases, disorders and conditions that can be treated or prevented, in whole or in part, by inhibition of KRAS activity are candidate indications for the KRAS inhibitor compounds and compositions provided herein and are encompassed by methods of the disclosure.

In some embodiments, there is further provided the use of the KRAS inhibitor compounds and compositions described herein in combination with one or more additional agents. The one or more additional agents may have some KRAS-modulating activity and/or they may function through distinct mechanisms of action. In some embodiments, such agents comprise radiation (e.g., localized radiation therapy or total body radiation therapy) and/or other treatment modalities of a non-pharmacological nature. When combination therapy is utilized, the KRAS inhibitor(s) and one additional agent(s) may be in the form of a single composition or multiple compositions, and the treatment modalities can be administered concurrently, sequentially, or through some other regimen. By way of example, in some embodiments there is provided a treatment regimen wherein a radiation phase is followed by a chemotherapeutic phase. A combination therapy can have an additive or synergistic effect.

In some embodiments, there is provided the use of a KRAS inhibitor compound or composition described herein in combination with bone marrow transplantation, peripheral blood stem cell transplantation, or other types of transplantation therapy.

In particular embodiments, there is provided the use of the inhibitors of KRAS function described herein in combination with immune checkpoint inhibitors. The blockade of immune checkpoints, which results in the amplification of antigen-specific T cell responses, has been shown to be a promising approach in human cancer therapeutics. Non-limiting examples of immune checkpoints (ligands and receptors), some of which are selectively upregulated in various types of tumor cells, that are candidates for blockade include PD1 (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors. Non-limiting examples of immune checkpoint inhibitors include ipulimumab, nivolumab and lambrolizumab.

In other embodiments, there are provided methods for treating a cancer in a subject, comprising administering to the subject a therapeutically effective amount of at least one KRAS inhibitor compound or composition thereof and at least one chemotherapeutic agent, such agents including, but not limited to alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). There is also provided the use of the KRAS inhibitors in combination with other agents known in the art (e.g., arsenic trioxide) and other chemotherapeutic or anti-cancer agents that may be appropriate for treatment.

In some embodiments drawn to methods of treating cancer, the administration of a therapeutically effective amount of a KRAS inhibitor in combination with at least one chemotherapeutic agent results in a cancer survival rate greater than the cancer survival rate observed by administering either agent alone. In further embodiments drawn to methods of treating cancer, the administration of a therapeutically effective amount of a KRAS inhibitor in combination with at least one chemotherapeutic agent results in a reduction of tumor size or a slowing of tumor growth greater than reduction of the tumor size or slowing of tumor growth observed by administration of either agent alone.

In further embodiments, there are provided methods for treating or preventing cancer in a subject, comprising administering to the subject a therapeutically effective amount of at least one KRAS inhibitor compound or composition and at least one signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and famesyl transferase inhibitors (FTIs).

In other embodiments, there are provided methods of augmenting the rejection of tumor cells in a subject comprising administering an KRAS inhibitor compound or composition in conjunction with at least one chemotherapeutic agent and/or radiation therapy, wherein the resulting rejection of tumor cells is greater than that obtained by administering either the KRAS inhibitor, the chemotherapeutic agent or the radiation therapy alone.

In further embodiments, there are provided methods for treating cancer in a subject, comprising administering to the subject a therapeutically effective amount of at least one KRAS inhibitor and at least one anti-cancer agent other than a KRAS inhibitor. It should be understood that, as used herein, a “KRAS inhibitor” refers to compounds provided herein, e.g., a compound of Formula A, a compound of Formula A-2, a compound of Table 2 or Table 3, or a pharmaceutically acceptable salt, ester, hydrate, or solvate thereof, or a stereoisomer thereof, and to pharmaceutical compositions thereof.

In some embodiments, there are provided methods of treating or preventing a KRAS-associated disease, disorder or condition in a subject in need thereof, comprising administering a therapeutically effective amount of at least one KRAS inhibitor or a pharmaceutical composition thereof to the subject, such that the KRAS-associated disease, disorder or condition is treated or prevented in the subject. In some embodiments, the KRAS-associated disease, disorder or condition is associated with at least one KRAS mutation, e.g., at least one of KRAS G12A, KRAS G12C, KRAS G12D, KRAS G12R, KRAS G12S, KRAS G12V, KRAS G13D and KRAS Q61H. In some embodiments, the KRAS-associated disease, disorder or condition is associated with at least two KRAS mutations, e.g., at least two of KRAS G12A, KRAS G12C, KRAS G12D, KRAS G12R, KRAS G12S, KRAS G12V, KRAS G13D and KRAS Q61H. In some embodiments, the compound is administered in an amount effective to reverse, slow or stop the progression of a KRAS-mediated cancer in the subject.

In some embodiments, the KRAS-associated disease, disorder or condition is a KRAS related cancer, tumor or hyperplastic or hyperproliferative disorder, such as, for example and without limitation, a cancer of the cardiac system, heart, lung, gastrointestinal system, genitourinary tract, liver, bone, nervous system, brain, gynecological system, hematologic tissues, skin, or adrenal glands, as described herein. In certain embodiments, the cancer, tumor or hyperplastic or hyperproliferative disorder is non-small cell lung cancer (NSCLC), small cell lung cancer, pancreatic cancer, biliary tract cancer, colorectal cancer, colon cancer, bile duct cancer, cervical cancer, bladder cancer, liver cancer or breast cancer.

In certain embodiments of methods of the disclosure, the inhibition, treatment, or prevention, in full or in part, of other diseases or disorders through degradation of KRAS protein using at least one of the compounds or compositions described herein is encompassed.

In some embodiments, methods provided herein further comprise administration of at least one additional therapeutic agent to the subject. The at least one additional therapeutic agent may be administered concomitantly or sequentially with the compound or composition described herein. In some embodiments, the at least one additional therapeutic agent is a chemotherapeutic agent or an anti-cancer agent. In an embodiment, the at least one additional therapeutic agent is an immune checkpoint inhibitor, such as, without limitation, ipulimumab, nivolumab or lambrolizumab.

In additional embodiments, methods provided herein further comprise administration of a tumor vaccine (e.g., a vaccine effective against melanoma); the tumor vaccine can comprise genetically modified tumor cells or a genetically modified cell line, including genetically modified tumor cells or a genetically modified cell line that has been transfected to express granulocyte-macrophage stimulating factor (GM-CSF). In particular embodiments, the vaccine includes one or more immunogenic peptides and/or dendritic cells.

In another broad aspect, there are provided kits comprising the compound or composition described herein. Kits may include a compound described herein, or a pharmaceutically acceptable salt, ester, hydrate, solvate or stereoisomer thereof, for use to treat, prevent or inhibit one or more KRAS-associated disease, disorder or condition. Kits may further comprise a buffer or excipient, and/or instructions for use. In some embodiments, kits further comprise at least one additional therapeutic agent, such as without limitation a chemotherapeutic agent, an immune- and/or inflammation-modulating agent, an anti-hypercholesterolemia agent, an anti-infective agent, or an immune checkpoint inhibitor.

### DETAILED DESCRIPTION

The number of subjects diagnosed with cancer and the number of deaths attributable to cancer continue to rise. Recent experimental evidence indicates that KRAS inhibitors may represent an important new treatment modality for treatment of many cancers and tumors. However, traditional treatment approaches including chemotherapy, radiotherapy and traditional enzymatic inhibitors are generally difficult for patients to tolerate and/or can become less effective as cancers and tumors evolve to circumvent such treatments.

There are provided herein, inter alia, bifunctional small molecule compounds that can inhibit KRAS (both mutant and wild-type proteins), as well as compositions thereof, and methods of using the compounds and compositions for the treatment and prevention of the diseases, disorders and conditions described herein. Compounds provided herein are useful as inhibitors of KRAS protein (both mutant and wild-type) and, therefore, useful in the treatment of diseases, disorders, and conditions in which KRAS activity plays a role. Specifically, compounds provided herein are proteolysis-targeting chimeras (Protacs) which can bind to a target protein of interest (KRAS mutant or wild-type) and to an E3 ligase. The compounds act to recruit the E3 ligase to the target protein (KRAS mutant or wild-type) and thereby modulate degradation of the target protein.

Without wishing to be limited by theory, Protacs can provide several advantages therapeutically compared to traditional enzymatic inhibitors. First, they need only bind to their targets with high selectivity to work (rather than inhibit the target protein's enzymatic activity). Further, previously undruggable proteins can be targeted, since a target catalytic pocket is not needed. Another advantage is that, due to their catalytic mechanism, Protacs can often be administered at lower doses compared to inhibitor analogues and traditional enzymatic inhibitor compounds. Off-target effects can also be reduced. Finally, acquired drug resistance is less likely to occur for Protacs. For example, treatment with Protacs may avoid or prevent mutation-driven drug resistance that would circumvent a traditional enzymatic inhibitor. KRAS inhibitor compounds of the disclosure may provide one or more of these advantages compared to other KRAS inhibitors.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The terms “about” and “approximately” are used to indicate that a value includes an inherent variation of error for the device or the method being employed to determine the value.

The term “derivative” as used herein, is understood as being a substance similar in structure to another compound but differing in some slight structural detail.

The term “KRAS protein” as used herein encompasses the wild-type KRAS as well as various mutated forms of KRAS protein. The multiple mutated forms of KRAS protein include, for example and without limitation, KRAS G12A, KRAS G12C, KRAS G12D, KRAS G12R, KRAS G12S, KRAS G12V, KRAS G13D and KRAS Q61H, etc. The term “KRAS-related diseases” as used in the present disclosure encompasses diseases caused by the wild-type KRAS and/or various mutated forms of KRAS protein, e.g., KRAS G12A, KRAS G12C, KRAS G12D, KRAS G12R, KRAS G12S, KRAS G12V, KRAS G13D and/or KRAS Q61H, etc.

The term “wild-type KRAS” as used herein refers to the non-mutated form of the mammalian KRAS protein. The assignment of amino acid codon and residue positions for human KRAS is based on the amino acid sequence identified by UniProtKB/Swiss-Prot P01116. The term “wild-type KRAS inhibitor” as used herein refers to compounds of the disclosure, as represented e.g. by Formula (A) herein, which are capable of negatively regulating or inhibiting the enzyme activity of wild-type KRAS, in whole or in part. As used herein, “wild-type KRAS-related diseases or conditions” refers to diseases or conditions that are associated with, mediated by, or involve wild-type KRAS. Non-limiting examples of wild-type KRAS-related diseases or conditions include wild-type KRAS-related cancers.

The term “KRAS G12A” as used in the present invention refers to a mutated form of the mammalian KRAS protein, which contains an amino acid substitution of an alanine for glycine at amino acid position 12. The assignment of amino acid codon and residue positions for human KRAS is based on the amino acid sequence identified by UniProtKB/Swiss-Prot P01116: Variant p.Gly12Ala. The term “KRAS G12A inhibitor” as used herein refers to compounds of the disclosure, as represented e.g. by Formula (A) herein, which are capable of negatively regulating or inhibiting the enzyme activity of KRAS G12A, in whole or in part. As used herein, “KRAS G12A-related diseases or conditions” refers to diseases or conditions that are associated with, mediated by, or involve the KRAS G12A mutation. Non-limiting examples of KRAS G12A-related diseases or conditions include KRAS G12A-related cancers.

The term “KRAS G12C” as used herein refers to a mutated form of the mammalian KRAS protein, which contains an amino acid substitution of a cysteine for a glycine at amino acid position 12. The assignment of amino acid codon and residue positions for human KRAS is based on the amino acid sequence identified by UniProtKB/Swiss-Prot P01116: Variant p.Gly12Cys. The term “KRAS G12C inhibitor” as used herein refers to compounds of the disclosure, as represented e.g. by Formula (A) herein, which are capable of negatively regulating or inhibiting the enzyme activity of KRAS G12C, in whole or in part. The term “KRAS G12C-related diseases or conditions” as used herein refers to diseases or conditions that are associated with or mediated by or involve the KRAS G12C mutation. Non-limiting examples of KRAS G12C-related diseases or conditions include KRAS G12C-related cancers.

The term “KRAS G12D” as used herein refers to a mutated form of the mammalian KRAS protein, which contains an amino acid substitution of an aspartic acid for a glycine at amino acid position 12. The assignment of amino acid codon and residue positions for human KRAS is based on the amino acid sequence identified by UniProtKB/Swiss-Prot P01116: Variant p.Gly12Asp. As used in this document, a “KRAS G12D inhibitor” refers to compounds of the disclosure, as shown e.g. by Formula (A), which are capable of negatively regulating or inhibiting the enzymatic activity of KRAS G12D, in whole or in part. The term “KRAS G12D-related diseases or conditions” as used herein refers to diseases or conditions that are associated with or mediated by or involve the KRAS G12D mutation. Non-limiting examples of KRAS G12D-related diseases or conditions include KRAS G12D-related cancers.

The term “KRAS G12R” as used herein refers to a mutated form of the mammalian KRAS protein, which contains an amino acid substitution of an arginine for a glycine at amino acid position 12. The assignment of amino acid codon and residue positions for human KRAS is based on the amino acid sequence identified by UniProtKB/Swiss-Prot P01116: Variant p.Gly12Arg. The term “KRAS G12R inhibitor” as used herein refers to compounds of the disclosure, as represented e.g. by Formula (A) herein, which are capable of negatively regulating or inhibiting the enzyme activity of KRAS G12R, in whole or in part. The term “KRAS G12R-related diseases or conditions” as used herein refers to diseases or conditions that are associated with or mediated by or involve the KRAS G12R mutation. Non-limiting examples of KRAS G12R-related diseases or conditions include KRAS G12R-related cancers.

The term “KRAS G12S” as used herein refers to a mutated form of the mammalian KRAS protein, which contains an amino acid substitution of a serine for a glycine at amino acid position 12. The assignment of amino acid codon and residue positions for human KRAS is based on the amino acid sequence identified by UniProtKB/Swiss-Prot P01116: Variant p.Gly12Ser. The term “KRAS G12S inhibitor” as used herein refers to compounds of the disclosure, as represented e.g. by Formula (A) herein, which are capable of negatively regulating or inhibiting the enzyme activity of KRAS G12S, in whole or in part. The term “KRAS G12S-related diseases or conditions” as used herein refers to diseases or conditions that are associated with or mediated by or involve the KRAS G12S mutation. Non-limiting examples of KRAS G12S-related diseases or conditions include KRAS G12S-related cancers.

The term “KRAS G12V” as used herein refers to a mutated form of the mammalian KRAS protein, which contains an amino acid substitution of a valine for a glycine at amino acid position 12. The assignment of amino acid codon and residue positions for human KRAS is based on the amino acid sequence identified byUniProtKB/Swiss-Prot P01116: Variant p.Gly12Val. The term “KRAS G12V inhibitor” as used herein refers to compounds of the disclosure, as represented e.g. by Formula (A), which are capable of negatively regulating or inhibiting the full or partial enzymatic activity of KRAS G12V. The term “KRAS G12V-related diseases or conditions” as used herein refers to diseases or conditions that are associated with or mediated by or involve the KRAS G12V mutation. Non-limiting examples of KRAS G12V-related diseases or conditions include KRAS G12V-related cancers.

The term “KRAS G13D” as used herein refers to a mutated form of the mammalian KRAS protein, which contains an amino acid substitution of an aspartic acid for a glycine at amino acid position 13. The assignment of amino acid codon and residue positions for human KRAS is based on the amino acid sequence identified by UniProtKB/Swiss-Prot P01116: Variant p.Gly13Asp. The term “KRAS G13D inhibitor” as used herein refers to compounds of the disclosure, as represented e.g. by Formula (A) herein, which are capable of negatively regulating or inhibiting the enzyme activity of KRAS G13D, in whole or in part. The term “KRAS G13D-related diseases or conditions” as used herein refers to diseases or conditions associated with, mediated by, or involve KRAS G13D. Non-limiting examples of KRAS G13D-related diseases or conditions include KRAS G13D-related cancers.

The term “KRAS Q61H” as used herein refers to a mutated form of the mammalian KRAS protein, which contains an amino acid substitution of a histidine for a glutamine at amino acid position 61. The assignment of amino acid codon and residue positions for human KRAS is based on the amino acid sequence identified by UniProtKB/Swiss-Prot P01116: Variant p. Gln61His. The term “KRAS Q61H inhibitor” as used herein refers to compounds of the disclosure, as represented e.g. by Formula (A) herein, which are capable of negatively regulating or inhibiting the enzyme activity of KRAS Q61H in whole or in part. The term “KRAS Q61H-related diseases or conditions” as used in the present invention refers to diseases or conditions that are associated with or mediated by or involve the KRAS Q61H mutation. Non-limiting examples of KRAS Q61H-related diseases or conditions include KRAS Q61H-related cancers.

Ubiquitin (Ub) is a small protein that exists in all eukaryotic cells and is highly conserved throughout eukaryotic evolution, with human and yeast ubiquitin sharing 96% sequence identity (the yeast and human sequences differing by only 3 amino acids). It contains 76 amino acids and has a molecular mass of approximately 8.5 kDa. Ubiquitin performs myriad functions through conjugation to a large range of target proteins; in general, ubiquitination affects cellular processes by regulating the degradation of proteins (via the proteasome and lysosome), coordinating the cellular localization of proteins, activating and inactivating proteins, and modulating protein-protein interactions. Ubiquitination refers to the process by which ubiquitin is covalently bound to target proteins under the catalytic action of a series of enzymes. The ubiquitination process typically involves the coordinated action of three types of ubiquitination enzymes: E1 ubiquitin-activating enzymes, E2 ubiquitin-conjugating enzymes, and E3 ubiquitin ligases. Common E3 ubiquitin ligases include VHL (Von Hippel-Lindau), CRBN (Cereblon), MDM2, cIAP, AhR, Nimbolide, CCW16, KB02, KEAP1, and so on.

The term “ubiquitylation” (also referred to as “ubiquitination” or “ubiquitinylation”) is an enzymatic post-translational modification in which a ubiquitin protein is attached to a substrate protein. In general, ubiquitination refers to the process of covalent binding of ubiquitin to a target protein under the catalysis of a series of enzymes. The ubiquitination process usually requires the cooperation of three ubiquitination enzymes: E1 ubiquitin activating enzyme, E2 ubiquitin binding enzyme, and E3 ubiquitin ligase (also referred to herein as “E3 ligase”; the terms “E3 ubiquitin ligase” and “E3 ligase” are used interchangeably herein). E3 ubiquitin ligases catalyze the final step of the ubiquitination cascade, most commonly creating an isopeptide bond between a ligand of the substrate/target protein and the C-terminal glycine of ubiquitin. Common E3 ubiquitin ligases include, for example and without limitation, VHL (Von Hippel-Lindau), CRBN (Cereblon), MDM2, c-IAP1, AhR, Nimbolide, CCW16, KB02, KEAP1, beta-TrCP1, DCAF15, DCAF16, RNF114, and others. Hundreds of E3 ubiquitin ligases are known, and it should be understood that any suitable E3 ligase may be targeted/bound by compounds of the present disclosure.

The term “proteolysis targeting chimera” or “Protac” refers to a heterobifunctional molecule, composed of two active domains and optionally a linker, which is capable of removing specific unwanted proteins. The active domains are protein-binding domains, one that binds to a target protein meant for degradation and one that binds to an E3 ubiquitin ligase. Recruitment of the E3 ligase to the target protein results in ubiquitination and subsequent degradation of the target protein via the proteasome. In this way Protacs act to induce selective intracellular proteolysis.

The term “prodrug” or its equivalent refers to a reagent that is directly or indirectly converted into an active form in vitro or in vivo (see, for example, R. B. Silverman, 1992, “The Organic Chemistry of Drug Design and Drug Action,” Academic Press, Chap. 8; Bundgaard, Hans; Editor. Neth. (1985), “Design of Prodrugs” 360 pp. Elsevier, Amsterdam; Stella, V.; Borchardt, R.; Hageman, M.; Oliyai, R.; Maag, H.; Tilley, J. (Eds.) (2007), “Prodrugs: Challenges and Rewards, XVIII, 1470 p. Springer). A prodrug can be used to change the biological distribution of specific drugs (for example, to make the drug usually not enter the protease reaction site) or its pharmacokinetics. A variety of groups have been used to modify compounds to form prodrugs, such as esters, ethers, phosphate esters/salts, etc. When a prodrug is administered to a subject, the group is cleaved in the subject by an enzymatic or non-enzymatic process, e.g., by reduction, oxidation or hydrolysis, or in another way, to release the active compound. As used herein, “prodrug” may include pharmaceutically acceptable salts or esters, or pharmaceutically acceptable solvates or chelates, as well as crystalline forms of a compound.

The terms “peptide”, “polypeptide” and “oligopeptide” refer to a compound formed by the dehydration and condensation of two or more amino acid residues, which are linked together by amide bonds. In general, the number of amino acids in a small peptide or oligopeptide is from 2 (dipeptide) to 20 (icosapeptide), although the number is not particularly limited.

The term “residue” refers to the main part of a molecule which remains after removing a certain group, such as an amino acid residue (such as the structure H₂NCH₂C(O)—, that is, the glycyl group, which is the part remaining after removing a hydroxyl group from glycine) or a peptide residue.

The present description refers to a number of chemical terms and abbreviations used by those skilled in the art. Nevertheless, definitions of selected terms are provided for clarity and consistency.

As used herein, the term “hydrocarbon” refers to an organic compound consisting entirely of hydrogen and carbon; it also refers to a group or a molecular fragment derived therefrom by removing one or more hydrogen atoms, which is also called a “hydrocarbon group”. The term “hydrocarbon group” includes saturated and unsaturated hydrocarbon groups, e.g., aliphatic and aromatic hydrocarbon group, e.g., alkyl groups, aryl groups, etc. Hydrocarbon groups may also include one or more heteroatom (atom which is not carbon or hydrogen); examples of such heterohydrocarbon groups include, without limitation, oxoalkyl groups, azalkyl groups, sulfoalkyl groups, phosphoroalkyl groups and mixed heterohydrocarbon groups with different heteroatoms. The chain length of hydrocarbon or heterohydrocarbon groups is not particularly limited but is generally from 1 to 20 carbon atoms, and heterohydrocarbon groups generally contain from 1 to 5 heteroatoms. It should be understood that the chemical valence of a heteroatom can be filled by hydrogen, oxygen, nitrogen, etc. in the corresponding bonding manner, as required.

As used herein, the term “alkyl” refers to saturated hydrocarbons having from one to thirty carbon atoms, including linear, branched, and cyclic alkyl groups. Examples of alkyl groups include, without limitation, methyl, ethyl, propyl, butyl, pentyl, hexyl, heptyl, octyl, nonyl, decyl, isopropyl, tert-butyl, sec-butyl, isobutyl, cyclopropyl, cyclopentyl, cyclohexyl, cycloheptyl, cyclooctyl, and the like. The term alkyl includes both unsubstituted alkyl groups and substituted alkyl groups. The terms “C₁-Cₙalkyl” and “C₁₋ₙ alkyl”, wherein n is an integer from 2 to 30, are used interchangeably to refer to an alkyl group having from 1 to the indicated “n” number of carbon atoms. Alkyl residues may be substituted or unsubstituted. In some embodiments, for example, alkyl may be substituted by hydroxyl, amino, carboxyl, carboxylic ester, amide, carbamate, or aminoalkyl. In some particular embodiments, “alkyl” is modified by a range of the number of carbon atoms and thus the size of the alkyl group is defined specifically. For example, a C₁₁-C₃₀ alkyl specifies an alkyl group containing at least 11 carbon atoms and not more than 30 carbon atoms.

As used herein, the term “acyclic” refers to an organic moiety without a ring system. The term “aliphatic group” includes organic moieties characterized by straight or branched-chains, typically having between 1 and 15 carbon atoms. Aliphatic groups include non-cyclic alkyl groups, alkenyl groups, and alkynyl groups.

As used herein, the term “alkenyl” refers to unsaturated hydrocarbons having from two to thirty carbon atoms, including linear, branched, and cyclic non aromatic alkenyl groups, and comprising between one to six carbon-carbon double bonds. Examples of alkenyl groups include, without limitation, vinyl, allyl, 1-propen-2-yl, 1-buten-3-yl, 1-buten-4-yl, 2-buten-4-yl, 1-penten-5-yl, 1,3-pentadien-5-yl, cyclopentenyl, cyclohexenyl, ethylcyclopentenyl, ethylcylohexenyl, and the like. The term alkenyl includes both unsubstituted alkenyl groups and substituted alkenyl groups. The terms “C₂-Cₙalkenyl” and “C₂₋ₙalkenyl”, wherein n is an integer from 3 to 30, are used interchangeably to refer to an alkenyl group having from 2 to the indicated “n” number of carbon atoms. In some particular embodiments, “alkenyl” is modified by a range of the number of carbon atoms and thus the size of the alkenyl group is defined specifically. For example, a C₁₁-C₃₀ alkenyl specifies an alkenyl group containing at least 11 carbon atoms and not more than 30 carbon atoms.

As used herein, the term “alkynyl” refers to unsaturated hydrocarbons having from two to thirty carbon atoms, including linear, branched, and cyclic non aromatic alkynyl groups, and comprising between one to six carbon-carbon triple bonds. Examples of alkynyl groups include, without limitation, ethynyl, 1-propyn-3-yl, 1-butyn-4-yl, 2-butyn-4-yl, 1-pentyn-5-yl, 1,3-pentadiyn-5-yl, and the like. The term alkynyl includes both unsubstituted alkynyl groups and substituted alkynyl groups. The terms “C₂-Cₙalkynyl” and “C₂₋ₙ alkynyl”, wherein n is an integer from 3 to 30, are used interchangeably to refer to an alkynyl group having from 2 to the indicated “n” number of carbon atoms. In some particular embodiments, “alkynyl” is modified by a range of the number of carbon atoms and thus the size of the alkynyl group is defined specifically. For example, a C₁₁-C₃₀ alkynyl specifies an alkynyl group containing at least 11 carbon atoms and not more than 30 carbon atoms.

Unless the number of carbons is otherwise specified, “lower” as in “lower aliphatic,” “lower alkyl,” “lower alkenyl,” and “lower alkylnyl”, as used herein means that the moiety has at least one (two for alkenyl and alkynyl) and equal to or less than 6 carbon atoms.

The terms “cycloalkyl”, “alicyclic”, “carbocyclic”, “cyclic group”, “alicyclic group”, “cyclic hydrocarbon group” and equivalent expressions refer to a group comprising a saturated or partially unsaturated carbocyclic ring in a single, spiro (sharing one atom), or fused (sharing at least one bond) carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbomyl, and the like. The term cycloalkyl includes both unsubstituted cycloalkyl groups and substituted cycloalkyl groups. The terms “C₃-Cₙcycloalkyl” and “C₃₋ₙ cycloalkyl”, wherein n is an integer from 4 to 15, are used interchangeably to refer to a cycloalkyl group having from 3 to the indicated “n” number of carbon atoms in the ring structure. Unless the number of carbons is otherwise specified, “lower cycloalkyl” groups as herein used, have at least 3 and equal to or less than 8 carbon atoms in their ring structure.

Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C₁₋₄ alkyl. Examples of substituted cycloalkyl residues are 4-methylcyclohexyl and 2,3-dimethylcyclopentyl. Examples of parent structures of bicyclic ring systems are norbornane, bicyclo[2.2.1]heptane, bicyclo[2.2.2]octane and bicyclo[3.2.1]octane.

The term “heterocycloalkyl” and equivalent expressions refers to a group comprising a saturated or partially unsaturated carbocyclic ring in a single, spiro (sharing one atom), or fused (sharing at least one bond) carbocyclic ring system having from three to fifteen ring members, including one to six heteroatoms (e.g., N, O, S, P) or groups containing such heteroatoms (e.g., NH, NRx (Rx is alkyl, acyl, aryl, heteroaryl or cycloalkyl), PO₂, SO, SO₂, and the like). Heterocycloalkyl groups may be C-attached or heteroatom-attached (e.g., via a nitrogen atom) where such is possible. Examples of heterocycloalkyl groups include, without limitation, pyrrolidino, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, quinolizinyl, and sugars, and the like. The term heterocycloalkyl includes both unsubstituted heterocycloalkyl groups and substituted heterocycloalkyl groups. The terms “C₃-Cₙheterocycloalkyl” and “C₃₋ₙ heterocycloalkyl”, wherein n is an integer from 4 to 15, are used interchangeably to refer to a heterocycloalkyl group having from 3 to the indicated “n” number of atoms in the ring structure, including at least one hetero group or atom as defined above. Unless the number of carbons is otherwise specified, “lower heterocycloalkyl” groups as herein used, have at least 3 and equal to or less than 8 carbon atoms in their ring structure.

The terms “aryl” and “aryl ring” refer to aromatic groups having “4n+2” (pi) electrons, wherein n is an integer from 1 to 7, in a conjugated monocyclic or polycyclic system (fused or not) and having six to fourteen ring atoms. In certain embodiments, n is an integer from 1 to 3. A polycyclic ring system includes at least one aromatic ring. Aryl may be directly attached, or connected via a C₁-C₃ alkyl group or a C₁-C₆ alkyl group (also referred to as arylalkyl or aralkyl). Examples of aryl groups include, without limitation, phenyl, benzyl, phenethyl, 1-phenylethyl, tolyl, naphthyl, biphenyl, triphenyl, terphenyl, indenyl, benzocyclooctenyl, benzocycloheptenyl, benzocycloheptyl, azulene, acenaphthene, azulenyl, acenaphthylenyl, fluorenyl, phenanthernyl, anthracene, anthracenyl, and the like. The term aryl includes both unsubstituted aryl groups and substituted aryl groups. The terms “C₆-Cₙaryl” and “C₆₋ₙ aryl”, wherein n is an integer from 6 to 30, are used interchangeably to refer to an aryl group having from 6 to the indicated “n” number of atoms in the ring structure, including at least one hetero group or atom as defined above. When the aryl group is connected to an alkyl group, the entire group is known as arylalkyl group or alkylaryl group.

The term “heteroaryl”, “aromatic heterocycle” or “heteroaromatic ring” as used herein covers substituted or unsubstituted nitrogen-containing six-membered aromatic heterocycle, substituted or unsubstituted five-membered aromatic heterocycle, wherein the substituent is selected from C₁₋₄ straight or branched hydrocarbyl, halogen-substituted C₁₋₄ straight or branched hydrocarbyl, F, Cl, Br, NO₂, CN, methylenedioxy, cyclopropyl, cyclopropylmethylenel, substituted or unsubstituted cyclobutyl, substituted or unsubstituted cyclopentyl. The nitrogen-containing six-membered aromatic heterocycle, and five-membered aromatic heterocycle, may be singly substituted or multiply substituted; the six-membered aromatic heterocycle may contain one N atom or multiple nitrogen atoms; and the five-membered aromatic heterocycle may contain one heteroatom or multiple heteroatoms. In some embodiments the heteroatoms are selected from O, N and S; the number of heteroatoms is selected from 1, 2 and 3; and the halogens are selected from F, Cl and Br.

In some embodiments, the substituent in the substituted phenyl, substituted nitrogen-containing six-membered aromatic heterocycle, substituted five-membered aromatic heterocycle, is selected from:
(a) C₁₋₈ straight or branched hydrocarbyl, halogen-substituted C₁₋₈ straight or branched hydrocarbyl, F, Cl, Br, NO₂, CN, methylenedioxy, OR¹, SR², NR³R¹, NR⁴COR², COOR⁵, CONR⁶R³, NR⁷COOR⁴, SO₂NR⁸R⁵, (CH₂)ₙNR⁹R⁶, (CH₂)ₙOR¹⁰, wherein R¹, R², R³, R⁴, R⁵, R⁶, R⁷, R⁸, R⁹ and R¹⁰ are independently selected from H, substituted or unsubstituted C₁₋₈ straight or branched hydrocarbyl, substituted or unsubstituted C₂₋₈ straight or branched alkenyl, substituted or unsubstituted C₂₋₈ straight or branched alkynyl, substituted or unsubstituted 3-7 membered cyclohydrocarbyl, substituted or unsubstituted 3-8 membered oxygen-containing cycloheterohydrocarbyl, substituted or unsubstituted 3-8 membered nitrogen-containing cycloheterohydrocarbyl, substituted or unsubstituted phenyl, substituted or unsubstituted six-membered aromatic heterocycle, substituted or unsubstituted five-membered aromatic heterocycle, where the substituent is selected from F, Cl, Br, CN, OR^{a1}, SR^{a2}, NR^{a3}R^{b1}, COOR^{a4}, CONR^{a5}R^{b2}, NR^{a6}COOR^{b3}, SO₂NR^{a7}R^{b4} and NR^{a8}COR^{b5}, where R^{a1}, R^{a2}, R^{a3}, R^{b1}, R^{a4}, R^{a5}, R^{b2}, R^{a6}, R^{b3}, R^{a7}, R^{b4}, R^{a8} and R^{b5} are independently selected from H, C₁₋₄ straight or branched alkyl, cyclopropyl, cyclopropylmethylene, cyclobutyl, cyclopentyl and cyclohexyl; the 3-8 membered oxygen-containing cycloheterohydrocarbyl or nitrogen-containing cycloheterohydrocarbyl may contain one heteroatom, or may simultaneously contain multiple heteroatoms; and n is selected from 1, 2 and 3;
(b) substituted or unsubstituted C₃₋₇ cycloalkyl, substituted or unsubstituted 3-8 membered oxygen-containing cycloheterohydrocarbyl, substituted or unsubstituted 3-8 membered nitrogen-containing cycloheterohydrocarbyl, wherein the substituent described is selected from C₁₋₅ straight or branched hydrocarbyl, F, Cl, Br, CN, OR^{a1}, SR^{a2}, NR^{a3}R^{b1}, COOR^{a4}, CONR^{a5}R^{b2}, NR^{a6}COOR^{b3}, SO₂NR^{a7}R^{b4} and NR^{a8}COR^{b5}, where R^{a1}, R^{a2}, R^{a3}, R^{b1}, R^{a4}, R^{a5}, R^{b2}, R^{a6}, R^{b3}, R^{a7}, R^{b4}, R^{a8} and R^{b5} are independently selected from H, C₁₋₄ straight or branched hydrocarbyl, cyclopropyl, cyclopropylmethylene, cyclobutyl, cyclopentyl and cyclohexyl; the 3-8 membered oxygen-containing cycloheterohydrocarbyl or nitrogen-containing cycloheterohydrocarbyl may contain one heteroatom, or may simultaneously contain multiple heteroatoms;
(c) substituted or unsubstituted phenyl, substituted or unsubstituted six-membered aromatic heterocycle, substituted or unsubstituted five-membered aromatic heterocycle, wherein the substituent is selected from F, Cl, Br, CN, OR^{a1}, SR^{a2}, NR^{a3}R^{b1}, COOR^{a4}, CONR^{a5}R^{b2}, NR^{a6}COOR^{b3}, SO₂NR^{a7}R^{b4} and NR^{a8}COR^{b5}, wherein R^{a1}, R^{a2}, R^{a3}, R^{b1}, R^{a4}, R^{a5}, R^{b2}, R^{a6}, R^{b3}, R^{a7}, R^{b4}, R^{a8} and R^{b5} are independently selected from H, C₁₋₄ straight or branched hydrocarbyl, cyclopropyl, cyclopropylmethylene, cyclobutyl, cyclopentyl and cyclohexyl; the 3-8 membered oxygen-containing cycloheterohydrocarbyl or nitrogen-containing cycloheterohydrocarbyl may contain one heteroatom, or may simultaneously contain multiple heteroatoms; the phenyl ring, six-membered aromatic heterocycle, or five-membered aromatic heterocycle can be mono-substituted or poly-substituted; the six-membered aromatic heterocycleand the five-membered aromatic heterocycle may contain 1 heteroatom, or may contain multiple heteroatoms, wherein the heteroatoms are selected from O, N and S.

In some embodiments, substituted or unsubstituted aromatic fused rings or fused heterocycles, substituted or unsubstituted non-aromatic fused rings or fused heterocycles, including substituted or unsubstituted naphthalene ring, substituted or unsubstituted six-membered benzoheterocycle, substituted or unsubstituted five-membered benzoheterocycle, wherein the substituent described is selected from C₁₋₄ straight or branched hydrocarbyl, halogen-substituted C₁₋₄ straight or branched hydrocarbyl, F, Cl, Br, NO₂, CN, methylenedioxy, OR^{s1}, SR^{s2}, NR^{s3}R^{t1}, NR^{s4}COR^{t2}, COOR^{s5}, CONR^{s6}R^{t3}, NR^{s7}COOR^{t4}, SO₂NR^{s8}R^{t5}, (CH₂)ₙNR^{s9}R^{t6} and (CH₂)ₙOR^{s10}, wherein R^{s1}, R^{s2}, R^{s3}, R^{t1}, R^{s4}, R^{t2}, R^{s5}, R^{s6}, R^{t3}, R^{s7}, R^{t4}, R^{s8}, R^{t5}, R^{s9}, R^{t6} and R^{s10} are independently selected from H, C₁₋₄ straight or branched hydrocarbyl, cyclopropyl, cyclopropylmethylene, cyclobutyl, and cyclopentyl; the naphthalene ring, six-membered benzoheterocycle, or five-membered benzoheterocycle can be mono-substituted or poly-substituted; the six-membered benzoheterocycle or five-membered benzoheterocycle may contain one heteroatom, or may contain multiple heteroatoms, wherein the heteroatoms are selected from O, N and S; n is selected from 1, 2 and 3; and the halogens are selected from F, Cl and Br.

The term “hydrocarbyl” includes, but is not limited to saturated hydrocarbyl, unsaturated hydrocarbyl, aromatic hydrocarbyl, oxyhydrocarbyl, azahydrocarbyl, thiahydrocarbyl, phosphahydrocarbyl, as well as mixed heterohydrocarbyl with various heteroatoms. The chain length of the hydrocarbyl or heterohydrocarbyl ranges from 1 to 20 atoms. When hydrocarbyl is a heterohydrocarbyl, it contains 1 to 5 heteroatoms, and the chemical valence of these heteroatoms can be satisfied by hydrogen, oxygen, nitrogen, etc., as needed, through appropriate bonding.

The term “amidohydrocarbyl” or “hydrocarbylamido” refers to a group where a hydrocarbyl group is linked to an acylamino group. The term “acylhydrocarbyl” or “hydrocarbylacyl” refers to a group where an acyl group is linked to a hydrocarbyl group. The term “carbonylhydrocarbyl” or “hydrocarbylcarbonyl” refers to a group where a hydrocarbyl group is linked to a carbonyl group.

The terms “cyclic group”, “alicyclic”, “cyclohydrocarbyl”, and equivalent expressions refer to a group containing saturated or partially unsaturated carbon rings within a monocyclic, spiro (sharing one atom) or fused (sharing at least one bond) carbon ring system having 3 to 15 carbon atoms. The term “cyclohydrocarbyl” includes a combination group of a cyclic group and a hydrocarbyl.

The term “heterocyclic ring” and equivalent descriptions used herein refer to a group containing saturated or unsaturated carbon ring in a monocyclic, spiro (sharing one atom) or fused (sharing at least one bond) carbon ring system having 3 to 15 carbon atoms, which includes 1 to 6 heteroatoms (e.g., N, O, S and P) or a group containing a heteroatom (e.g., NH, NRx (where Rx is alkyl, acyl, aryl, heteroaryl or cycloalkyl), PO₂, SO, SO₂, etc.). Heterocyclohydrocarbyl can be linked to C or heteroatom (e.g., via N atom). “Heterocycle” or “heterocyclic” covers heterocycloalkyl and heteroaryl. The examples of heterocyclic ring include but are not limited to acridinyl, azocinyl, benzimidazolyl, benzofuranyl, benzothiophenyl, benzoxazolyl, benzothiazolyl, benzotriazolyl, benzotetrazolyl, benzoisoxazolyl, benzoisothiazolyl, 4aH-carbazolyl, carbolinyl, chromanyl, chromenyl, cinnolinyl, decahydroquinolinyl, 2H, 6H-1, 5, 2-dithiazinyl, dihydrofurano[2, 3-b]tetrahydrofuran, furanyl, furazanyl, imidazolidinyl, imidazolinyl, imidazolyl, 1H-indazolyl, dihydroindolyl, 3H-indazolyl, isoquinolinyl, isothiazolyl, isoxazolyl, methylenedioxybenzyl, morpholinyl, naphthyridinyl, octahydroisoquinolinyl, oxadiazolyl, 1, 2, 3-oxadiazoly, 1, 2, 4-oxadiazoly, 1, 2, 5-oxadiazoly, 1, 3, 4-oxadiazoly, oxazolidinyl, oxazolyl, pyrimidinyl, phenanthridinyl, phenanthrolinyl, phenazinyl, phenothiazinyl, phenoxazinyl, phthalazinyl, piperazinyl, piperidyl, piperidonyl, 4-piperidonyl, piperonyl, pteridyl, purinyl, pyranyl, pyrazinyl, pyrazolidinyl, pyrazolinyl, pyrazolyl, pyridazinyl, pyrido-oxazole, pyrido-imidazole, pyrido-thiazole, pyridyl, pyrryl, quinazolinyl, quinolyl, 4H-quinolizinyl, quinoxalinyl, quinuclidinyl, tetrahydrofuranyl, tetrahydroisoquinolinyl, tetrahydroquinolinyl, tetrazolyl, 6H-1, 2, 5-thiadiazinyl, 1, 2, 3-thiadiazolyl, 1, 2, 4-thiadiazolyl, 1, 2, 5-thiadiazolyl, 1, 3, 4-thiadiazolyl, thianthrenyl, thiazolyl, thiophenyl, thieno-thiazolyl, thieno-oxazolyl, thieno-imidazolyl, triazinyl, 1, 2, 3-triazolyl 1, 2, 4-triazolyl, 1, 2, 5-triazolyl, 3, 4-triazolyl, xanthenyl, etc. The term “cycloalkyl” includes unsubstituted heterocyclyls and substituted heterocyclyls. The term “heterocyclohydrocarbyl” refers to a combination group of a heterocyclic group and a hydrocarbyl.

The term “amine” or “amino,” as used herein, refers to an unsubstituted or substituted moiety of the formula —NRaRb, in which Ra and Rb are each independently hydrogen, alkyl, aryl, or heterocyclyl, or Ra and Rb, taken together with the nitrogen atom to which they are attached, form a heterocyclic ring. For example, an amine or amino may be an unsubstituted or substituted fragment of a general formula —N, including —NH₂, —NHR, or —NRR′, where R and R′ are the same or different and are substituted or unsubstituted and saturated or unsaturated alkyl or hydrocarbon groups. The term amino includes compounds or moieties in which a nitrogen atom is covalently bonded to at least one carbon or heteroatom. Thus, the terms “alkylamino” and “dialkylamino” as used herein mean an amine group having respectively one and at least two C₁-C₆ alkyl groups attached thereto. The terms “arylamino” and “diarylamino” include groups wherein the nitrogen is bound to at least one or two aryl groups, respectively. The terms “amide”, “amide group” or “aminocarbonyl” include compounds or moieties which contain a nitrogen atom which is bound to the carbon of a carbonyl or a thiocarbonyl group. For example, an amide group may have the structure —C(═O)NH₂, —C(═O)NHR, or —C(═O)NRR′, in which the amino group is directly connected to the acyl group. The term “acyl hydrocarbon group” or “acyl alkyl group” refers to the combined group of acyl and hydrocarbon/alkyl, in which the carbon atom of the acyl group is connected to the hydrocarbon/alkyl group. The term “acylamino” refers to an amino group directly attached to an acyl group as defined herein, i.e., —C(═O)N—. The term “acrylhydrocarbyl” refers to a combination of an acyl group and a hydrocarbyl group, where the carbon atom on the acyl group is linked to the hydrocarbyl group.

The term “bicycle” or “bicyclic” refers to a ring system with two rings that has two ring carbon atoms in common, and which can be located at any position along either ring, generally referring to bicyclic hydrocarbon radical, bicyclic aromatic carbon atom ring structure radical, and a saturated or partially unsaturated bicyclic carbon atom ring structure radical in which one or more carbon atom ring members have been replaced, where allowed by structural stability, with a heteroatom, such as an O, S or N atom. The bicyclic system can be a fused-ring system, such as bicyclo[4.4.0]decane or naphthalene, or a bridged-ring system, such as bicyclo[2.2.2]octane.

The term “tricycle” or “tricyclic” refers to a ring system with three rings that has three ring carbon atoms in common, and which can be located at any position along each ring; generally referring to tricyclic hydrocarbon radical, tricyclic aromatic carbon atom ring structure radical, and a saturated or partially unsaturated tricyclic carbon atom ring structure radical in which one or more carbon atom ring members have been replaced, where allowed by structural stability, with a heteroatom, such as an O, S or N atom. A tricyclic system can have three rings arranged as a fused ring, such as anthracene or tetradecahydroanthracene, or a bridged ring, such as in adamantine or tricycle[3.3.1.1]decane.

The term “multi-cycle”, “multicycle”, “multi-cyclic”, or “multi-cyclic” means a ring system with more than three rings having more than three ring carbon atoms in common, and which can be located at any position along either ring. The term generally refers to a multicyclic hydrocarbon radical, a multicyclic aromatic carbon atom ring structure radical, and a saturated or partially unsaturated multicyclic carbon atom ring structure radical in which one or more carbon atom ring members have been replaced, where allowed by structural stability, with a heteroatom, such as an O, S or N atom.

The term “fused ring” or “fused” refers to a polycyclic ring system that contains fused rings. Typically, a fused ring system contains 2 or 3 rings and/or up to 18 ring atoms. As defined above, cycloalkyl radicals, aryl radicals and heterocyclyl radicals may form fused ring systems. Thus, a fused ring system may be aromatic, partially aromatic or not aromatic and may contain heteroatoms. A spiro ring system is not a fused-polycyclic by this definition, but fused polycyclic ring systems of the invention may themselves have spiro rings attached thereto via a single ring atom of the system. The term “benzo-fused ring” refers to a fused ring system in which at least one of the rings is a benzene ring. Examples of fused ring systems include, but are not limited to, naphthyl (e.g. 2-naphthyl), indenyl, phenanthryl, anthracyl, pyrenyl, benzimidazolyl, benzothiazolyl, etc. The terms “fused ring” and “fused-cyclic” are used interchangeably herein.

The term “spiro” or “spirocyclic” refers to organic compounds that exhibit a twisted structure involving two or more rings (ring systems) where 2 or 3 rings are linked through one shared atom. Spirocyclic compounds can consist of entirely carbon rings (all-carbon), such as spiro [5.5] undecane, or heterocyclic compounds (containing one or more non-carbon atoms), including but not limited to carbon spirocyclic compounds, heterocyclic spirocyclic compounds, and polycyclic compounds.

The term “bridged ring” or “bridged” refers to carbon or heterocyclic moieties sharing two or more atoms in two or more ring structures, where the shared atoms can be C, N, S, or other heteroatoms arranged in chemically reasonable substitution patterns. Alternatively, “bridged ring” compounds also refer to carbocyclic or heterocyclic structures in which an atom at any position on the main ring is bonded to a second atom on the main ring via a chemical bond or an atom other than a bond, and it does not actually form a part of the main ring structure. The first and second atoms can be adjacent to each other or non-adjacent within the main ring. Other carbon ring or heterocyclic bridged ring structures are also foreseeable, including bridged rings where the bridging atoms are either C or heteroatoms arranged in chemically reasonable substitution patterns, as known in the field.

The term “nitro” means —NO₂; the terms “halo” and “halogen” refer to bromine, chlorine, fluorine or iodine substituents; the terms “thiol”, “thio”, and “mercapto” mean —SH; and the terms “hydroxyl” and “hydroxy” mean —OH. The term “alkylthio” refers to an alkyl group, having a sulfhydryl group attached thereto. Suitable alkylthio groups include groups having 1 to about 12 carbon atoms, preferably from 1 to about 6 carbon atoms. The term “alkylcarboxyl” as used herein means an alkyl group having a carboxyl group attached thereto.

The terms “alkoxy” and “lower alkoxy” as used herein mean an alkyl group having an oxygen atom attached thereto. Representative alkoxy groups include groups having 1 to about 6 carbon atoms, e.g., methoxy, ethoxy, propoxy, tert-butoxy and the like. Examples of alkoxy groups include but are not limited to methoxy, ethoxy, isopropyloxy, propoxy, butoxy, pentoxy, fluoromethoxy, difluoromethoxy, trifluoromethoxy, chloromethoxy, dichloromethoxy, trichloromethoxy groups, and the like. The term “alkoxy” includes both unsubstituted and substituted alkoxy groups, etc., as well as halogenated alkoxy/perhalogenated alkyloxy groups. Similarly, the term “hydrocarboxy” or “oxyhydrocarboxy” refers to the group or structure where the hydrocarbon group is connected to the oxygen atom. Lower alkoxy means the alkyl group in the alkoxy is a lower alkyl group.

The terms “carbonyl” and “carboxy” include compounds and moieties which contain a carbon connected with a double bond to an oxygen atom (C(═O)). “Carbonyl” is the component of functional groups such as aldehydes, ketones, and carboxylic acids. Examples of moieties which contain a carbonyl include aldehydes, ketones, carboxylic acids, amides, esters, anhydrides, etc.

The term “acyl” refers to a carbonyl group that is attached through its carbon atom to a hydrogen (i.e., formyl), an aliphatic group (e.g., C₁-C₂₉ alkyl, C₁-C₂₉ alkenyl, C₁-C₂₉ alkynyl, e.g., acetyl), a cycloalkyl group (C₃-C₈cycloalkyl), a heterocyclic group (C₃-C₈heterocycloalkyl and C₅-C₆heteroaryl), an aromatic group (C₆aryl, e.g., benzoyl), and the like. Acyl groups may be unsubstituted or substituted acyl groups (e.g., salicyloyl). As used herein, the term “acyl” refers to the fragment remaining after dehydroxylation of carboxylic acid, which is —C(═O)Rₐ. The term “acyl group” as used herein refers to a group where at least one carbon or heteroatom is covalently bonded to the carbon atom in —C═O within a compound or fragment.

The term “amidoalkyl” or “hydrocarbonamide/alkylamide” refers to the group formed by the combination of hydrocarbon/alkyl group and amide group. The term “acyl hydrocarbon group” or “hydrocarbonyl group” refers to the group formed by the combination of hydrocarbon group and acyl group. The term “carbonyl hydrocarbon group” or “hydrocarbon carbonyl group” refers to the group formed by the combination of hydrocarbon group and carbonyl group.

It should be understood that “substitution” or “substituted with” includes the implicit proviso that such substitution is in accordance with the permitted valence of the substituted atom and the substituent, and that the substitution results in a stable compound, i.e., a compound which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more. The term “substituted”, when used in association with any of the foregoing groups refers to a group substituted at one or more position with substituents such as acyl, amino (including simple amino, mono and dialkylamino, mono and diarylamino, and alkylarylamino), acylamino (including carbamoyl, and ureido), alkylcarbonyloxy, arylcarbonyloxy, alkoxycarbonyloxy, alkoxycarbonyl, carboxy, carboxylate, aminocarbonyl, mono and dialkylaminocarbonyl, cyano, azido, halogen, hydroxyl, nitro, trifluoromethyl, thio, alkylthio, arylthio, alkylthiocarbonyl, thiocarboxylate, lower alkyl, lower alkenyl, lower alkynyl, cycloalkyl, heterocycloalkyl, aryl, heteroaryl, lower alkoxy, aryloxy, aryloxycarbonyloxy, benzyloxy, benzyl, sulfinyl, alkylsulfinyl, sulfonyl, sulfate, sulfonate, sulfonamide, phosphate, phosphonato, phosphinato, oxo, guanidine, imino, formyl and the like. Any of the above substituents can be further substituted if permissible, e.g., if the group contains an alkyl group, an aryl group, or other.

The terms “substituted”, “with substituent” and “with substitution” mean that the parent compound or part thereof has at least one substituent group. Unless otherwise indicated, a “substituent” group can be at one or more substitutable positions of the parent group, and when there is more than one substituent present at different positions of a given structure, the substituents can be the same or different at each position. In certain embodiments, the terms “substituent” and “substituted group” include, but are not limited to, halogen (F, Cl, Br or I), hydroxyl, mercapto, thiol, amino, nitro, carbonyl, carboxyl, alkyl, alkoxy, alkylamino, aryl, aryloxy, arylamino, acyl, sulfinyl, sulfonyl, phosphoryl, phosphonyl and other organic parts routinely used and accepted in organic chemistry.

Where multiple substituents are indicated as being attached to a structure, it is to be understood that the substituents can be the same or different. Thus for example “Rₘoptionally substituted with 1, 2 or 3 R_{q} groups” indicates that Rₘ is substituted with 1, 2, or 3 R_{q} groups where the R_{q} groups can be the same or different.

The terms “unsubstituted” and “without substitution” mean that a compound or part thereof has no substituent except the undetermined chemical saturation of hydrogen atom.

The term “solvate” refers to a physical association of a compound with one or more solvent molecules, whether organic or inorganic. This physical association includes hydrogen bonding. In certain instances, a solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, ethanolates, methanolates, hemiethanolates, and the like.

The term “hydrate” refers to a compound that is bonded to one or more water (H₂O) molecule, e.g., by a hydrogen bond.

The term “pharmaceutically acceptable” as used herein refers to drugs, medicaments, inert ingredients etc., which the term describes, suitable for use in contact with the tissues of humans and lower animals without undue toxicity, incompatibility, instability, irritation, allergic response, and the like, commensurate with a reasonable benefit/risk ratio.

A “pharmaceutically acceptable salt” of a compound means a salt of a compound that is pharmaceutically acceptable. Desirable are salts of a compound that retain or improve the biological effectiveness and properties of the free acids and bases of the parent compound as defined herein or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include:
(1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, β-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like;
(2) base addition salts, formed when an acidic proton present in the parent compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Pharmaceutically acceptable salts may be synthesized from a parent compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

The term “ester” as used herein refers to a group or segment that can be represented by the general formula —RCOOR′. Usually, the group can be obtained by the reaction of carboxylic acid and alcohol (elimination of a molecule of water). Non-limiting examples for —R— include a lower alkyl or aryl, such as methylene, ethylene, isopropylene, phenylene, benzylene, etc. Non-limiting examples for R′ include a lower alkyl or aryl, such as methyl, ethyl, propyl, isopropyl, butyl, phenyl, benzyl, etc. The term “ester alkyl” means that R′ is an alkyl, one end of which is directly connected with the oxygen on the ester, and the other end is covalently bonded with at least one carbon or heteroatom in a compound or fragment.

As used herein, a “stereoisomer” of a compound refers to the isomer produced by the different spatial arrangement of atoms or groups in a molecule. Isomers caused by the same order of atoms or atomic groups in the molecule but with different spatial arrangement are called stereoisomers. Stereoisomers are mainly divided into two categories: stereoisomers caused by bond length, bond angle, intramolecular double bond, ring, and the like are called configuration stereoisomers. In general, isomers cannot or are difficult to convert into each other. Stereoisomers caused only by the rotation of a single bond are called conformational stereoisomers, sometimes also known as rotational isomers. When the rotation in the rotating isomer is blocked and cannot rotate, it becomes a “stereoisomer”, for example, in the biphenyl structure, when α- and α′-positions bear large and different substituents, the rotation of the single bond between the two phenyl rings stops due to the hindrance between the substituents, producing two stereoisomers.

### Compounds

In certain embodiments, there are provided bifunctional compounds, and/or pharmaceutically acceptable salts, esters, hydrates, solvates, and stereoisomers thereof, comprising a KRAS protein targeting group (K) and an E3 ligase binding group (T). In some such embodiments, bifunctional compounds of the disclosure further comprise a bivalent linking group that connects K and T together via a covalent linkage. In alternative embodiments, the linking group is absent and K and T are connected together directly.

Unless specified otherwise, the terms K and T are used herein with their inclusive meanings. For example, the term K includes all groups or parts of a structure that may target or recognize the KRAS protein; it may be an independent molecule or group that binds KRAS protein, or, alternatively, a group that combines with other molecules or structures to recognize the target protein. K is therefore intended to include all molecules or groups that can be used, alone or in combination with other molecules, to recognize KRAS protein, partially or completely. Similarly, the term T includes all groups or parts of a structure that may be used to bind to an E3 ubiquitin ligase (such as, without limitation, a ligand of an E3 ligase or a portion thereof). The term T encompasses all possible moieties that can be used as an E3 ubiquitin ligase ligand, which may include independent ligands capable of binding to the E3 ubiquitin ligase or moieties that incorporate ligand molecules or groups, as well as molecules or groups of other structures. T is therefore intended to include all molecules or groups that can be used, alone or in combination with other molecules, to bind to an E3 ubiquitin ligase, partially or completely.

Further, it should be understood that the number and the position of K and T groups in a compound of the disclosure are provided for illustration purposes only and are not intended to be particularly limited. A compound may include more than one K and/or T group, and groups may be connected together in different orientations and positions, as long as the bifunctional compound can still act to inhibit the target protein, e.g., by binding to the target protein and the E3 ligase and modulating degradation of the target protein.

In certain embodiments of bifunctional compounds of the disclosure, the KRAS protein targeting group (K) and the E3 ligase binding group (T) are connected directly to each other. In alternative embodiments, bifunctional compounds of the disclosure comprise a bivalent linking group (L) that connects the KRAS protein targeting group (K) and the E3 ligase binding group (T) together. The structure of L is not particularly limited, and structures provided herein are exemplary only and not intended to limit the scope of L. In general, when L is present in a bifunctional compound of the disclosure, it can be any bivalent structural fragment, i.e., having at least two connecting points, which can connect K and T covalently to form a bifunctional compound.

As used herein, the term “compounds of the disclosure” and equivalent expressions refers to bifunctional compounds provided herein as being useful for at least one purpose of the disclosure, e.g., those encompassed by structural Formula (A) or Formula (A-2), and includes specific compounds mentioned herein such as those in Tables 1-2 as well as their pharmaceutically acceptable salts, esters, hydrates, solvates and stereoisomers.

As would be understood by a person of ordinary skill in the art, the recitation of “a compound” is intended to include salts, esters, solvates, hydrates, oxides, and inclusion complexes of that compound as well as any stereoisomeric form or polymorphic form, or a mixture of any such forms of that compound in any ratio. Thus, in accordance with some embodiments, a compound as described herein, including in the contexts of pharmaceutical compositions and methods of treatment, is provided as the salt form.

It should be understood that compounds described herein may contain one or more chiral centers and/or double bonds and therefore, may exist as stereoisomers, such as double-bond isomers (i.e., geometric isomers), enantiomers, or diastereomers. Chemical structures disclosed herein are intended to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan, e.g., chiral chromatography (such as chiral HPLC), immunoassay techniques, or the use of covalently (such as Mosher's esters) and non-covalently (such as chiral salts) bound chiral reagents to respectively form a diastereomeric mixture which can be separated by conventional methods, such as chromatography, distillation, crystallization or sublimation, the chiral salt or ester is then exchanged or cleaved by conventional means, to recover the desired isomers. The compounds may also exist in several tautomeric forms including the enol form, the keto form, and mixtures thereof. The chemical structures depicted herein are also intended to encompass all possible tautomeric forms of the illustrated compounds.

Compounds may exist in unsolvated forms as well as solvated forms, including hydrated forms. In general, compounds may be hydrated or solvated. Certain compounds may exist in multiple crystalline or amorphous forms. In general, all physical forms are intended to be encompassed herein.

Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

For compounds provided herein, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present invention include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include without limitation metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

For compounds provided herein, it is intended that, in some embodiments, compounds may contain unnatural proportions of atomic isotopes at one or more of the atoms that constitute such compounds. Unnatural proportions of an isotope may be defined as ranging from the amount found in nature to an amount consisting of 100% of the atom in question. For example, compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants can have altered pharmacokinetic and pharmacodynamic characteristics which can contribute to enhanced safety, tolerability or efficacy during treatment. All isotopic variations of compounds provided herein, whether radioactive or not, are intended to be encompassed herein.

Isotopic enrichment is a process by which the relative abundance of the isotopes of a given element are altered, thus producing a form of the element that has been enriched (i.e., increased) in one particular isotope and reduced or depleted in its other isotopic forms. As used herein, an “isotope-enriched” compound or derivative refers to a compound in which one or more specific isotopic form has been increased, i.e., one or more of the elements has been enriched (i.e., increased) in one or more particular isotope. Generally, in an isotope-enriched compound or derivative, a specific isotopic form of an element at a specific position of the compound is increased. It should be understood however that isotopic forms of two or more elements in the compound may be increased. Further, an isotope-enriched compound may be a mixture of isotope-enriched forms that are enriched for more than one particular isotope, more than one element, or both. As used herein, an “isotope-enriched” compound or derivative possesses a level of an isotopic form that is higher than the natural abundance of that form. The level of isotope-enrichment will vary depending on the natural abundance of a specific isotopic form. In some embodiments, the level of isotope-enrichment for a compound, or for an element in a compound, may be from about 2 to about 100 molar percent (%), e.g., about 2%, about 5%, about 17%, about 30%, about 51%, about 83%, about 90%, about 95%, about 96%, about 97%, about 98%, greater than about 98%, about 99%, or 100%.

As used herein, an “element of natural abundance” and an “atom of natural abundance” refers to the element or atom respectively having the atomic mass most abundantly found in nature. For example, hydrogen of natural abundance is ¹H (protium); nitrogen of natural abundance is ¹⁴N; oxygen of natural abundance is ¹⁶O; carbon of natural abundance is ¹²C; and so on. A “non-isotope enriched” compound is a compound in which all the atoms or elements in the compound are isotopes of natural abundance, i.e., all the atoms or elements have the atomic mass most abundantly found in nature.

### Compositions

In certain embodiments, there are provided pharmaceutical compositions comprising a compound of the disclosure, e.g., a compound of Formula (A), or a pharmaceutically acceptable salt, ester, hydrate, solvate or stereoisomer thereof, and a pharmaceutically acceptable excipient, carrier or diluent. In an embodiment, there is provided a pharmaceutical composition comprising a compound of Formula (A) or a compound in any one of Tables 1-2, or a pharmaceutically acceptable salt thereof, and a pharmaceutically acceptable excipient, carrier, or diluent.

The preparation of pharmaceutical compositions can be carried out as known in the art (see, for example, Remington: The Science and Practice of Pharmacy, 20^{th} Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical inhuman or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, dispersants and dispensing agents, depending on the nature of the mode of administration and dosage forms. It should be understood that, as used herein, a pharmaceutical composition comprises a compound disclosed herein (or a pharmaceutically acceptable salt, ester, hydrate, solvate, or stereoisomer thereof) and a pharmaceutically acceptable excipient, carrier, diluent, adjuvant, or vehicle. In certain embodiments, the amount of a compound in a composition is such that it is effective as an inhibitor of a KRAS protein in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Other pharmaceutically acceptable excipients may include one or more of the following: binders, fillers, disintegrants, lubricants, and glidants. Pharmaceutically acceptable carriers or diluents may include one or more of the following: creams, emulsions, gels, liposomes, and nanoparticles.

Examples of disintegrating agents include starch, alginic acids, and certain complex silicates. Examples of lubricants include magnesium stearate, sodium lauryl sulphate, talc, as well as high molecular weight polyethylene glycols.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

In some embodiments, pharmaceutical compositions provided herein are suitable for oral administration. For example, a pharmaceutical composition may be in the form of a hard shell gelatin capsule, a soft shell gelatin capsule, a cachet, a pill, a tablet, a lozenge, a powder, a granule, a pellet, a pastille, or a dragee. Alternatively, a pharmaceutical composition may be in the form of a solution, an aqueous liquid suspension, a non-aqueous liquid suspension, an oil-in-water liquid emulsion, a water-in-oil liquid emulsion, an elixir, or a syrup. Pharmaceutical compositions may or may not be enteric coated. In some embodiments, pharmaceutical compositions are formulated for controlled release, such as delayed or extended release.

In further embodiments, compounds and compositions thereof may be formulated in multi-dose forms, i.e., in the form of multi-particulate dosage forms (e.g., hard gelatin capsules or conventional tablets prepared using a rotary tablet press) comprising one or more bead or minitab populations for oral administration. The conventional tablets rapidly disperse on entry into the stomach. The one or more coated bead or minitab populations may be compressed together with appropriate excipients into tablets (for example, a binder, a diluent/filler, and a disintegrant for conventional tablets.

Tablets, pills, beads, or minitabs of the compounds and compositions of the compounds may be coated or otherwise compounded to provide a dosage form affording the advantage of controlled release, including delayed or extended release, or to protect from the acid conditions of the stomach. For example, the tablet or pill can include an inner dosage and an outer dosage component, the latter being in the form of a coating over the former. The two components can be separated by a polymer layer that controls the release of the inner dosage.

In certain embodiments, the layer may comprise at least one enteric polymer. In further embodiments, the layer may comprise at least one enteric polymer in combination with at least one water-insoluble polymer. In still further embodiments, the layer may comprise at least one enteric polymer in combination with at least one water-soluble polymer. In yet further embodiments, the layer may comprise at least one enteric polymer in combination with a pore-former.

In certain embodiments, the layer may comprise at least one water-insoluble polymer. In still further embodiments, the layer may comprise at least one water-insoluble polymer in combination with at least one water-soluble polymer. In yet further embodiments, the layer may comprise at least one water-insoluble polymer in combination with a pore-former.

Representative examples of water-soluble polymers include polyvinylpyrrolidone (PVP), hydroxypropyl methylcellulose (HPMC), hydroxypropylcellulose (HPC), polyethylene glycol, and the like.

Representative examples of enteric polymers include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methylmethacrylate copolymers and shellac. These polymers may be used as a dry powder or an aqueous dispersion. Some commercially available materials that may be used are methacrylic acid copolymers sold under the trademark Eudragit (LI 00, S I 00, L30D) manufactured by Rohm Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

Representative examples of useful water-insoluble polymers include ethylcellulose, polyvinyl acetate (for example, Kollicoat SR #30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Any of the above polymers may be further plasticized with one or more pharmaceutically acceptable plasticizers. Representative examples of plasticizers include triacetin, tributyl citrate, triethyl citrate, acetyl tri-n-butyl citrate diethyl phthalate, castor oil, dibutyl sebacate, acetylated monoglycerides and the like or mixtures thereof. The plasticizer, when used, may comprise about 3 to 30 wt. % and more typically about 10 to 25 wt. % based on the polymer. The type of plasticizer and its content depends on the polymer or polymers and nature of the coating system (e.g., aqueous or solvent based, solution or dispersion based and the total solids).

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Pharmaceutical compositions can also include carriers to protect the composition against rapid degradation or elimination from the body, such as a controlled release formulation, including liposomes, hydrogels, and microencapsulated delivery systems. For example, a time delay material such as glyceryl monostearate or glyceryl stearate alone, or in combination with a wax, may be employed. Any drug delivery apparatus may be used to deliver compounds and compositions of the disclosure, including implants (e.g., implantable pumps) and catheter systems, slow infusion pumps and devices, all of which are well known to the skilled artisan.

Pharmaceutical compositions may also be in the form of a sterile injectable aqueous or oleagenous (oily) suspension. This suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyhydric alcohol (e.g., glycerol, propylene glycol, propanediol, liquid polyethylene glycol, and liquid polyethylene glycol), and suitable mixtures thereof. In addition, sterile, fixed oils are conventionally employed as a solvent or suspending medium. For this purpose any mild fixed oil may be employed, including synthetic mono- or diglycerides. Moreover, fatty acids such as oleic acid, find use in the preparation of injectables. Prolonged absorption of particular injectable formulations can be achieved by including an agent (e.g., aluminum monostearate or gelatin) that delays absorption.

Many methods for the preparation of such formulations are generally known to those skilled in the art. Sterile injectable solutions can be prepared by incorporating an active compound, such as a compound of Formula (A) provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the invention may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a KRAS-associated disease, disorder or condition, such as a cancer or a tumor. Dosages are discussed further below.

In some embodiments, the pharmaceutical composition is provided in a single-use container (e.g., a single-use vial, ampoule, syringe, or autoinjector), whereas a multi-use container (e.g., a multi-use vial) is provided in other embodiments.

In some embodiments, the pharmaceutical composition is provided in a disposable container (e.g., a disposable vial, ampoule, syringe, or auto-injector), whereas in other embodiments, there is provided a repeatedly usable container (e.g., a repeatedly usable vial).

Pharmaceutical compositions provided herein can be formulated to be compatible with the intended method or route of administration; exemplary routes of administration are set forth herein and include, without limitation, parenteral, intraperitoneal, intradermal, intracardiac, intraventricular, intracranial, intracerebrospinal, intrasynovial, intrathecal, intramuscular, intravitreal, intravenous, intra-arterial, oral, intraoral, sublingual, transdermal, intratracheal, intrarectal, subcutaneous, and topical administration. Furthermore, the pharmaceutical compositions may be used in combination with other therapeutically active agents or compounds as described herein in order to treat or prevent the KRAS-associated diseases, disorders and conditions as contemplated herein.

Pharmaceutical compositions containing the active ingredient (e.g., a KRAS inhibitor) may be in a form suitable for oral use, for example, as tablets, capsules, pastilles, troches, lozenges, aqueous or oily suspensions, dispersible powders or granules, emulsions, hard or soft capsules, or syrups, solutions, beads, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin, gum arabic or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methycellulose, carboxymethylcellulose, protamine sulfate, or lactide/glycolide copolymers, polylactide/glycolide copolymers, or ethylenevinylacetate copolyrners in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methykellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions typically comprise a therapeutically effective amount of a KRAS inhibitor compound provided herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bi sulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-MoqJholino)ethanesulfonic acid (MES), 2-(N-Morpholino)ethanesulfonic acid sodium salt (MES), 3-(N-Morpholino)propanesulfonic acid (MOPS), and Ntris[Hydroxymethyl]methyl-3-aminopropanesulfonic acid (TAPS). After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form.

In some embodiments, there are provided pharmaceutical compositions that comprise an effective amount of a compound and/or composition described herein, and a pharmaceutically acceptable excipient, carrier or diluent. In an embodiment, there are provided pharmaceutical compositions for the treatment or prevention of a KRAS-associated disease, disorder or condition, such as a cancer or a tumor, comprising a compound described herein, or a pharmaceutically acceptable salt, ester, hydrate, solvate or stereoisomer thereof, and a pharmaceutically acceptable carrier. In another embodiment, there is provided a pharmaceutical composition for the prevention or treatment of a KRAS-associated disease, disorder or condition, such as a cancer or a tumor, the composition comprising a compound described herein, or a pharmaceutically acceptable salt thereof, and a pharmaceutically acceptable carrier.

### Methods of Use of Compounds and Compositions

In certain embodiments, there are provided methods for prevention or treatment of a KRAS-associated disease, disorder or condition in a subject by administering an effective amount of a compound or composition described herein. In a related aspect, there are provided methods for prevention or treatment of a KRAS-associated hyperplastic or hyperproliferative disorder, e.g., a cancer or a tumor, in a subject in need thereof by administering an effective amount of a compound or composition described herein.

In an embodiment, there is provided herein a method of treating a subject (e.g., a human) with cancer or a disorder mediated by KRAS (wild-type or mutant) comprising the step of administering to the subject a therapeutically effective amount of an KRAS inhibitor compound provided herein, e.g., a bifunctional compound provided herein or a pharmaceutically acceptable composition thereof.

There is also provided a method of treating a subject (e.g., a human) with cancer or a hyperproliferative disorder mediated by KRAS (wild-type or mutant) comprising the step of administering to the subject a therapeutically effective amount of a compound provided herein, e.g., a compound provided herein or a pharmaceutically acceptable composition thereof. In certain embodiments, the amount of a compound in a composition is such that it is effective as an inhibitor of KRAS in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject. In some embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In further embodiments, there are provided methods for treating or preventing cancer in a subject, comprising administering to the subject a therapeutically effective amount of at least one compound of the disclosure and at least one additional signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and famesyl transferase inhibitors (FTIs). There are also provided methods of augmenting the rejection of tumor cells in a subject comprising administering a compound of the disclosure in conjunction with at least one chemotherapeutic agent and/or radiation therapy, wherein the resulting rejection of tumor cells is greater than that obtained by administering either the compound, the chemotherapeutic agent or the radiation therapy alone. In further embodiments, there are provided methods for treating cancer in a subject, comprising administering to the subject a therapeutically effective amount of at least one compound of the disclosure and at least one immunomodulator.

In further embodiments, there are provided methods for treating, inhibiting or preventing a hyperproliferative or hyperplastic disease or disorder in a subject, comprising administering to the subject an effective amount of at least one compound or pharmaceutical composition of the disclosure.

The terms “patient” and “subject” are used interchangeably herein to refer to a human or a non-human animal (e.g., a mammal). Non-limiting examples of subjects include humans, monkeys, cows, rabbits, sheep, goats, pigs, dogs, cats, rats, mice, and transgenic species thereof. In some embodiments, a subject is in need of treatment by the methods provided herein, and is selected for treatment based on this need. A subject in need of treatment is art-recognized, and includes subjects that have been identified as having a disease or condition (e.g., cancer, tumor, hyperproliferative disorder), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder). In some embodiments, a subject has a cancer or tumor carrying a KRAS mutation, e.g., G12D, G12A, G12C, G12R, G12S, G12V, G13D or Q61H. In certain embodiments, a subject is a human.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “administration”, “administer” and the like, as they apply to, for example, a subject, cell, tissue, organ, or biological fluid, refer to contact of, for example, an inhibitor of KRAS, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, “treatment” and the like refer to a course of action (such as administering an inhibitor of KRAS or a pharmaceutical composition comprising same) initiated after a disease, disorder or condition, or a symptom thereof, has been diagnosed, observed, and the like, so as to eliminate, alleviate, reduce, suppress, mitigate, improve, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. Specifically, the term “treatment”, as used in the present application, means that a therapeutic substance including a compound or composition according to the present disclosure is administered to a patient in need thereof. In certain embodiments, the term “treatment” also relates to the use of a compound or composition according to the present disclosure, optionally in combination with one or more anticancer agents, to alleviate one or more symptoms associated with wild-type KRAS or KRAS mutations, to slow down the development of one or more symptoms related to wild-type KRAS or KRAS mutations, to reduce the severity of one or more symptoms related to wild-type KRAS or KRAS mutations, to inhibit the clinical manifestations related to wild-type KRAS or KRAS mutations, and/or to inhibit the expression of adverse symptoms associated with wild-type KRAS or KRAS mutations.

The terms “prevent”, “preventing”, “prevention” and the like refer to a course of action (such as administering a KRAS inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof: generally in the context of a subject predisposed to having a particular disease, disorder or condition. In certain instances, the terms also refer to slowing the progression of the disease, disorder or condition or inhibiting progression thereof to a harmful or otherwise undesired state. Specifically, the term “prevention”, as used in the present application, means that a therapeutic substance including a compound or composition according to the present disclosure is administered to a subject to prevent the occurrence of diseases related to KRAS.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a KRAS inhibitor (or, e.g., a metabolite thereof) at a particular time post-administration may be indicative of whether a therapeutically effective amount has been used. In some embodiments, the terms “therapeutically effective amount” and “effective amount” refer to the amount or dose of a therapeutic agent, such as a compound, upon single or multiple dose administration to a subject, which provides the desired therapeutic, diagnostic, or prognostic effect in the subject. An effective amount can be readily determined by an attending physician or diagnostician using known techniques and by observing results obtained under analogous circumstances. In determining the effective amount or dose of compound administered, a number of factors are considered including, but not limited to: the size, age, and general health of the subject; the specific disease involved; the degree of or involvement or the severity of the disease or condition to be treated; the response of the individual subject; the particular compound administered; the mode of administration; the bioavailability characteristics of the preparation administered; the dose regimen selected; the use of concomitant medication(s); and other relevant considerations.

The term “substantially pure” is used herein to indicate that a component makes up greater than about 50% of the total content of the composition, and typically greater than about 60% of the total content. More typically, “substantially pure” refers to compositions in which at least 75%, at least 85%), at least 90% or more of the total composition is the component of interest. In some cases, the component of interest will make up greater than about 90%), or greater than about 95%) of the total content of the composition.

As used herein, the terms “KRAS-associated disease, disorder or condition” and “disease, disorder or condition mediated by KRAS” and “KRAS-related disease” are used interchangeably to refer to any disease, disorder or condition for which wild-type KRAS or a KRAS mutation is known to play a role, and/or for which treatment with a KRAS inhibitor may be beneficial. In general, KRAS-associated or mediated diseases, disorders and conditions are those in which KRAS activity plays a biological, mechanistic, or pathological role. Non-limiting examples of KRAS-associated diseases, disorders and conditions include oncology-related disorders (cancers, tumors, etc.), including hyperproliferative disorders, hyperplastic diseases, and malignant tumors, such as lung cancer, non-small cell lung cancer (NSCLC), pancreatic cancer, biliary tract cancer, colorectal cancer, colon cancer, cholangiocarcinoma, cervical cancer, bladder cancer, liver cancer or breast cancer. For example, a KRAS inhibitor (i.e., a bifunctional compound or composition of the disclosure) may be used to prevent or treat a proliferative condition, cancer or tumor.

In some embodiments, a KRAS inhibitor is used to prevent or treat one or more of non-small cell lung cancer, pancreatic cancer, biliary tract cancer, colorectal cancer, bile duct cancer, cervical cancer, bladder cancer, liver cancer and breast cancer.

KRAS-inhibitor compounds and compositions provided herein may be administered to a subject in any appropriate manner known in the art. Suitable routes of administration include, without limitation: oral, parenteral (e.g., intramuscular, intravenous, subcutaneous (e.g., injection or implantation), intraperitoneal, intracisternal, intraarticular, intraperitoneal, intracerebral (e.g., intraparenchymal and intracerebroventricular); extra-gastrointestinal, intranasal, vaginal, sublingual, intraocular, rectal, topical (e.g., transdermal), intraoral, buccal and inhalation. Depot injections, which are generally administered subcutaneously or intramuscularly, may also be utilized to release the KRAS inhibitors disclosed herein over a defined period of time. In certain embodiments, KRAS inhibitor compounds and compositions are administered orally to a subject in need thereof.

KRAS inhibitor compounds and compositions provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. In general, dosing parameters dictate that the dosage amount be less than an amount that could be irreversibly toxic to the subject (the maximum tolerated dose (MID)) and not less than an amount required to produce a measurable effect on the subject. Such amounts are determined by, for example, the pharmacokinetic and pharmacodynamic parameters associated with ADME, taking into consideration the route of administration and other factors.

In some embodiments, a KRAS inhibitor may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. For administration of an oral agent, the compositions can be provided in the form of tablets, capsules and the like containing from 1.0 to 1000 milligrams of the active ingredient, particularly 1, 3, 5, 10, 15, 20, 25, 50, 75, 100, 150, 200, 250, 300, 400, 500, 600, 750, 800, 900, or 1000 milligrams of the active ingredient.

In some embodiments, the dosage of the desired KRAS inhibitor is contained in a “unit dosage form”. The phrase “unit dosage form” refers to physically discrete units, each unit containing a predetermined amount of the KRAS inhibitor, either alone or in combination with one or more additional agents, sufficient to produce the desired effect. It will be appreciated that the parameters of a unit dosage form will depend on the particular agent(s) and the effect to be achieved.

### Kits

There are also provided herein kits comprising a KRAS inhibitor compound or composition of the disclosure. Kits are generally in the form of a physical structure housing various components and may be used, for example, in practicing the methods provided herein. For example, a kit may include one or more KRAS inhibitor disclosed herein (provided in, e.g., a sterile container), which may be in the form of a pharmaceutical composition suitable for administration to a subject. The KRAS inhibitor can be provided in a form that is ready for use (e.g., a tablet or capsule) or in a form requiring, for example, reconstitution or dilution (e.g., a powder) prior to administration. When the KRAS inhibitors are in a form that needs to be reconstituted or diluted by a user, the kit may also include diluents (e.g., sterile water), buffers, pharmaceutically acceptable excipients, and the like, packaged with or separately from the KRAS inhibitors. When combination therapy is contemplated, the kit may contain several therapeutic agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may also contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise stated, the materials and instruments used in this invention are commercially available.

### Compound Synthesis

For the compounds disclosed herein, their synthetic method can be carried out in a stepwise or modular manner. Scheme A provides the synthetic methods for some illustrative intermediates. Scheme B discloses the synthetic steps of illustrative compounds. For each compound, different intermediates or starting materials may be selected based on the synthesis of the illustrative compounds and the design of the compounds themselves.

### Example 1. Scheme A

### Synthesis of IM1

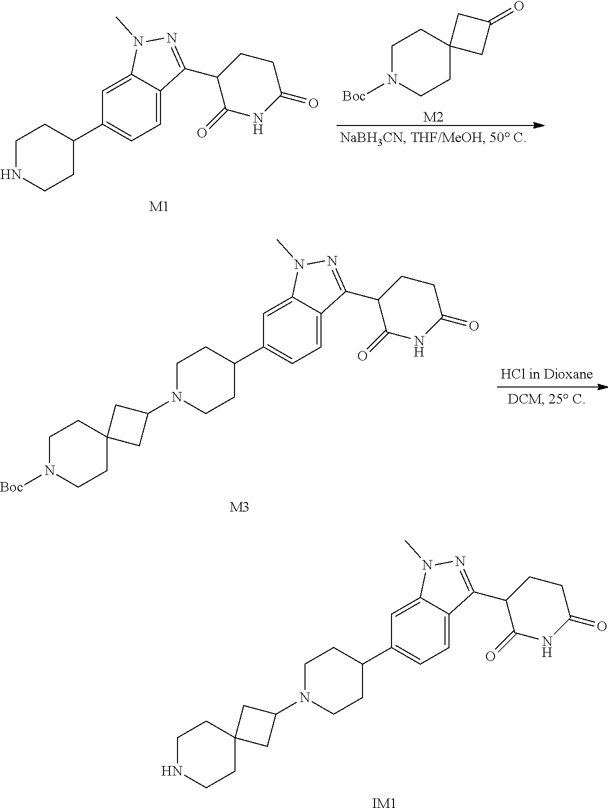

M2 (87.98 mg, 367.66 μmol, 1.2 eq) was added to a solution of M1 (100 mg, 306.38 μmol, 1 eq) in THF (1 mL) and MeOH (4 mL), followed by addition of AcOH (18.40 mg, 306.38 μmol, 1 eq) and NaBH₃CN (57.76 mg, 919.14 μmol, 3 eq). The reaction mixture was stirred at 50° C. for 16 h, then cooled to room temperature. The mixture was concentrated under reduced pressure and the residue was purified by column chromatography (MeOH/DCM=0%-10%) to afford M3 (140 mg, 83.0% yield).

4M HCl in dioxane (0.1 mL) was added to a solution of M3 (100 mg, 181.92 μmol, 1 eq) in DCM (2 mL). The reaction mixture was stirred at 25° C. for 30 min, then concentrated under reduced pressure to afford IM1 (80 mg, 95.0% yield). m/z, (ESI⁺): 450.56.

### Synthesis of IM2

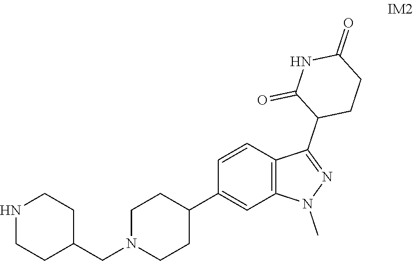 [SMILES: Cn1nc(C2CCC(=O)NC2=O)c2ccc(C3CCN(CC4CCNCC4)CC3)cc21]

IM2 was synthesized according to the procedure of IM1 with tert-Butyl 4-formylpiperidine-1-carboxylate as starting material. m/z, (ESI⁺): 424.4.

### Synthesis of IM3

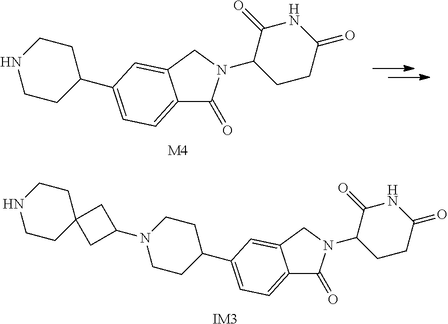

IM3 was synthesized according to the procedure of IM1 with M4 as starting material. m/z, (ESI⁺): 451.55.

### Synthesis of IM4

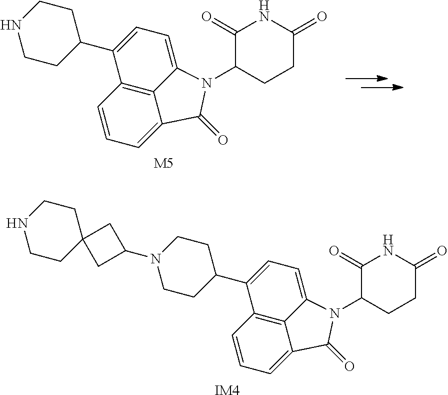

IM4 was synthesized according to the procedure of IM1 with M5 as starting material. m/z, (ESI⁺): 487.31.

### Synthesis of IM5

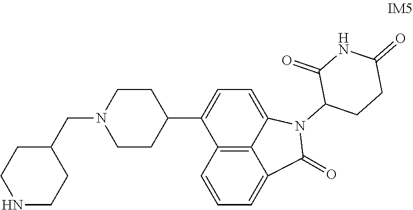 [SMILES: O=C1CCC(N2C(=O)c3cccc4c(C5CCN(CC6CCNCC6)CC5)ccc2c34)C(=O)N1]

IM5 was synthesized according to the procedure of IM4 with tert-Butyl 4-formylpiperidine-1-carboxylate as starting material. m/z, (ESI⁺): 461.46.

### Synthesis of IM6

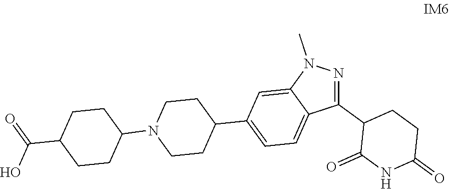 [SMILES: Cn1nc(C2CCC(=O)NC2=O)c2ccc(C3CCN(C4CCC(C(=O)O)CC4)CC3)cc21]

IM6 was synthesized according to the procedure of IM1 with 4-oxocyclohexanecarboxylic acid as starting material. m/z, (ESI⁺): 453.12.

### Synthesis of IM7

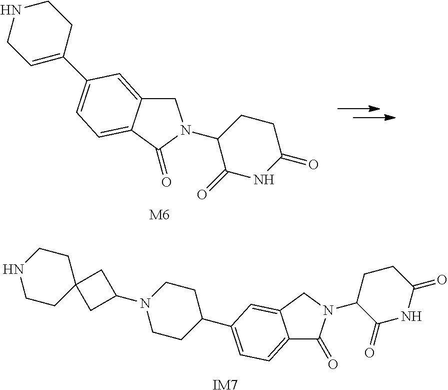

IM7 was synthesized according to the procedure of IM1 with M6 as starting material. m/z, (ESI⁺): 449.2.

### Synthesis of IM8

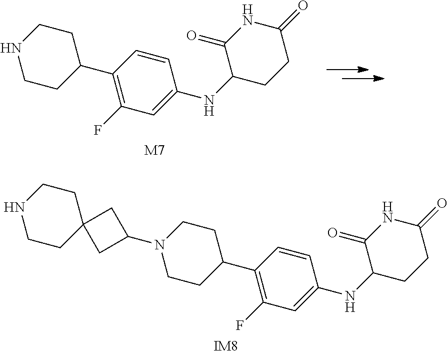

IM8 was synthesized according to the procedure of IM1 with M7 as starting material. m/z, (ESI⁺): 429.41.

### Synthesis of IM9

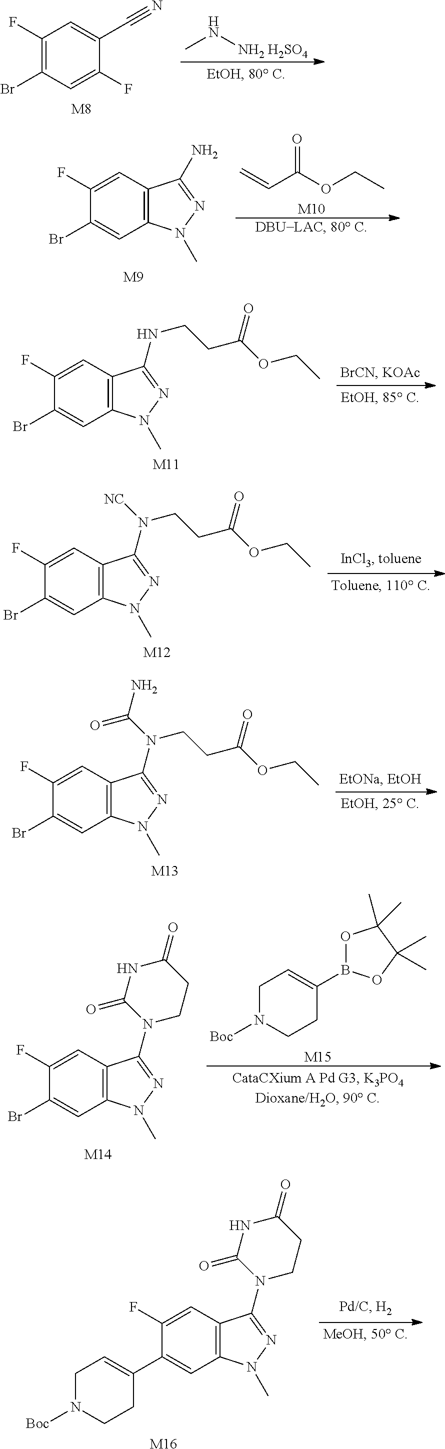

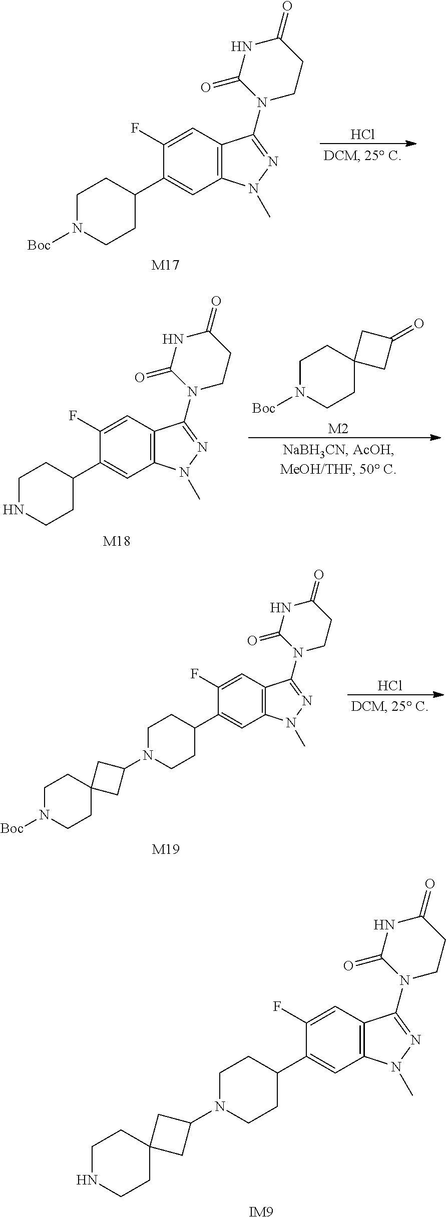

K₂CO₃ (34.17 g, 247.25 mmol, 2.2 eq) was added to a solution of M8 (24.5 g, 112.39 mmol, 1 eq) in EtOH (300 mL), followed by addition of methylhydrazine sulfate (28.80 g, 224.77 mmol, 2 eq). The reaction mixture was stirred at 80° C. for 18 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (EA/PE=0%-5%) to afford M9 (17 g, 62.0% yield). ¹H NMR (400 MHz, CD₃OD) δ 7.68 (d, J=5.4 Hz, 1H), 7.51 (d, J=8.6 Hz, 1H), 3.79 (s, 3H).

M9 (5 g, 20.49 mmol, 1 eq) was added to M10(14.36 g, 143.41 mmol, 7 eq), followed by addition of DBU-LAC (5.45 g, 22.54 mmol, 1.1 eq). The reaction mixture was stirred at 90° C. for 3 days, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (EA/PE=0%-25%) to afford M11 (3.86 g, 54.7% yield).

Cyanogen bromide (4.40 g, 41.55 mmol, 5 eq) was added to a solution of M11 (2.86 g, 8.31 mmol, 1 eq) in EtOH (30 mL), followed by addition of sodium acetate (4.09 g, 49.86 mmol, 6 eq). The mixture was stirred at 85° C. for 16 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (PE/DCM=0%-20%) to afford M12 (1.5 g, 48.9% yield). ¹H NMR (400 MHz, CDCl₃) δ 7.80 (d, J=8.4 Hz, 1H), 7.56 (d, J=5.3 Hz, 1H), 4.23 (q, J=7.2 Hz, 2H), 4.14 (t, J=6.9 Hz, 2H), 3.94 (s, 3H), 2.95 (t, J=6.8 Hz, 2H), 1.31 (t, J=7.1 Hz, 3H).

Acetaldoxime (959.95 mg, 16.25 mmol, 3 eq) was added to a solution of M12 (2 g, 5.42 mmol, 1 eq) in toluene (30 mL), followed by addition of InCl₃ (119.72 mg, 541.73 μmol, 0.1 eq). The reaction mixture was stirred at 110° C. for 1 h, then cooled to room temperature and concentrated under reduced pressure. The residue was purified by column chromatography (MeOH/DCM=0%-2%) to afford M13 (2 g, 95.4% yield). ¹H NMR (400 MHz, CDCl₃) δ 7.67 (d, J=5.1 Hz, 1H), 7.41 (d, J=8.2 Hz, 1H), 4.99 (s, 2H), 4.15 (t, J=7.1 Hz, 2H), 4.11-4.05 (m, 2H), 4.03 (s, 3H), 2.72 (t, J=7.1 Hz, 2H), 1.22 (t, J=7.2 Hz, 3H).

Sodium ethoxide (537.19 mg, 10.33 mmol, 2 eq) was added to a solution of M13 (2 g, 5.17 mmol, 1 eq) in EtOH (20 mL). The reaction mixture was stirred at 25° C. for 3 h, then treated with water, and the pH was adjusted to 2-3 with 2N HCl aqueous solution. The precipitate was filtered and dried under reduced pressure to afford M14 (1.4 g, 79.5% yield).

M15 (1.90 g, 6.16 mmol, 1.5 eq) was added to a solution of M14 (1.4 g, 4.10 mmol, 1 eq) in dioxane (16 mL) and water (4 mL), followed by addition of cataCXium A Pd G3 (149.38 mg, 205.20 μmol, 0.05 eq) and K₃PO₄ (2.61 g, 12.31 mmol, 3 eq). The reaction mixture was stirred at 90° C. for 18 h under nitrogen atmosphere, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-2%) to afford M16 (1.7 g, 93.4% yield). ¹H NMR (400 MHz, CDCl₃) δ 7.88 (s, 1H), 7.41 (d, J=10.7 Hz, 1H), 7.22 (d, J=5.7 Hz, 1H), 6.02 (s, 1H), 4.05 (s, 3H), 3.71 (t, J=5.4 Hz, 2H), 2.95 (t, J=6.7 Hz, 2H), 2.59 (s, 2H), 1.77 (s, 4H), 1.56 (s, 9H).

10% Pd/C (0.5 g) was added to a solution of M16 (1.7 g, 3.83 mmol, 1 eq) in MeOH (50 mL). The reaction mixture was stirred at 50° C. for 24 h under hydrogen atmosphere, then cooled to room temperature and filtered. The filtrate was concentrated under reduced pressure and the residue was purified by column chromatography (MeOH/DCM=0%-1%) to afford M17 (380 mg, 22.2% yield).

2M HCl EtOAc solution (0.5 mL) was added to a solution of M17 (150 mg, 336.71 μmol, 1 eq) in DCM (0.5 mL). The reaction mixture was stirred at 25° C. for 15 min, then concentrated under reduced pressure to afford M18 (128 mg, 99.6% yield).

Et₃N (166.96 mg, 1.65 mmol, 5 eq) was added to a solution of M18 (126 mg, 329.99 μmol, 1 eq) in THF (1 mL) and MeOH (1 mL), followed by addition of AcOH (19.8 mg, 330 μmol, 1 eq), M2 (94.76 mg, 395.99 μmol, 1.2 eq) and NaBH₃CN (61.38 mg, 989.96 μmol, 3 eq). The reaction mixture was stirred at 50° C. for 18 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-10%) to afford M19 (160 mg, 85.3% yield).

2N HCl aqueous solution (1.0 mL) was added to a solution of M19 (160 mg, 281.35 μmol, 1 eq) in DCM (1 mL). The reaction mixture was stirred at 25° C. for 1 h, then concentrated under reduced pressure to afford IM9 (150 mg, 98.5% yield). m/z, (ESI⁺) 469.2.

### Synthesis of IM10

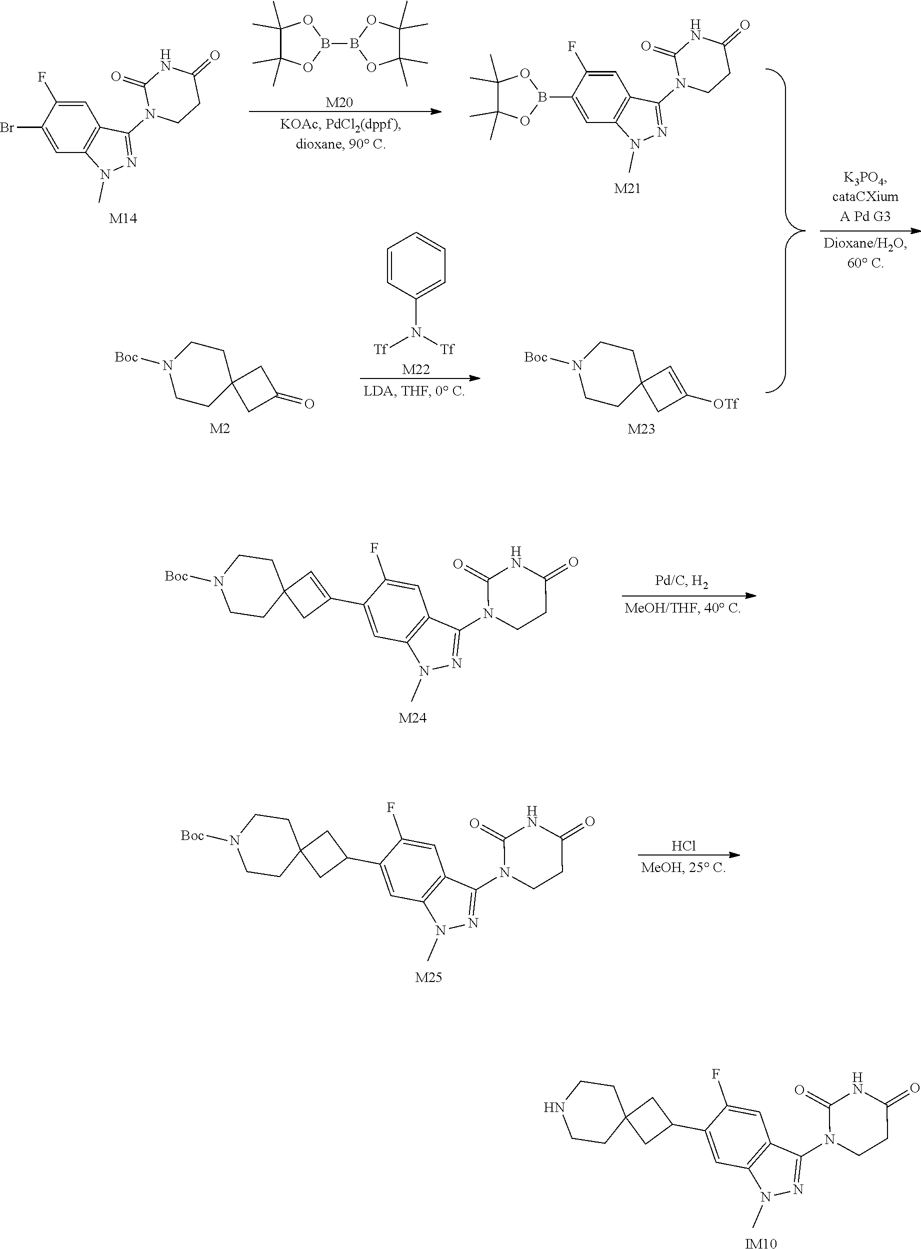

M20 (625.29 mg, 2.46 mmol, 1.2 eq) was added to a solution of M14 (700 mg, 2.05 mmol, 1 eq) in dioxane (10 mL), followed by addition of potassium acetate (652.53 mg, 6.16 mmol, 3 eq) and PdCl₂(dppf) (151.44 mg, 205.20 μmol, 0.1 eq). The reaction mixture was stirred at 90° C. for 3 h under nitrogen atmosphere, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-10%) to afford M21 (600 mg, 75.3% yield).

A solution of M2 (100 mg, 417.87 μmol, 1 eq) in THF (1.3 mL) was cooled to 0° C., followed by addition of LDA (2 M, 313.40 μL, 1.5 eq). The reaction mixture was stirred at 0° C. for 30 min, followed by addition of M22 (164.21 mg, 459.65 μmol, 1.1 eq). The reaction mixture was warmed to room temperature and stirred for 1 h, then treated with water and DCM, and stirred for 10 min. The separated organic layer was washed with water and brine, then dried over Na₂SO₄ and filtered. The filtrate was concentrated under reduced pressure and the residue was purified by column chromatography (MeOH/DCM=0%-1%) to afford M23 (22 mg, 14.2% yield). m/z, (ESI⁻): 370.3.

M21 (34.50 mg, 88.86 μmol, 1.5 eq) was added to a solution of M23 (22 mg, 59.24 μmol, 1 eq) in dioxane (2 mL) and water (0.3 mL), followed by addition of cataCXium A Pd G3 (8.62 mg, 11.85 μmol, 0.2 eq) and K₃PO₄ (37.72 mg, 177.72 μmol, 3 eq). The reaction mixture was stirred at 60° C. for 2 h under nitrogen atmosphere, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-3%) to afford M24 (10 mg, 34.9% yield).

10% Pd/C (5 mg) was added to a solution of M24 (10 mg, 20.68 μmol, 1 eq) in THF (2 mL) and MeOH (1 mL). The reaction mixture was stirred at 40° C. for 15 h under hydrogen atmosphere, then cooled to room temperature and filtered. The filtrate was concentrated under reduced pressure to afford M25 (8 mg, 79.7% yield). m/z, (ESI⁺): 486.5.

A solution of HCl in EtOAc (2 M, 164.76 μL, 20 eq) was added to another solution of M25 (8.0 mg, 16.48 μmol, 1 eq) in MeOH (0.5 mL). The reaction mixture was stirred at 25° C. for 30 min, then concentrated under reduced pressure to afford IM10 (7 mg, 98.7% yield). m/z, (ESI⁺): 386.5.

### Synthesis of IM11

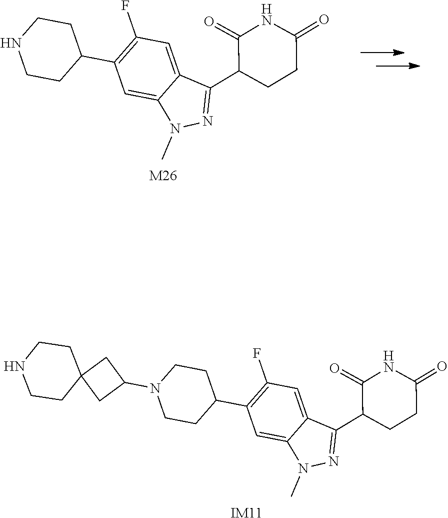

IM11 was synthesized according to the procedure of IM1 with M26 as starting material.

### Synthesis of IM12

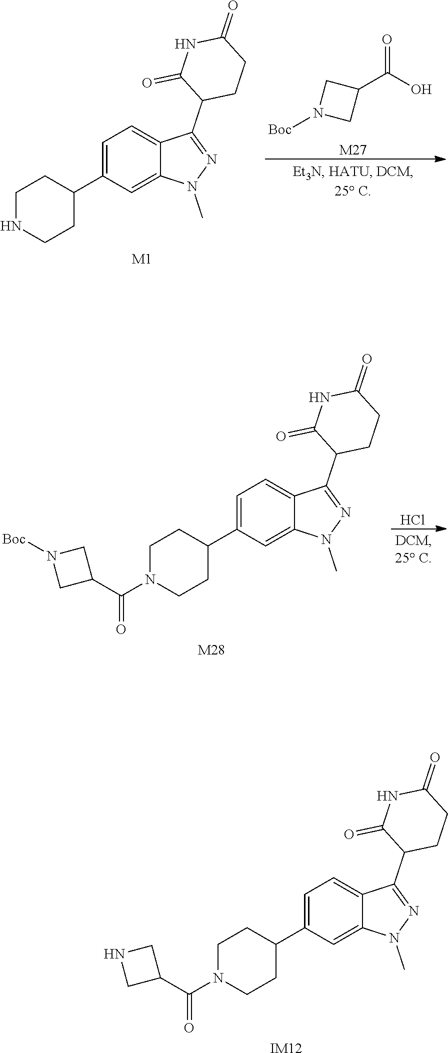

M27 (122.00 mg, 606.31 μmol, 2 eq) was added to a solution of M1 (110 mg, 303.15 μmol, 1 eq) in DCM (6 mL), followed by addition of Et₃N (92.03 mg, 909.46 μmol, 3 eq) and HATU (228.74 mg, 606.31 μmol, 2 eq). The reaction mixture was stirred at 25° C. for 2 h, then treated with water and EtOAc, stirred for 10 min. The separated organic layer was washed with water and brine, then dried over Na₂SO₄ and filtered. The filtrate was concentrated under reduced pressure and the residue was purified by column chromatography (MeOH/DCM=0%-4%) to afford M28 (150 mg, 97.1% yield). m/z, (ESI⁺): 510.5.

A solution of HCl in EtOAc (2 M, 2.94 mL, 20 eq) was added to another solution of M28 (150 mg, 294.35 μmol, 1 eq) in MeOH (4 mL). The reaction mixture was stirred at 25° C. for 30 min, then concentrated under reduced pressure to afford IM12 (140 mg, 96.0% yield). m/z, (ESI⁺) 410.5.

### Synthesis of IM13

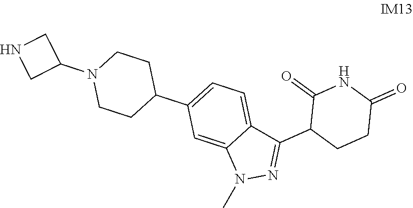 [SMILES: Cn1nc(C2CCC(=O)NC2=O)c2ccc(C3CCN(C4CNC4)CC3)cc21]

IM13 was synthesized according to the procedure of IM1 with tert-butyl 3-oxoazetidine-1-carboxylate as starting material.

### Synthesis of IM14

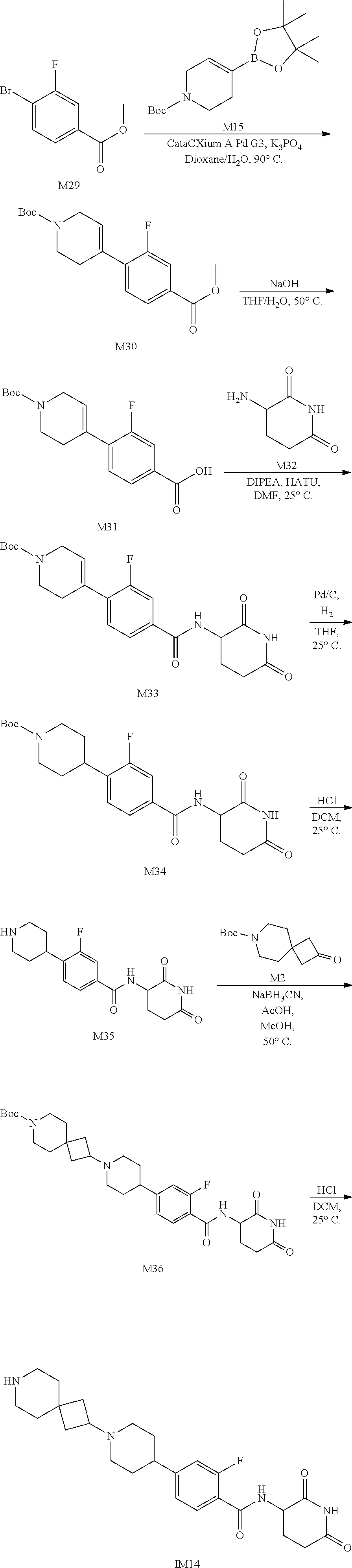

M15 (3.45 g, 11.16 mmol, 1.3 eq) was added to a solution of M29 (2 g, 8.58 mmol, 1 eq) in dioxane (5 mL) and water (1 mL), followed by addition of cataCXium A Pd G3 (124.96 mg, 171.65 μmol, 0.02 eq) and K₃PO₄ (5.46 g, 25.75 mmol, 3 eq). The reaction mixture was stirred at 90° C. for 2 h under nitrogen atmosphere, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (EA/PE=0%-10%) to afford M30 (2.6 g, 90.3% yield).

NaOH (620.21 mg, 15.51 mmol, 2 eq) was added to a solution of M30 (2.6 g, 7.75 mmol, 1 eq) in THF (40 mL) and water (10 mL). The reaction mixture was stirred at 50° C. for 2 h under nitrogen atmosphere, then cooled to room temperature. pH of the reaction mixture was adjusted to 2-3 with 2 N HCl aqueous solution. The reaction mixture was treated with water and DCM, then stirred for 10 min. The separated organic layer was washed with water and brine, then dried over Na₂SO₄ and filtered. The filtrate was concentrated under reduced pressure to afford M31 (2.2 g, 88.31% yield).

M32 (307.32 mg, 1.87 mmol, 1.2 eq) was added to a solution of M31 (500 mg, 1.56 mmol, 1 eq) in DMF (5 mL), followed by addition of DIPEA (241.31 mg, 1.87 mmol, 1.2 eq) and HATU (704.43 mg, 1.87 mmol, 1.2 eq). The reaction mixture was stirred at 25° C. for 16 h, then treated with water and EtOAc, stirred for 10 min. The separated organic layer was washed with water and brine, then dried over Na₂SO₄ and filtered. The filtrate was concentrated under reduced pressure and the residue was purified by column chromatography (MeOH/DCM=0%-3%) to afford M33 (300 mg, 44.7% yield).

10% Pd/C (100 mg) was added to a solution of M33 (300 mg, 695.32 μmol, 1 eq) in THF (20 mL). The reaction mixture was stirred at 25° C. for 15 h under hydrogen atmosphere, then filtered. The filtrate was concentrated under reduced pressure to afford M34 (270 mg, 80.6% yield). m/z, (ESI⁻): 432.2.

2M solution of HCl in EtOAc (280.3 μL, 560.59 μmol, 1 eq) was added to another solution of M34 (270 mg, 560.59 μmol, 1 eq) in DCM (2 mL). The reaction mixture was stirred at 25° C. for 5 min, then concentrated under reduced pressure to afford M35 (200 mg, 96.5% yield).

Et₃N (98.50 mg, 973.45 μmol, 2 eq) was added to a solution of M35 (200 mg, 486.73 μmol, 1 eq) in MeOH (5 mL), followed by addition of AcOH (59.38 mg, 973.45 μmol, 2 eq), M2 (232.96 mg, 973.45 μmol, 2 eq) and NaBH₃CN (61.17 mg, 973.45 μmol, 2 eq). The reaction mixture was stirred at 50° C. for 16 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-10%) to afford M36 (130 mg, 48.0% yield). m/z, (ESI⁻): 555.4.

2M solution of HCl in EtOAc (116.77 μL, 233.53 μmol, 1 eq) was added to another solution of M36 (130 mg, 233.53 μmol, 1 eq) in DCM (2 mL). The reaction mixture was stirred at 25° C. for 10 min, then concentrated under reduced pressure to afford IM14 (120 mg, 97.05% yield).

### Synthesis of IM15

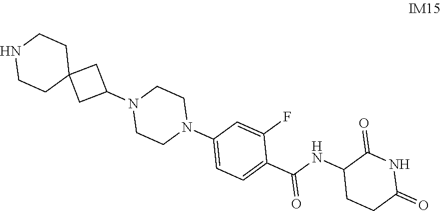 [SMILES: O=C1CCC(NC(=O)c2ccc(N3CCN(C4CC5(CCNCC5)C4)CC3)cc2F)C(=O)N1]

IM15 was synthesized according to the procedure of IM14 with tert-Butyl 1-piperazinecarboxylate as starting material. m/z, (ESI⁺): 458.6.

### Synthesis of IM16

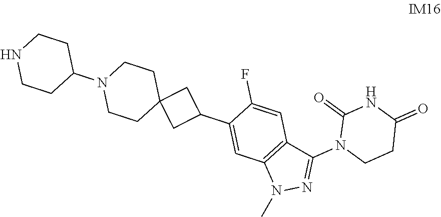 [SMILES: Cn1nc(N2CCC(=O)NC2=O)c2cc(F)c(C3CC4(CCN(C5CCNCC5)CC4)C3)cc21]

IM16 was synthesized according to the procedure of IM9 with IM10 as starting material. m/z, (ESI⁺): 469.6.

### Synthesis of IM17

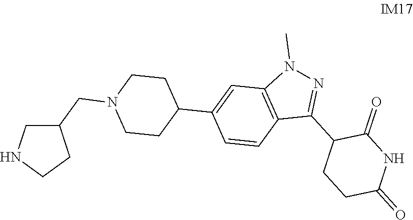 [SMILES: Cn1nc(C2CCC(=O)NC2=O)c2ccc(C3CCN(CC4CCNC4)CC3)cc21]

IM17 was synthesized according to the procedure of IM1 with tert-butyl 3-formylpyrrolidine-1-carboxylate as starting material. m/z, (ESI⁺): 410.5.

### Synthesis of IM18

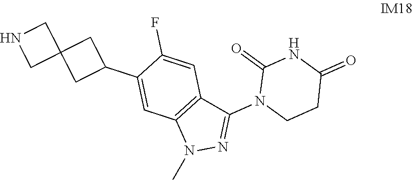 [SMILES: Cn1nc(N2CCC(=O)NC2=O)c2cc(F)c(C3CC4(CNC4)C3)cc21]

IM18 was synthesized according to the procedure of IM10 with tert-Butyl 6-oxo-2-azaspiro[3.3]heptane-2-carboxylate as starting material. m/z, (ESI⁺): 358.4.

### Synthesis of IM19

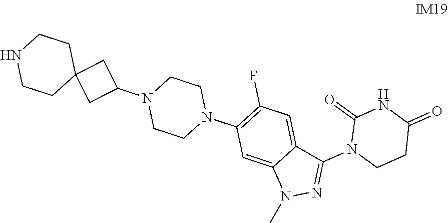 [SMILES: Cn1nc(N2CCC(=O)NC2=O)c2cc(F)c(N3CCN(C4CC5(CCNCC5)C4)CC3)cc21]

IM19 was synthesized according to the procedure of IM9 with tert-Butyl 1-piperazinecarboxylate as starting material. m/z, (ESI⁺): 470.6.

### Synthesis of IM20

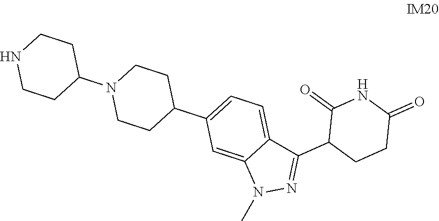 [SMILES: Cn1nc(C2CCC(=O)NC2=O)c2ccc(C3CCN(C4CCNCC4)CC3)cc21]

IM20 was synthesized according to the procedure of IM1 with tert-butyl 4-oxopiperidine-1-carboxylate as starting material. m/z, (ESI⁺): 410.70.

### Synthesis of IM21

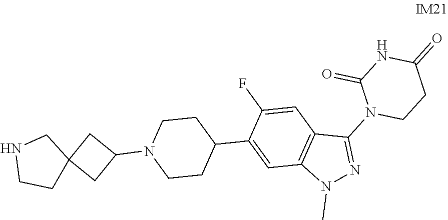 [SMILES: Cn1nc(N2CCC(=O)NC2=O)c2cc(F)c(C3CCN(C4CC5(CCNC5)C4)CC3)cc21]

IM21 was synthesized according to the procedure of IM9 with tert-butyl 2-oxo-6-azaspiro[3.4]octane-6-carboxylate as starting material. m/z, (ESI⁺): 455.6.

### Synthesis of IM22

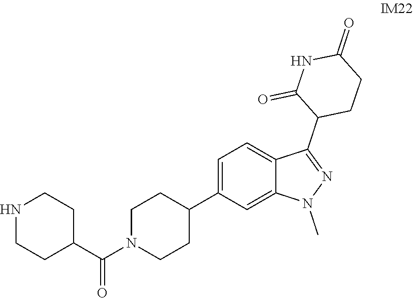 [SMILES: Cn1nc(C2CCC(=O)NC2=O)c2ccc(C3CCN(C(=O)C4CCNCC4)CC3)cc21]

IM22 was synthesized according to the procedure of IM1 with 1-(tert-butoxycarbonyl)piperidine-4-carboxylic acid as starting material.

### Synthesis of IM23

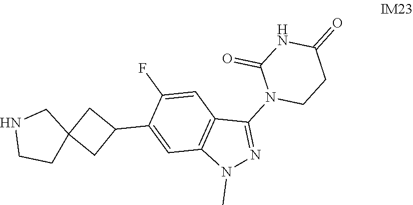 [SMILES: Cn1nc(N2CCC(=O)NC2=O)c2cc(F)c(C3CC4(CCNC4)C3)cc21]

IM23 was synthesized according to the procedure of IM18 with tert-butyl 2-oxo-6-azaspiro[3.4]octane-6-carboxylate as starting material. m/z, (ESI⁺): 372.8.

### Synthesis of IM24

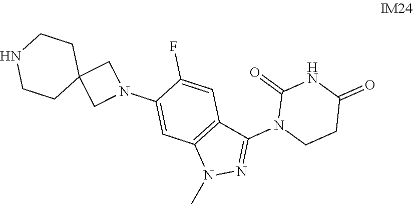 [SMILES: Cn1nc(N2CCC(=O)NC2=O)c2cc(F)c(N3CC4(CCNCC4)C3)cc21]

IM24 was synthesized according to the procedure of IM9 with tert-butyl 2,7-diazaspiro[3.5]nonane-7-carboxylate as starting material. m/z, (ESI⁺): 386.4.

### Synthesis of IM25

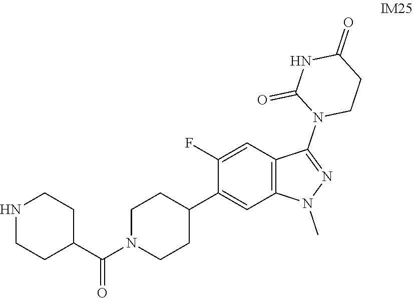 [SMILES: Cn1nc(N2CCC(=O)NC2=O)c2cc(F)c(C3CCN(C(=O)C4CCNCC4)CC3)cc21]

IM25 was synthesized according to the procedure of IM9 with 1-(tert-butoxycarbonyl)-4-piperidinecarboxylic acid as starting material.

### Synthesis of IM26

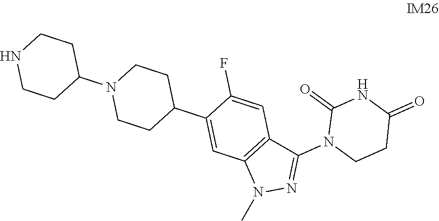 [SMILES: Cn1nc(N2CCC(=O)NC2=O)c2cc(F)c(C3CCN(C4CCNCC4)CC3)cc21]

IM26 was synthesized according to the procedure of IM9 with tert-butyl 4-oxopiperidine-1-carboxylate as starting material.

### Synthesis of IM27

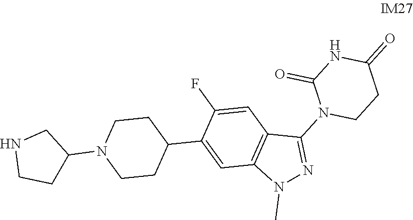 [SMILES: Cn1nc(N2CCC(=O)NC2=O)c2cc(F)c(C3CCN(C4CCNC4)CC3)cc21]

IM27 was synthesized according to the procedure of IM9 with tert-butyl 3-oxopyrrolidine-1-carboxylate as starting material. m/z, (ESI⁺): 415.5.

### Synthesis of IM28

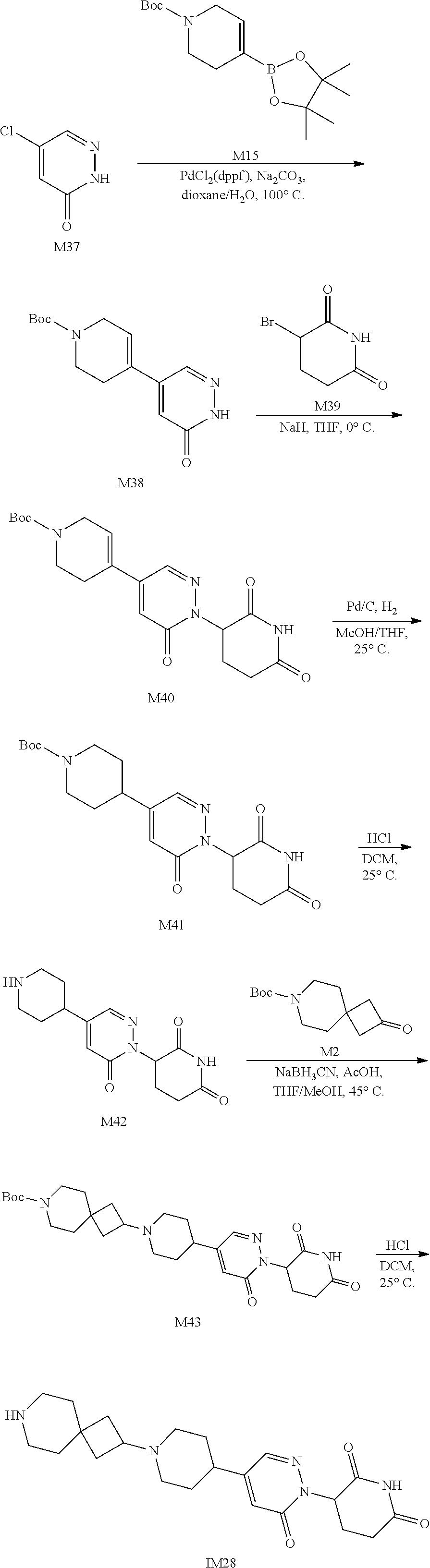

M15 (3.91 g, 12.64 mmol, 1.1 eq) was added to a solution of M37(1.5 g, 11.49 mmol, 1 eq) in dioxane (35 mL) and water (7 mL), followed by addition of potassium acetate (3.65 g, 34.47 mmol, 3.0 eq) and PdCl₂(dppf) (840.83 mg, 1.15 mmol, 0.1 eq). The reaction mixture was stirred at 100° C. for 16 h under nitrogen atmosphere, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-5%) to afford M38 (2.7 g, 84.7% yield).

A solution of M38 (1 g, 3.61 mmol, 1 eq) in THF (10 mL) was cooled to 0° C. under nitrogen atmosphere, followed by addition of 60% NaH (865.43 mg, 21.64 mmol, 6.0 eq). The reaction mixture was stirred at 0° C. for 30 min, followed by addition of another solution of M39 (1.38 g, 7.21 mmol, 2.0 eq) in THF (10 mL). The reaction mixture was warmed to room temperature and stirred for 3 h, then treated with water and EtOAc, stirred for 10 min. The separated organic layer was washed with water and brine, then dried over Na₂SO₄ and filtered. The filtrate was concentrated under reduced pressure and the residue was purified by column chromatography (MeOH/DCM=0%-4%) to afford M40 (1.1 g, 78.6% yield). m/z, (ESI⁺). 389.4.

10% Pd/C (0.4 g) was added to a solution of M40 (1.1 g, 2.83 mmol, 1 eq) in THF (25 mL) and MeOH (25 mL). The reaction mixture was stirred at 25° C. for 16 h under hydrogen atmosphere, then filtered. The filtrate was concentrated under reduced pressure to afford M41 (1.1 g, 99.5% yield).

4M solution of HCl in dioxane (4 M, 10 mL, 14.20 eq) was added to another solution of M41 (1.1 g, 2.82 mmol, 1 eq) in DCM (10 mL). The reaction mixture was stirred at 25° C. for 1 h, then concentrated under reduced pressure to afford M42 (750 mg, 81.5% yield). m/z, (ESI⁺): 291.3.

Et₃N (309.66 mg, 3.06 mmol, 5.0 eq) was added to a solution of M42 (200 mg, 612.04 μmol, 1 eq) in THF (4 mL) and MeOH (4 mL), followed by addition of AcOH (84.46 mg, 1.22 mmol, 2.0 eq), M2 (175.76 mg, 734.44 μmol, 1.2 eq) and NaBH₃CN (192.30 mg, 3.06 mmol, 5.0 eq). The reaction mixture was stirred at 45° C. for 2 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-7%) to afford M43 (200 mg, 63.6% yield).

4M solution of HCl in dioxane (4 M, 5 mL, 51.36 eq) was added to another solution of M43 (200 mg, 389.39 μmol, 1 eq) in DCM (3 mL). The reaction mixture was stirred at 25° C. for 1 h, then concentrated under reduced pressure to afford IM28 (200 mg, 98.2% yield). m/z, (ESI⁺): 414.5.

### Synthesis of IM29

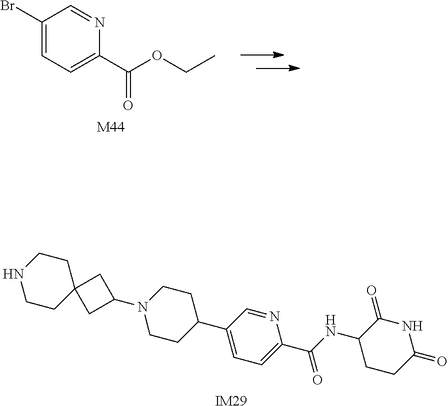

IM29 was synthesized according to the procedure of IM14 with M44 as starting material. m/z, (ESI⁺): 440.5.

### Synthesis of IM30

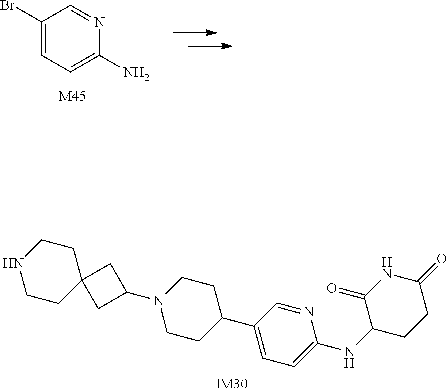

IM30 was synthesized according to the procedure of IM28 with M45 as starting material. m/z, (ESI⁺): 412.6.

### Synthesis of IM31

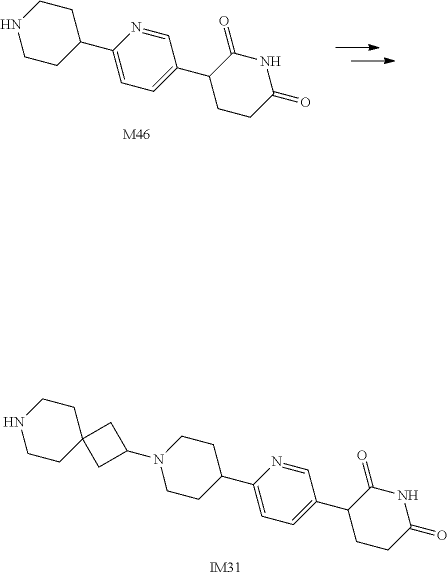

IM31 was synthesized according to the procedure of IM1 with M46 as starting material. m/z, (ESI⁺): 397.5.

### Synthesis of IM32

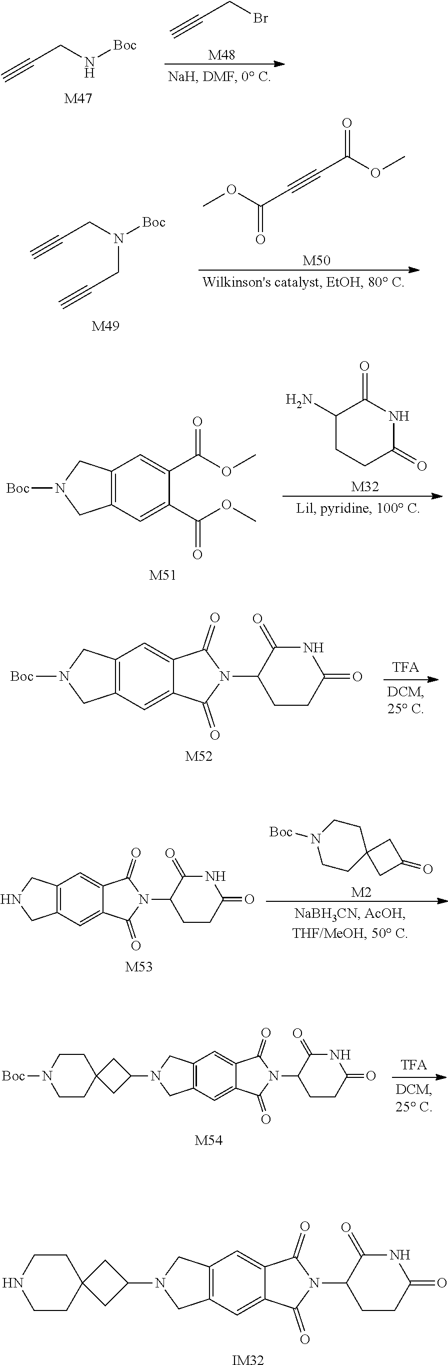

M48 (4.60 g, 38.66 mmol, 1.2 eq) was added to a solution of M47 (5 g, 32.22 mmol, 1 eq) in DMF (15 mL). The reaction mixture was cooled to 0° C. under nitrogen atmosphere, followed by addition of 60% NaH (1.55 g, 38.66 mmol, 1.2 eq). The reaction mixture was stirred at 0° C. for 3 h, then warmed to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (EA/PE=0%-20%) to afford M49 (4.8 g, 77.1% yield).

M50 (3.88 g, 27.32 mmol, 1.1 eq) was added to a solution of M49 (4.8 g, 24.84 mmol, 1 eq) in EtOH (20 mL), followed by addition of Wilkinson catalyst (2.30 g, 2.48 mmol, 0.1 eq). The reaction mixture was stirred at 80° C. for 16 h, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-2%) to afford M51 (1.7 g, 20.4% yield).

M32 (638.04 mg, 3.88 mmol, 1.3 eq) was added to a solution of M51 (1 g, 2.98 mmol, 1 eq) in pyridine (5 mL), followed by addition of LiI (1.20 g, 8.95 mmol, 3 eq). The reaction mixture was stirred at 100° C. for 16 h, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-2%) to afford M52 (0.4 g, 33.6% yield).

TFA (3 mL) was added to a solution of M52 (490 mg, 1.23 mmol, 1 eq) in DCM (6 mL). The reaction mixture was stirred at 25° C. for 30 min, then concentrated under reduced pressure to afford M53 (240 mg, 58.3% yield).

M2 (160 mg, 668.6 μmol, 1.0 eq) was added to a solution of M53 (200 mg, 668.6 μmol, 1.0 eq) in THF (2 mL) and MeOH (2 mL), followed by addition of DIPEA (86.41 mg, 668.6 μmol, 1.0 eq), AcOH (40.15 mg, 668.6 μmol, 1.0 eq) and NaBH₃CN (42.01 mg, 668.6 μmol, 1.0 eq). The reaction mixture was stirred at 50° C. for 5 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-7%) to afford M54 (30 mg, 8.5% yield). m/z, (ESI⁺) 523.7.

TFA (1 mL) was added to a solution of M54 (30 mg, 57.41 μmol, 1 eq) in DCM (2 mL). The reaction mixture was stirred at 25° C. for 30 min, then concentrated under reduced pressure to afford IM32 (37 mg, 99.08% yield). m/z, (ESI⁺): 423.5.

### Synthesis of IM33

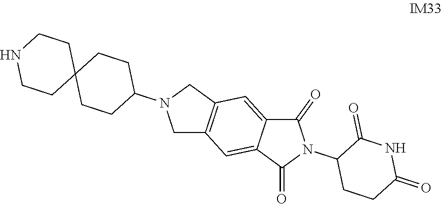 [SMILES: O=C1CCC(N2C(=O)c3cc4c(cc3C2=O)CN(C2CCC3(CCNCC3)CC2)C4)C(=O)N1]

IM33 was synthesized according to the procedure of IM32 with tert-Butyl 9-oxo-3-azaspiro[5.5]undecane-3-carboxylate as starting material. m/z, (ESI⁺): 451.5.

### Synthesis of IM34

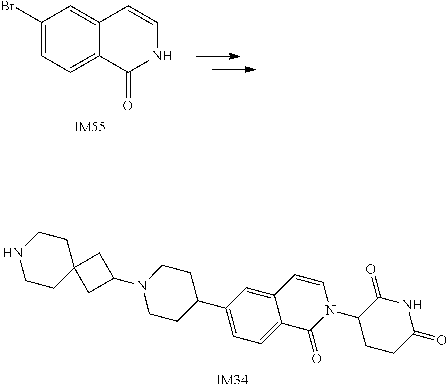

IM34 was synthesized according to the procedure of IM28 with M55 as starting material. m/z, (ESI⁺): 463.5.

### Synthesis of a

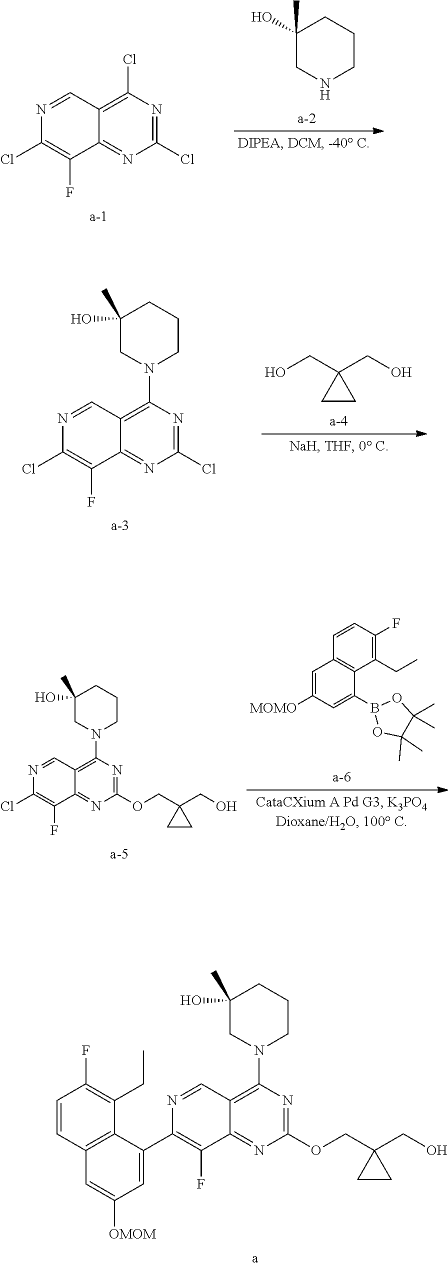

A solution of a-1 (200 mg, 792.20 μmol, 1 eq) in DCM (5 mL) was cooled to −40° C., followed by addition of DIPEA (102.38 mg, 792.20 μmol, 1 eq) and a-2 (91.24 mg, 792.20 μmol, 1 eq). The reaction mixture was stirred at −40° C. for 30 min, then warmed to room temperature. The reaction mixture was concentrated under reduced pressure and the residue was purified by column chromatography (MeOH/DCM=0%-2%) to afford a-3 (240 mg, 91.5% yield). m/z, (ESI⁺): 331.1.

a-4 (296.06 mg, 2.90 mmol, 4 eq) was added to a solution of a-3 (240 mg, 724.70 μmol, 1 eq) in THF (5 mL). The reaction mixture was cooled to 0° C., followed by addition of 60% NaH (86.97 mg, 2.17 mmol, 3 eq). The reaction mixture was stirred at 0° C. for 30 min, then warmed to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-3%) to afford a-5 (220 mg, 76.5% yield). m/z, (ESI⁺): 397.1.

a-6 (299.55 mg, 831.56 μmol, 1.5 eq) was added to a solution of a-5 (220 mg, 554.38 μmol, 1 eq) in dioxane (5 mL) and water (0.5 mL), followed by addition of cataCXium A Pd G3 (80.64 mg, 110.88 μmol, 0.2 eq) and K₃PO₄ (353.03 mg, 1.66 mmol, 3 eq). The reaction mixture was stirred at 100° C. for 3 h under nitrogen atmosphere, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-2%) to afford a (150 mg, 45.5% yield). m/z, (ESI⁺): 595.2.

### Synthesis of b

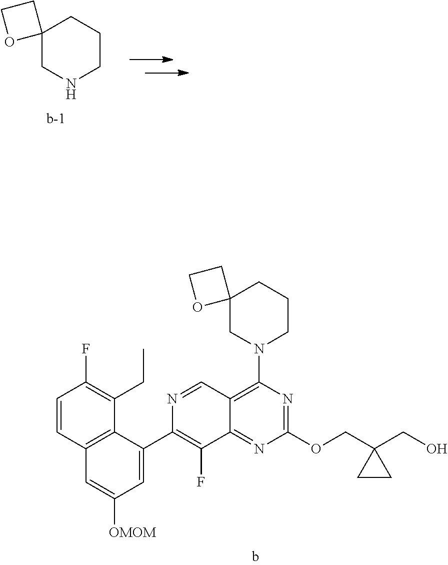

b was synthesized according to the procedure of a with b-1 as starting material. m/z, (ESI⁺): 607.9.

### Synthesis of c

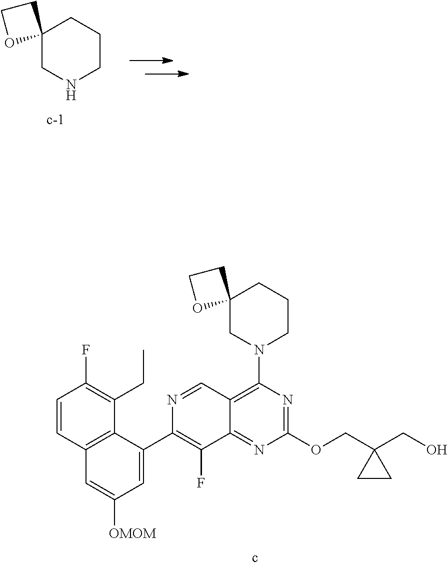

c was synthesized according to the procedure of a with c-1 as starting material. m/z, (ESI⁺): 607.7.

### Synthesis of d

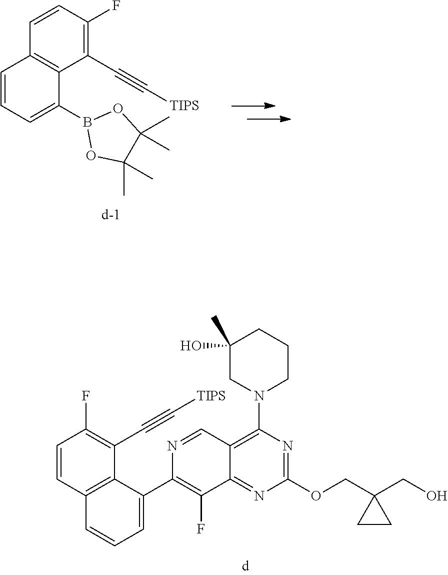

d was synthesized according to the procedure of a with d-1 as starting material.

### Synthesis of e

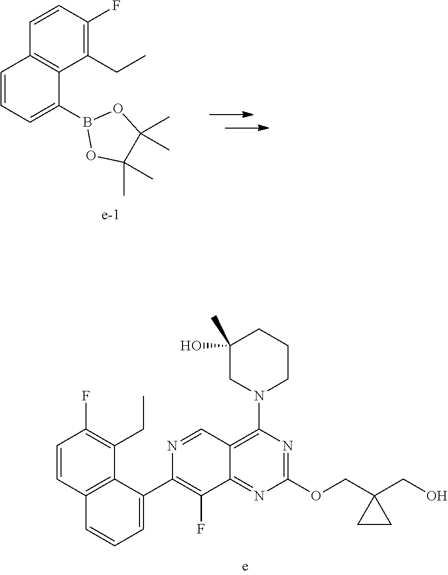 [SMILES: CCc1c(F)ccc2cccc(B3OC(C)(C)C(C)(C)O3)c12 ; CCc1c(F)ccc2cccc(-c3ncc4c(N5CCC[C@@](C)(O)C5)nc(OCC5(CO)CC5)nc4c3F)c12]

e was synthesized according to the procedure of a with e-1 as starting material. ¹H NMR (400 MHz, CDCl₃) δ 9.22 (d, J=9.7 Hz, 1H), 7.98 (dd, J=7.4, 2.2 Hz, 1H), 7.83 (dd, J=8.9, 5.9 Hz, 1H), 7.59-7.45 (m, 2H), 7.33 (t, J=9.4 Hz, 1H), 4.69 (dd, J=11.9, 5.7 Hz, 1H), 4.52 (t, J=15.8 Hz, 2H), 4.38 (dd, J=11.9, 9.5 Hz, 1H), 3.98 (s, 1H), 3.61-3.27 (m, 4H), 2.77 (d, J=40.6 Hz, 1H), 2.59 (dd, J=14.6, 7.5 Hz, 1H), 2.33-2.24 (m, 1H), 2.14 (t, J=12.6 Hz, 1H), 1.92 (d, J=13.6 Hz, 1H), 1.80 (d, J=17.0 Hz, 1H), 1.39 (s, 3H), 0.90 (q, J=7.2 Hz, 3H), 0.77-0.59 (m, 4H).

### Synthesis of f

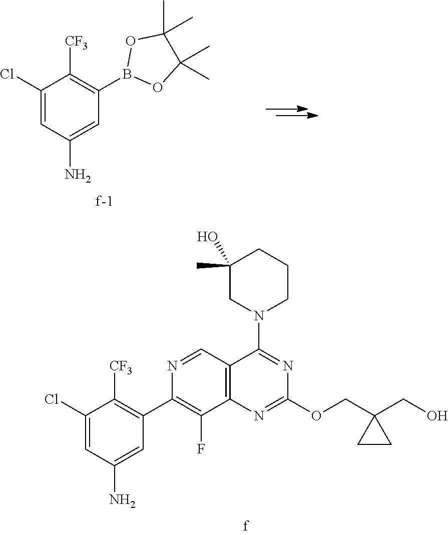 [SMILES: CC1(C)OB(c2cc(N)cc(Cl)c2C(F)(F)F)OC1(C)C ; C[C@@]1(O)CCCN(c2nc(OCC3(CO)CC3)nc3c(F)c(-c4cc(N)cc(Cl)c4C(F)(F)F)ncc23)C1]

f was synthesized according to the procedure of a with f-1 as starting material. m/z, (ESI⁺): 556.0.

### Synthesis of g

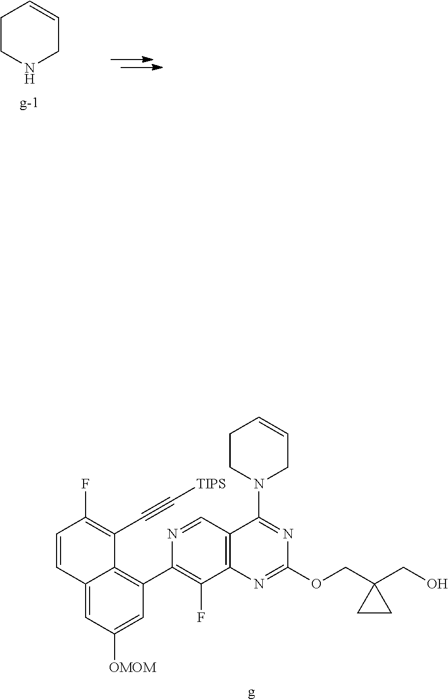

g was synthesized according to the procedure of a with g-1 as starting material. m/z, (ESI⁺): 715.8.

### Synthesis of h

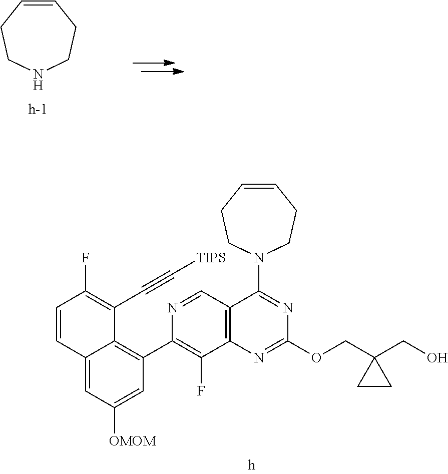

h was synthesized according to the procedure of a with h-1 as starting material. m/z, (ESI⁺): 729.8.

### Synthesis of i

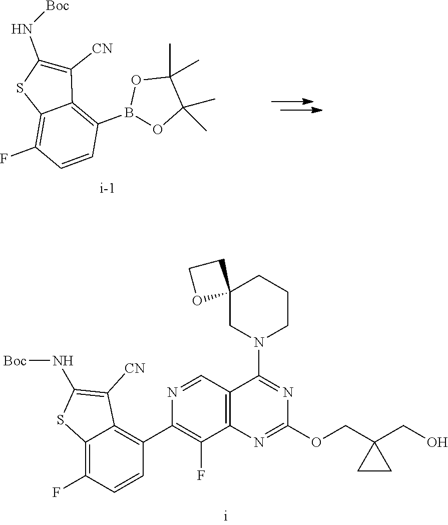 [SMILES: CC(C)(C)OC(=O)Nc1sc2c(F)ccc(B3OC(C)(C)C(C)(C)O3)c2c1C#N ; CC(C)(C)OC(=O)Nc1sc2c(F)ccc(-c3ncc4c(N5CCC[C@]6(CCO6)C5)nc(OCC5(CO)CC5)nc4c3F)c2c1C#N]

i was synthesized according to the procedure of a with i-1 as starting material. m/z, (ESI⁺): 665.8.

### Synthesis of j

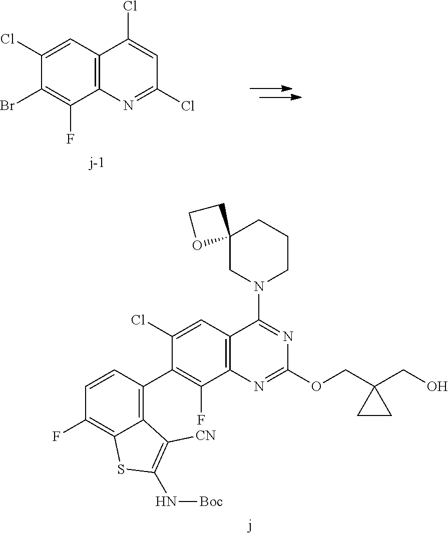 [SMILES: Fc1c(Br)c(Cl)cc2c(Cl)cc(Cl)nc12 ; CC(C)(C)OC(=O)Nc1sc2c(F)ccc(-c3c(Cl)cc4c(N5CCC[C@]6(CCO6)C5)nc(OCC5(CO)CC5)nc4c3F)c2c1C#N]

j was synthesized according to the procedure of i with j-1 as starting material. m/z, (ESI⁺): 698.72.

### Synthesis of k

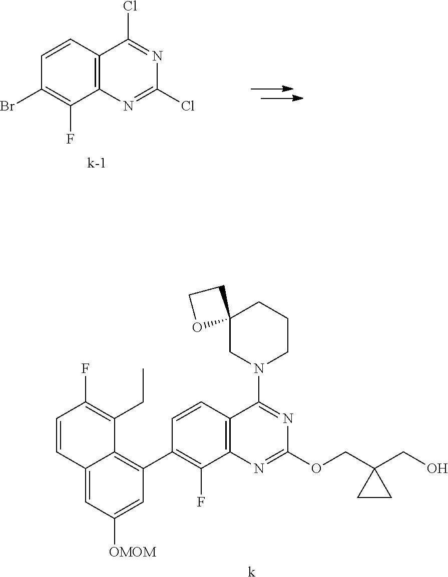

k was synthesized according to the procedure of c with k-1 as starting material. m/z, (ESI⁺): 606.8.

### Synthesis of l

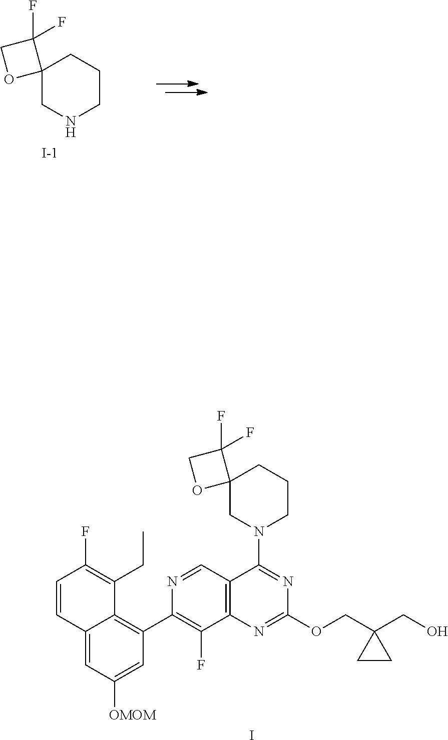

l was synthesized according to the procedure of a with 1-1 as starting material. m/z, (ESI⁺): 643.7.

### Example 2. Scheme B

### Synthesis of Compound 85

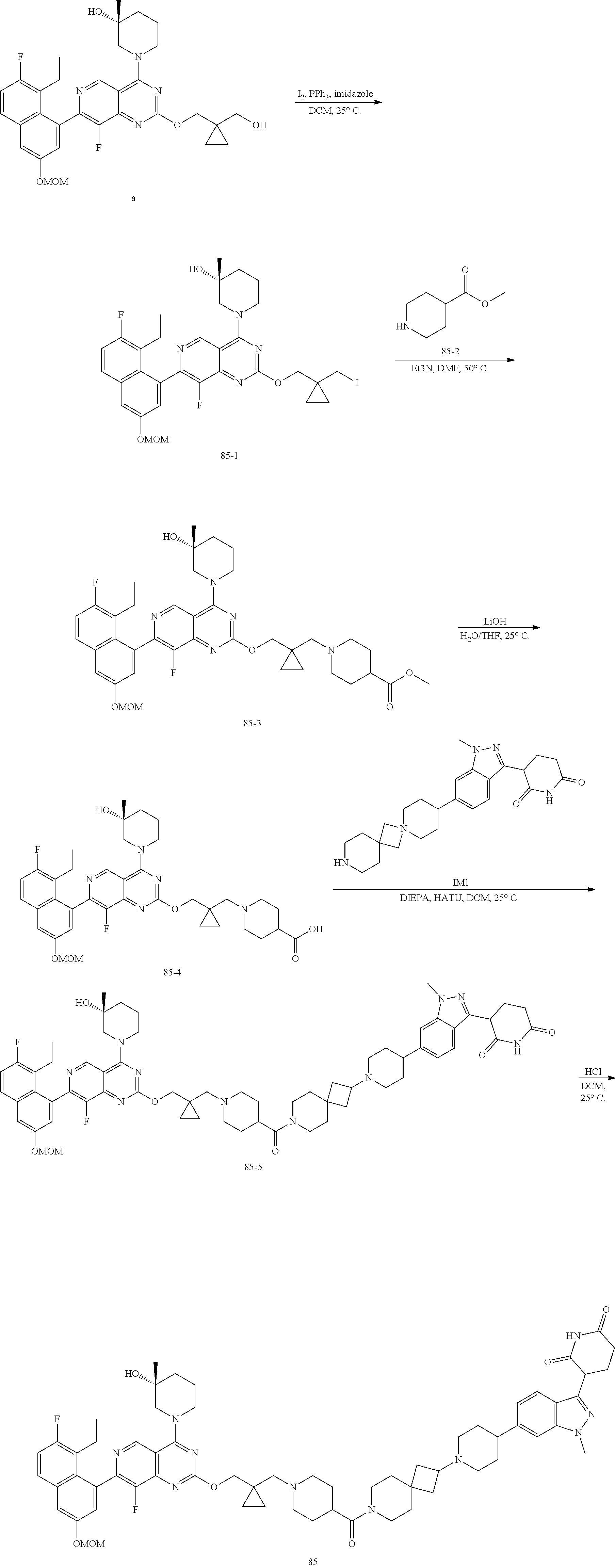

PPh₃ (1.59 g, 6.05 mmol, 40 eq) was added to a solution of a (90 mg, 151.35 μmol, 1 eq) in DCM (10 mL), followed by addition of imidazole (515.20 mg, 7.57 mmol, 50 eq) and I₂ (384.14 mg, 1.51 mmol, 10 eq). The mixture was stirred at 25° C. for 1 hour (h), then concentrated under reduced pressure. The residue was purified by column chromatography (MeOH/DCM=0%-1%) to afford 85-1 (80 mg, 75.0% yield). m/z, (ESI⁺). 705.3.

Et₃N (28.72 mg, 283.87 μmol, 5.0 eq) was added to a solution of 85-1 (40 mg, 56.77 μmol, 1 eq) in DMF (5 mL), followed by addition of 85-2 (16.26 mg, 113.55 μmol, 2.0 eq). The reaction mixture was stirred at 50° C. for 16 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-3%) to afford 85-3 (30 mg, 73.4% yield).

LiOH (4.99 mg, 208.39 μmol, 5.0 eq) was added to a solution of 85-3 (30 mg, 41.68 μmol, 1 eq) in THF (4 mL) and water (1 mL). The reaction mixture was stirred at 25° C. for 1 h, then pH was adjusted to 5-6 with 3N HCl aqueous solution, followed by addition of EtOAc and stirred for 10 min. The separated organic layer was washed with water and brine, then dried over Na₂SO₄ and filtered. The filtrate was concentrated under reduced pressure to afford 85-4 (29 mg, 98.6% yield).

IM1 (31 mg, 63.76 μmol, 1.5 eq) was added to a solution of 85-4 (30 mg, 42.51 μmol, 1 eq) in DCM (5 mL), followed by addition of DIPEA (16.48 mg, 127.52 μmol, 3 eq) and HATU (24.05 mg, 63.76 μmol, 1.5 eq). The reaction mixture was stirred at 25° C. for 1 h, then concentrated under reduced pressure. The residue was purified by column chromatography (MeOH/DCM=0%-5%) to afford 85-5 (80 mg, 75.0% yield).

A solution of HCl in EtOAc (3.21 mg, 87.92 μmol, 5.0 eq) was added to another solution of 85-5 (20 mg, 17.58 μmol, 1 eq) in DCM (5 mL). The reaction mixture was stirred at 25° C. for 30 min, then concentrated under reduced pressure. The residue was purified by preparative liquid chromatography (0.05% NH₃ in H₂O/MeCN) to afford 85 (7.4 mg, 38.0% yield). ¹H NMR (500 MHz, CD₃OD) δ 0.50 (s, 2H), 0.72 (s, 2H), 0.82 (d, J=5.0 Hz, 3H), 0.90 (d, J=5.0 Hz, 1H), 1.30 (s, 3H), 1.50-1.55 (m, 1H), 1.58-1.69 (m, 6H), 1.71-1.90 (m, 8H), 1.91-2.07 (m, 6H), 2.10-2.25 (m, 4H), 2.29-2.54 (m, 5H), 2.58-2.66 (m, 1H), 2.70-2.87 (m, 4H), 3.05-3.19 (m, 4H), 3.42-3.57 (m, 6H), 3.58-3.71 (m, 1H), 4.01 (s, 3H), 4.22-4.31 (m, 1H), 4.33-4.38 (m, 1H), 4.40-4.49 (m, 2H), 4.50-4.58 (m, 1H), 7.05-7.12 (m, 2H), 7.25 (d, J=10.0 Hz, 1H), 7.28-7.32 (m, 1H), 7.36 (s, 1H), 7.60-7.71 (m, 2H), 9.21 (s, 1H). m/z, (ESI⁺): 1093.9.

### Synthesis of Compound 86

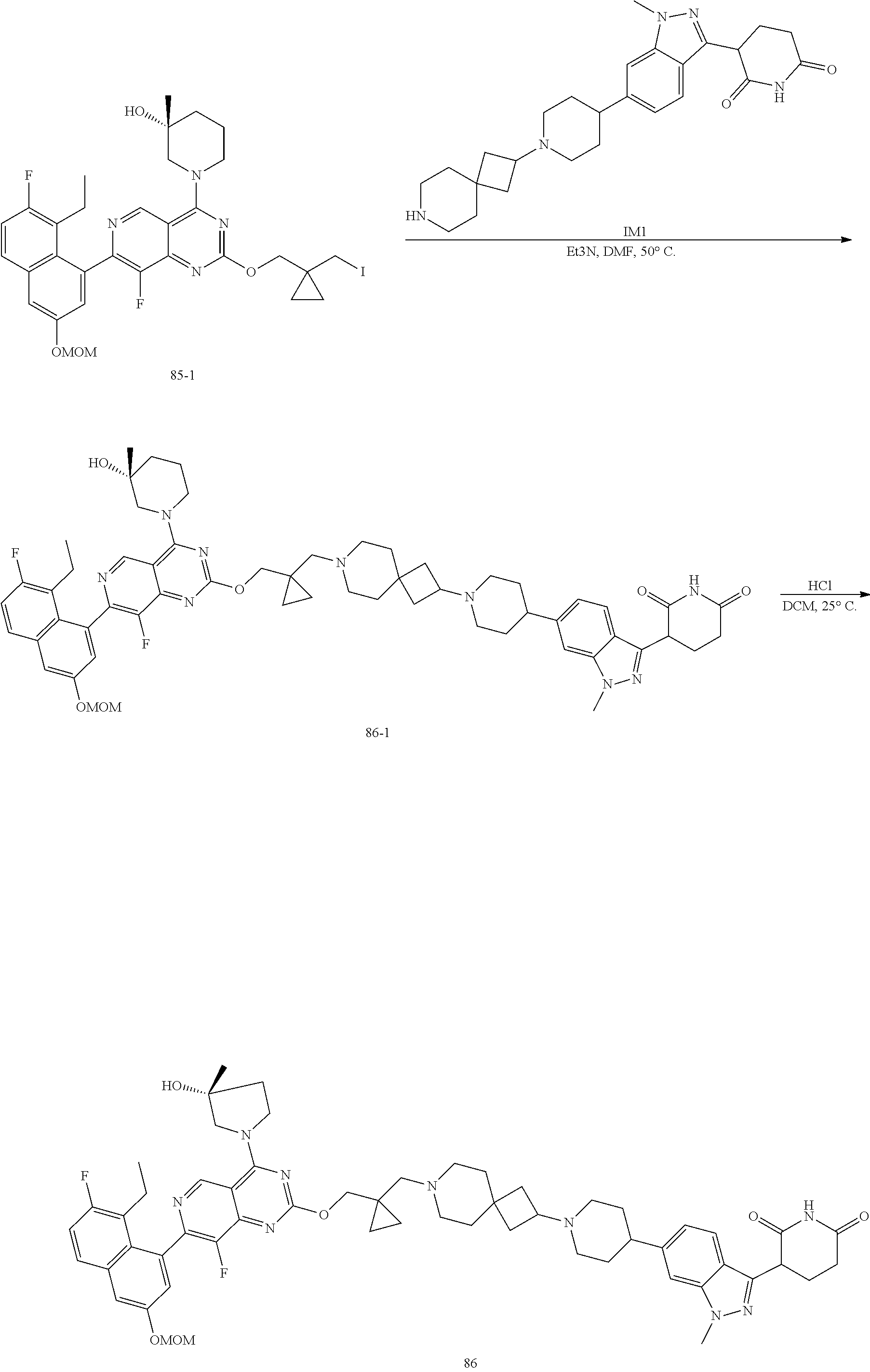

IM1 (41.39 mg, 85.16 μmol, 1.5 eq) was added to a solution of 85-1 (40 mg, 56.77 μmol, 1 eq) in DMF (5 mL), followed by addition of Et₃N (28.72 mg, 283.87 μmol, 5.0 eq). The reaction mixture was stirred at 50° C. for 16 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-3%) to afford 86-1 (20 mg, 34.3% yield).

A solution of HCl in EtOAc (3.56 mg, 97.45 μmol, 5.0 eq) was added to another solution of 86-1 (20 mg, 19.49 μmol, 1 eq) in DCM (5 mL). The reaction mixture was stirred at 25° C. for 30 min, then concentrated under reduced pressure. The residue was purified by preparative liquid chromatography (0.1% TFA in H₂O/MeCN) to afford 86 (7.0 mg, 25.4% yield). ¹H NMR (500 MHz, CD₃OD) δ 0.82 (d, J=5.0 Hz, 3H), 0.88-0.92 (m, 2H), 1.01 (s, 2H), 1.31 (s, 3H), 1.77-1.91 (m, 3H), 1.99-2.20 (m, 9H), 2.22-2.37 (m, 5H), 2.41-2.50 (m, 2H), 2.52-2.60 (m, 1H), 2.70-2.81 (m, 2H), 2.93-3.15 (m, 5H), 3.41-3.49 (m, 1H), 3.54-3.66 (m, 3H), 3.72-3.85 (m, 3H), 4.02 (s, 3H), 4.30-4.41 (m, 2H), 4.44-4.55 (m, 2H), 4.59-4.67 (m, 1H), 7.04-7.13 (m, 2H), 7.26 (d, J=10.0 Hz, 1H), 7.31 (s, 1H), 7.40 (s, 1H), 7.64-7.73 (m, 2H), 9.28 (s, 1H). m/z, (ESI⁺): 982.9.

### Synthesis of Compound 101

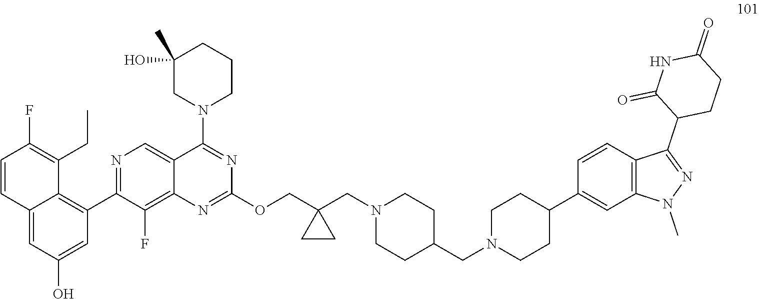 [SMILES: CCc1c(F)ccc2cc(O)cc(-c3ncc4c(N5CCC[C@@](C)(O)C5)nc(OCC5(CN6CCC(CN7CCC(c8ccc9c(C%10CCC(=O)NC%10=O)nn(C)c9c8)CC7)CC6)CC5)nc4c3F)c12]

101 was synthesized according to the procedure of 86 with IM2 as starting material. m/z, (ESI⁺): 606.8. ¹H NMR (500 MHz, CD₃OD) δ 9.25 (d, J=5.2 Hz, 1H), 7.72-7.60 (m, 2H), 7.36 (s, 1H), 7.28 (d, J=2.9 Hz, 1H), 7.23 (t, J=9.4 Hz, 1H), 7.07 (d, J=8.3 Hz, 1H), 7.03 (s, 1H), 4.59 (d, J=13.1 Hz, 1H), 4.48 (s, 2H), 4.38-4.26 (m, 2H), 4.06-3.85 (m, 5H), 3.73 (d, J=12.0 Hz, 2H), 3.62 (dd, J=27.0, 13.3 Hz, 1H), 3.51-3.39 (m, 2H), 3.20-2.99 (m, 6H), 2.83-2.65 (m, 2H), 2.51-2.39 (m, 2H), 2.35-2.06 (m, 10H), 1.94-1.68 (m, 5H), 1.37-1.22 (m, 5H), 1.00 (s, 2H), 0.92-0.76 (m, 5H). m/z, (ESI⁺): 956.7.

### Synthesis of Compound 130

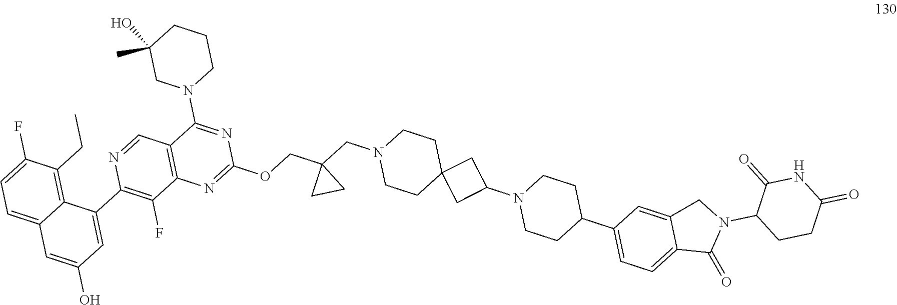 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c(c8)CN(C8CCC(=O)NC8=O)C9=O)CC6)C7)CC5)nc4c3F)c12]

130 was synthesized according to the procedure of 86 with IM3 as starting material. ¹H NMR (400 MHz, CD₃OD) δ 9.27 (d, J=1.7 Hz, 1H), 7.81 (d, J=8.0 Hz, 1H), 7.70 (dd, J=9.1, 5.8 Hz, 1H), 7.53 (s, 1H), 7.47 (d, J=8.0 Hz, 1H), 7.33 (d, J=2.7 Hz, 1H), 7.28 (t, J=9.4 Hz, 1H), 7.07 (t, J=3.2 Hz, 1H), 5.26-5.17 (m, 1H), 4.66-4.45 (m, 5H), 4.34-4.31 (m, 1H), 3.82-3.76 (m, 3H), 3.75-3.54 (m, 4H), 3.51-3.41 (m, 2H), 3.12-2.88 (m, 6H), 2.83-2.80 (m, 1H), 2.54-2.51 (m, 4H), 2.30-1.97 (m, 12H), 1.86-1.83 (m, 4H), 1.36-1.28 (m, 4H), 1.04-1.01 (m, 2H), 0.93-0.78 (m, 5H). m/z, (ESI⁺): 983.5.

### Synthesis of Compound 137

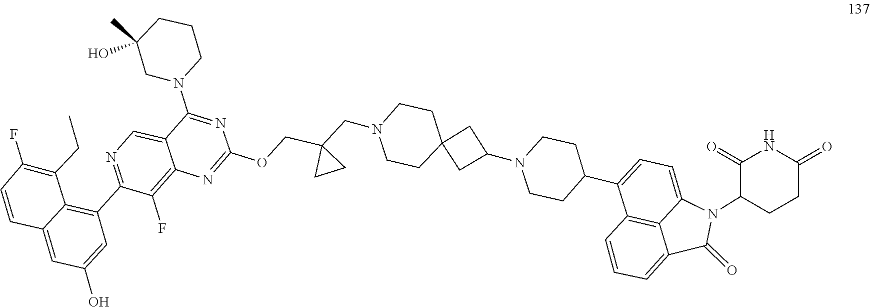 [SMILES: CCc1c(F)ccc2cc(O)cc(-c3ncc4c(N5CCC[C@@](C)(O)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12]

137 was synthesized according to the procedure of 86 with IM4 as starting material. ¹H NMR (400 MHz, CD₃OD) δ 9.19 (d, J=2.6 Hz, 1H), 8.37 (d, J=11.0 Hz, 3H), 8.08 (dd, J=7.0, 3.5 Hz, 1H), 7.82 (t, J=7.7 Hz, 1H), 7.62 (dd, J=9.1, 5.8 Hz, 1H), 7.37 (d, J=7.5 Hz, 1H), 7.25 (d, J=2.6 Hz, 1H), 7.21 (t, J=9.4 Hz, 1H), 7.12-6.95 (m, 2H), 5.39 (dd, J=12.7, 5.4 Hz, 1H), 4.60-4.35 (m, 3H), 4.28 (t, J=12.2 Hz, 1H), 3.66-3.50 (m, 2H), 3.48-3.34 (m, 5H), 3.18-3.01 (m, 3H), 2.98-2.55 (m, 5H), 2.48-2.36 (m, 1H), 2.34-1.68 (m, 18H), 1.25 (d, J=9.7 Hz, 4H), 0.92 (s, 2H), 0.82-0.68 (m, 5H). m/z, (ESI⁺): 1019.67.

### Synthesis of Compound 138

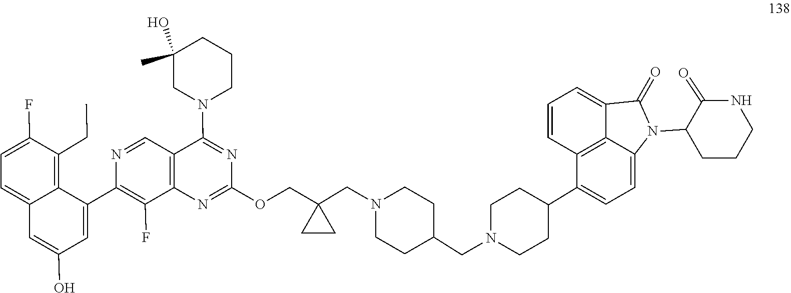 [SMILES: CCc1c(F)ccc2cc(O)cc(-c3ncc4c(N5CCC[C@@](C)(O)C5)nc(OCC5(CN6CCC(CN7CCC(c8ccc9c%10c(cccc8%10)C(=O)N9C8CCCNC8=O)CC7)CC6)CC5)nc4c3F)c12]

138 was synthesized according to the procedure of 86 with IM5 as starting material. ¹H NMR (400 MHz, CD₃OD) δ 9.26 (d, J=4.3 Hz, 1H), 8.42 (d, J=8.3 Hz, 1H), 8.13 (d, J=6.9 Hz, 1H), 7.87 (t, J=7.7 Hz, 1H), 7.65 (t, J=7.6 Hz, 1H), 7.40 (d, J=7.5 Hz, 1H), 7.25 (dd, J=19.9, 10.6 Hz, 2H), 7.07 (d, J=8.0 Hz, 2H), 5.46-5.32 (m, 2H), 4.57-4.61 (m, 2H), 4.48-4.54 (m, 3H), 4.29-4.35 m, 2H), 3.92-3.97 (m, 2H), 3.83-3.44 (m, 6H), 3.09-3.13 (m, 2H), 2.94-2.98 (m, 2H), 2.82-2.85 (m, 2H), 2.41-2.44 (m, 2H), 2.17-2.21 (m, 6H), 2.01-2.04 (m, 2H), 1.81-1.87 (m, 4H), 1.58-1.61 (m, 1H), 1.29-1.33 (m, 6H), 0.92-0.78 (m, 4H). m/z, (ESI⁺): 993.5.

### Synthesis of Compound 141

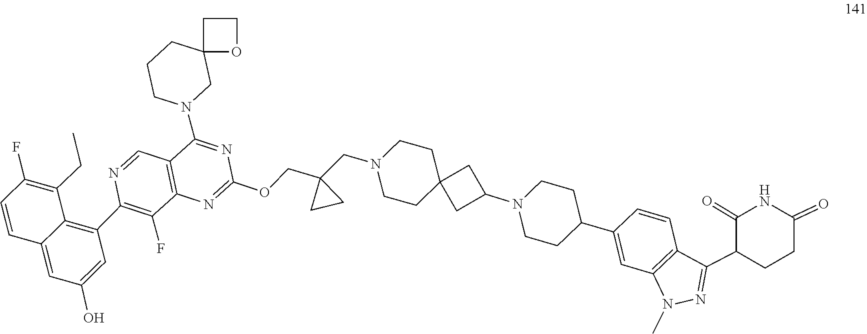 [SMILES: CCc1c(F)ccc2cc(O)cc(-c3ncc4c(N5CCCC6(CCO6)C5)nc(OCC5(CN6CCC7(CC6)CC(N6CCC(c8ccc9c(C%10CCC(=O)NC%10=O)nn(C)c9c8)CC6)C7)CC5)nc4c3F)c12]

141 was synthesized according to the procedure of 86 with b as starting material. H NMR (500 MHz, CD₃OD) δ 9.25 (d, J=4.1 Hz, 1H), 7.69 (dd, J=18.1, 8.4 Hz, 2H), 7.40 (s, 1H), 7.31 (s, 1H), 7.26 (t, J=9.2 Hz, 1H), 7.10 (d, J=8.4 Hz, 1H), 7.06 (s, 1H), 4.62-4.45 (m, 3H), 4.43-4.30 (m, 2H), 4.03 (s, 3H), 3.91-3.59 (m, 6H), 3.49 (dd, J=25.2, 16.2 Hz, 2H), 3.06 (ddd, J=58.1, 28.3, 14.7 Hz, 6H), 2.85-2.42 (m, 6H), 2.38-1.96 (m, 16H), 1.84 (dd, J=22.4, 8.6 Hz, 3H), 1.02 (s, 2H), 0.89 (s, 2H), 0.82 (d, J=3.7 Hz, 3H). m/z, (ESI⁺): 994.8.

### Synthesis of Compound 179a

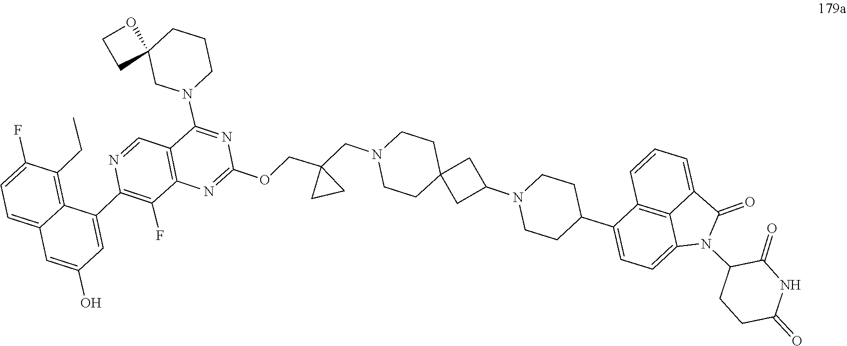 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12]

179a was synthesized according to the procedure of 137 with c as starting material. ¹H NMR (400 MHz, CD₃OD) δ 9.30 (d, J=9.3 Hz, 1H), 8.44-8.40 (m, 1H), 8.11-8.15 (m, 1H), 7.91-7.85 (m, 1H), 7.67-7.70 (m, 1H), 7.40-7.42 (m, 1H), 7.33-7.24 (m, 2H), 7.12-7.04 (m, 2H), 5.42-5.47 (m, 2H), 4.47-4.62 (m, 6H), 4.10-4.14 (m, 1H), 3.82-3.87 (m, 1H), 3.61-3.39 (m, 5H), 3.09-3.18 (m, 3H), 3.06-2.85 (m, 4H), 2.81-2.84 (m, 1H), 2.68-2.75 (s, 2H), 2.60-2.42 (m, 3H), 2.38-1.78 (m, 17H), 1.31-1.45 (m=16.3 Hz, 3H), 0.87-0.77 (m, 4H). m/z, (ESI⁺): 1031.5.

### Synthesis of Compound 194

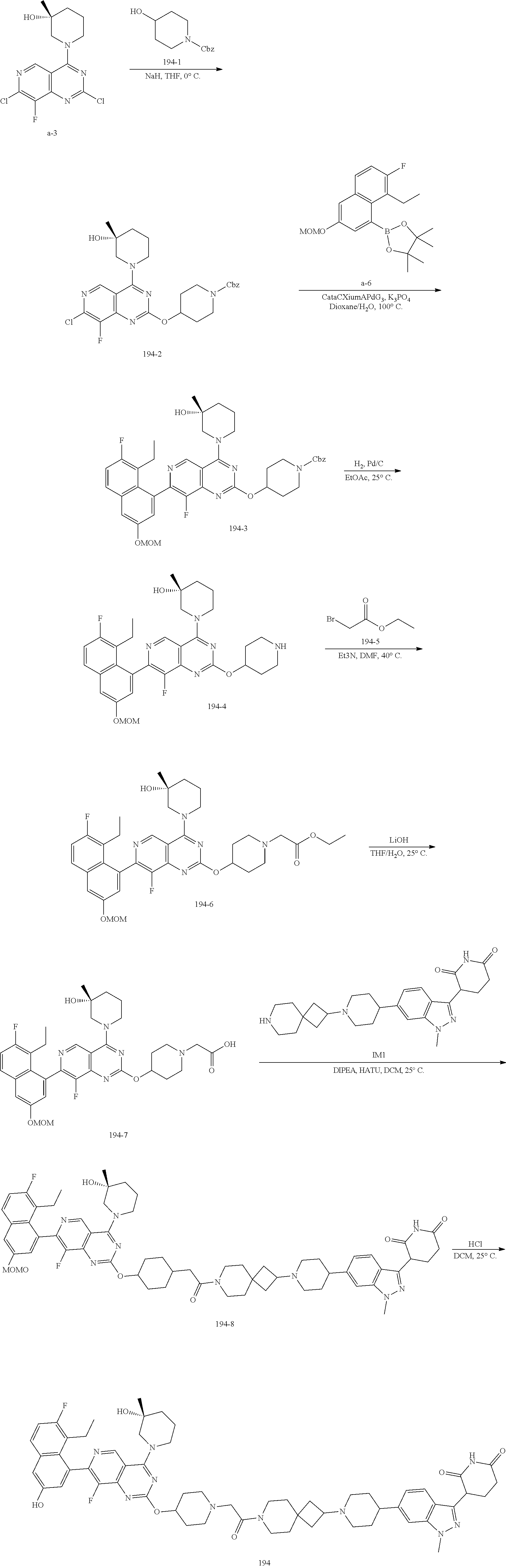

194-1 (71.04 mg, 301.96 μmol, 1 eq) was added to a solution of a-3 (100 mg, 301.96 μmol, 1 eq) in THF (5 mL). The reaction mixture was cooled to 0° C. under nitrogen atmosphere, followed by addition of 60% NaH (24.15 mg, 603.92 μmol, 2 eq), then stirred for 1 h. The reaction mixture was warmed to room temperature, and the residue was purified by column chromatography (MeOH/DCM=0%-2%) to afford 194-2 110 mg, 68.7% yield).

a-6 (114.19 mg, 316.99 μmol, 1.2 eq) was added to a solution of 194-2 (140 mg, 264.16 μmol, 1 eq) in dioxane (10 mL) and water (1 mL), followed by addition of cataCXium A Pd G3 (38.46 mg, 52.83 μmol, 0.2 eq) and K₃PO₄ (168.00 mg, 792.47 μmol, 3 eq). The reaction mixture was stirred at 100° C. for 4 h under nitrogen atmosphere, then cooled to room temperature. The reaction mixture was treated with water and DCM, then stirred for 10 min. The separated organic layer was washed with water and brine, then dried over Na₂SO₄ and filtered. The filtrate was concentrated under reduced pressure and the residue was purified by column chromatography (MeOH/DCM=0%-2%) to afford 194-3 (140 mg, 72.8% yield).

10% Pd/C (24 mg) was added to a solution of 194-3 (140 mg, 192.36 μmol, 1 eq) in EtOAc (5 mL). The reaction mixture was stirred at 25° C. for 16 h under hydrogen atmosphere, then filtered. The filtrate was concentrated under reduced pressure to afford 194-4 (100 mg, 87.6% yield).

194-5 (33.76 mg, 202.14 μmol, 1.2 eq) was added to a solution of 194-4 (100 mg, 168.45 μmol, 1 eq) in DMF (5 mL), followed by addition of Et₃N (51.14 mg, 505.34 μmol, 3 eq). The reaction mixture was stirred at 40° C. for 16 h, then cooled to room temperature. The reaction mixture was treated with water and EtOAc, then stirred for 10 min. The separated organic layer was washed with water and brine, then dried over Na₂SO₄ and filtered. The filtrate was concentrated under reduced pressure and the residue was purified by column chromatography (MeOH/DCM=0%-2%) to afford 194-6 (60 mg, 52.4% yield).

LiOH (12.68 mg, 529.61 μmol, 3 eq) was added to a solution of 194-6 (120 mg, 176.54 μmol, 1 eq) in THF (3 mL) and water (3 mL). The reaction mixture was stirred at 25° C. for 1 h, then pH was adjusted to 5-6 with 3N HCl aqueous solution, followed by addition of EtOAc and stirred for 10 min. The separated organic layer was washed with water and brine, then dried over Na₂SO₄ and filtered. The filtrate was concentrated under reduced pressure to afford 194-7 (100 mg, 86.9% yield).

IM1 (71.60 mg, 147.31 μmol, 1.2 eq) was added to a solution of 194-7 (80 mg, 122.76 μmol, 1 eq) in DMF (5 mL), followed by addition of DIPEA (79.33 mg, 613.78 μmol, 5 eq) and HATU (69.47 mg, 184.13 μmol, 1.5 eq). The reaction mixture was stirred at 25° C. for 1 h, then concentrated under reduced pressure. The residue was purified by column chromatography (MeOH/DCM=0%-5%) to afford 194-8 (60 mg, 45.1% yield).

A solution of HCl in EtOAc (0.1 mL) was added to a solution of 194-8 (60 mg, 55.39 μmol, 1 eq) in DCM (5 mL). The reaction mixture was stirred at 25° C. for 30 min, then concentrated under reduced pressure. The residue was purified by preparative liquid chromatography (0.10% TFA in H₂O/MeCN) to afford 194 (45 mg, 75.9% yield). ¹H NMR (500 MHz, CD₃OD) δ 0.82 (q, J=7.9 Hz, 3H), 0.90 (t, J=6.8 Hz, 1H), 1.30 (d, J=11.4 Hz, 10H), 1.67 (d, J=5.8 Hz, 1H), 1.73 (s, 2H), 1.80 (d, J=9.6 Hz, 2H), 1.85-1.91 (m, 1H), 2.05 (dd, J=17.2, 8.8 Hz, 2H), 2.13-2.23 (m, 6H), 2.30-2.36 (m, 2H), 2.43 (q, J=10.7, 8.0 Hz, 4H), 2.77 (tt, J=14.4, 5.5 Hz, 2H), 2.98 (t, J=12.8 Hz, 2H), 3.08 (t, J=12.5 Hz, 1H), 3.36 (d, J=5.5 Hz, 1H), 3.41-3.48 (m, 3H), 3.56-3.61 (m, 2H), 3.63-3.68 (m, 3H), 3.78 (q, J=8.5 Hz, 2H), 4.02 (s, 3H), 4.28-4.42 (m, 4H), 4.62 (d, J=13.0 Hz, 1H), 7.06 (t, J=3.2 Hz, 1H), 7.10 (d, J=8.5 Hz, 1H), 7.27 (t, J=9.3 Hz, 1H), 7.32 (d, J=2.6 Hz, 1H), 7.39 (s, 1H), 7.67-7.73 (m, 2H), 9.27 (s, 1H). m/z, (ESI⁺): 1038.53.

### Synthesis of Compound 195

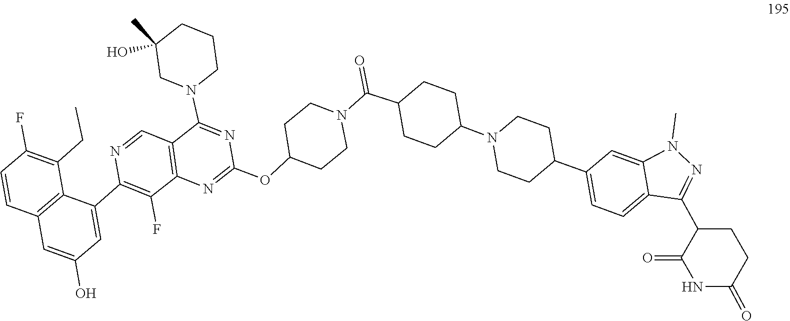 [SMILES: CCc1c(F)ccc2cc(O)cc(-c3ncc4c(N5CCC[C@@](C)(O)C5)nc(OC5CCN(C(=O)C6CCC(N7CCC(c8ccc9c(C%10CCC(=O)NC%10=O)nn(C)c9c8)CC7)CC6)CC5)nc4c3F)c12]

195 was synthesized according to the procedure of 194 with IM5 as starting material. ¹H NMR (500 MHz, CD₃OD) δ 0.84 (m, 3H), 1.29 (s, 3H), 1.32 (d, J=7.7 Hz, 4H), 1.75-2.05 (m, 9H), 2.09-2.28 (m, 10H), 2.30-2.36 (m, 1H), 2.47 (m, 2H), 2.68-2.82 (m, 2H), 3.03-3.14 (m, 2H), 3.26 (m, 2H), 3.65 (m, 5H), 3.81-3.92 (m, 2H), 4.02 (s, 3H), 4.32-4.49 (m, 2H), 4.73 (m, 1H), 5.53 (s, 1H), 7.07-7.15 (m, 2H), 7.27 (m, 1H), 7.33 (d, J=2.7 Hz, 1H), 7.39 (s, 1H), 7.69 (m, 2H), 9.33 (d, J=6.2 Hz, 1H). m/z, (ESI⁺): 984.9.

### Synthesis of Compound 196

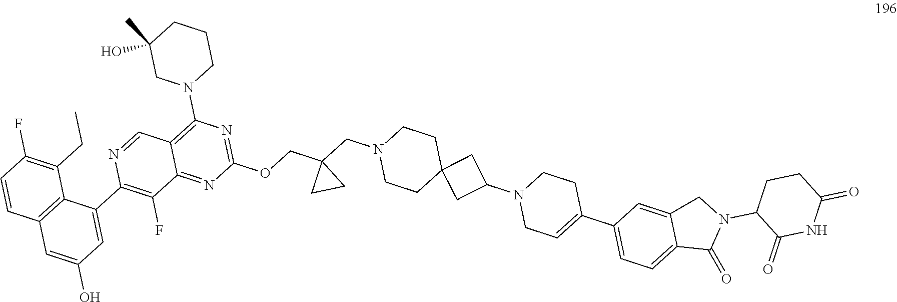 [SMILES: CCc1c(F)ccc2cc(O)cc(-c3ncc4c(N5CCC[C@@](C)(O)C5)nc(OCC5(CN6CCC7(CC6)CC(N6CC=C(c8ccc9c(c8)CN(C8CCC(=O)NC8=O)C9=O)CC6)C7)CC5)nc4c3F)c12]

196 was synthesized according to the procedure of 86 with IM7 as starting material. ¹H NMR (500 MHz, CD₃OD) δ 0.82 (q, J=8.1 Hz, 3H), 0.90 (d, J=5.0 Hz, 2H), 1.01 (s, 2H), 1.30 (d, J=11.1 Hz, 6H), 1.76-1.92 (m, 3H), 2.04 (s, 8H), 2.15-2.26 (m, 4H), 2.49 (ddd, J=31.8, 16.1, 10.1 Hz, 2H), 2.79 (d, J=17.4 Hz, 1H), 2.92 (ddd, J=18.1, 12.9, 4.9 Hz, 3H), 3.01-3.14 (m, 2H), 3.43 (d, J=15.0 Hz, 2H), 3.57-3.74 (m, 3H), 3.79 (d, J=14.5 Hz, 2H), 3.92 (t, J=8.5 Hz, 1H), 4.07 (s, 1H), 4.35 (t, J=14.8 Hz, 1H), 4.52 (d, J=12.7 Hz, 3H), 4.63 (d, J=13.4 Hz, 1H), 5.17 (dd, J=13.3, 5.4 Hz, 1H), 6.29 (s, 1H), 7.07 (t, J=3.5 Hz, 1H), 7.27 (t, J=9.4 Hz, 1H), 7.31-7.49 (m, 2H), 7.66-7.72 (m, 2H), 7.81 (d, J=7.9 Hz, 1H), 9.28 (s, 1H). m/z, (ESI⁺): 980.48.

### Synthesis of Compound 197

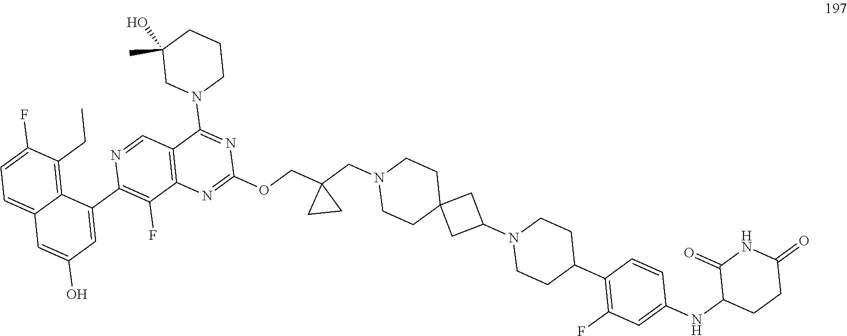 [SMILES: CCc1c(F)ccc2cc(O)cc(-c3ncc4c(N5CCC[C@@](C)(O)C5)nc(OCC5(CN6CCC7(CC6)CC(N6CCC(c8ccc(NC9CCC(=O)NC9=O)cc8F)CC6)C7)CC5)nc4c3F)c12]

197 was synthesized according to the procedure of 86 with IM8 as starting material. ¹H NMR (400 MHz, DMSO-d₆) δ 10.80 (s, 1H), 10.37-10.24 (m, 1H), 10.06-9.91 (m, 1H), 9.27 (d, J=2.5 Hz, 1H), 8.90-8.78 (m, 1H), 7.78 (dd, J=9.1, 6.0 Hz, 1H), 7.43-7.25 (m, 2H), 7.03 (d, J=2.6 Hz, 1H), 6.95 (t, J=8.8 Hz, 1H), 6.60-6.42 (m, 2H), 4.41-4.28 (m, 3H), 4.21-4.06 (m, 1H), 3.69-3.55 (m, 4H), 3.53-3.33 (m, 12H), 3.22 (d, J=5.3 Hz, 2H), 3.03-2.82 (m, 4H), 2.79-2.58 (m, 2H), 2.37-2.28 (m, 2H), 2.28-1.98 (m, 6H), 1.97-1.82 (m, 6H), 1.77-1.61 (m, 3H), 1.24 (s, 1H), 1.18 (d, J=10.9 Hz, 2H), 0.90 (s, 2H), 0.83-0.68 (m, 4H). m/z, (ESI⁺): 961.5.

### Synthesis of Compound 198

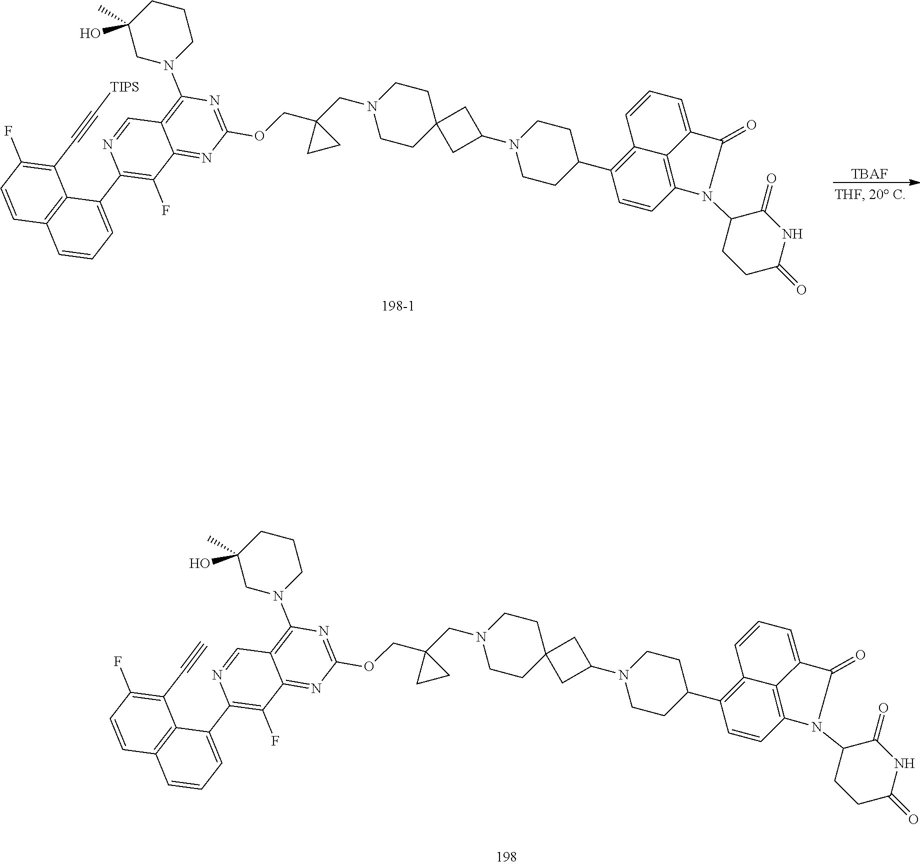

198-1 was synthesized according to the procedure of 137 with d as starting material.

TBAF (40.73 mg, 155.78 μmol, 2 eq) was added to a solution of 198-1 (90 mg, 77.89 μmol, 1 eq) in THF (2 mL). The mixture was stirred at 25° C. for 10 min, then concentrated under reduced pressure. The residue was purified by preparative liquid chromatography (0.1% TFA in H₂O/MeCN) to afford 198 (25.1 mg, 31.3% yield). ¹H NMR (400 MHz, CD₃OD) δ 0.88-0.90 (m, 4H), 1.26-1.31 (m, 10H), 1.60-2.37 (m, 12H), 2.82-3.13 (m, 5H), 3.42-3.89 (m, 9H), 4.03-4.16 (m, 1H), 4.31-4.72 (m, 5H), 5.43-5.46 (m, 1H), 7.08 (t, J=7.6 Hz, 1H), 7.34-7.48 (m, 2H), 7.65-7.71 (m, 2H), 7.88 (t, J=8.0 Hz, 1H), 8.07-8.14 (m, 3H), 8.42 (t, J=8.4 Hz, 1H), 9.15-9.27 (m, 1H). m/z, (ESI⁺): 999.18.

### Synthesis of Compound 199

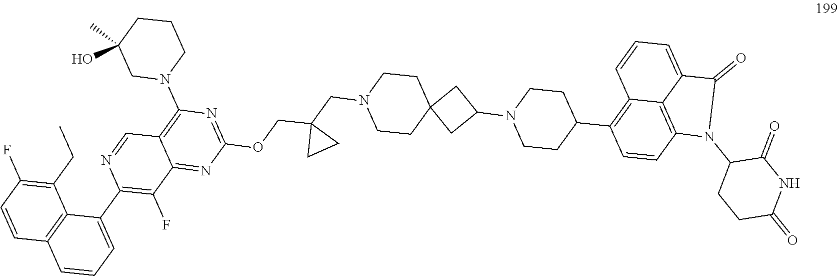 [SMILES: CCc1c(F)ccc2cccc(-c3ncc4c(N5CCC[C@](C)(O)C5)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12]

199 was synthesized according to the procedure of 137 with e as starting material. ¹H NMR (400 MHz, CD₃OD) δ 9.35 (s, 1H), 8.46 (d, J=8.3 Hz, 1H), 8.16 (d, J=7.0 Hz, 1H), 8.09 (d, J=8.2 Hz, 1H), 7.92 (dd, J=16.8, 8.8 Hz, 2H), 7.59 (t, J=7.6 Hz, 1H), 7.52 (s, 1H), 7.46-7.37 (m, 2H), 7.11 (d, J=7.5 Hz, 1H), 5.53-5.41 (m, 1H), 4.69 (d, J=13.2 Hz, 1H), 4.58-4.50 (m, 2H), 4.40 (t, J=12.8 Hz, 1H), 3.84 (s, 3H), 3.69 (d, J=12.3 Hz, 4H), 3.48 (t, J=13.0 Hz, 2H), 3.22-2.78 (m, 9H), 2.58 (d, J=28.9 Hz, 2H), 2.42-2.04 (m, 16H), 1.93-1.82 (m, 3H), 1.35 (s, 3H), 0.93 (s, 2H), 0.88 (d, J=7.4 Hz, 2H). m/z, (ESI⁺): 1003.29.

### Synthesis of Compound 200

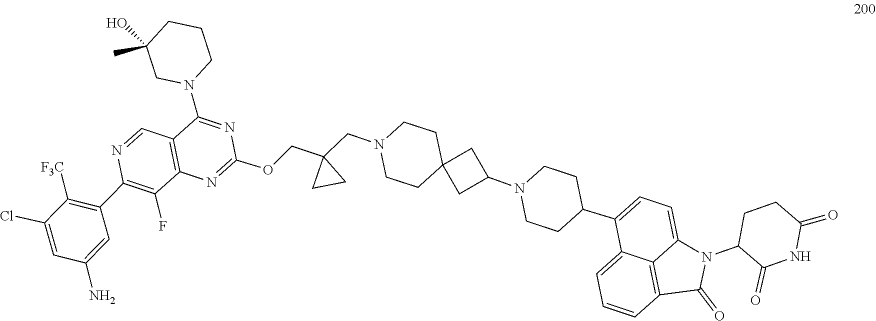 [SMILES: C[C@@]1(O)CCCN(c2nc(OCC3(CN4CCC5(CC4)CC(N4CCC(c6ccc7c8c(cccc68)C(=O)N7C6CCC(=O)NC6=O)CC4)C5)CC3)nc3c(F)c(-c4cc(N)cc(Cl)c4C(F)(F)F)ncc23)C1]

200 was synthesized according to the procedure of 137 with f as starting material. ¹H NMR (400 MHz, CD₃OD) δ: 0.88 (s, 2H), 1.00 (s, 2H), 1.29 (s, 4H), 1.80 (dd, J=22.1, 12.8 Hz, 3H), 1.93-2.41 (m, 14H), 2.58 (s, 1H), 2.84 (t, J=12.8 Hz, 2H), 2.90-3.04 (m, 2H), 3.10 (t, J=10.8 Hz, 3H), 3.40 (t, J=11.9 Hz, 1H), 3.57-3.72 (m, 4H), 3.73-3.88 (m, 3H), 4.32 (d, J=13.3 Hz, 1H), 4.47 (s, 2H), 4.61 (d, J=12.4 Hz, 1H), 5.34-5.50 (m, 1H), 6.50 (s, 1H), 6.90 (s, 1H), 7.08 (d, J=7.4 Hz, 1H), 7.41 (d, J=7.5 Hz, 1H), 7.86 (t, J=7.7 Hz, 1H), 8.11 (d, J=7.0 Hz, 1H), 8.42 (d, J=8.4 Hz, 1H), 9.21 (s, 1H). m/z, (ESI⁺): 1024.39.

### Synthesis of Compound 201

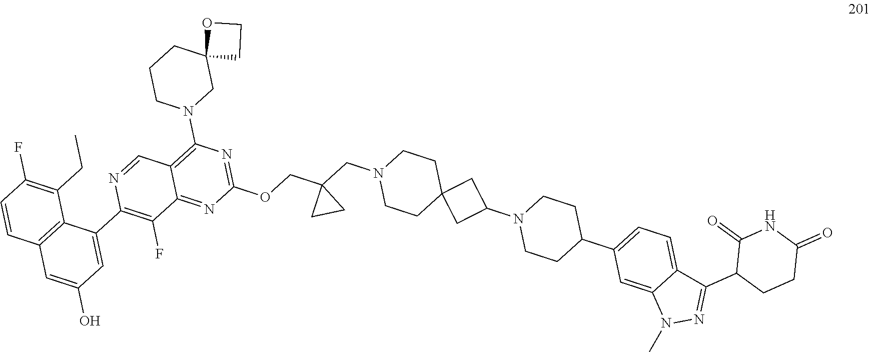 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c(C%10CCC(=O)NC%10=O)nn(C)c9c8)CC6)C7)CC5)nc4c3F)c12]

201 was synthesized according to the procedure of 86 with c as starting material. ¹H NMR (400 MHz, CD₃OD) δ 0.81-0.82 (m, 3H), 0.88 (s, 2H), 1.01 (s, 2H), 1.82-2.37 (m, 21H), 2.42-2.60 (m, 5H), 2.73-2.77 (m, 2H), 2.92-3.13 (m, 5H), 3.60-3.63 (m, 2H), 3.76-3.86 (m, 4H), 4.02 (s, 3H), 4.35-4.38 (m, 1H), 4.44-4.57 (m, 5H), 7.08-7.10 (m, 2H), 7.25 (t, J=9.2 Hz, 1H), 7.31 (s, 1H), 7.38 (s, 1H), 7.66-7.71 (m, 1H), 9.29-9.31 (m, 1H). m/z, (ESI⁺): 994.36.

### Synthesis of Compound 202

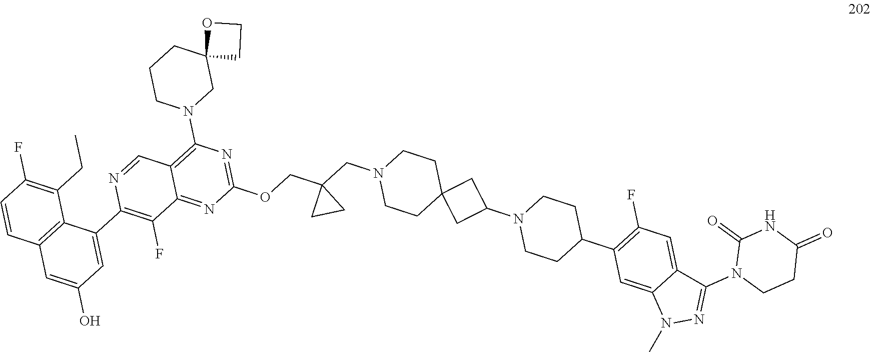 [SMILES: CCc1c(F)ccc2cc(O)cc(-c3ncc4c(N5CCC[C@]6(CCO6)C5)nc(OCC5(CN6CCC7(CC6)CC(N6CCC(c8cc9c(cc8F)c(N8CCC(=O)NC8=O)nn9C)CC6)C7)CC5)nc4c3F)c12]

202 was synthesized according to the procedure of 201 with IM9 as starting material. ¹H NMR (400 MHz, CD₃OD) δ 0.79-0.84 (m, 3H), 0.88 (s, 2H), 1.01 (s, 2H), 1.78-2.52 (m, 22H), 2.86 (t, J=6.8 Hz, 2H), 2.96-3.13 (m, 4H), 3.37-3.46 (m, 2H), 3.61-3.64 (m, 2H), 3.76-3.86 (m, 4H), 4.02-4.05 (m, 5H), 4.49-4.77 (m, 6H), 7.06-7.08 (m, 1H), 7.25 (t, J=9.2 Hz, 1H), 7.31 (s, 1H), 7.40-7.43 (m, 2H), 7.66-7.69 (m, 1H), 9.29-9.31 (m, 1H). m/z, (ESI⁺): 1013.47.

### Synthesis of Compound 203

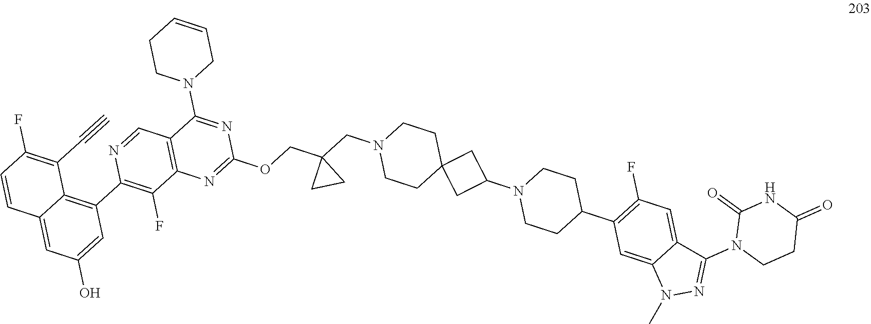 [SMILES: C#Cc1c(F)ccc2cc(O)cc(-c3ncc4c(N5CC=CCC5)nc(OCC5(CN6CCC7(CC6)CC(N6CCC(c8cc9c(cc8F)c(N8CCC(=O)NC8=O)nn9C)CC6)C7)CC5)nc4c3F)c12]

203 was synthesized according to the procedure of 202 with g as starting material. ¹H NMR (400 MHz, CD₃OD) δ: 0.92 (d, J=5.5 Hz, 2H), 1.05 (s, 2H), 2.10 (t, J=44.0 Hz, 12H), 2.54 (s, 3H), 2.90 (t, J=6.6 Hz, 2H), 2.96-3.22 (m, 4H), 3.41 (d, J=13.7 Hz, 2H), 3.51 (s, 1H), 3.65 (d, J=12.2 Hz, 2H), 3.78 (t, J=8.3 Hz, 3H), 4.02-4.12 (m, 5H), 4.14-4.23 (m, 1H), 4.29 (d, J=13.1 Hz, 1H), 4.54 (q, J=12.1 Hz, 2H), 4.62 (s, 2H), 5.88 (d, J=10.2 Hz, 1H), 6.09 (d, J=10.1 Hz, 1H), 7.28 (d, J=2.5 Hz, 1H), 7.38 (d, J=11.0 Hz, 2H), 7.45 (d, J=11.3 Hz, 2H), 7.84-7.95 (m, 1H), 9.12 (s, 1H). ¹⁹F NMR (376 MHz, CD₃OD) δ: −139.41 (s, 1F), −128.90 (dd, J=11.1, 6.0 Hz, 1F), −111.60-−113.39 (m, 1F), −77.07 (s, 13F). m/z, (ESI⁺): 965.49.

### Synthesis of Compound 204

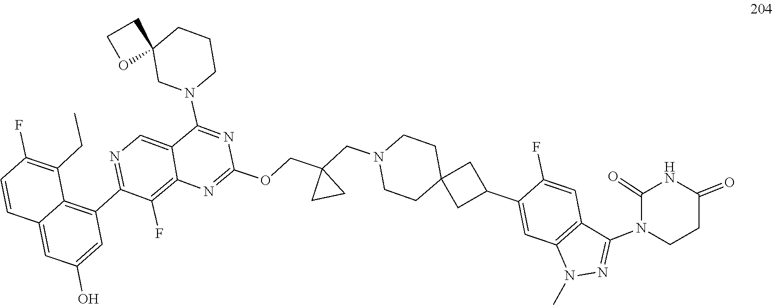 [SMILES: CCc1c(F)ccc2cc(O)cc(-c3ncc4c(N5CCC[C@]6(CCO6)C5)nc(OCC5(CN6CCC7(CC6)CC(c6cc8c(cc6F)c(N6CCC(=O)NC6=O)nn8C)C7)CC5)nc4c3F)c12]

204 was synthesized according to the procedure of 201 with IM10 as starting material. ¹H NMR (400 MHz, CD₃OD) δ 9.30 (d, J=6.1 Hz, 1H), 7.73-7.61 (m, 1H), 7.38 (d, J=5.9 Hz, 1H), 7.35-7.28 (m, 2H), 7.30-7.20 (m, 1H), 7.08 (s, 1H), 4.81-4.63 (m, 1H), 4.61-4.38 (m, 5H), 4.07-3.96 (m, 5H), 3.92-3.66 (m, 4H), 3.52-3.34 (m, 2H), 3.20-3.09 (m, 1H), 3.00 (t, J=11.9 Hz, 1H), 2.86 (t, J=6.6 Hz, 2H), 2.63-2.40 (m, 4H), 2.39-2.27 (m, 3H), 2.26-1.75 (m, 9H), 1.41-1.20 (m, 1H), 1.06-0.95 (m, 2H), 0.93-0.85 (m, 2H), 0.85-0.76 (m, 3H). ¹⁹F NMR (376 MHz, CD₃OD) δ−77.30 (s, 6F), −120.98 (s, 1F), −127.46 (t, J=8.5 Hz, 1F), −139.06 (dd, J=57.3, 7.4 Hz, 1F). m/z, (ESI⁺): 930.7.

### Synthesis of Compound 205

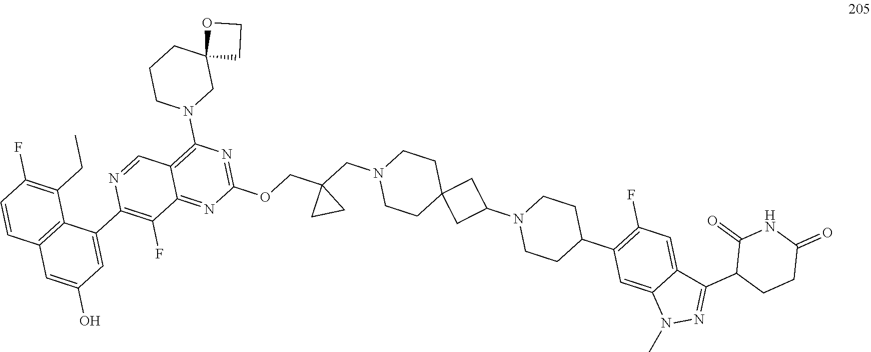 [SMILES: CCc1c(F)ccc2cc(O)cc(-c3ncc4c(N5CCC[C@]6(CCO6)C5)nc(OCC5(CN6CCC7(CC6)CC(N6CCC(c8cc9c(cc8F)c(C8CCC(=O)NC8=O)nn9C)CC6)C7)CC5)nc4c3F)c12]

205 was synthesized according to the procedure of 201 with IM11 as starting material. ¹H NMR (400 MHz, CD₃OD) δ 0.52 (s, 2H), 0.73 (s, 2H), 0.82 (t, J=6.8 Hz, 3H), 1.57-1.67 (m, 6H), 1.83-2.03 (m, 11H), 2.16-2.53 (m, 10H), 2.67-2.83 (m, 3H), 2.94-3.13 (m, 3H), 3.39-3.61 (m, 1H), 3.79-3.87 (m, 1H), 4.02 (s, 3H), 4.29-4.33 (m, 1H), 4.40-4.68 (m, 9H), 7.07-7.08 (m, 1H), 7.24 (t, J=9.6 Hz, 1H), 7.31 (s, 1H), 7.39 (t, J=10.8 Hz, 2H), 7.65-7.69 (m, 1H), 9.24-9.27 (m, 1H). m/z, (ESI⁺): 1012.33.

### Synthesis of Compound 206

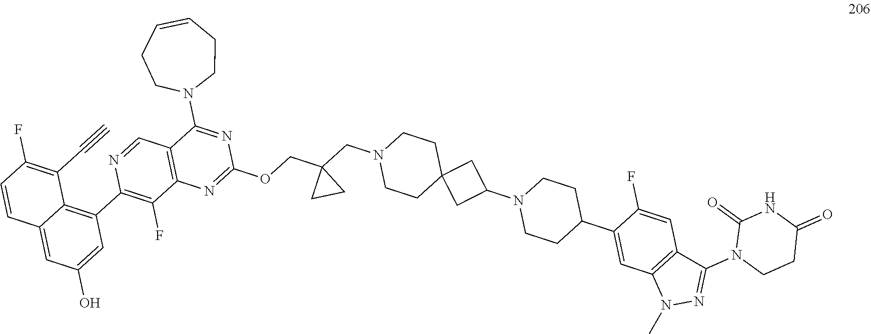 [SMILES: C#Cc1c(F)ccc2cc(O)cc(-c3ncc4c(N5CCC=CCC5)nc(OCC5(CN6CCC7(CC6)CC(N6CCC(c8cc9c(cc8F)c(N8CCC(=O)NC8=O)nn9C)CC6)C7)CC5)nc4c3F)c12]

206 was synthesized according to the procedure of 202 with h as starting material. ¹H NMR (400 MHz, CD₃OD) δ 0.87-0.92 (m, 2H), 1.01 (d, J=5.6 Hz, 2H), 1.90-2.34 (m, 14H), 2.53-2.60 (m, 1H), 2.72 (s, 4H), 2.87 (t, J=6.7 Hz, 2H), 3.01 (d, J=13.2 Hz, 3H), 3.06-3.20 (m, 3H), 3.48 (dd, J=6.8, 2.1 Hz, 2H), 3.58-3.65 (m, 2H), 3.71-3.83 (m, 3H), 4.03 (d, J=3.9 Hz, 3H), 4.06 (d, J=6.7 Hz, 2H), 4.25-4.36 (m, 4H), 4.46-4.55 (m, 2H), 5.80 (d, J=3.1 Hz, 2H), 7.24 (d, J=2.6 Hz, 1H), 7.31-7.37 (m, 2H), 7.39-7.45 (m, 2H), 7.87 (dd, J=9.2, 5.7 Hz, 1H), 9.16 (s, 1H). ¹⁹F NMR (376 MHz, CD₃OD) δ−139.21 (d, J=11.9 Hz), −129.35-−128.55 (m, 1H), −111.51 (s, 1H) −76.99 (m, 1H) (s, 12H). m/z, (ESI⁺): 980.38.

### Synthesis of Compound 207

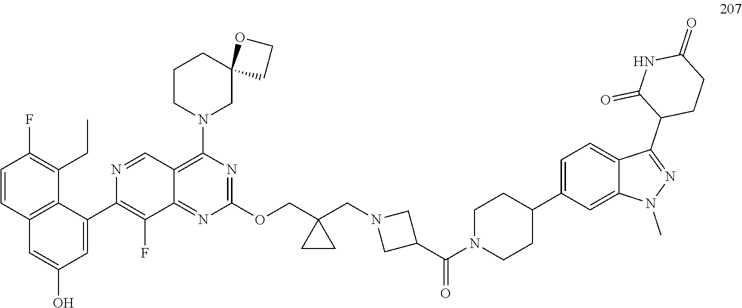 [SMILES: CCc1c(F)ccc2cc(O)cc(-c3ncc4c(N5CCC[C@]6(CCO6)C5)nc(OCC5(CN6CC(C(=O)N7CCC(c8ccc9c(C%10CCC(=O)NC%10=O)nn(C)c9c8)CC7)C6)CC5)nc4c3F)c12]

207 was synthesized according to the procedure of 201 with IM12 as starting material. ¹H NMR (400 MHz, CD₃OD) δ 0.57 (s, 2H), 0.66 (s, 2H), 0.82 (q, J=7.2 Hz, 3H), 1.60-1.93 (m, 7H), 2.16-2.31 (m, 3H), 2.39-2.51 (m, 4H), 2.57-2.79 (m, 5H), 2.94 (t, J=12.4 Hz, 1H), 3.15 (t, J=12.4 Hz, 1H), 3.38-3.42 (m, 2H), 3.59-3.83 (m, 5H), 3.97 (s, 3H), 4.29-4.45 (m, 4H), 4.50-4.65 (m, 5H), 7.01-7.08 (m, 2H), 7.23 (t, J=9.2 Hz, 1H), 7.28 (s, 1H), 7.33 (s, 1H), 7.58-7.67 (m, 2H), 9.19-9.24 (m, 1H). m/z, (ESI⁺): 954.32.

### Synthesis of Compound 208

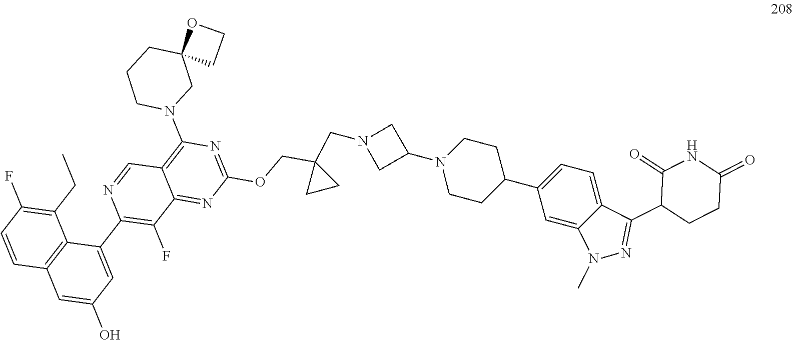 [SMILES: CCc1c(F)ccc2cc(O)cc(-c3ncc4c(N5CCC[C@]6(CCO6)C5)nc(OCC5(CN6CC(N7CCC(c8ccc9c(C%10CCC(=O)NC%10=O)nn(C)c9c8)CC7)C6)CC5)nc4c3F)c12]

208 was synthesized according to the procedure of 201 with IM13 as starting material. ¹H NMR (400 MHz, CD₃OD) δ 0.56 (s, 2H), 0.66 (s, 2H), 0.80 (q, J=7.2 Hz, 3H), 1.79-2.03 (m, 8H), 2.16-2.34 (m, 3H), 2.40-2.52 (m, 4H), 2.61-2.77 (m, 5H), 2.90-2.92 (m, 2H), 3.03 (s, 3H), 3.38-3.50 (m, 1H), 3.65-3.67 (m, 2H), 3.77-3.86 (m, 1H), 3.99 (s, 3H), 4.30-4.47 (m, 4H), 4.51-4.67 (m, 4H), 7.05-7.08 (m, 2H), 7.24 (t, J=9.6 Hz, 1H), 7.29 (s, 1H), 7.33 (s, 1H), 7.62-7.69 (m, 2H), 9.25 (d, J=8.0 Hz, 1H). m/z, (ESI⁺): 926.61.

### Synthesis of Compound 209

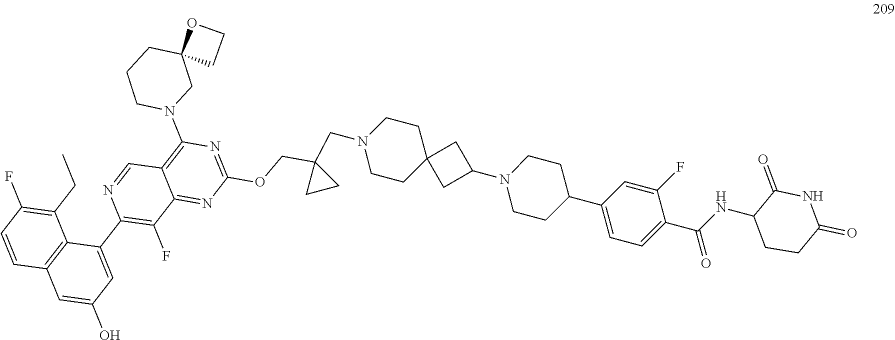 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(C(=O)NC9CCC(=O)NC9=O)c(F)c8)CC6)C7)CC5)nc4c3F)c12]

209 was synthesized according to the procedure of 201 with IM14 as starting material. ¹H NMR (400 MHz, CD₃OD) δ 0.51 (s, 2H), 0.73 (s, 2H), 0.81 (q, J=6.0 Hz, 3H), 1.54-2.03 (m, 18H), 2.14-2.21 (m, 2H), 2.26-2.33 (m, 2H), 2.37-2.53 (m, 8H), 2.61-2.86 (m, 4H), 3.01-3.04 (m, 2H), 3.39-3.51 (m, 1H), 3.79-3.87 (m, 1H), 4.38-4.68 (m, 7H), 7.07-7.14 (m, 2H), 7.18-7.20 (m, 1H), 7.25 (t, J=9.2 Hz, 1H), 7.30 (s, 1H), 7.65-7.69 (m, 1H), 7.7 (t, J=8.0 Hz, 1H), 9.25 (d, J=10.4 Hz, 1H). m/z, (ESI⁺):1001.33.

### Synthesis of Compound 210

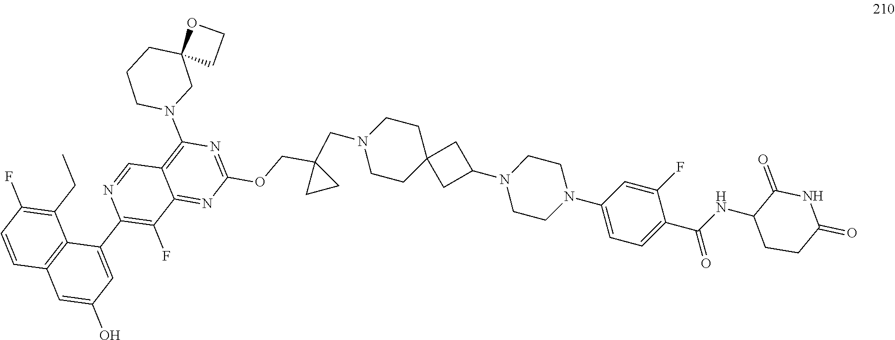 [SMILES: CCc1c(F)ccc2cc(O)cc(-c3ncc4c(N5CCC[C@]6(CCO6)C5)nc(OCC5(CN6CCC7(CC6)CC(N6CCN(c8ccc(C(=O)NC9CCC(=O)NC9=O)c(F)c8)CC6)C7)CC5)nc4c3F)c12]

210 was synthesized according to the procedure of 201 with IM15 as starting material. ¹H NMR (400 MHz, CD₃OD) δ 9.25 (d, J=10.3 Hz, 1H), 7.76 (t, J=9.0 Hz, 1H), 7.67 (dd, J=9.1, 5.8 Hz, 1H), 7.34-7.17 (m, 2H), 7.08 (d, J=2.6 Hz, 1H), 6.81 (dd, J=9.0, 2.4 Hz, 1H), 6.72 (d, J=2.4 Hz, 1H), 4.81-4.75 (m, 1H), 4.74-4.33 (m, 7H), 3.83 (dd, J=18.9, 13.6 Hz, 1H), 3.53-3.38 (m, 1H), 3.37-3.33 (m, 3H), 2.87-2.62 (m, 3H), 2.61-2.08 (m, 17H), 2.07-1.87 (m, 4H), 1.86-1.75 (m, 1H), 1.67-1.45 (m, 6H), 0.82 (q, J=7.0 Hz, 3H), 0.78-0.65 (m, 2H), 0.56-0.45 (m, 2H). ¹⁹F NMR (376 MHz, CD₃OD) δ−112.56 (dd, J=15.8, 9.4 Hz, 1F), −121.11(s, 1F), −139.01 (d, J=38.0 Hz, 1F). m/z, (ESI⁺): 1002.35.

### Synthesis of Compound 211

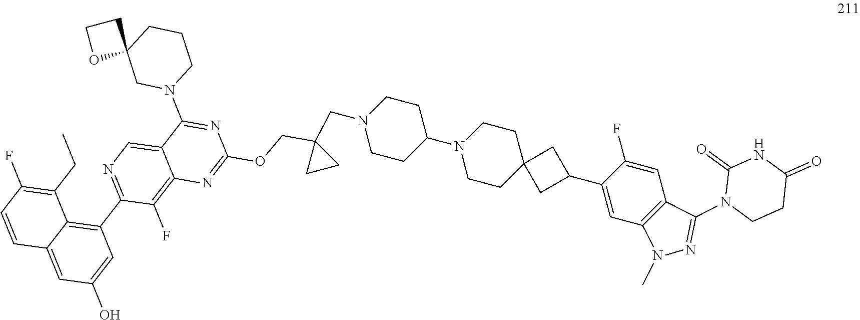 [SMILES: CCc1c(F)ccc2cc(O)cc(-c3ncc4c(N5CCC[C@]6(CCO6)C5)nc(OCC5(CN6CCC(N7CCC8(CC7)CC(c7cc9c(cc7F)c(N7CCC(=O)NC7=O)nn9C)C8)CC6)CC5)nc4c3F)c12]

211 was synthesized according to the procedure of 201 with IM16 as starting material. ¹H NMR (400 MHz, CD₃OD) δ 9.35 (d, J=15.5 Hz, 1H), 7.62-7.46 (m, 1H), 7.36 (d, J=10.4 Hz, 1H), 7.28 (s, 1H), 7.25-7.12 (m, 2H), 7.12-7.05 (m, 1H), 4.80-4.45 (m, 6H), 4.22-3.94 (m, 8H), 3.89 (dd, J=13.7, 3.7 Hz, 1H), 3.80-3.39 (m, 5H), 3.22-2.97 (m, 4H), 2.92 (t, J=6.8 Hz, 2H), 2.63-2.10 (m, 14H), 2.10-1.76 (m, 7H), 1.06 (s, 2H), 0.97-0.79 (m, 5H). ¹⁹F NMR (376 MHz, CD₃OD) δ−77.02 (s, 12F), −120.90 (s, 1F), −127.24 (s, 1F), −139.43-−139.94 (m, 1F). m/z, (ESI⁺):1013.37.

### Synthesis of Compound 212

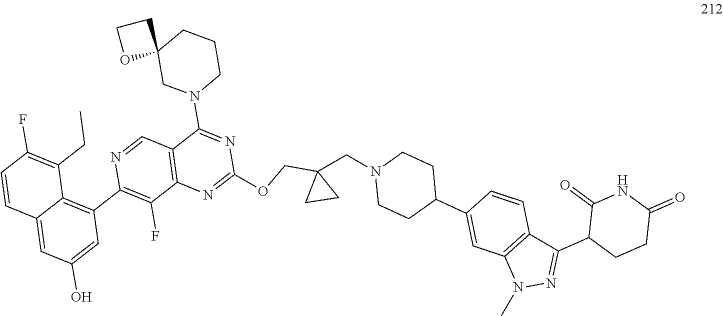 [SMILES: CCc1c(F)ccc2cc(O)cc(-c3ncc4c(N5CCC[C@]6(CCO6)C5)nc(OCC5(CN6CCC(c7ccc8c(C9CCC(=O)NC9=O)nn(C)c8c7)CC6)CC5)nc4c3F)c12]

212 was synthesized according to the procedure of 201 with M1 as starting material. ¹H NMR (400 MHz, CD₃OD) δ 0.91-0.97 (m, 3H), 1.04-1.14 (m, 2H), 1.30-1.42 (m, 6H), 1.69 (dd, J=23.3, 16.4 Hz, 2H), 1.87-2.11 (m, 3H), 2.22-2.35 (m, 7H), 2.43-2.52 (m, 4H), 2.79 (t, J=7.3 Hz, 2H), 3.09-3.28 (m, 4H), 3.39 (d, J=4.8 Hz, 2H), 3.86 (d, J=2.3 Hz, 3H), 4.02-4.10 (m, 2H), 4.39 (dd, J=9.2, 5.1 Hz, 1H), 4.49-4.67 (m, 5H), 7.11 (q, J=2.7 Hz, 2H), 7.27 (t, J=9.5 Hz, 1H), 7.33-7.39 (m, 2H), 7.66 (d, J=8.2 Hz, 1H), 7.71 (t, J=6.7 Hz, 1H), 9.33 (d, J=5.3 Hz, 1H). ¹⁹F NMR (376 MHz, CD₃OD) δ−138.85 (d, J=52.5 Hz, 1H), −120.87, (s, 1H), −77.24 (s, 12H). m/z, (ESI⁺):872.34.

### Synthesis of Compound 213

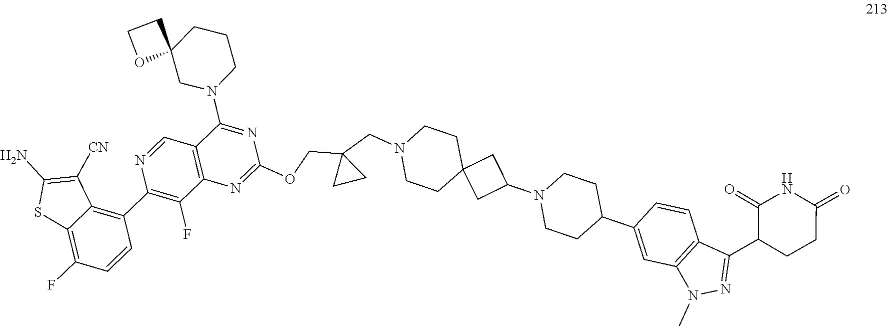 [SMILES: Cn1nc(C2CCC(=O)NC2=O)c2ccc(C3CCN(C4CC5(CCN(CC6(COc7nc(N8CCC[C@]9(CCO9)C8)c8cnc(-c9ccc(F)c%10sc(N)c(C#N)c9%10)c(F)c8n7)CC6)CC5)C4)CC3)cc21]

213 was synthesized according to the procedure of 86 with i as starting material. ¹H NMR (400 MHz, CD₃OD) δ 0.88-0.93 (m, 4H), 1.32-1.42 (m, 4H), 1.58-1.69 (m, 2H), 1.89-2.16 (m, 8H), 2.15-2.24 (m, 4H), 2.28-2.36 (m, 4H), 2.42-2.50 (m, 2H), 2.76 (q, J=9.4, 7.8 Hz, 2H), 2.94-3.06 (m, 4H), 3.08-3.18 (m, 2H), 3.47 (d, J=8.6 Hz, 2H), 3.62 (t, J=10.1 Hz, 2H), 3.81 (dd, J=25.4, 10.5 Hz, 4H), 4.03 (s, 3H), 4.48-4.56 (m, 2H), 4.57-4.66 (m, 2H), 7.01-7.14 (m, 2H), 7.39 (s, 1H), 7.41-7.50 (m, 1H), 7.71 (d, J=8.4 Hz, 1H), 9.30 (s, 1H). ¹⁹F NMR (376 MHz, CD₃OD) δ−136.69 (s, 1F), −113.71-−113.60 (m, 1F), −73.10 (s, 30F). m/z, (ESI⁺): 997.45.

### Synthesis of Compound 214

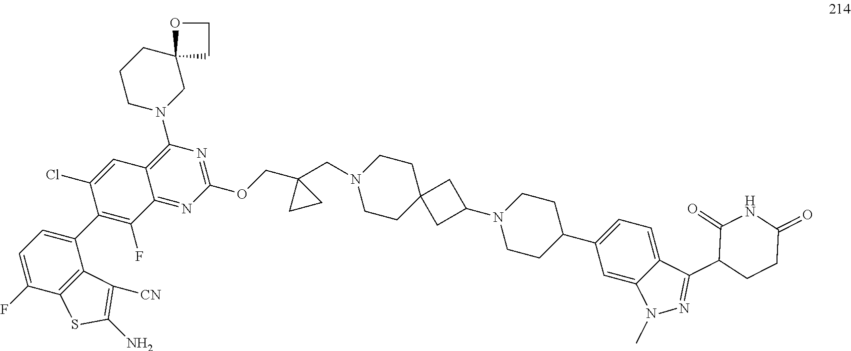 [SMILES: Cn1nc(C2CCC(=O)NC2=O)c2ccc(C3CCN(C4CC5(CCN(CC6(COc7nc(N8CCC[C@]9(CCO9)C8)c8cc(Cl)c(-c9ccc(F)c%10sc(N)c(C#N)c9%10)c(F)c8n7)CC6)CC5)C4)CC3)cc21]

214 was synthesized according to the procedure of 86 with j as starting material. ¹H NMR (400 MHz, CD₃OD) δ 0.87 (s, 2H), 1.00 (s, 2H), 1.29-1.30 (m, 5H), 1.78-2.35 (m, 16H), 2.44-2.54 (m, 3H), 2.70-2.81 (m, 1H), 2.93-3.14 (m, 4H), 3.30-3.76 (m, 7H), 4.02 (s, 3H), 4.23-4.63 (m, 7H), 7.04-7.11 (m, 2H), 7.22-7.25 (m, 1H), 7.38 (s, 1H), 7.69-7.71 (m, 1H), 8.25-8.29 (m, 1H). ¹⁹F NMR (376 MHz, CD₃OD) δ−124.32 (d, J=47.3 Hz 1H), −118.38, (s, 1H), −77.04 (s, 18H). m/z, (ESI⁺): 1029.25.

### Synthesis of Compound 215

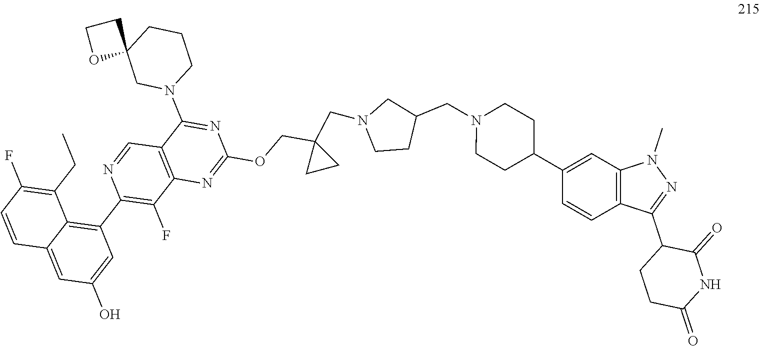 [SMILES: CCc1c(F)ccc2cc(O)cc(-c3ncc4c(N5CCC[C@]6(CCO6)C5)nc(OCC5(CN6CCC(CN7CCC(c8ccc9c(C%10CCC(=O)NC%10=O)nn(C)c9c8)CC7)C6)CC5)nc4c3F)c12]

215 was synthesized according to the procedure of 201 with IM17 as starting material. ¹H NMR (400 MHz, CD₃OD) δ 0.78 (d, J=4.9 Hz, 2H), 0.95 (d, J=6.5 Hz, 2H), 1.67 (dd, J=18.4, 11.5 Hz, 3H), 1.82-1.97 (m, 7H), 2.23 (t, J=6.3 Hz, 3H), 2.29-2.40 (m, 5H), 2.44-2.56 (m, 5H), 2.63 (s, 3H), 2.77 (dd, J=9.0, 5.5 Hz, 3H), 3.10-3.25 (m, 5H), 3.84 (t, J=13.4 Hz, 2H), 4.00-4.04 (m, 4H), 4.34-4.42 (m, 2H), 4.43-4.61 (m, 8H), 7.08 (ddd, J=10.1, 7.4, 4.1 Hz, 2H), 7.23-7.30 (m, 1H), 7.3-7.38 (m, 2H), 7.62-7.74 (m, 2H), 9.27-9.35 (m, 1H). ¹⁹F NMR (376 MHz, CD₃OD) δ−139.34 (d, J=50.9 Hz 1F), −120.98 (t, J=8.5 Hz 1F), −76.90, (s, 0.52F). m/z, (ESI⁺): 955.42.

### Synthesis of Compound 216

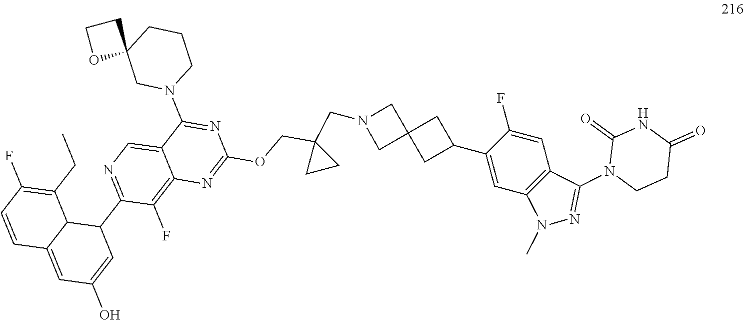 [SMILES: CCC1=C(F)C=CC2=CC(O)=CC(c3ncc4c(N5CCC[C@]6(CCO6)C5)nc(OCC5(CN6CC7(CC(c8cc9c(cc8F)c(N8CCC(=O)NC8=O)nn9C)C7)C6)CC5)nc4c3F)C21]

216 was synthesized according to the procedure of 201 with IM18 as starting material. ¹H NMR (400 MHz, CD₃OD) δ 9.27 (d, J=6.9 Hz, 1H), 7.68 (dd, J=9.0, 5.8 Hz, 1H), 7.40-7.17 (m, 4H), 7.09 (d, J=2.6 Hz, 1H), 4.72-4.33 (m, 6H), 4.07-3.94 (m, 5H), 3.90-3.78 (m, 1H), 3.71-3.57 (m, 3H), 3.53-3.37 (m, 3H), 2.86 (t, J=6.7 Hz, 2H), 2.76-2.58 (m, 4H), 2.58-2.43 (m, 3H), 2.43-2.24 (m, 3H), 2.24-2.12 (m, 1H), 2.10-1.88 (m, 2H), 1.88-1.76 (m, 1H), 0.87-0.76 (m, 3H), 0.74-0.65 (m, 2H), 0.65-0.56 (m, 2H). ¹⁹F NMR (376 MHz, CD₃OD) δ−121.11 (d, J=8.1 Hz, 1F), −127.63 (t, J=8.2 Hz, 1F), −139.05 (d, J=43.8 Hz, 1F). m/z, (ESI⁺): 902.59.

### Synthesis of Compound 217

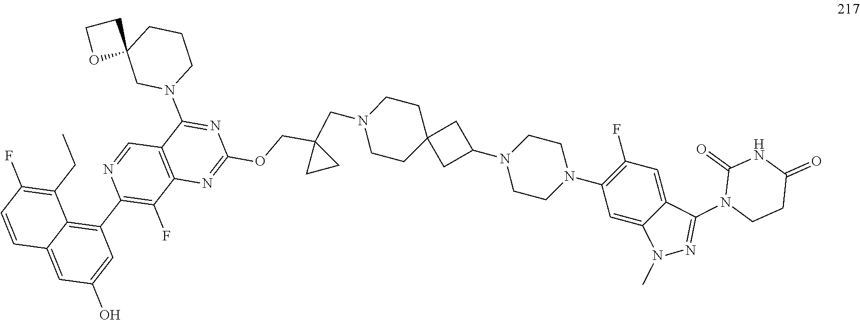 [SMILES: CCc1c(F)ccc2cc(O)cc(-c3ncc4c(N5CCC[C@]6(CCO6)C5)nc(OCC5(CN6CCC7(CC6)CC(N6CCN(c8cc9c(cc8F)c(N8CCC(=O)NC8=O)nn9C)CC6)C7)CC5)nc4c3F)c12]

217 was synthesized according to the procedure of 201 with IM19 as starting material. ¹H NMR (400 MHz, CD₃OD) δ: 0.94 (t, J=3.8 Hz, 4H), 0.98 (s, 2H), 1.03 (d, J=6.2 Hz, 3H), 1.11 (s, 2H), 1.73 (s, 3H), 1.97 (s, 2H), 2.06 (s, 3H), 2.32 (s, 4H), 2.45-2.49 (m, 2H), 2.63 (s, 2H), 2.81 (d, J=5.8 Hz, 5H), 2.98 (s, 2H), 3.14 (s, 2H), 3.67 (d, J=5.6 Hz, 2H), 3.93 (d, J=10.5 Hz, 2H), 4.10 (s, 3H), 4.14 (s, 2H), 7.15 (d, J=6.8 Hz, 1H), 7.20 (d, J=3.5 Hz, 1H), 7.38 (t, J=9.4 Hz, 1H), 7.44-7.49 (m, 2H), 7.81 (t, J=7.5 Hz, 1H), 8.54 (s, 3H), 9.41 (d, J=6.4 Hz, 1H). ¹⁹F NMR (376 MHz, CD₃OD) δ: −139.08-−138.92 (m, 1F), −130.72-−130.67 (m, 1F) −121.12 (s, 1F), −76.94 (s, 9F). m/z, (ESI⁺): 1014.32.

### Synthesis of Compound 218

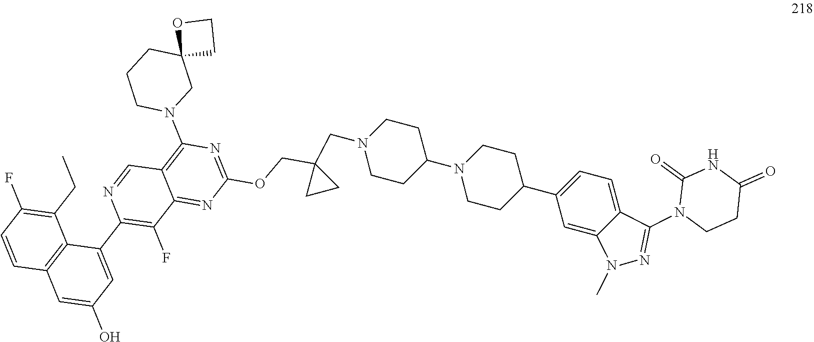 [SMILES: CCc1c(F)ccc2cc(O)cc(-c3ncc4c(N5CCC[C@]6(CCO6)C5)nc(OCC5(CN6CCC(N7CCC(c8ccc9c(N%10CCC(=O)NC%10=O)nn(C)c9c8)CC7)CC6)CC5)nc4c3F)c12]

218 was synthesized according to the procedure of 201 with IM20 as starting material. ¹H NMR (400 MHz, CD₃OD) δ 0.82 (q, J=7.2 Hz, 3H), 0.91 (s, 2H), 1.03 (s, 2H), 1.82-2.55 (m, 19H), 2.69-2.84 (m, 2H), 3.02 (t, J=10.8 Hz, 1H), 3.12-3.25 (m, 4H), 3.40-3.51 (m, 2H), 3.59-3.74 (m, 3H), 3.83-3.88 (m, 1H), 4.02 (s, 3H), 4.05-4.12 (m, 2H), 4.35-4.39 (m, 1H), 4.47-4.75 (m, 6H), 7.04-7.09 (m, 2H), 7.20 (t, J=10.4 Hz, 1H), 7.27 (s, 1H), 7.34 (s, 1H), 7.61-7.65 (m, 1H), 7.70 (d, J=8.4 Hz, 1H), 9.32 (d, J=13.2 Hz, 1H). m/z, (ESI⁺): 954.44.

### Synthesis of Compound 219

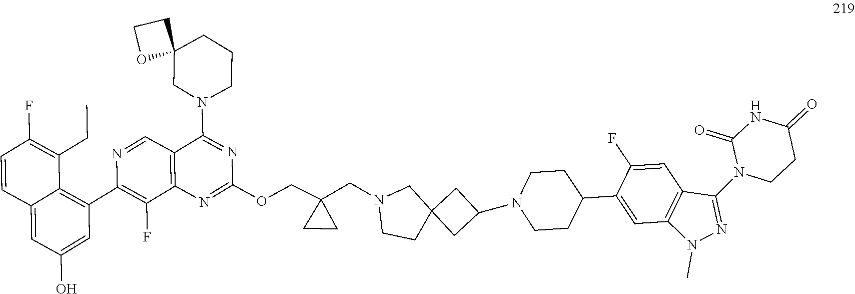 [SMILES: CCc1c(F)ccc2cc(O)cc(-c3ncc4c(N5CCC[C@]6(CCO6)C5)nc(OCC5(CN6CCC7(CC(N8CCC(c9cc%10c(cc9F)c(N9CCC(=O)NC9=O)nn%10C)CC8)C7)C6)CC5)nc4c3F)c12]

219 was synthesized according to the procedure of 201 with IM21 as starting material. ¹H NMR (400 MHz, CD₃OD) δ 0.66 (d, J=18.3 Hz, 2H), 0.85-0.88 (m, 2H), 1.75-2.01 (m, 11H), 1.77-2.11 (m, 16H), 2.15-2.46 (m, 7H), 2.47-2.64 (m, 4H), 3.02 (d, J=26.6 Hz, 6H), 3.45-3.54 (m, 1H), 3.87 (t, J=13.9 Hz, 1H), 4.01-4.13 (m, 6H), 4.53 (d, J=31.2 Hz, 4H), 7.12 (dd, J=7.6, 2.7 Hz, 1H), 7.22-7.33 (m, 2H), 7.36-7.45 (m, 2H), 7.67 (td, J=9.2, 5.8 Hz, 1H), 9.31 (d, J=12.6 Hz, 1H). ¹⁹F NMR (376 MHz, CD₃OD) δ−138.91 (dd, J=103.6, 35.4 Hz 1F), −129.00 (ddd, J=53.0, 11.2, 5.6 Hz 1F), −121.03 (d, J=9.5 Hz 1F), −76.91 (s, 0.7F). m/z, (ESI⁻): 998.26.

### Synthesis of Compound 220

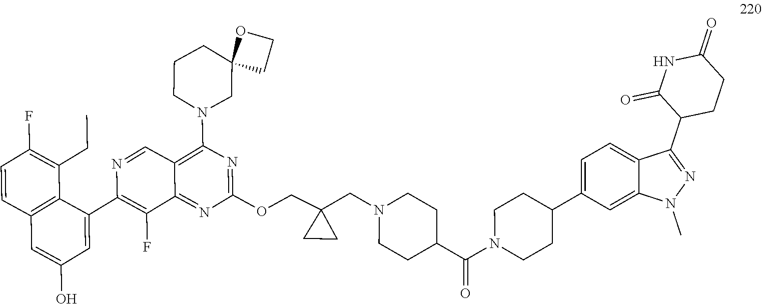 [SMILES: CCc1c(F)ccc2cc(O)cc(-c3ncc4c(N5CCC[C@]6(CCO6)C5)nc(OCC5(CN6CCC(C(=O)N7CCC(c8ccc9c(C%10CCC(=O)NC%10=O)nn(C)c9c8)CC7)CC6)CC5)nc4c3F)c12]

220 was synthesized according to the procedure of 201 with IM22 as starting material. ¹H NMR (400 MHz, CD₃OD) δ 0.68 (s, 2H), 0.79-0.90 (m, 5H), 1.61-1.73 (m, 2H), 1.82-2.06 (m, 9H), 2.16-2.55 (m, 8H), 2.67-3.03 (m, 7H), 3.40-3.57 (m, 2H), 3.79-3.86 (m, 1H), 3.98 (s, 3H), 4.15-4.19 (m, 1H), 4.32-4.36 (m, 1H), 4.41-4.72 (m, 10H), 7.05-7.10 (m, 2H), 7.20-7.34 (m, 3H), 7.62-7.68 (m, 2H), 9.27 (d, J=13.2 Hz, 1H). ¹⁹F NMR (376 MHz, CD₃OD) δ−139.12 (d, J=47.3 Hz 1H), −121.03, (s, 1H), −76.89 (s, 1H). m/z, (ESI⁺): 982.30.

### Synthesis of Compound 221

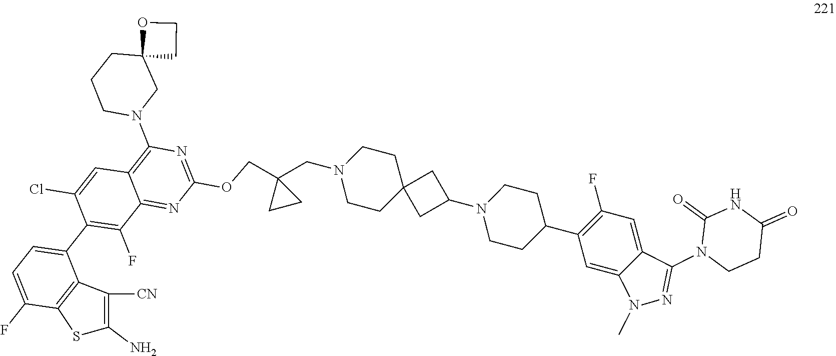 [SMILES: Cn1nc(N2CCC(=O)NC2=O)c2cc(F)c(C3CCN(C4CC5(CCN(CC6(COc7nc(N8CCC[C@]9(CCO9)C8)c8cc(Cl)c(-c9ccc(F)c%10sc(N)c(C#N)c9%10)c(F)c8n7)CC6)CC5)C4)CC3)cc21]

221 was synthesized according to the procedure of 214 with IM9 as starting material. ¹H NMR (400 MHz, CD₃OD) δ 0.87 (s, 2H), 1.01 (s, 2H), 1.81-2.37 (m, 17H), 2.45-2.55 (m, 3H), 2.87 (t, J=6.8 Hz 2H), 2.97-3.14 (m, 4H), 3.54-3.88 (m, 8H), 4.03-4.08 (m, 5H), 4.02 (s, 3H), 4.22-4.66 (m, 6H), 7.06 (t, J=8.8 Hz 1H), 7.22-7.26 (m, 1H), 7.40-7.44 (m, 2H), 8.25-8.29 (m, 1H). ¹⁹F NMR (376 MHz, CD₃OD) δ−128.32 (s, 1H), −124.32 (s, 1H), −118.41, (s, 1H), −77.05 (s, 18H). m/z, (ESI⁺): 1048.26.

### Synthesis of Compound 222

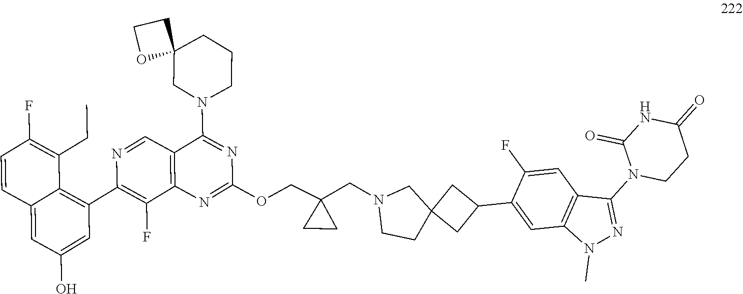 [SMILES: CCc1c(F)ccc2cc(O)cc(-c3ncc4c(N5CCC[C@]6(CCO6)C5)nc(OCC5(CN6CCC7(CC(c8cc9c(cc8F)c(N8CCC(=O)NC8=O)nn9C)C7)C6)CC5)nc4c3F)c12]

222 was synthesized according to the procedure of 201 with IM23 as starting material. ¹H NMR (400 MHz, CD₃OD) δ 0.48-0.60 (m, 2H), 0.69 (s, 2H), 1.76-2.01 (m, 3H), 2.04-2.49 (m, 13H), 2.62 (t, J=10.2 Hz, 2H), 2.79 (dd, J=14.8, 8.3 Hz, 5H), 3.33-3.44 (m, 1H), 3.64-3.80 (m, 2H), 3.89 (d, J=5.7 Hz, 3H), 3.95 (t, J=6.7 Hz, 2H), 4.42 (d, J=3.2 Hz, 2H), 4.45-4.55 (m, 4H), 4.60 (d, J=13.7 Hz, 2H), 7.01 (d, J=2.3 Hz, 1H), 7.18 (t, J=8.0 Hz, 1H), 7.20-7.25 (m, 2H), 7.28 (d, J=5.9 Hz, 1H), 7.61 (dd, J=9.0, 5.8 Hz, 1H), 9.20 (d, J=8.1 Hz, 1H). ¹⁹F NMR (376 MHz, CD₃OD) δ−139.01 (d, J=43.9 Hz 1F), −127.47 (dd, J=10.8, 5.7 Hz 1F), −121.07 (t, J=7.7 Hz 1F), −76.91 (s, 1.5F). m/z, (ESI⁺): 914.51.

### Synthesis of Compound 223

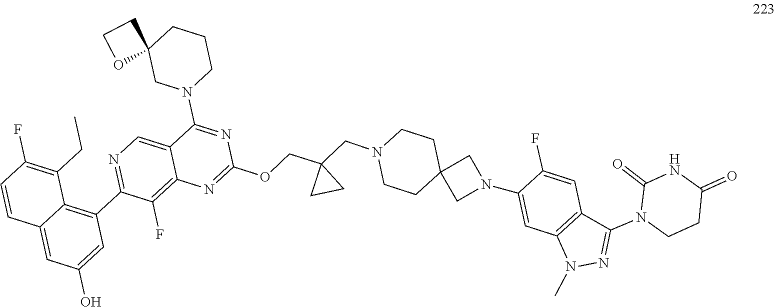

223 was synthesized according to the procedure of 201 with IM24 as starting material. ¹H NMR (400 MHz, CD₃OD) δ 6 9.26 (d, J=10.1 Hz, 1H), 7.68 (dd, J=9.0, 5.7 Hz, 1H), 7.33-7.15 (m, 3H), 7.09 (d, J=2.7 Hz, 1H), 6.37 (d, J=7.2 Hz, 1H), 4.73-4.31 (m, 7H), 3.99 (t, J=6.7 Hz, 2H), 3.92-3.78 (m, 4H), 3.77-3.67 (m, 4H), 3.55-3.38 (m, 1H), 2.84 (t, J=6.7 Hz, 2H), 2.60-2.37 (m, 8H), 2.34-1.90 (m, 4H), 1.91-1.73 (m, 5H), 0.83 (q, J=7.0 Hz, 3H), 0.78-0.66 (m, 2H), 0.57-0.45 (m, 2H). ¹⁹F NMR (376 MHz, CD₃OD) δ−121.08 (s, 1F), −137.79 (t, J=9.6 Hz, 1F), −139.02 (d, J=38.9 Hz, 1F). m/z, (ESI⁺): 931.61.

### Synthesis of Compound 224

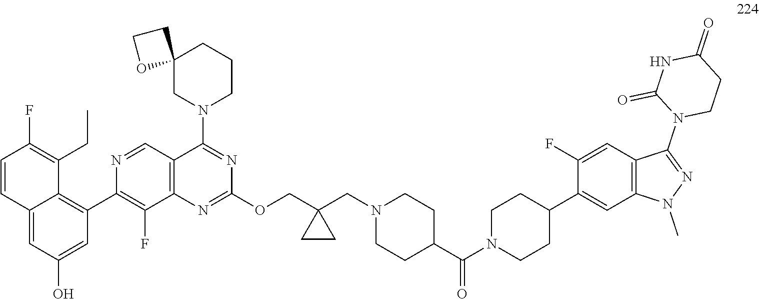 [SMILES: CCc1c(F)ccc2cc(O)cc(-c3ncc4c(N5CCC[C@]6(CCO6)C5)nc(OCC5(CN6CCC(C(=O)N7CCC(c8cc9c(cc8F)c(N8CCC(=O)NC8=O)nn9C)CC7)CC6)CC5)nc4c3F)c12]

224 was synthesized according to the procedure of 201 with IM25 as starting material. ¹H NMR (400 MHz, CD₃OD) δ: 0.54 (s, 2H), 0.77 (s, 2H), 0.82-0.93 (m, 3H), 1.76 (d, J=12.1 Hz, 7H), 1.94-2.13 (m, 6H), 2.18-2.37 (m, 2H), 2.52 (dd, J=23.3, 14.7 Hz, 5H), 2.76 (dd, J=26.7, 13.4 Hz, 2H), 2.90 (t, J=6.7 Hz, 2H), 3.17 (p, J=1.7 Hz, 2H), 3.45-3.62 (m, 1H), 3.91 (dd, J=22.1, 13.6 Hz, 1H), 4.01 (s, 3H), 4.06 (t, J=6.7 Hz, 2H), 4.20 (s, 1H), 4.41-4.53 (m, 2H), 4.56-4.69 (m, 5H), 4.71-4.82 (m, 2H), 7.14 (d, J=2.6 Hz, 1H), 7.27 (td, J=9.3, 4.3 Hz, 1H), 7.32 (d, J=3.1 Hz, 1H), 7.39 (d, J=10.8 Hz, 1H), 7.42 (d, J=5.7 Hz, 1H), 7.69 (dd, J=9.0, 5.8 Hz, 1H), 9.29 (d, J=8.4 Hz, 1H). ¹⁹F NMR (376 MHz, CD₃OD) δ: −139.07-−138.97(m, 1F),−129.19-−129.22 (m, 1F) −121.12 (s, 1F). m/z, (ESI⁺): 1001.51.

### Synthesis of Compound 225

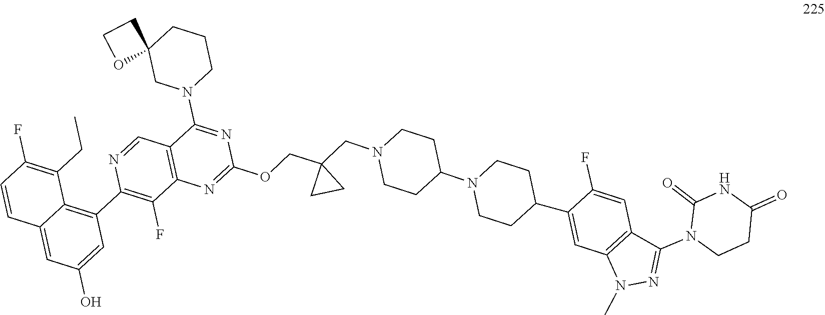

225 was synthesized according to the procedure of 201 with IM26 as starting material. ¹H NMR (400 MHz, CD₃OD) δ: 0.56 (s, 2H), 0.78 (s, 2H), 0.86 (q, J=7.2 Hz, 3H), 0.93 (t, J=6.5 Hz, 2H), 1.65 (s, 4H), 1.83-2.11 (m, 12H), 2.24 (d, J=8.3 Hz, 1H), 2.33 (d, J=13.0 Hz, 1H), 2.39 (t, J=7.4 Hz, 1H), 2.52 (d, J=22.7 Hz, 8H), 2.90 (t, J=6.7 Hz, 2H), 3.03 (d, J=11.9 Hz, 1H), 3.17 (dt, J=3.5, 1.8 Hz, 2H), 3.49-3.54 (m, 1H), 3.58 (dd, J=5.6, 1.6 Hz, 1H), 3.83-3.92 (m, 1H), 4.03-4.08 (m, 5H), 4.42-4.57 (m, 4H), 7.12 (d, J=2.6 Hz, 1H), 7.29 (d, J=9.5 Hz, 1H), 7.33 (d, J=2.7 Hz, 1H), 7.38 (d, J=10.6 Hz, 1H), 7.43 (d, J=5.7 Hz, 1H), 7.70 (dd, J=9.1, 5.8 Hz, 1H), 9.30 (d, J=9.8 Hz, 1H). ¹⁹F NMR (376 MHz, CD₃OD) δ: −139.07-−138.96(m, 1F),−129.09-−129.04 (m, 1F) −121.06 (s, 1F). m/z, (ESI⁺): 973.52.

### Synthesis of Compound 226

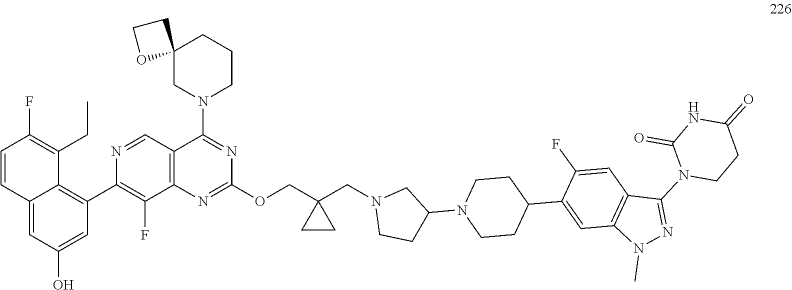 [SMILES: CCc1c(F)ccc2cc(O)cc(-c3ncc4c(N5CCC[C@]6(CCO6)C5)nc(OCC5(CN6CCC(N7CCC(c8cc9c(cc8F)c(N8CCC(=O)NC8=O)nn9C)CC7)C6)CC5)nc4c3F)c12]

226 was synthesized according to the procedure of 201 with IM27 as starting material. ¹H NMR (400 MHz, CD₃OD) δ 0.68 (s, 2H), 0.73-0.80 (m, 2H), 0.83 (td, J=7.7, 3.1 Hz, 3H), 1.24-1.39 (m, 2H), 1.83-2.38 (m, 13H), 2.43-2.64 (m, 3H), 2.66-2.82 (m, 4H), 3.02-3.25 (m, 4H), 3.41-3.53 (m, 3H), 3.91 (s, 3H), 4.06 (td, J=6.8, 2.2 Hz, 2H), 4.41-4.53 (m, 2H), 6.95-7.03 (m, 1H), 7.19-7.30 (m, 2H), 7.32-7.39 (m, 2H), 7.66 (dt, J=9.2, 6.3 Hz, 1H), 8.56 (s, 1H), 9.28 (dd, J=14.3, 3.0 Hz, 1H). ¹⁹F NMR (376 MHz, CD₃OD) δ−139.47-−138.63 (m 1F), −129.25-−128.71 (m 1F), −121.47-−120.70 (m 1F). m/z, (ESI⁺): 960.04.

### Synthesis of Compound 227

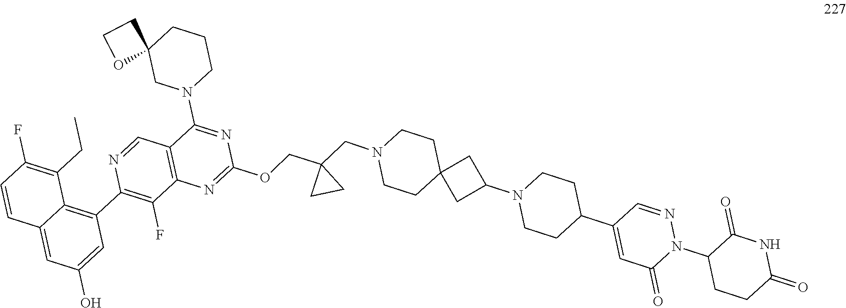 [SMILES: CCc1c(F)ccc2cc(O)cc(-c3ncc4c(N5CCC[C@]6(CCO6)C5)nc(OCC5(CN6CCC7(CC6)CC(N6CCC(c8cnn(C9CCC(=O)NC9=O)c(=O)c8)CC6)C7)CC5)nc4c3F)c12]

227 was synthesized according to the procedure of 201 with IM28 as starting material. ¹H NMR (400 MHz, CD₃OD) δ: 0.55 (s, 2H), 0.76 (s, 2H), 0.85 (q, J=7.0 Hz, 3H), 1.21 (t, J=7.0 Hz, 1H), 1.66 (td, J=25.4, 24.0, 12.6 Hz, 9H), 1.77-2.02 (m, 8H), 2.17-2.27 (m, 2H), 2.29-2.38 (m, 2H), 2.42-2.67 (m, 10H), 2.69-2.95 (m, 4H), 3.05 (d, J=11.0 Hz, 2H), 3.41-3.56 (m, 1H), 3.87 (dd, J=18.1, 13.5 Hz, 1H), 4.41-4.54 (m, 3H), 4.58 (s, 4H), 5.75 (dd, J=12.0, 5.4 Hz, 1H), 6.81 (dd, J=11.0, 2.0 Hz, 1H), 7.11 (d, J=2.6 Hz, 1H), 7.28 (t, J=9.4 Hz, 1H), 7.34 (d, J=2.7 Hz, 1H), 7.71 (dd, J=9.0, 5.8 Hz, 1H), 7.99 (dd, J=10.4, 2.2 Hz, 1H), 9.29 (d, J=10.4 Hz, 1H). ¹⁹F NMR (376 MHz, CD₃OD) δ: −139.06-−138.96 (m, 1F), −121.19s, 1F). m/z, (ESI⁺): 958.48.

### Synthesis of Compound 228

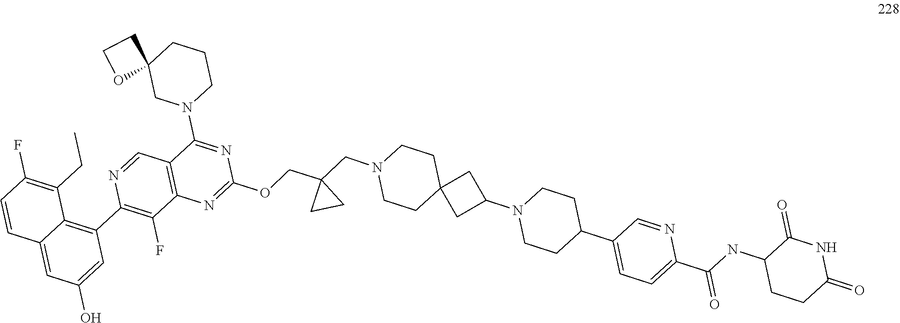 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(C(=O)NC9CCC(=O)NC9=O)nc8)CC6)C7)CC5)nc4c3F)c12]

228 was synthesized according to the procedure of 201 with IM29 as starting material. ¹H NMR (400 MHz, CD₃OD) δ: 0.84 (d, J=7.4 Hz, 2H), 0.86-0.868 (m, 2H), 1.64 (d, J=7.7 Hz, 1H), 1.73-1.91 (m, 10H), 1.94-2.02 (m, 4H), 2.03-2.29 (m, 9H), 2.30-2.44 (m, 3H), 2.54 (tt, J 8.7, 4.0 Hz, 4H), 2.66-3.07 (m, 10H), 3.17 (dt, J=3.4, 1.7 Hz, 3H), 3.44-3.52 (m, 1H), 4.43-4.56 (m, 4H), 7.11 (t, J=2.4 Hz, 1H), 7.29 (t, J=9.4 Hz, 1H), 7.35 (d, J=2.7 Hz, 1H), 7.72 (dd, J=9.1, 5.8 Hz, 1H), 7.89 (dd, J=8.1, 2.3 Hz, 1H), 8.10 (d, J=8.1 Hz, 1H), 8.60 (d, J=2.2 Hz, 1H), 9.31 (d, J=8.9 Hz, 1H). ¹⁹F NMR (376 MHz, CD₃OD) δ−121.04 (t, J=8.0 Hz, 1F), −139.07 (d, J=46.5 Hz, 1F). m/z, (ESI⁺): 985.46.

### Synthesis of Compound 229

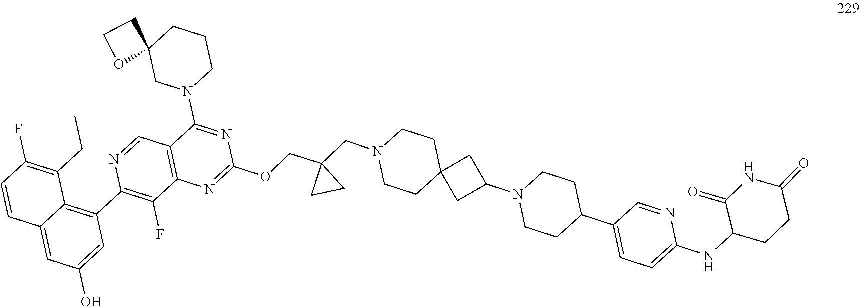

229 was synthesized according to the procedure of 201 with IM30 as starting material. ¹H NMR (400 MHz, CD₃OD) δ: 0.69 (s, 2H), 0.86 (s, 2H), 0.94 (d, J=6.5 Hz, 3H), 1.64 (s, 2H), 1.78 (s, 10H), 1.89 (s, 4H), 2.21 (dd, J=20.0, 12.3 Hz, 8H), 2.36 (dd, J=14.2, 9.9 Hz, 5H), 2.54 (s, 6H), 2.72-2.90 (m, 8H), 3.63 (s, 1H), 3.87 (t, J=13.7 Hz, 1H), 6.66 (d, J=8.6 Hz, 1H), 7.11 (t, J=2.3 Hz, 1H), 7.29 (t, J=9.4 Hz, 1H), 7.35 (d, J=2.6 Hz, 1H), 7.40-7.46 (m, 1H), 7.72 (dd, J=9.0, 5.8 Hz, 1H), 7.88 (d, J=2.4 Hz, 1H), 8.57 (s, 1H), 9.30 (d, J=9.4 Hz, 1H). ¹⁹F NMR (376 MHz, CD₃OD) δ: −139.00-−139.12 (m, 1F), −121.04 (s, 1F). m/z, (ESI⁺): 956.48.

### Synthesis of Compound 230

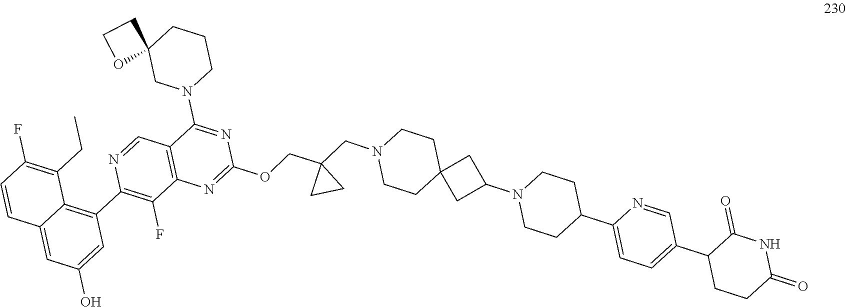 [SMILES: CCc1c(F)ccc2cc(O)cc(-c3ncc4c(N5CCC[C@]6(CCO6)C5)nc(OCC5(CN6CCC7(CC6)CC(N6CCC(c8ccc(C9CCC(=O)NC9=O)cn8)CC6)C7)CC5)nc4c3F)c12]

230 was synthesized according to the procedure of 201 with IM31 as starting material. ¹H NMR (400 MHz, CD₃OD) δ0.91 (s, 3H), 1.05 (s, 3H), 1.85 (tt, J=12.0, 5.0 Hz, 2H), 1.94-2.02 (m, 3H), 2.03-2.18 (m, 10H), 2.22 (d, J=7.4 Hz, 7H), 2.30-2.43 (m, 5H), 2.49-2.63 (m, 5H), 2.67-2.90 (m, 4H), 2.95-3.18 (m, 7H), 3.39-3.54 (m, 3H), 3.65 (d, J=12.2 Hz, 3H), 3.72-3.93 (m, 6H), 4.02 (dd, J=12.6, 4.9 Hz, 1H), 4.47-4.70 (m, 7H), 7.11 (t, J=3.0 Hz, 1H), 7.30 (s, 1H), 7.35 (d, J=2.7 Hz, 1H), 7.43 (d, J=9.0 Hz, 1H), 7.72 (dd, J=9.0, 5.8 Hz, 1H), 7.81 (d, J=9.8 Hz, 1H), 8.50 (s, 1H), 9.33 (d, J=7.6 Hz, 1H). ¹⁹F NMR (376 MHz, CD₃OD) δ −139.38-−139.18 (m 1F), −120.95 (t, J=8.1 Hz 1F), −77.08 (s, 21F). m/z, (ESI⁺): 941.78.

### Synthesis of Compound 231

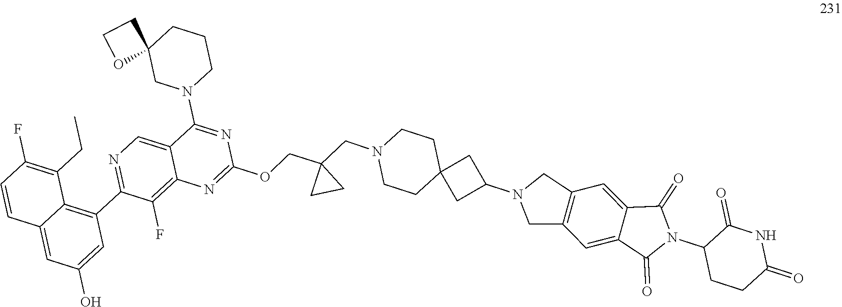 [SMILES: CCc1c(F)ccc2cc(O)cc(-c3ncc4c(N5CCC[C@]6(CCO6)C5)nc(OCC5(CN6CCC7(CC6)CC(N6Cc8cc9c(cc8C6)C(=O)N(C6CCC(=O)NC6=O)C9=O)C7)CC5)nc4c3F)c12]

231 was synthesized according to the procedure of 201 with IM32 as starting material. ¹H NMR (400 MHz, CD₃OD) δ 9.31 (d, J=7.1 Hz, 1H), 7.93 (s, 2H), 7.71 (dd, J=9.0, 5.7 Hz, 1H), 7.34 (d, J=2.7 Hz, 1H), 7.28 (t, J=9.4 Hz, 1H), 7.09 (t, J=2.9 Hz, 1H), 5.19 (dd, J=12.6, 5.4 Hz, 1H), 4.82-4.64 (m, 4H), 4.63-4.38 (m, 5H), 4.19-4.02 (m, 1H), 3.94-3.67 (m, 3H), 3.53-3.35 (m, 2H), 3.17-2.96 (m, 2H), 2.96-2.57 (m, 5H), 2.57-1.70 (m, 17H), 1.07-0.98 (m, 2H), 0.93-0.85 (m, 2H), 0.87-0.78 (m, 3H). m/z, (ESI⁺): 967.31.

### Synthesis of Compound 232

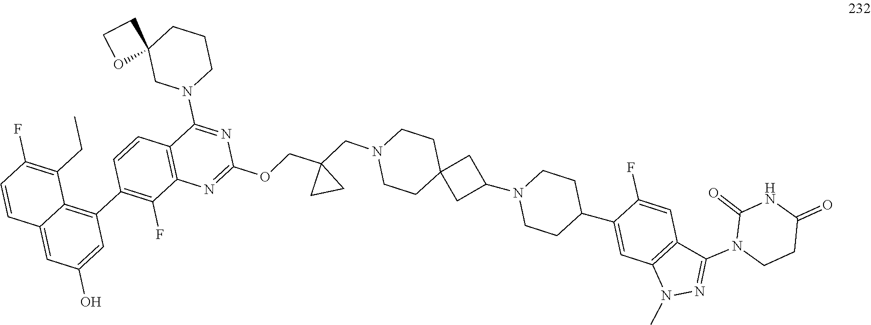

232 was synthesized according to the procedure of 202 with k as starting material. ¹H NMR (400 MHz, CD₃OD) δ 0.85 (t, J=7.4 Hz, 3H). 0.94 (s, 2H), 1.07 (s, 2H), 2.08 (s, 4H), 2.23 (t, J=15.3 Hz, 10H), 2.40 (d, J=18.4 Hz, 4H), 2.56 (d, J=7.9 Hz, 2H), 2.96-3.19 (m, 5H), 3.66 (d, J=11.9 Hz, 3H), 3.80 (d, J=6.5 Hz, 5H), 4.07 (d, J=3.9 Hz, 6H), 4.17 (d, J=6.4 Hz, 1H), 4.51-4.63 (m, 3H), 6.98-7.12 (m, 2H), 7.25-7.32 (m, 2H), 7.42-7.51 (m, 3H), 7.67-7.73 (m, 1H), 8.19 (dd, J=34.0, 9.0 Hz, 1H). ¹⁹F NMR (376 MHz, CD₃OD) δ−129.85 (s, 1F), 128.96 (t, J=8.3 Hz 1F), −120.88 (s, 1F), −77.08 (s, 15F). m/z, (ESI⁺):1012.40.

### Synthesis of Compound 233

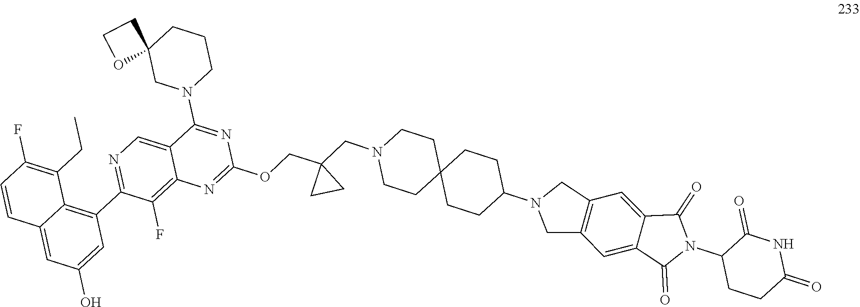 [SMILES: CCc1c(F)ccc2cc(O)cc(-c3ncc4c(N5CCC[C@]6(CCO6)C5)nc(OCC5(CN6CCC7(CCC(N8Cc9cc%10c(cc9C8)C(=O)N(C8CCC(=O)NC8=O)C%10=O)CC7)CC6)CC5)nc4c3F)c12]

233 was synthesized according to the procedure of 201 with IM33 as starting material. ¹H NMR (400 MHz, CD₃OD) δ 9.30 (d, J=7.1 Hz, 1H), 7.92 (s, 2H), 7.69 (dd, J=9.1, 5.8 Hz, 1H), 7.32 (d, J=2.7 Hz, 1H), 7.26 (t, J=9.4 Hz, 1H), 7.08 (t, J=2.8 Hz, 1H), 5.17 (dd, J=12.6, 5.4 Hz, 1H), 4.70 (dd, J=34.7, 13.7 Hz, 1H), 4.61-4.37 (m, 5H), 3.90-3.63 (m, 3H), 3.60-3.34 (m, 3H), 3.21-3.05 (m, 2H), 2.95-2.61 (m, 3H), 2.59-1.44 (m, 21H), 1.43-1.20 (m, 2H), 1.05-0.96 (m, 2H), 0.91-0.85 (m, 2H), 0.86-0.76 (m, 3H). ¹⁹F NMR (376 MHz, CD₃OD) δ−77.15 (s, 12F), −120.92 (s, 1F), −138.80-−139.53 (m, 1F). m/z, (ESI⁺): 995.28.

### Synthesis of Compound 234

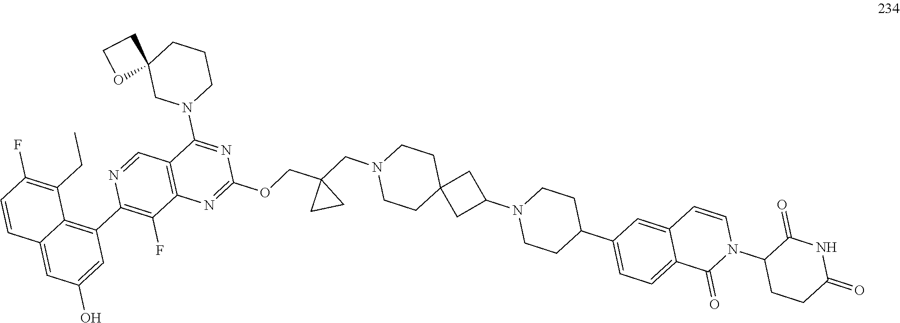 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9c(=O)n(C%10CCC(=O)NC%10=O)ccc9c8)CC6)C7)CC5)nc4c3F)c12]

234 was synthesized according to the procedure of 201 with IM34 as starting material. ¹H NMR (400 MHz, CD₃OD) δ 0.59 (s, 2H), 0.80 (s, 2H), 0.86 (d, J=6.3 Hz, 3H), 0.93 (t, J=7.1 Hz, 2H), 1.72 (d, J=9.2 Hz, 8H), 1.86 (d, J=11.8 Hz, 4H), 1.93-2.12 (m, 9H), 2.24 (d, J=10.3 Hz, 2H), 2.34 (t, J=11.1 Hz, 2H), 2.53 (d, J=12.5 Hz, 6H), 2.73-2.91 (m, 6H), 3.10-3.17 (m, 2H), 3.87 (dd, J=17.2, 13.5 Hz, 1H), 4.43-4.55 (m, 3H), 6.75 (d, J=7.5 Hz, 1H), 7.12 (d, J=2.6 Hz, 1H), 7.29 (t, J=9.3 Hz, 1H), 7.35 (d, J=7.3 Hz, 2H), 7.49 (dd, J=8.5, 1.7 Hz, 1H), 7.55 (s, 1H), 7.71 (dd, J=9.1, 5.8 Hz, 1H), 8.25 (d, J=8.4 Hz, 1H), 9.30 (d, J=10.0 Hz, 1H). ¹⁹F NMR (376 MHz, CD₃OD) δ−138.98-−139.08 (m, 1F), −121.10 (s, 1F). m/z, (ESI⁺): 1007.32.

### Synthesis of Compound 235

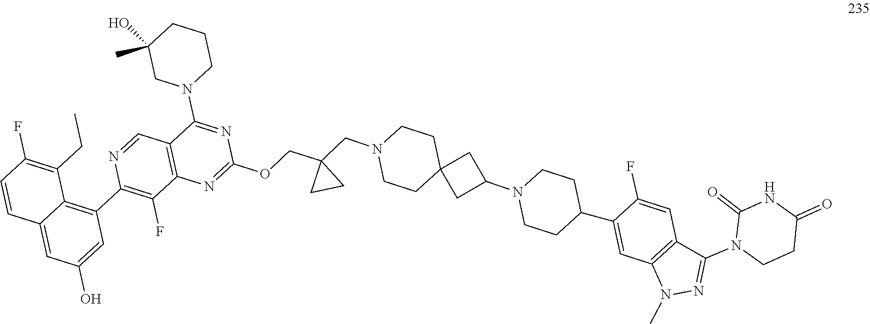

235 was synthesized according to the procedure of 202 with a as starting material. ¹H NMR (400 MHz, CD₃OD) 0.57 (s, 2H), 0.77 (s, 2H), 0.85 (q, J=7.3 Hz, 3H), 1.68 (dd, J=23.8, 11.6 Hz, 7H), 1.78-2.10 (m, 12H), 2.23 (dq, J=14.5, 7.2 Hz, 3H), 2.42-2.73 (m, 7H), 2.90 (t, J=6.7 Hz, 2H), 2.99 (d, J=11.7 Hz, 1H), 3.11 (d, J=11.0 Hz, 2H), 3.50 (q, J=11.0, 10.3 Hz, 1H), 3.60-3.77 (m, 1H), 4.03-4.08 (m, 5H), 4.29 (t, J=13.3 Hz, 1H), 4.40-4.51 (m, 2H), 4.54 (d, J=13.1 Hz, 1H), 4.63 (s, 2H), 7.09 (d, J=2.6 Hz, 1H), 7.28 (t, J=9.4 Hz, 1H), 7.33 (d, J=2.7 Hz, 1H), 7.38 (d, J=10.7 Hz, 1H), 7.44 (d, J=5.8 Hz, 1H), 7.70 (dd, J=9.1, 5.8 Hz, 1H), 9.24 (d, J=3.8 Hz, 1H). ¹⁹F NMR (376 MHz, CD₃OD) −139.20 (d, J=14.8 Hz, 1F). -129.67-−128.26 (m, 1F), −121.20 (q, J=11.6, 9.0 Hz, 1F). m/z, (ESI⁺): 1001.58.

### Synthesis of Compound 236

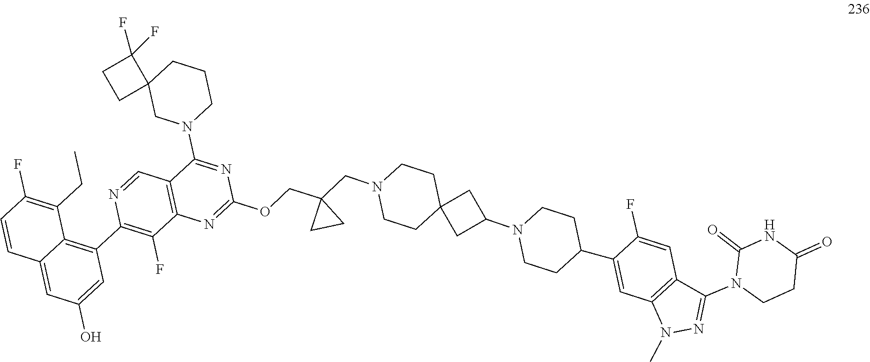

236 was synthesized according to the procedure of 202 with 1 as starting material. ¹H NMR (400 MHz, CD₃OD) δ 0.59 (s, 2H), 0.79 (s, 2H), 0.85 (d, J=6.8 Hz, 3H), 1.56-1.76 (m, 7H), 1.83-2.29 (m, 15H), 2.46-2.68 (m, 6H), 2.90 (t, J=6.8 Hz, 3H), 2.97-3.06 (m, 1H), 3.12 (d, J=11.1 Hz, 2H), 4.06 (d, J=3.2 Hz, 6H), 4.41-4.57 (m, 3H), 4.80 (s, 2H), 7.13 (d, J=2.6 Hz, 1H), 7.29 (s, 1H), 7.34 (d, J=2.7 Hz, 1H), 7.38 (d, J=10.6 Hz, 1H), 7.44 (d, J=5.7 Hz, 1H), 7.71 (dd, J=9.0, 5.8 Hz, 1H), 9.20 (s, 1H). ¹⁹F NMR (376 MHz, CD₃OD) δ−138.77-−138.81(m, 1F), −129.04-−129.09 (m, 1F), −120.99-−121.03 (s, 1F), −114.31-−115.87 (m, 2F). m/z, (ESI⁺): 1049.55.

### Synthesis of Compound 237

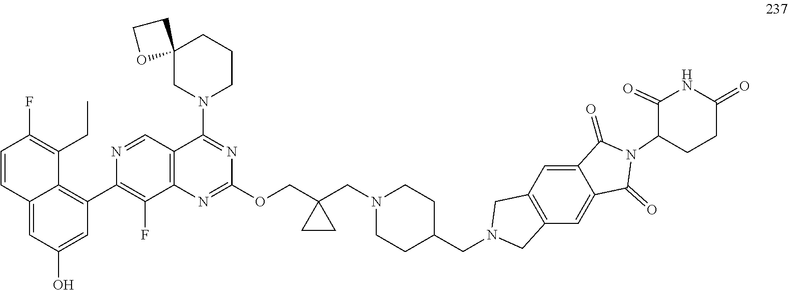 [SMILES: CCc1c(F)ccc2cc(O)cc(-c3ncc4c(N5CCC[C@]6(CCO6)C5)nc(OCC5(CN6CCC(CN7Cc8cc9c(cc8C7)C(=O)N(C7CCC(=O)NC7=O)C9=O)CC6)CC5)nc4c3F)c12]

237 was synthesized according to the procedure of 231 with tert-butyl 4-formylpiperidine-1-carboxylate as starting material. ¹H NMR (400 MHz, CD₃OD) δ ppm 9.21 (d, J=7.1 Hz, 1H), 7.69-7.56 (m, 3H), 7.24 (d, J=2.6 Hz, 1H), 7.18 (t, J=9.3 Hz, 1H), 7.01 (d, J=2.6 Hz, 1H), 5.06 (dd, J=12.6, 5.4 Hz, 1H), 4.70-4.28 (m, 6H), 3.94 (s, 4H), 3.74 (dd, J=17.2, 13.6 Hz, 1H), 3.46-3.25 (m, 4H), 2.87-2.51 (m, 7H), 2.50-2.18 (m, 6H), 2.17-1.50 (m, 10H), 0.81-0.67 (m, 5H), 0.67-0.48 (m, 2H); ¹⁹F NMR (376 MHz, CD₃OD) δ ppm −121.04(t, J=7.6 Hz, 1F), −139.01 (d, J=47.6 Hz, 1F). m/z, (ESI⁺): 941.51.

### Synthesis of Compound 238

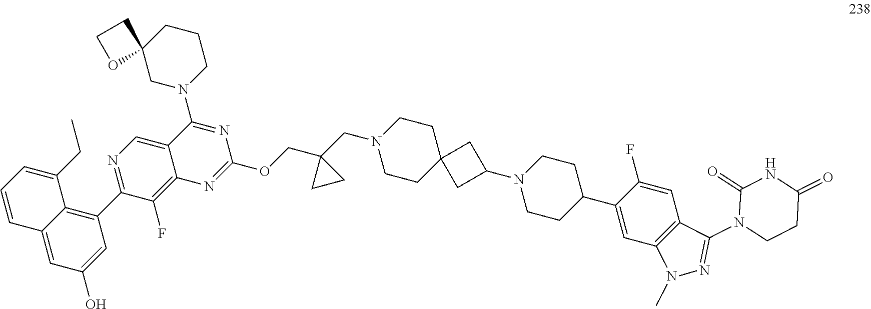 [SMILES: CCc1cccc2cc(O)cc(-c3ncc4c(N5CCC[C@]6(CCO6)C5)nc(OCC5(CN6CCC7(CC6)CC(N6CCC(c8cc9c(cc8F)c(N8CCC(=O)NC8=O)nn9C)CC6)C7)CC5)nc4c3F)c12]

238 was synthesized according to the procedure of 202 with 2-(8-ethyl-3-(methoxymethoxy)naphthalen-1-yl)-4,4,5,5-tetramethyl-1,3,2-dioxaborolane as starting material. ¹H NMR (400 MHz, CD₃OD) δ ppm 9.20 (s, 1H), 7.58 (d, J=8.2 Hz, 1H), 7.36 (d, J=5.7 Hz, 1H), 7.34-7.27 (m, 2H), 7.24 (d, J=2.7 Hz, 1H), 7.12 (d, J=7.1 Hz, 1H), 6.99 (d, J=2.7 Hz, 1H), 4.69-4.23 (m, 7H), 4.03-3.92 (m, 5H), 3.78 (dd, J=21.9, 13.6 Hz, 1H), 3.49-3.33 (m, 1H), 3.12-3.00 (m, 2H), 2.99-2.87 (m, 1H), 2.86-2.68 (m, 3H), 2.67-2.12 (m, 10H), 2.10-1.70 (m, 12H), 1.69-1.49 (m, 6H), 0.91-0.82 (m, 3H), 0.77-0.63 (m, 2H), 0.60-0.37 (m, 2H). ¹⁹F NMR (376 MHz, CD₃OD) δ ppm −129.11(dd, J=10.1, 5.8 Hz, 1F), −139.28 (d, J=49.2 Hz, 1F). m/z, (ESI⁺): 995.55.

### Synthesis of Compound 239

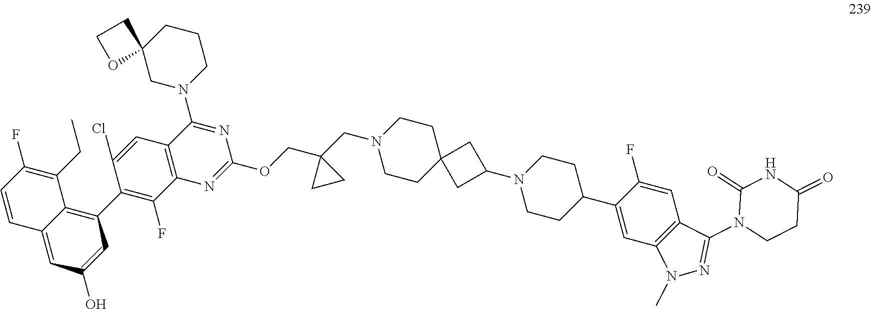

239 was synthesized according to the procedure of 202 with j-1 as starting material. ¹H NMR (400 MHz, CD₃OD) δ 0.85 (s, 2H), 0.94 (t, J=6.7 Hz, 2H), 1.64 (d, J=6.9 Hz, 2H), 1.75-1.85 (m, 5H), 1.87-1.98 (m, 4H), 2.01 (d, J=12.6 Hz, 3H), 2.10-2.27 (m, 6H), 2.29-2.43 (m, 6H), 2.52 (s, 1H), 2.58-2.70 (m, 2H), 2.90 (t, J=6.7 Hz, 3H), 3.17 (dt, J=3.4, 1.7 Hz, 2H), 3.52 (t, J=1.7 Hz, 1H), 3.63 (s, 1H), 3.76 (dt, J=13.2, 6.3 Hz, 2H), 4.03-4.12 (m, 8H), 4.45 (d, J=12.2 Hz, 3H), 4.66 (s, 1H), 6.93 (d, J=2.6 Hz, 1H), 6.97 (s, 1H), 7.24-7.32 (m, 2H), 7.40 (d, J=10.7 Hz, 1H), 7.44 (d, J=5.7 Hz, 1H), 7.70 (dd, J=9.1, 5.9 Hz, 1H), 8.26 (s, 1H). ¹⁹F NMR (376 MHz, CD₃OD) δ−129.08 (t, J=8.4 Hz, 1F), −122.47 (d, J=23.7 Hz, 1F), −121.25 (t, J=13.3 Hz, 1F). 19F NMR (376 MHz, CD₃OD) δ−129.05 (t, J=8.4 Hz, 1F), −122.35 (d, J=45.9 Hz, 1F), −121.27 (s, 1F). m/z, (ESI⁺): 1046.3.

### Synthesis of Compound 240

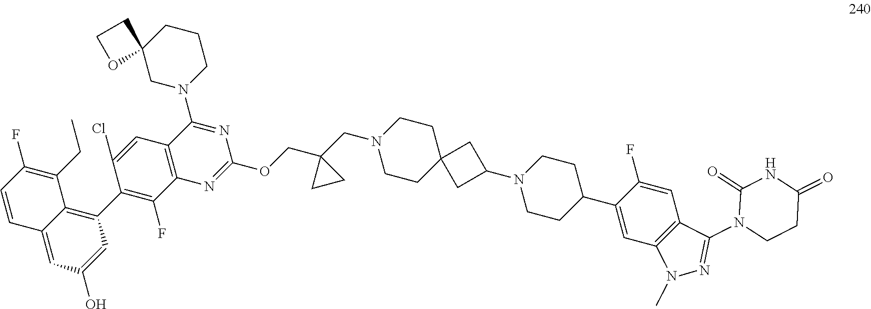

240 was synthesized according to the procedure of 239. ¹H NMR (400 MHz, CD₃OD) δ 0.60-0.69 (m, 2H), 0.82-0.84 (m, 2H), 1.49 (d, J=4.5 Hz, 2H), 1.71-1.84 (m, 7H), 1.91-2.00 (m, 5H), 2.11 (s, 5H), 2.35 (dd, J=21.1, 10.8 Hz, 3H), 2.55 (s, 2H), 2.65 (dt, J=15.8, 7.8 Hz, 3H), 2.90 (t, J=6.7 Hz, 4H), 3.02 (d, J=11.6 Hz, 2H), 3.11-3.20 (m, 3H), 3.63 (d, J=4.3 Hz, 2H), 4.07 (d, J=3.6 Hz, 6H), 4.29 (d, J=13.5 Hz, 1H), 4.42-4.48 (m, 2H), 4.64 (dd, J=14.6, 7.8 Hz, 3H), 6.96 (d, J=2.5 Hz, 1H), 7.25-7.34 (m, 2H), 7.39 (d, J=10.7 Hz, 1H), 7.45 (d, J=5.7 Hz, 1H), 7.71 (dd, J=9.0, 5.8 Hz, 1H), 8.30 (s, 1H). ¹⁹F NMR (376 MHz, CD₃OD) δ−129.08 (t, J=8.4 Hz, 1F), −122.47 (d, J=23.7 Hz, 1F), −121.25 (t, J=13.3 Hz, 1F). m/z, (ESI⁺): 1046.3.

### Synthesis of Compound 241

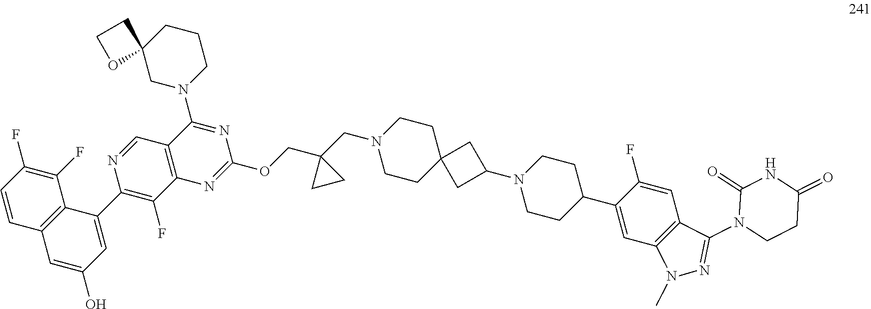 [SMILES: Cn1nc(N2CCC(=O)NC2=O)c2cc(F)c(C3CCN(C4CC5(CCN(CC6(COc7nc(N8CCC[C@]9(CCO9)C8)c8cnc(-c9cc(O)cc%10ccc(F)c(F)c9%10)c(F)c8n7)CC6)CC5)C4)CC3)cc21]

241 was synthesized according to the procedure of 202 with 2-(7,8-difluoro-3-(methoxymethoxy)naphthalen-1-yl)-4,4,5,5-tetramethyl-1,3,2-dioxaborolane as starting material. ¹H NMR (400 MHz, CD₃OD) δ 9.30 (d, J=14.3 Hz, 1H), 7.60 (dd, J=9.4, 4.9 Hz, 1H), 7.44-7.33 (m, 3H), 7.31 (d, J=2.4 Hz, 1H), 7.25 (d, J=2.3 Hz, 1H), 4.70 (dd, J=13.7, 6.7 Hz, 1H), 4.62-4.36 (m, 5H), 4.07-3.92 (m, 5H), 3.88-3.66 (m, 4H), 3.66-3.52 (m, 2H), 3.50-3.31 (m, 2H), 3.13-2.90 (m, 4H), 2.84 (t, J=6.7 Hz, 2H), 2.63-2.40 (m, 3H), 2.39-1.86 (m, 15H), 1.85-1.72 (m, 1H), 1.05-0.94 (m, 2H), 0.90-0.79 (m, 2H). ¹⁹F NMR (376 MHz, CD₃OD) δ−77.12 (s, 12F), −128.95(d, J=10.7 Hz, 1F), −141.22 (d, J=35.6 Hz, 1F), −145.30 (d, J=24.9 Hz, 1F), −146.70 (s, 1F). m/z, (ESI⁺): 1046.3.

### Synthesis of Compound 242

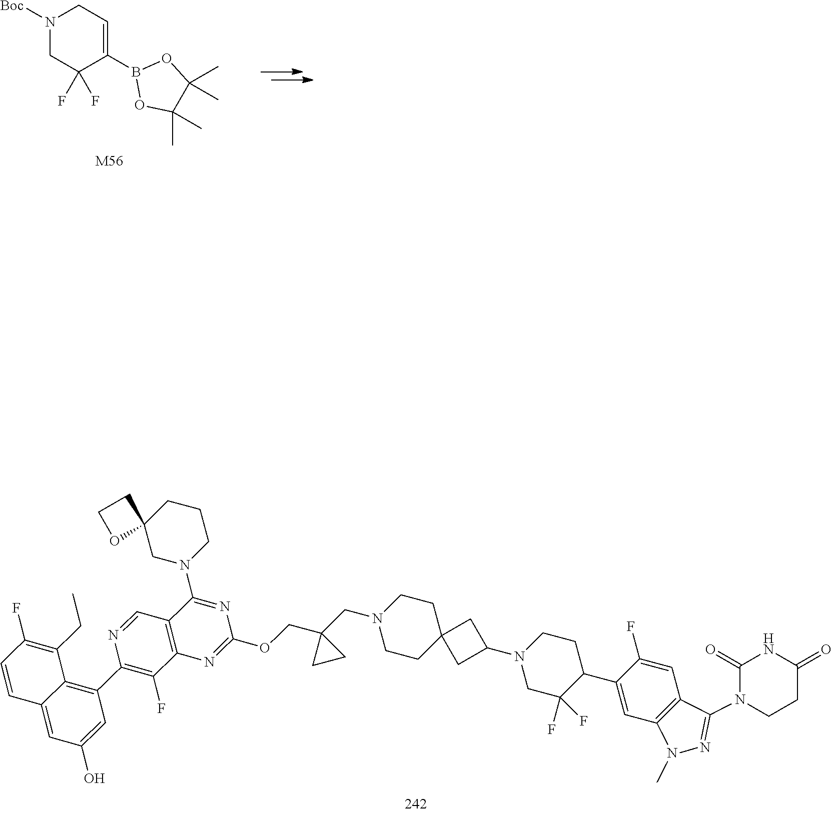 [SMILES: CC(C)(C)OC(=O)N1CC=C(B2OC(C)(C)C(C)(C)O2)C(F)(F)C1 ; "M56" ; CCc1c(F)ccc2cc(O)cc(-c3ncc4c(N5CCC[C@]6(CCO6)C5)nc(OCC5(CN6CCC7(CC6)CC(N6CCC(c8cc9c(cc8F)c(N8CCC(=O)NC8=O)nn9C)C(F)(F)C6)C7)CC5)nc4c3F)c12]

242 was synthesized according to the procedure of 202 with M56 as starting material. ¹H NMR (400 MHz, CD₃OD) 0.58 (s, 2H), 0.79 (s, 2H), 1.70 (ddd, J=27.2, 11.5, 5.2 Hz, 7H), 1.97 (dd, J=7.8, 5.0 Hz, 2H), 2.09 (d, J=11.1 Hz, 4H), 2.25 (d, J=12.1 Hz, 2H), 2.30-2.41 (m, 3H), 2.45-2.66 (m, 8H), 2.91 (t, J=6.7 Hz, 4H), 3.11 (d, J=11.5 Hz, 1H), 3.19-3.28 (m, 1H), 3.52 (s, 3H), 3.87 (dd, J=18.2, 13.6 Hz, 1H), 4.08 (d, J=3.1 Hz, 5H), 4.49 (q, J=11.3 Hz, 3H), 4.60 (dd, J=8.6, 6.3 Hz, 2H), 4.66 (d, J=13.3 Hz, 2H), 7.12 (d, J=2.6 Hz, 1H), 7.29 (t, J=9.3 Hz, 1H), 7.34 (d, J=2.7 Hz, 1H), 7.44 (d, J=10.2 Hz, 1H), 7.62 (d, J=5.4 Hz, 1H), 7.72 (dd, J=9.0, 5.8 Hz, 1H), 9.30 (d, J=9.3 Hz, 1H). ¹⁹F NMR (376 MHz, CD₃OD) −139.03 (d, J=40.1 Hz 1H), −127.34 (d, J=12.4 Hz 1H), −121.11 (t, J=7.9 Hz 1H), −114.34-−111.36 (m, 1H), −103.65 (d, J=240.9 Hz 1H). m/z (ESI⁺): 1049.44.

### Synthesis of Compound 243

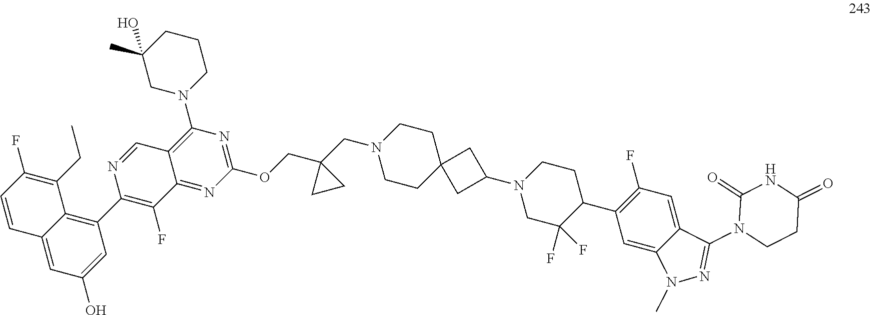 [SMILES: CCc1c(F)ccc2cc(O)cc(-c3ncc4c(N5CCC[C@@](C)(O)C5)nc(OCC5(CN6CCC7(CC6)CC(N6CCC(c8cc9c(cc8F)c(N8CCC(=O)NC8=O)nn9C)C(F)(F)C6)C7)CC5)nc4c3F)c12]

243 was synthesized according to the procedure of 242 with 85-1 as starting material. ¹H NMR (400 MHz, CD₃OD) δ 0.57 (s, 2H), 0.77 (s, 2H), 1.28-1.37 (m, 5H), 1.60-1.76 (m, 7H), 1.76-2.01 (m, 5H), 2.08 (q, J=10.1, 8.3 Hz, 3H), 2.17-2.27 (m, 2H), 2.31 (d, J=10.3 Hz, 2H), 2.41-2.74 (m, 7H), 2.90 (t, J=6.7 Hz, 3H), 3.10 (d, J=11.5 Hz, 1H), 3.44-3.53 (m, 1H), 3.22 (s, 1H), 3.57-3.73 (m, 2H), 4.07 (d, J=2.6 Hz, 5H), 4.27 (d, J=13.1 Hz, 1H), 4.46 (d, =5.4 Hz, 2H), 4.53 (s, 1H), 4.67 (s, 1H), 7.09 (d, J=2.6 Hz, 1H), 7.28 (t, J=9.4 Hz, 1H), 7.33 (d, J=2.7 Hz, 1H), 7.43 (d, J=10.2 Hz, 1H), 7.61 (d, J=5.4 Hz, 1H), 7.71 (dd, J=9.1, 5.8 Hz, 1H), 9.24 (d, J=3.2 Hz, 1H). ¹⁹F NMR (376 MHz, CD₃OD) δ−139.14 (d, J=16.2 Hz 1H), −127.29 (d, J=11.6 Hz 1H), −121.13 (q, J=10.8, 8.7 Hz 1H), −113.19-−112.04 (m, 1H), −104.26-−102.95 (m, 1H). m/z (ESI⁺):1037.55.

### Synthesis of Compound 244

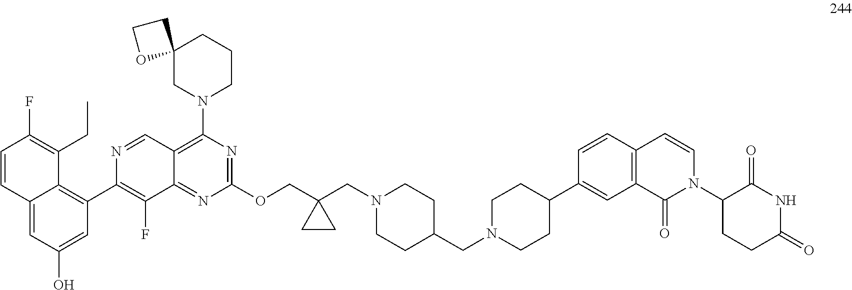 [SMILES: CCc1c(F)ccc2cc(O)cc(-c3ncc4c(N5CCC[C@]6(CCO6)C5)nc(OCC5(CN6CCC(CN7CCC(c8ccc9ccn(C%10CCC(=O)NC%10=O)c(=O)c9c8)CC7)CC6)CC5)nc4c3F)c12]

244 was synthesized according to the procedure of 234 with tert-butyl 4-formylpiperidine-1-carboxylate as starting material. ¹H NMR (400 MHz, CD₃OD) δ 0.66 (s, 2H), 0.86 (q, J=7.2 Hz, 5H), 1.70-2.04 (m, 11H), 2.15-2.30 (m, 5H), 2.36 (q, J=12.7, 10.6 Hz, 4H), 2.46-2.64 (m, 4H), 2.81 (dd, J=20.1, 7.1 Hz, 5H), 2.90 (dd, J=13.5, 4.5 Hz, 1H), 3.15 (dd, J=16.7, 6.3 Hz, 2H), 3.40-3.55 (m, 2H), 3.87 (dd, J=16.3, 13.6 Hz, 1H), 4.42-4.55 (m, 3H), 4.59 (dd, J=16.0, 7.6 Hz, 2H), 4.64 (d, J=6.7 Hz, 3H), 6.74 (d, J=7.5 Hz, 1H), 7.12 (d, J=2.7 Hz, 1H), 7.29 (t, J=9.3 Hz, 1H), 7.33-7.36 (m, 2H), 7.46-7.51 (m, 1H), 7.54 (s, 1H), 7.71 (dd, J=9.1, 5.8 Hz, 1H), 8.25 (d, J=8.4 Hz, 1H), 9.31 (d, J=7.8 Hz, 1H). ¹⁹F NMR (376 MHz, CD₃OD) δ−121.05 (t, J=8.0 Hz 1H), −138.99 (d, J=45.0 Hz 1H). m/z (ESI⁺): 981.57.

### Example 3. Protein Degradation Study

Aspc-1 (Cobioer, CBP60546) cells in exponential growth phase were inoculated on a 6-well cell culture plate (Corning, 3516) at density of 1E6/well; and cells in the plate were cultured in a 37° C. incubator containing 5%-carbon dioxide. The next day, test compounds were dissolved in DMSO (Sigma, RNBF5902) for the preparation of a 10-mM concentration stock solution. The stock solution was diluted with complete medium (RPMI-1640 supplemented with 10% fetal bovine serum, 1% penicillin-streptomycin), yielding working solutions of various concentrations. The plate, after the addition of compounds, was placed in a 5% carbon dioxide incubator, and incubated at 37° C. for 24 hours (final DMSO concentration was 0.1%). The plate was then removed from the incubator, and the cells were washed twice with precooled PBS (Gibco, 14190250), followed by addition of 80 μL RIPA (CST, 9806S) lysis buffer (containing protease inhibitor (Invitrogen™, AM2696)) to each well. The adherent cells were scraped off with a cell scraper, and the cell lysate was transferred to a 1.5 mL centrifuge tube followed with a 30-minute incubation on ice. The lysate was then clarified with centrifugation at 12000 rpm for 10 min at 4° C. The supernatant was transferred into a new 1.5 mL centrifuge tube, and protein concentration was measured with BCA protein detection kit (Thermo Fisher, 23225). G12D protein level was assessed by western blot. A mixture of cell lysate (40 μL) and loading buffer (10 μL, 5×SDS (Beyotime, P0015L)) was denatured in a water-bath at 95° C. for 10 min. The denatured protein sample, at 30 μg/well, was loaded into a corresponding well of a 4-20% Bis-Tris gel (Kingsley, M00656). Protein samples were initially electrophoresed at 80 V for 30 minutes, followed by 120 V for 40 minutes, until the bromophenol blue strip ran to an appropriate position. After electrophoresis, iBlot2 (Life Technologies, IB21001) was used to transfer the separated samples onto PVDF film. The PVDF membrane was washed with double-distilled water and then blocked in TBST (Thermo Scientific, 28360) buffer containing 5% non-fat milk, for 2 hours. Blocked membrane was rinsed with tris-buffered saline tween-2 (Thermo Scientific, 28360, TBST) three times, 10 minutes each. Membrane was then incubated with 1:1000 diluted RAS Gi2D primary antibody (CST, cat #14429S) overnight at 4° C. Membrane was rinsed in a way described previously followed by 1 hour incubation of secondary antibody at room temperature. After washing the film for three times with TBST, ECL color developing solution was applied, and image was captured with Biorad Chemi Doc gel imager. The band gray value was quantified (Image Lab), and protein degradation level was calculated according to the following formulas:

Level of RAS G12D protein expression=(RAS G12D_{-compound}/β-Actin)_{-compound}/(RAS G12D_{-DMSO}/β-Actin_{-DMSO})

Level of degradation (%)=(1−Rate of RAS G12D protein expression)×100

The degradation level of Ras protein in the presence of test compounds in Aspc-1 cells is summarized in Table 4. Symbols of −, +, ++, +++, ++++, +++++ indicate that the degradation level of Ras protein induced by test compounds was 10% or less, 10 to 30%, 30 to 60%, 60 to 70%, 70 to 75%, and greater than 75%, respectively. “NT” indicates not tested.

TABLE 4
Degradation of Ras protein by test compounds.

| Compound | Rate of protein degradation (%) | | | | |
|---|---|---|---|---|---|
| No. | 1 nM | 0.005 μM | 0.010 μM | 0.05 μM | 0.1 μM |
| 85 | NT | NT | +++ | NT | +++ |
| 86 | NT | NT | +++++ | NT | +++++ |
| 101 | NT | NT | ++++ | NT | ++++ |
| 130 | ++++ | ++++ | NT | ++ | NT |
| 138 | ++ | ++ | NT | ++ | NT |
| 197 | + | ++ | NT | +++ | NT |
| 198 | NT | ++ | NT | ++ | NT |
| 199 | NT | ++ | NT | ++ | NT |
| 200 | NT | ++++ | NT | +++++ | NT |
| 201 | NT | +++++ | NT | +++++ | NT |
| 202 | NT | +++++ | NT | NT | NT |
| 203 | NT | + | NT | NT | NT |
| 204 | NT | + | NT | NT | NT |
| 205 | NT | +++ | NT | +++++ | NT |
| 206 | NT | + | NT | + | NT |
| 207 | NT | ++ | NT | +++ | NT |
| 208 | NT | ++ | NT | ++ | NT |
| 209 | NT | − | NT | − | NT |
| 212 | NT | − | NT | + | NT |
| 215 | NT | + | NT | ++ | NT |
| 217 | NT | − | NT | ++ | NT |
| 218 | NT | + | NT | ++ | NT |
| 219 | NT | ++ | NT | +++ | NT |
| 220 | NT | ++ | NT | ++ | NT |
| 222 | NT | − | NT | − | NT |
| 224 | NT | + | NT | + | NT |
| 225 | NT | ++ | NT | ++ | NT |
| 226 | NT | + | NT | ++ | NT |
| 227 | NT | − | NT | + | NT |
| 229 | NT | + | NT | ++ | NT |
| 230 | NT | − | NT | − | NT |
| 231 | NT | + | NT | ++ | NT |
| 232 | NT | + | NT | ++ | NT |
| 233 | NT | + | NT | ++ | NT |
| 234 | NT | ++ | NT | ++++ | NT |
| 235 | NT | ++ | NT | +++++ | NT |
| 236 | NT | − | NT | + | NT |
| 237 | NT | + | NT | + | NT |
| 238 | NT | +++++ | NT | ++ | NT |
| 239 | NT | + | NT | − | NT |
| 240 | NT | + | NT | + | NT |
| 241 | NT | +++ | NT | ++ | NT |
| 242 | NT | ++ | NT | +++++ | NT |
| 243 | NT | ++ | NT | +++++ | NT |
| 244 | NT | ++ | NT | ++ | NT |
| 179a | NT | +++++ | NT | +++++ | NT |

Table 5 shows the half-maximum degradation concentrations (DC50s) and Dmax of various test compounds on G12D in Aspc-1 cells. Symbols of +++++, ++++, +++, ++, +, and − represent DC50s equal or less than 1 nM, 1-10 nM, 11-50 nM, 51-100 nM, 101-200 nM, and greater than 1000 nM, respectively. “NT” indicates not tested. Among them, Compound 179a had the lowest DC50 value, followed by compound 137.

TABLE 5
DC50s for test compounds.

| Compound No. | AsPc-1 DC50 (nM) | AsPc-1 Dmax |
|---|---|---|
| 85 | ++++ | NT |
| 86 | ++++ | 78% |
| 101 | ++++ | NT |
| 130 | +++++ | 73% |
| 137 | +++++ | 62% |
| 194 | − | NT |
| 196 | ++++ | NT |
| 201 | +++++ | 80% |
| 202 | ++++ | 81% |
| 205 | +++++ | 78% |
| 208 | ++ | 53% |
| 220 | ++++ | 55% |
| 234 | +++ | 65% |
| 235 | ++++ | 71% |
| 238 | +++ | 73% |
| 241 | +++ | 71% |
| 242 | ++++ | 76% |
| 243 | ++++ | 74% |
| 179a | +++++ | 70% |

### Example 4. Cell Proliferation Study

Aspc-1(Cobioer, CBP60546), GP2D(Cobioer, CBP60683), Mia paca2 (Cobioer, CBP60136), NCI-H358 (Cobioer, CBP60544), H727 (Cobioer, CBP60182), MKN-1 (Cobioer, CBP60486), and PSN1 (Cobioer, CBP61215) cells in exponential growth phase were seeded into a 96-well plate (Greiner, 655090) at density of 3E3/well. Cells were cultured overnight at 37° C. with 5% CO₂. On the next day, cells were treated with compound at various concentrations for 72 hours in 37° C., 5% CO₂ incubator (final DMSO concentration was 0.1%). After treatment, 100 μL Cell Tier Glo (Promega, G7573) were added to every well on the cell plate and incubated at room temperature (RT) for 10 minutes. Cell growth status was measured with Cell Tier Glo (Promega, G7573) following manufacturer's directions. Inhibition level was calculated according to the following formula:

$$\% inhibition = 100 - [ \frac{Test compound signal - Minimun Signal}{Maximum Signal - Minimum Signal} \times 100 ]$$

where the “Maximum Signal” are signal of wells containing DMSO without compound; the “Minimum Signal” are signal of wells recorded at Day 0 prior to commencement of the experiment; and the “Test compound signal” are signal of wells containing compound at established concentrations.

The inhibition curve was obtained with GraphPad 7.0 software using four-parameter equation. The results are shown in Table 6.

TABLE 6
Results of cell proliferation study.

| | G12C | | G12V | G12D | | G12R | WT |
|---|---|---|---|---|---|---|---|
| Compound | NCI-H358 | Mia paca2 | NCI-H727 | GP2D | AsPc-1 | PSN-1 | MKN1 |
| No. | IC50(nM) | IC50(nM) | IC50(nM) | IC50(nM) | IC50(nM) | IC50(nM) | IC50(nM) |
| 85 | NT | NT | 2.456 | 242.44 | 487.8 | 487.8 | 405.4 |
| 86 | 17.6 | 3.384 | 2.453 | 0.158 | 15.6 | 459.3 | 400.4 |
| 101 | 23.9 | 9.514 | 16.4 | 0.214 | 108.51 | 463.3 | 409 |
| 130 | 64.8 | NT | 4.453 | 0.219 | 12.37 | NT | NT |
| 137 | 438 | NT | 0.46 | NT | 65.6 | NT | NT |
| 200 | >1000 | NT | 45.79 | 0.991 | >1000 | NT | NT |
| 201 | 9.923 | NT | 2.091 | 0.256 | 4.981 | NT | NT |
| 202 | 17.08 | NT | 3.607 | 0.06 | 7.834 | NT | NT |
| 205 | 6.241 | NT | 0.309 | 0.289 | 2.32 | NT | NT |
| 207 | NT | NT | NT | 3.53 | 198 | NT | NT |
| 208 | 13.67 | NT | 1.855 | 0.719 | 101 | NT | NT |
| 234 | 17.9 | NT | 4.4 | NT | 43.2 | NT | NT |
| 235 | 10.3 | NT | 0.8 | NT | 63.5 | NT | NT |
| 179a | 126.5 | NT | 18.46 | 0.123 | 57.75 | NT | NT |

### Example 5. Pharmacokinetics

The pharmacokinetics of each compound were determined following intravenous (IV) infusions or oral administration (PO, oral administration) to 3 male ICR mice. Compound was formulated in 5% DMSO+5% Solutol+90% (20% SBE-β-CD in saline) for intravenous administration or oral administration. Blood samples were collected at various time points and placed in internal standard containing tubes. The samples were vortexed for 1 min and then centrifuged at 4° C., 12000 rpm for 5 min. Supernatant samples were analyzed by LC/MS/MS for each compound. The compound concentrations in plasma following IV or PO administration were fit using a non-compartmental model (Phoenix WinNonlin). The results are shown in Table 7.

TABLE 7
PK parameters

| Compound | | AUC₀₋ₗₐₛₜ | |
|---|---|---|---|
| No. | Dose regimen | (hr · ng/mL) | T_{1/2} (h) |
| 86 | IV (3 mg/kg) | 1331 | 14.3 |
| 86 | PO (10 mg/kg) | 103 | 6.52 |
| 137 | IV (3 mg/kg) | 1282 | 7.97 |
| 202 | IV (3 mg/kg) | 1503 | 30.4 |
| 202 | PO (10 mg/kg) | 104 | 16.9 |

### Example 6. Tumor Xenograft Studies

All studies were conducted in accordance with all applicable regulations and guidelines of the Institutional Animal Care and Use Committee (IACUC). Mice were maintained under pathogen-free conditions, and food and water were provided ad libitum. 6-8-week-old female, Balb/c nude mice (Nu/Nu) were injected subcutaneously with GP2D cells in 100 μL of PBS and Matrigel matrix in the right hind flank with 5×10⁶ cells 50:50 cells: Matrigel. Mouse health was monitored daily, and caliper measurements began when tumors were palpable. Tumor volume measurements were determined utilizing the formula 0.5×L×W² in which L refers to length and W refers to width of each tumor. When tumors reached an average tumor volume of ˜200 mm³, mice were randomized into treatment groups. The animals were divided into at least five mice per group. Mice were treated with test compound. Animals were monitored daily, and body weights and tumors were measured twice per week. The results are shown in Table 8. “TGI” means tumor growth inhibition value.

TABLE 8
Results of efficacy study in mice.

| Compound | Dose regimen | Days | TGI (%) |
|---|---|---|---|
| 86 | IP, 10 mg/kg, q3d | 21 | 75.8 |
| 202 | PO, 30 mg/kg, bid | 14 | 75.8 |

The contents of all documents and references cited herein are hereby incorporated by reference in their entirety.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

What is claimed is:

1. A bifunctional compound of Formula (A), or a pharmaceutically acceptable salt, ester, hydrate, solvate, or stereoisomer thereof:
K-L-T (A)
wherein:
K is a targeting group that binds specifically to a KRAS protein;
T is an E3-ligase binding group; and
L is absent or is a bivalent linking group that connects K and T together via a covalent linkage; wherein the targeting group K has the structure of Formula (I): 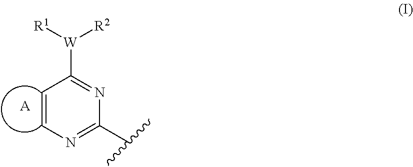
wherein:
A is a substituted or unsubstituted aromatic ring, heteroaromatic ring, carbocyclic ring, or carbon heterocyclic ring;
W is C, O or N, wherein, when W is O, R¹ is absent, and R² is independently H or alkyl; when W is C, R¹ and R² are independently H, hydroxyl, halogen, alkyl, alkoxy, or alkanoyl; and when W is N, R¹ and R² are independently H, substituted or unsubstituted alkyl, or alkanoyl, or,
R¹, R² and the W linked to them form a substituted or unsubstituted aryl, heteroaryl, cycloalkyl, heterocycloalkyl, or one of the following groups: 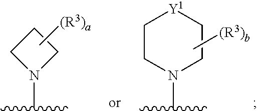
Y¹ is O, N, C, —CH₂CH₂—, —CH═CH—, —CH—, —OCH₂—, or absent;
R³ can be substituted at any substitutable position on the ring and/or the position where H is located on Y¹;
a is an integer from 0 to 6, specifically 0, 1, 2, 3, 4, 5, or 6;
b is an integer from 0 to 8, specifically 0, 1, 2, 3, 4, 5, 6, 7, or 8;
R³ is independently H, alkyl, hydroxy, halogen, amino, —CF₃, —NH(C₁-C₃ alkyl), —N(C₁-C₃ alkyl)₂, ═O, —CN, —O—(C₁-C₃ alkyl), —(C₁-C₃ alkyl)-OH, —C(═O)OH, —C(═O)(C₁-C₃ alkyl), —C(═O)O(C₁-C₃ alkyl), aryl, arylalkyl, cycloalkyl, or heterocycloalkyl; or,
any two R³s linked to the same atom and the ring to which they are linked form a spiro ring, wherein the spiro ring is optionally substituted by alkyl, hydroxy, halogen, amino, ═O, or —CN; or,
any two adjacent R³s and the ring to which they are linked form a fused ring, wherein the fused ring is optionally substituted by alkyl, hydroxy, halogen, amino, ═O, or —CN; or,
any two non-adjacent R³ groups together with the ring to which they are connected form a bridge ring.

2. (canceled)

3. The bifunctional compound of claim 1, wherein the targeting group K has the structure of Formula (I-a): 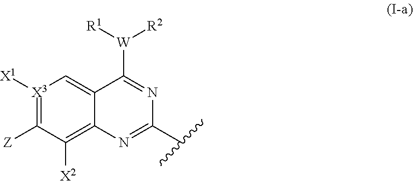
wherein:
X¹ and X² are independently H, F, Cl, CF₃, NH₂, or substituted or unsubstituted C₁-C₄ alkyl;
X³ is C or N; and
Z is substituted or unsubstituted cycloalkyl, substituted or unsubstituted aryl, or substituted or unsubstituted polycyclic aromatic hydrocarbon.

4. The bifunctional compound of claim 3, wherein Z has the structure of 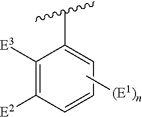
wherein:
E¹ is hydrogen, hydroxy, amino, halogen atom, C₁-C₃ alkyl or absent, and E¹ is optionally substituted at any substitutable position on the ring, optionally wherein the C₁-C₃ alkyl is methyl, ethyl, propyl, or isopropyl, optionally wherein the halogen is Cl or F;
n is an integer from 0 to 3;
E² and E³ are independently hydroxy, amino, halogen, or substituted or unsubstituted C₁-C₄ alkyl, or
E², E³ and a substituted or unsubstituted phenyl ring linked to them form a substituted or unsubstituted bicyclic, tricyclic, fused, spirocyclic, or bridged ring.

5.-6. (canceled)

7. The bifunctional compound of claim 4, wherein Z is one of the following structures:

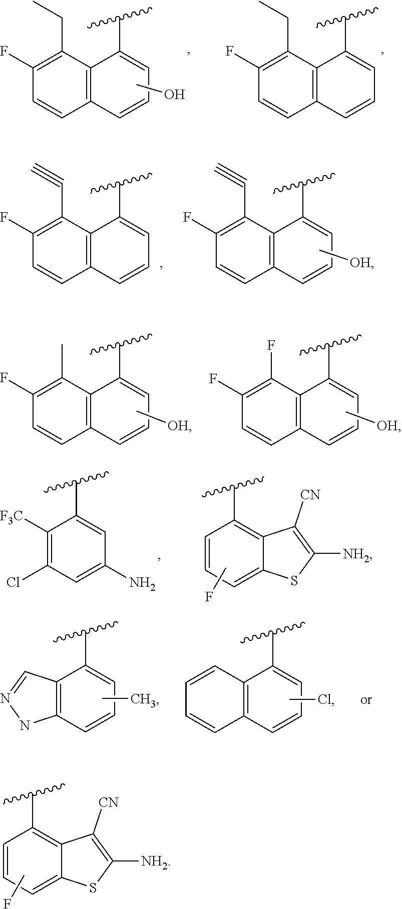

8. The bifunctional compound of claim 1, wherein the structure formed by R¹ and R² when bonded to W is:

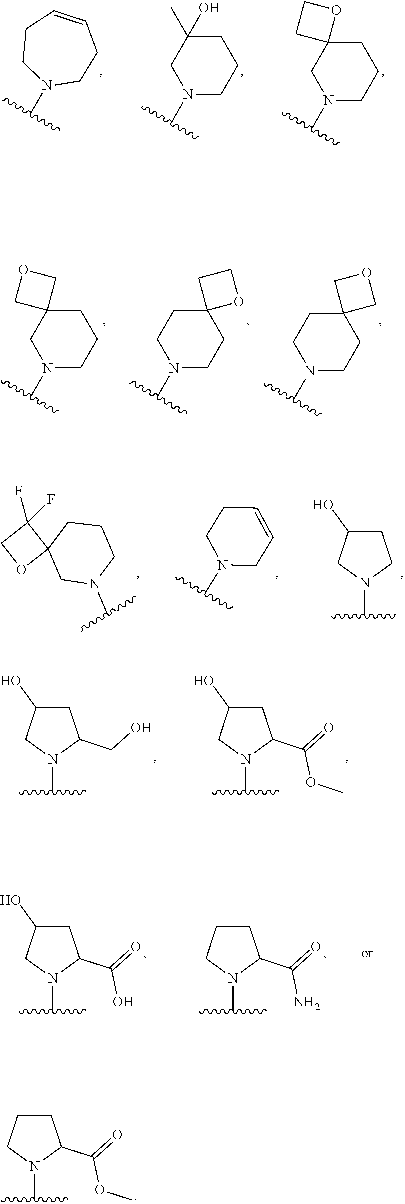

9. The bifunctional compound of claim 1, wherein the targeting group K has the structure of Formula (I-d): 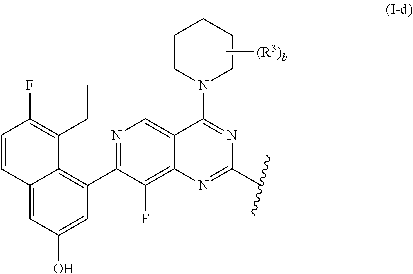
wherein:
any two R³s linked to the same atom and the ring to which they are linked form a spiro ring, wherein the spiro ring formed by the two R³s is an oxaalkyl ring, and the spiro ring is optionally substituted by alkyl, hydroxyl, halogen, amino, ═O, or —CN.

10. The bifunctional compound of claim 1, wherein the targeting group K has one of the following structures:

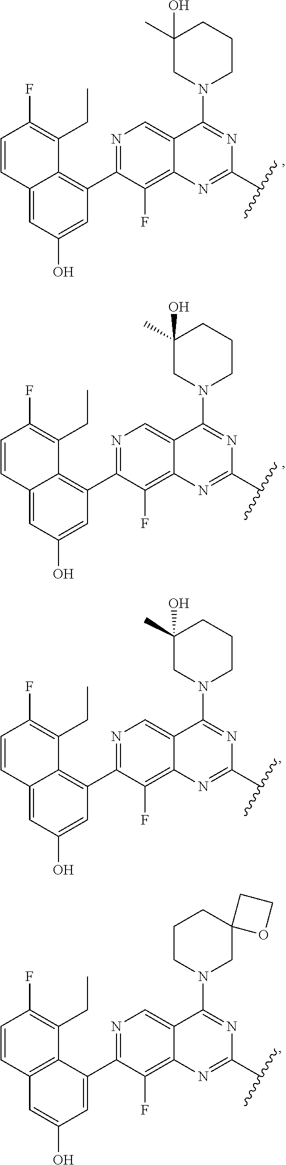 [SMILES: *c1nc(N2CCCC(C)(O)C2)c2cnc(-c3cc(O)cc4ccc(F)c(CC)c34)c(F)c2n1 ; *c1nc(N2CCC[C@](C)(O)C2)c2cnc(-c3cc(O)cc4ccc(F)c(CC)c34)c(F)c2n1 ; *c1nc(N2CCC[C@@](C)(O)C2)c2cnc(-c3cc(O)cc4ccc(F)c(CC)c34)c(F)c2n1 ; *c1nc(N2CCCC3(CCO3)C2)c2cnc(-c3cc(O)cc4ccc(F)c(CC)c34)c(F)c2n1]

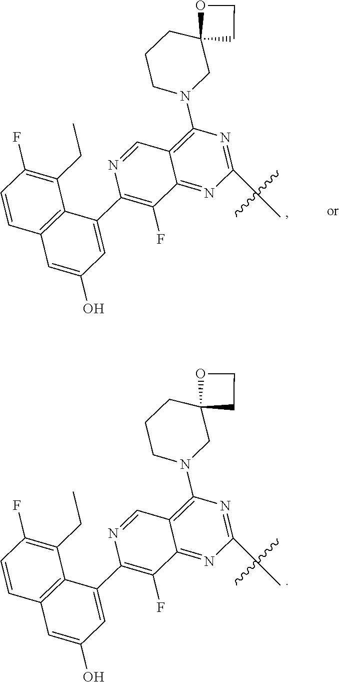 [SMILES: *c1nc(N2CCC[C@]3(CCO3)C2)c2cnc(-c3cc(O)cc4ccc(F)c(CC)c34)c(F)c2n1 ; *c1nc(N2CCC[C@@]3(CCO3)C2)c2cnc(-c3cc(O)cc4ccc(F)c(CC)c34)c(F)c2n1]

11. The bifunctional compound of claim 1, wherein the bivalent linking group L has the structure L¹-L²-L³, wherein L¹, L², and L³ are all independently present or absent, optionally wherein L¹, L², and L³ are independently selected from bivalent groups comprising substituted or unsubstituted hydrocarbyl, hydrocarbyloxy, oxyhydrocarbyl, cyclohydrorocarbyl, heterocyclohydrocarbyl, acylhydrocarbyl, hydrocarbylacyl, carbonylhydrocarbyl, hydrocarbylcarbonyl, amidohydrocarbyl, hydrocarbylamido, aryl, and oligopeptide group, each having a bivalent connecting site;
wherein the hydrocarbyl is saturated hydrocarbyl, unsaturated hydrocarbyl, aromatic hydrocarbyl, oxyhydrocarbyl, azahydrocarbyl, thiahydrocarbyl, phosphahydrocarbyl, or mixed heterohydrocarbyl with various heteroatoms, wherein the chain length of the hydrocarbyl or heterohydrocarbyl ranges from 1 to 20 atoms; and
the heterocyclic ring in the heterocyclohydrocarbyl is substituted or unsubstituted monocyclic, spirocyclic, or fused ring.

12.-13. (canceled)

14. (canceled)

15. The bifunctional compound of claim 11, wherein L¹ is —O—, —NH₂—, or one of the structures shown in (IIa), (IIb), (IIc), (IId), (IIe), (IIf), (IIg), (IIh), (IIi), (IIj), or (IIk): 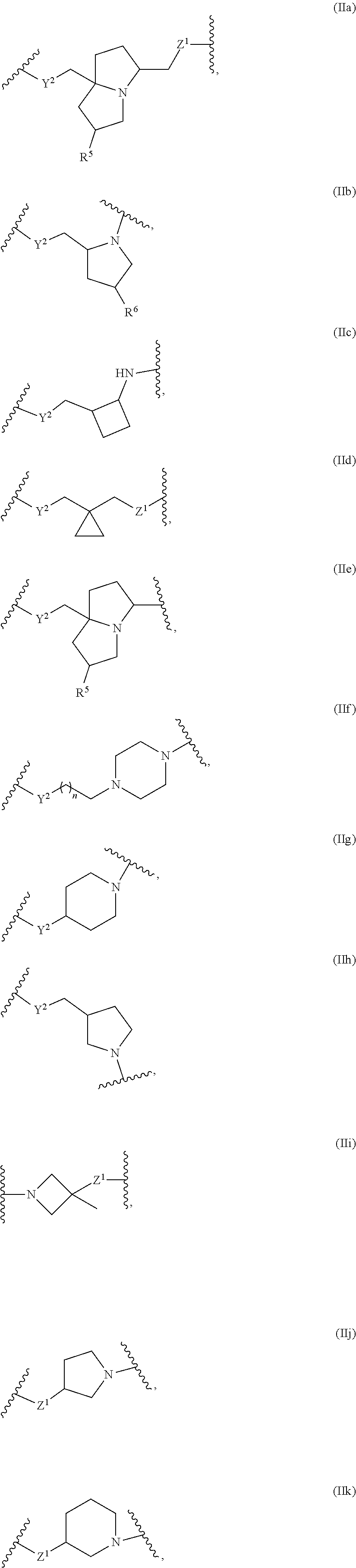
wherein:
Y² and Z¹ are independently oxygen (O), nitrogen (NH), or sulfur (S);
n is an integer from 0-20; and
R⁵ and R⁶ are independently hydrogen, halogen, hydroxyl, alkoxy, amino, or amine.

16. The bifunctional compound of claim 11, wherein L¹ is: 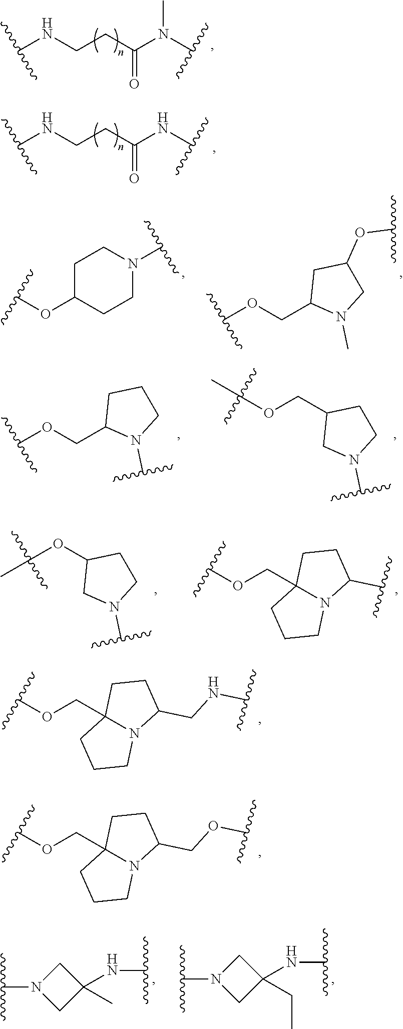 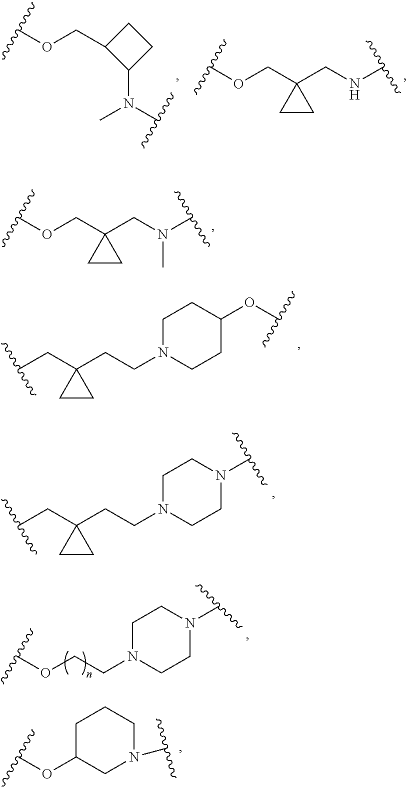 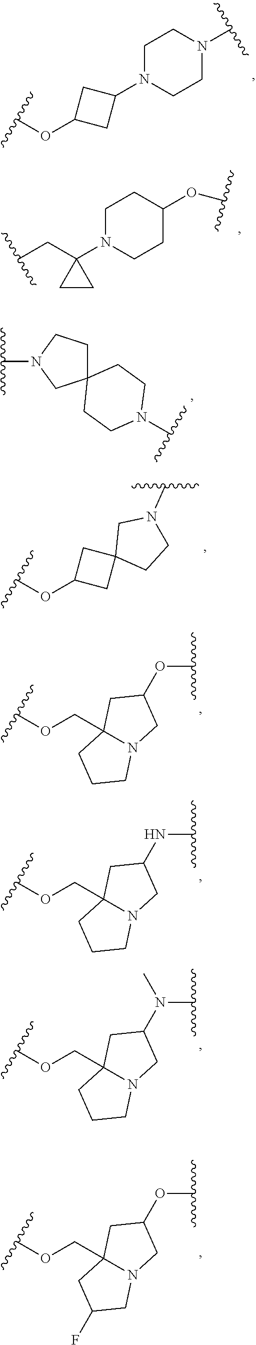 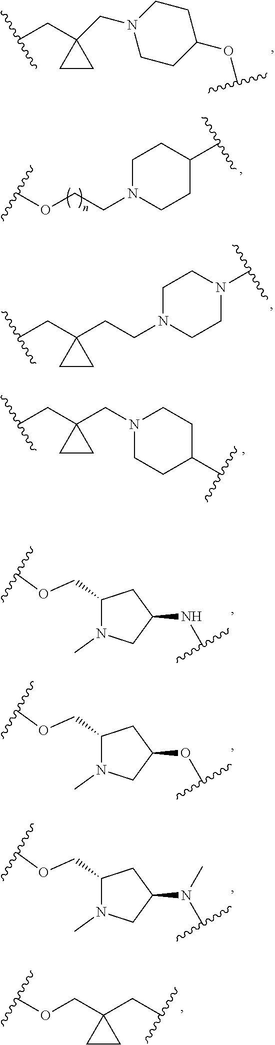
or absent;
wherein n is an integer from 0-20, n is an integer from 0-5, n is an integer from 1-2, n is 1, or n is 2.

17. The bifunctional compound of claim 11, wherein L² and L³ are independently: 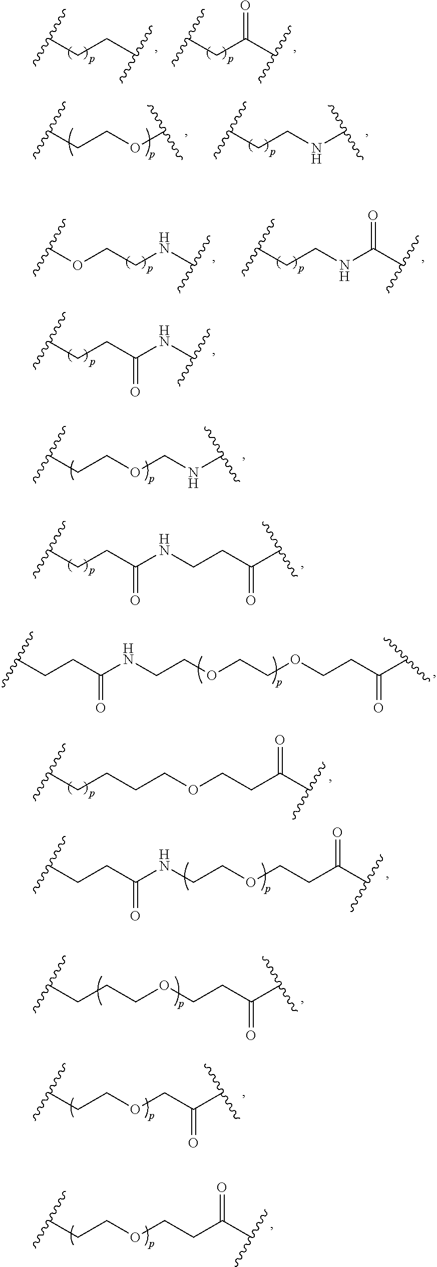 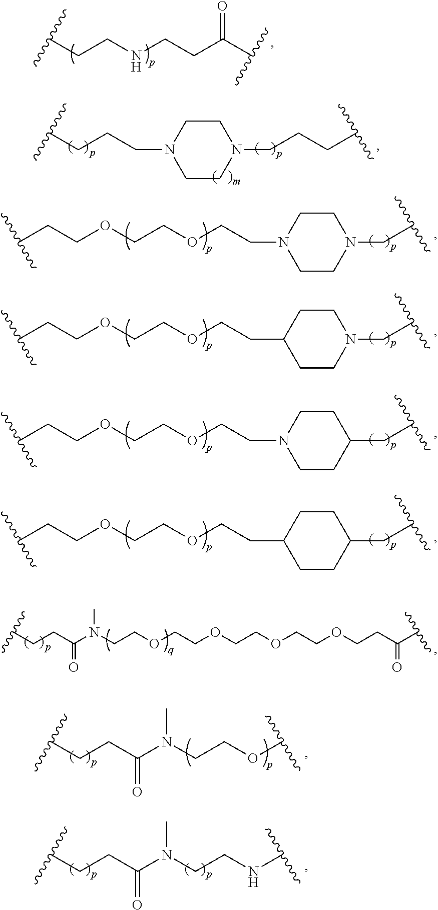 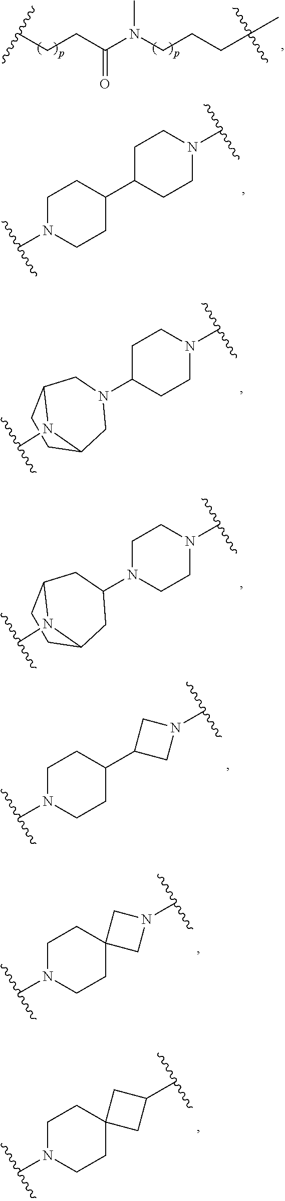 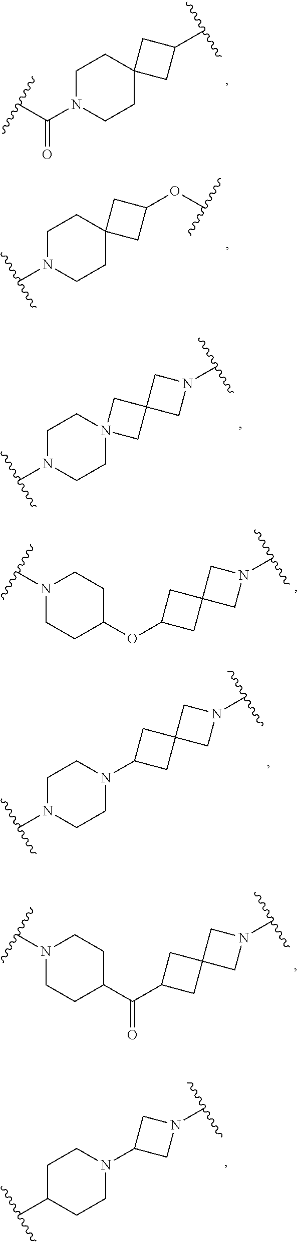 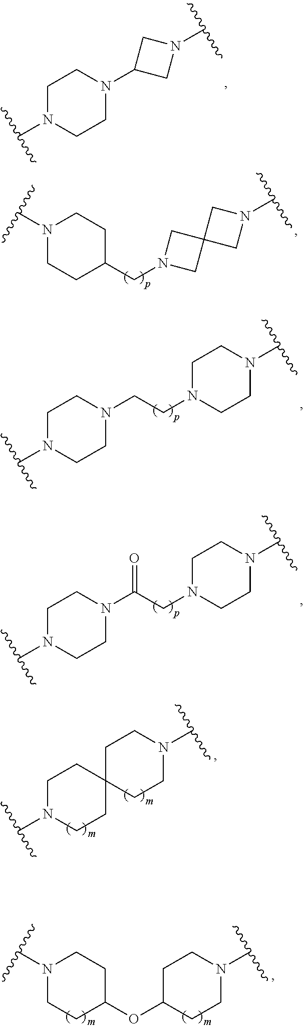 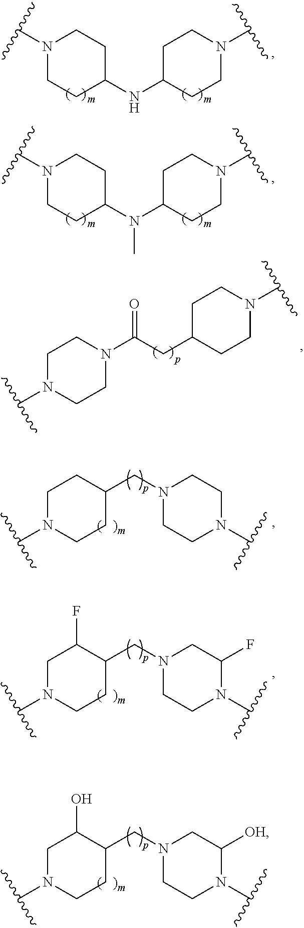 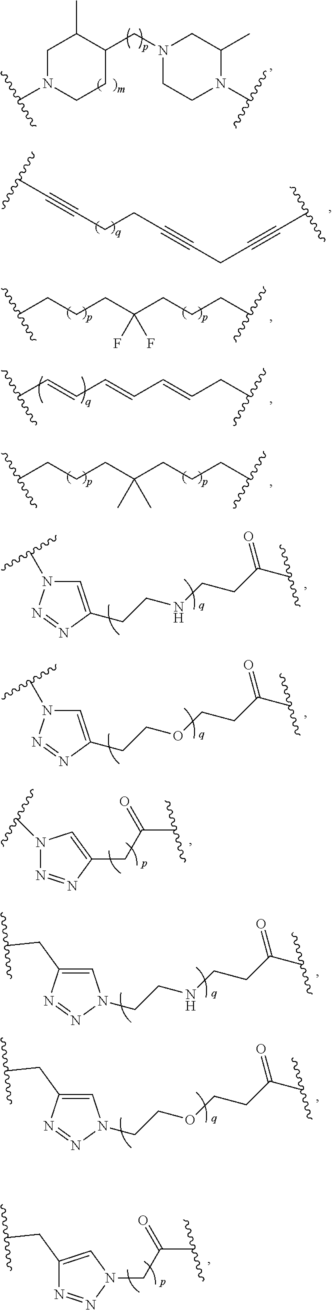 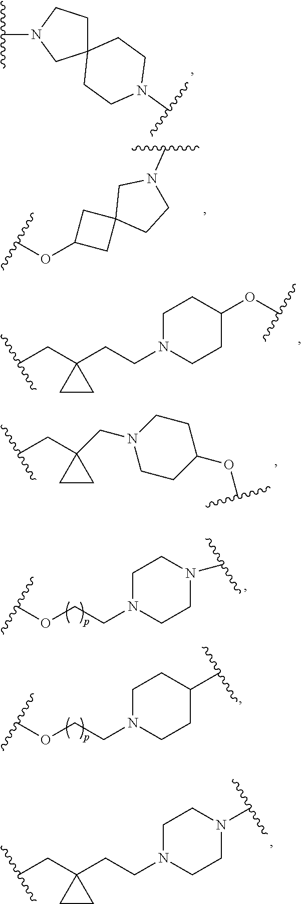 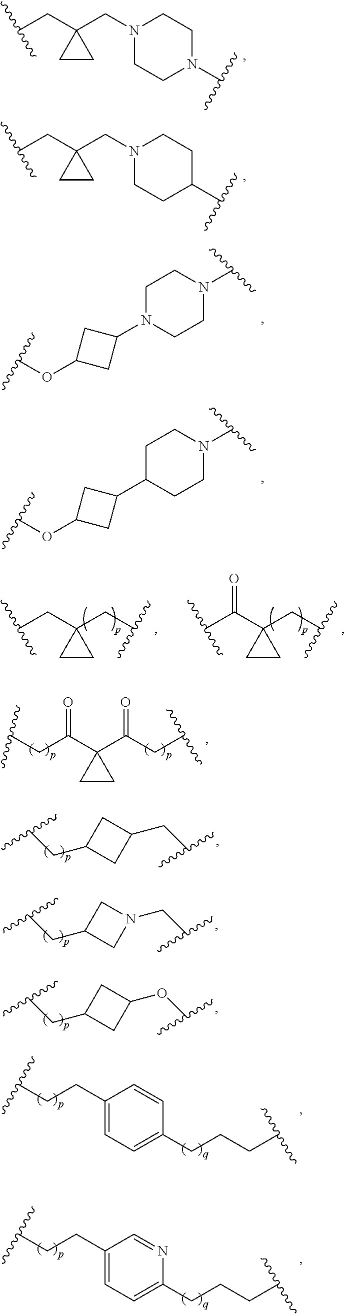 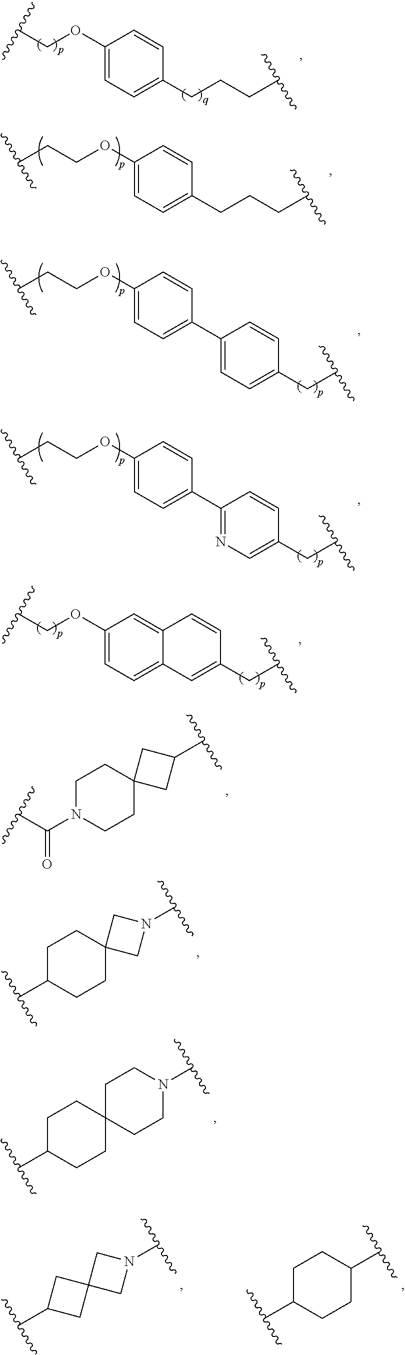
or absent;
wherein:
p is an integer from 0-20, or p is an integer from 0-10;
in is an integer from 0-8; and
q is an integer from 0-10, or q is an integer from 0-5.

18.-19. (canceled)

20. The bifunctional compound of claim 1, wherein the E3-ligase binding group T binds to a ligand which is Von Hippel-Lindau (VHL), Cereblon (CRBN), MDM2, cIAP, AhR, Nimbolide, CCW16, KB02 or KEAP1.

21. The bifunctional compound of claim 1, wherein the E3-ligase binding group T is: 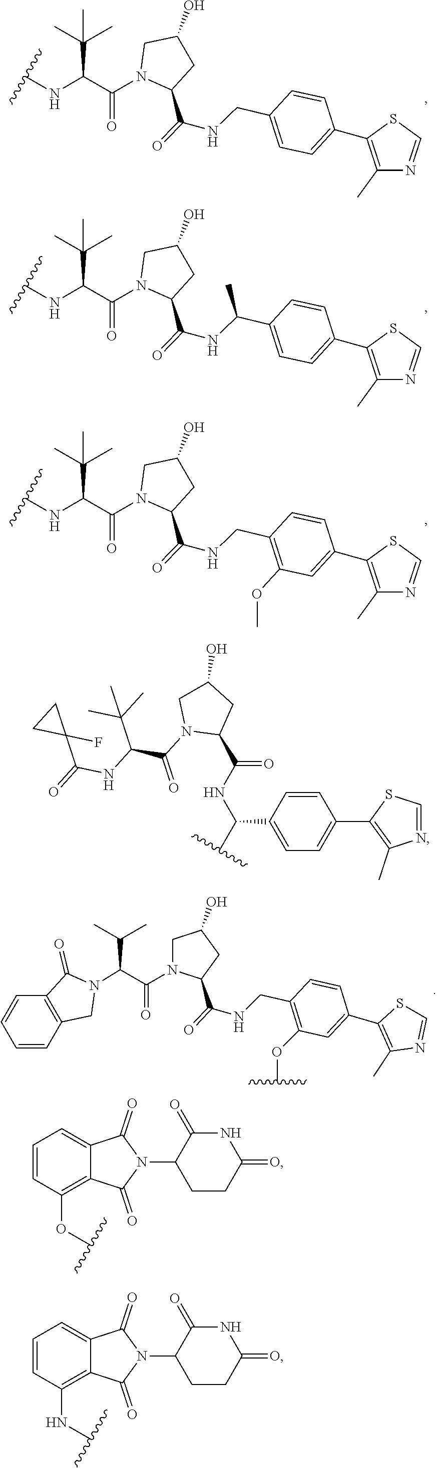 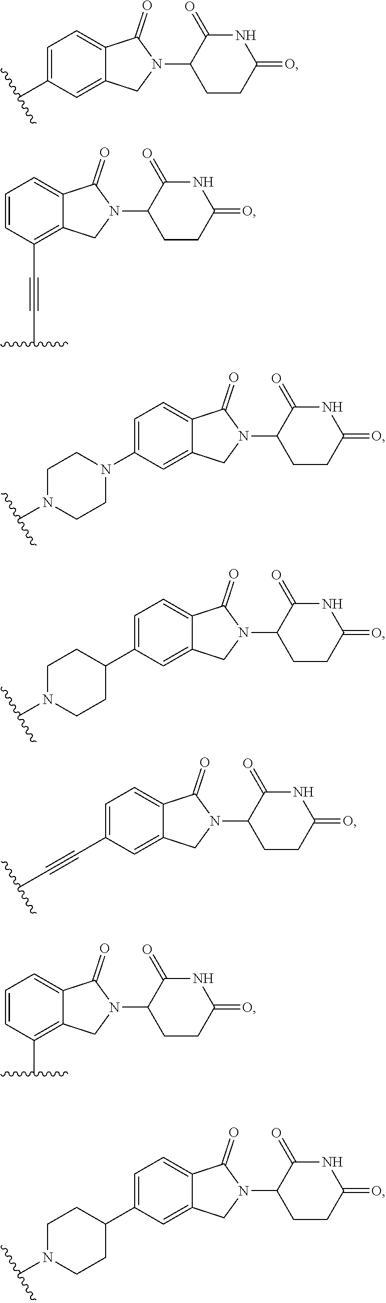 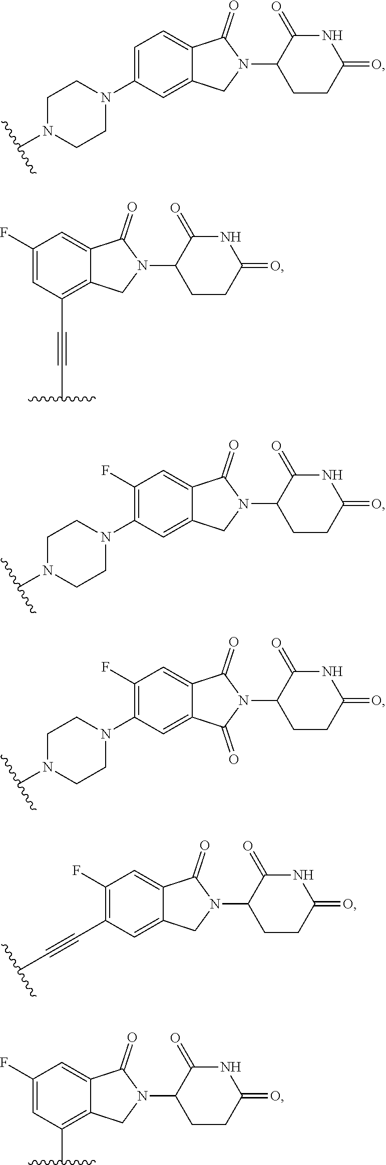 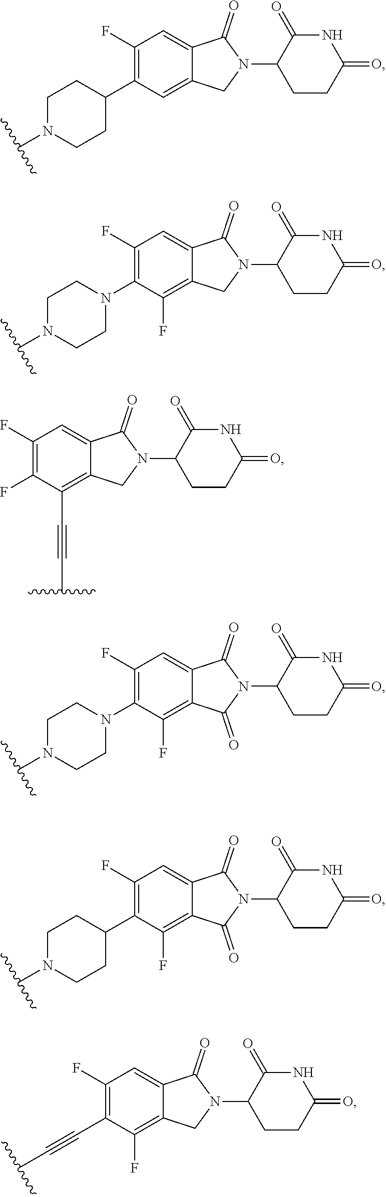 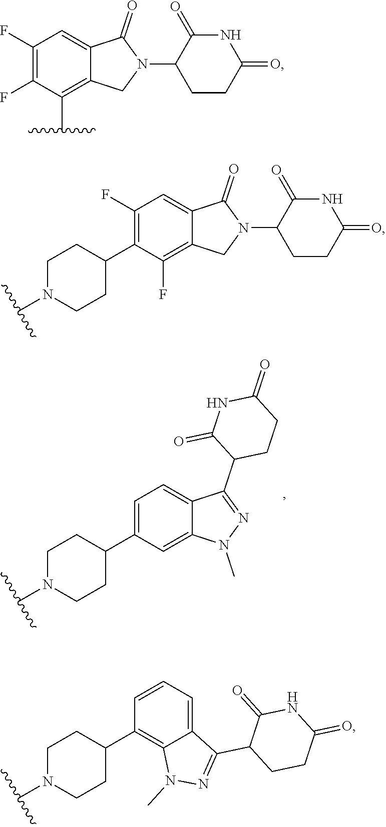 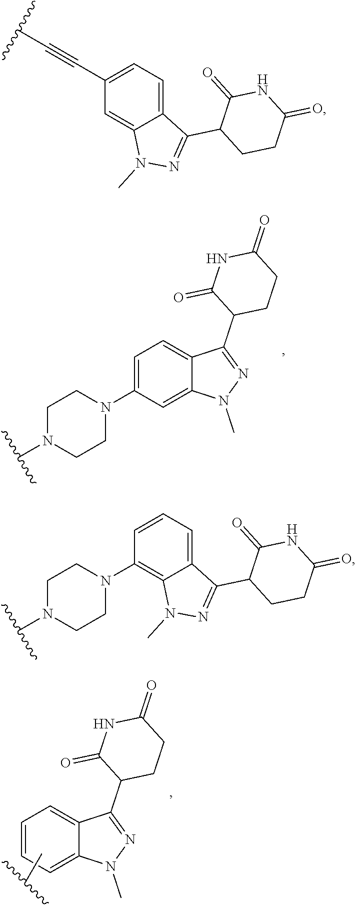 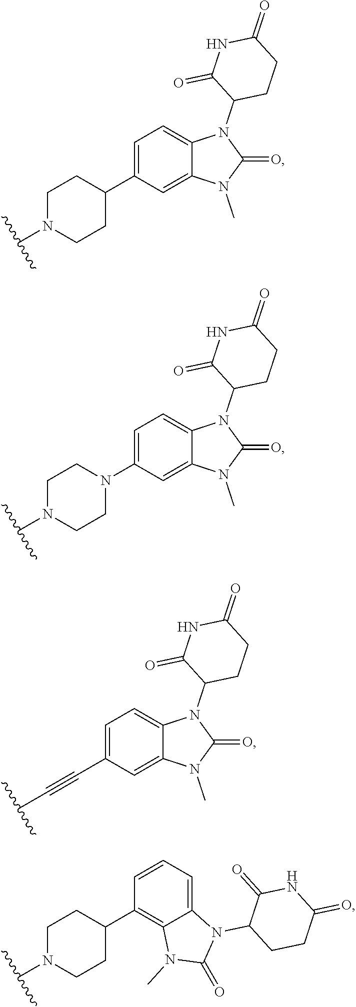 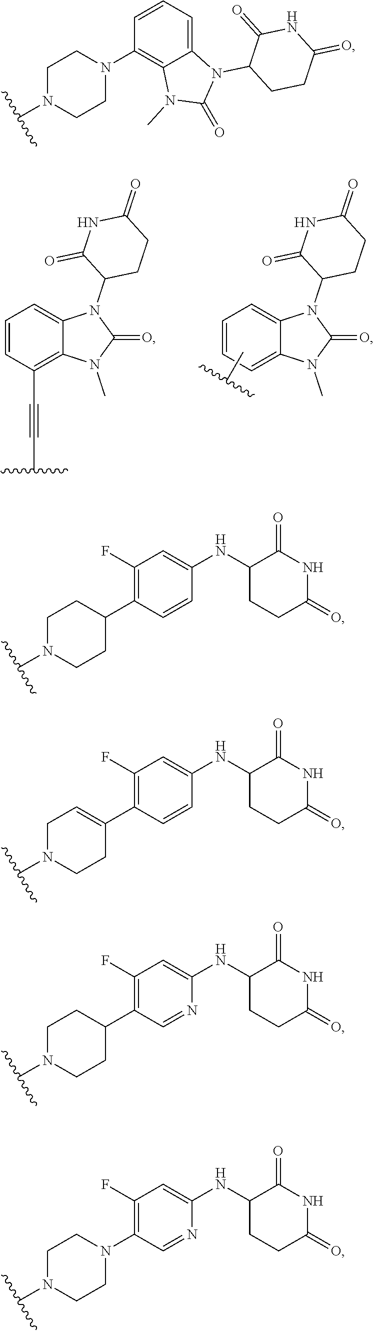 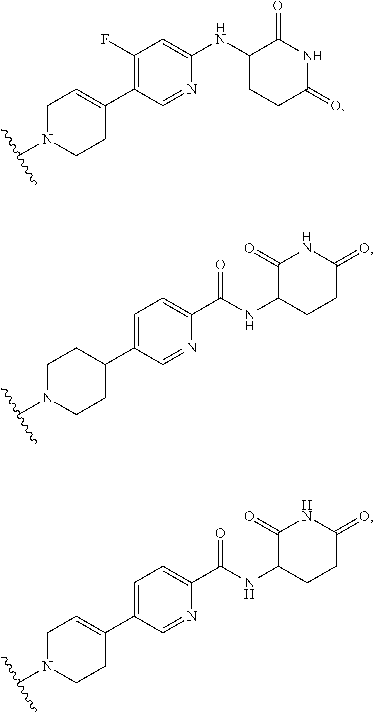 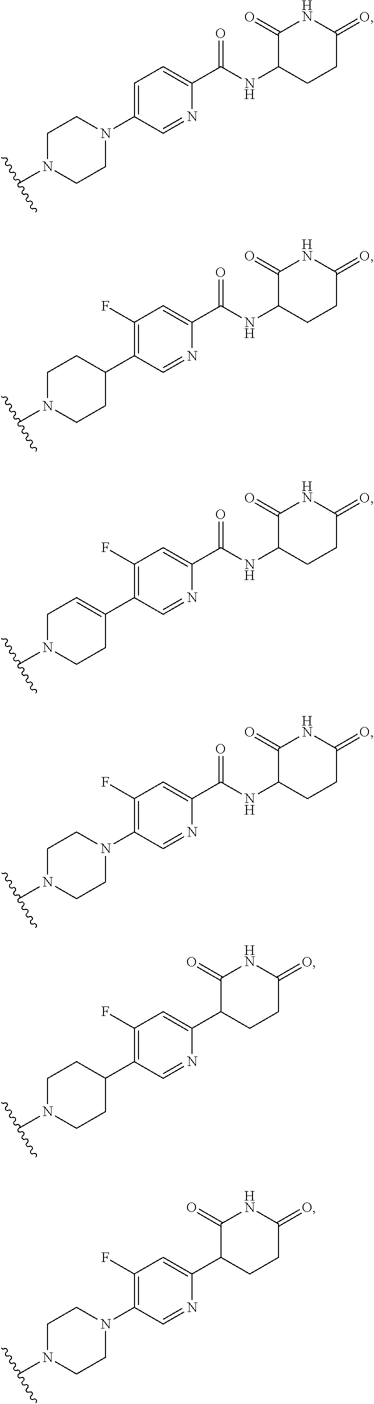 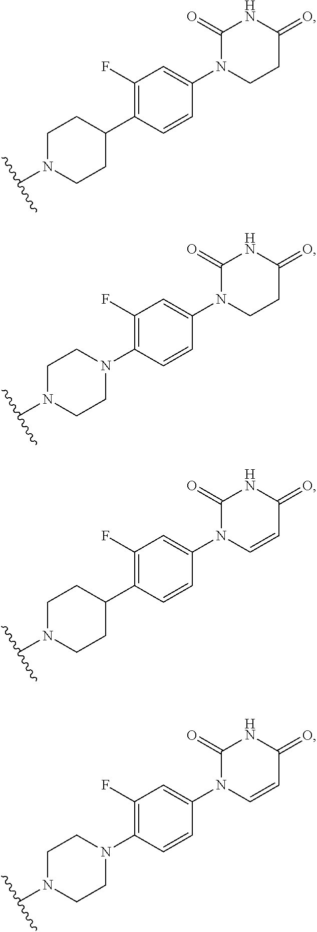 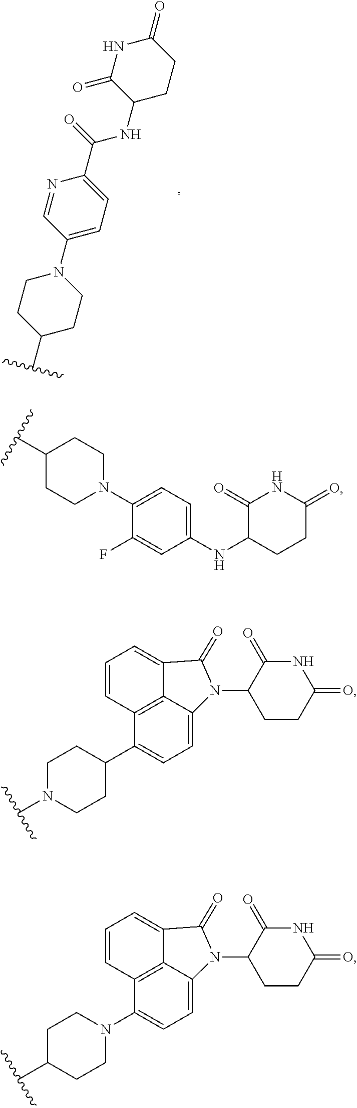 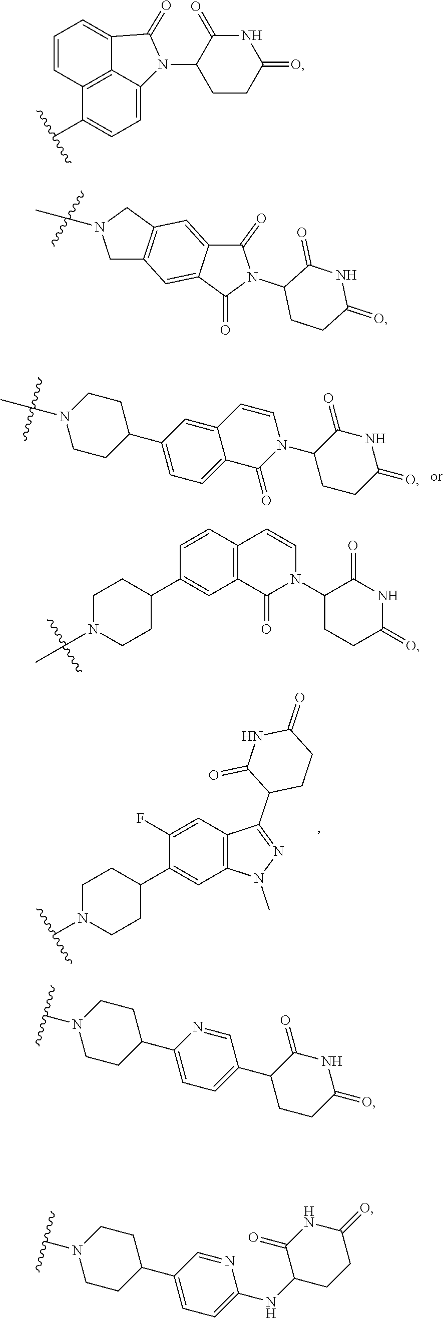 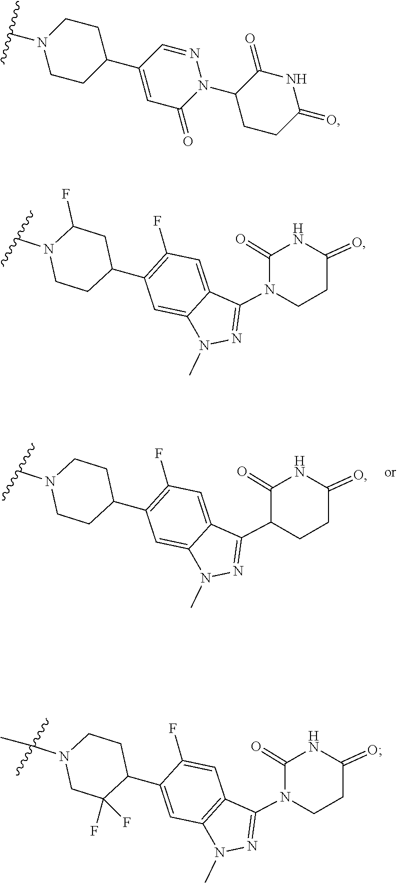
wherein, when the structure contains one or more chiral center, the one or more chiral center is independently R-configuration, S-configuration, or a mixture of R and S configurations.

22. The bifunctional compound of claim 1, wherein the compound has the structure of Formula (A-2), or a pharmaceutically acceptable salt, ester, stereoisomer, hydrate or solvate thereof: 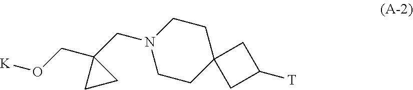
wherein:
K is 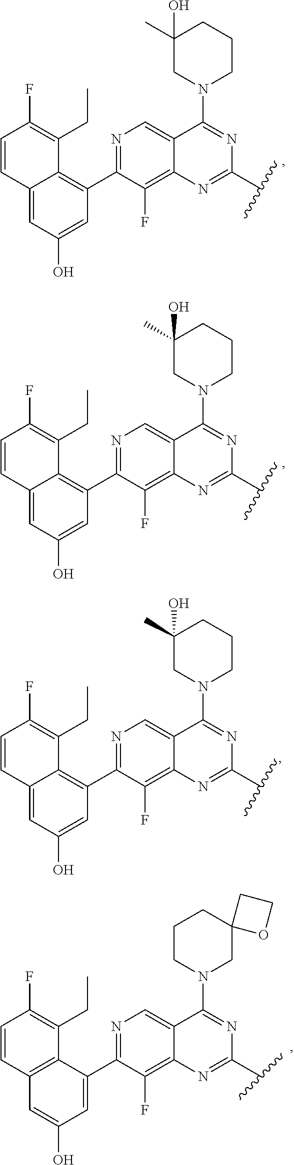 [SMILES: *c1nc(N2CCCC(C)(O)C2)c2cnc(-c3cc(O)cc4ccc(F)c(CC)c34)c(F)c2n1 ; *c1nc(N2CCC[C@](C)(O)C2)c2cnc(-c3cc(O)cc4ccc(F)c(CC)c34)c(F)c2n1 ; *c1nc(N2CCC[C@@](C)(O)C2)c2cnc(-c3cc(O)cc4ccc(F)c(CC)c34)c(F)c2n1 ; *c1nc(N2CCCC3(CCO3)C2)c2cnc(-c3cc(O)cc4ccc(F)c(CC)c34)c(F)c2n1] 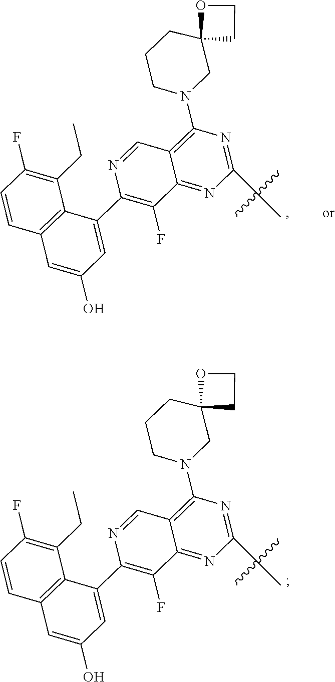 [SMILES: *c1nc(N2CCC[C@]3(CCO3)C2)c2cnc(-c3cc(O)cc4ccc(F)c(CC)c34)c(F)c2n1 ; *c1nc(N2CCC[C@@]3(CCO3)C2)c2cnc(-c3cc(O)cc4ccc(F)c(CC)c34)c(F)c2n1]
and

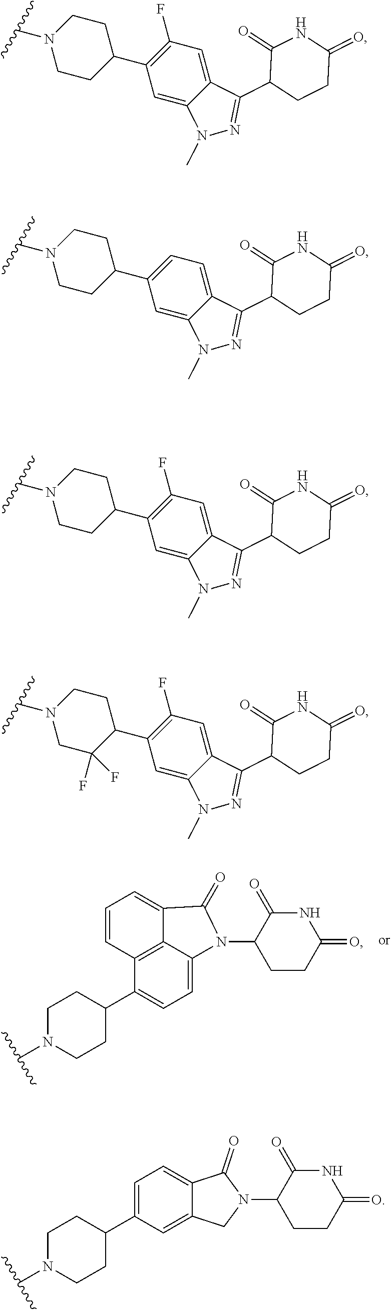 [SMILES: *N1CCC(c2cc3c(cc2F)c(C2CCC(=O)NC2=O)nn3C)CC1 ; *N1CCC(c2ccc3c(C4CCC(=O)NC4=O)nn(C)c3c2)CC1 ; *N1CCC(c2cc3c(cc2F)c(C2CCC(=O)NC2=O)nn3C)CC1 ; *N1CCC(c2cc3c(cc2F)c(C2CCC(=O)NC2=O)nn3C)C(F)(F)C1 ; *N1CCC(c2ccc3c4c(cccc24)C(=O)N3C2CCC(=O)NC2=O)CC1 ; *N1CCC(c2ccc3c(c2)CN(C2CCC(=O)NC2=O)C3=O)CC1]

23. The bifunctional compound of claim 1, wherein the compound is a compound shown in Table 2 or Table 3, or a pharmaceutically acceptable salt, ester, stereoisomer, hydrate or solvate thereof.

24. A pharmaceutical composition comprising the compound or the pharmaceutically acceptable salt, ester, hydrate, solvate, or stereoisomer thereof of claim 1, and a pharmaceutically acceptable excipient, carrier or diluent.

25.-32. (canceled)

33. The pharmaceutical composition of claim 24, further comprising at least one additional therapeutic agent, wherein the at least one additional therapeutic agent is optionally an anti-cancer agent, optionally a chemotherapeutic agent or an immune checkpoint inhibitor.

34.-35. (canceled)

36. A method for treating or preventing a KRAS-associated disease, disorder or condition in a subject in need thereof, comprising administering a therapeutically effective amount of the bifunctional compound of claim 1 to the subject, such that the KRAS-associated disease, disorder or condition is treated or prevented in the subject.

37. The method of claim 36, wherein the KRAS-associated disease, disorder or condition is:
a wild-type KRAS-associated disease, disorder or condition; a KRAS G12A-associated disease, disorder or condition; a KRAS G12C-associated disease, disorder or condition; a KRAS G12D-associated disease, disorder or condition; a KRAS G12R-associated disease, disorder or condition; a KRAS G12S-associated disease, disorder or condition; a KRAS G12V-associated disease, disorder or condition; a KRAS G13D-associated disease, disorder or condition; a KRAS Q61H-associated disease, disorder or condition; or a combination thereof; and/or
a hyperplastic or a hyperproliferative disorder.

38. (canceled)

39. The method of claim 37, wherein the hyperplastic or hyperproliferative disorder is a malignant tumor or cancer, wherein the tumor or cancer is a cardiac, lung, gastrointestinal, urogenital or genitourinary tract, liver, bone, nervous system, gynecological, hematologic, skin or dermatologic, biliary tract, or adrenal gland cancer or tumor.

40.-62. (canceled)

63. A method for treating or preventing a cancer, tumor, or hyperplastic disorder in a subject in need thereof, comprising administering a therapeutically effective amount of the compound or the pharmaceutically acceptable salt, ester, hydrate, solvate, or stereoisomer thereof of claim 1 to the subject, such that the cancer, tumor, or hyperplastic disorder is treated or prevented in the subject, optionally wherein the cancer, tumor, or hyperplastic disorder is associated with wild-type KRAS or with a KRAS mutation which is G12D, G12A, G12C, G12R, G12S, G12V, G13D and/or Q61H.

64.-88. (canceled)
